(19) United States
(12) Patent Application Publication
(10) Pub. No.: US 2020/0208125 A1
(43) Pub. Date: Jul. 2, 2020

(54) Productivity and Bioproduct Formation in Phototropin Knock/Out Mutants in Microalgae

(71) Applicants: NMC, INC., Los Alamos, NM (US); LOS ALAMOS NATIONAL SECURITY, LLC, Los Alamos, NM (US)

(72) Inventors: Sangeeta Negi, Los Alamos, NM (US); Richard Thomas Sayre, Los Alamos, NM (US); Shawn Robert Starkenburg, Los Alamos, NM (US)

(21) Appl. No.: 16/820,062

(22) Filed: Mar. 16, 2020

Related U.S. Application Data
(63) Continuation-in-part of application No. 15/831,178, filed on Dec. 4, 2017, now Pat. No. 10,590,398.
Application No. 15/831,178 is a continuation of application No. PCT/IB2016/054466, filed on Jul. 26, 2016.
(60) Provisional application No. 62/171,176, filed on Jun. 4, 2015.

(51) Int. Cl. C12N 9/12; C12N 1/12; C07K 14/405

(52) U.S. Cl. CPC C12N 9/12; C12N 2800/80; C07K 14/405; C12N 1/12

(57) ABSTRACT

Phototropin is a blue light receptor, which mediates a variety of blue-light elicited physiological processes in plants and algae. In higher plants these processes include phototropism, chloroplast movement and stomatal opening. In the green alga Chlamydomonas reinhardtii, phototropin plays a vital role in progression of the sexual life cycle and in the control of the eye spot size and light sensitivity Phototropin is also involved in blue-light mediated changes in the synthesis of chlorophylls, carotenoids, chlorophyll binding proteins. We compared the transcriptome of phototropin knock out (PHOT KO) mutant and wild-type parent to analyze differences in gene expression in high light grown cultures (500 μmol photons m⁻²s⁻¹). Our results indicate the up-regulation of genes involved in photosynthetic electron transport chain, carbon fixation pathway, starch, lipid, and cell cycle control genes. With respect to photosynthetic electron transport genes, genes encoding proteins of the cytochrome b6f and ATP synthase complex were up regulated potentially facilitating proton-coupled electron transfer. In addition genes involved in limiting steps in the Calvin cycle Ribulose-1,5-bisphosphate carboxylase/oxygenase (RuBisCO), Sidoheptulose 1,7 bisphosphatase (SBPase), Glyceraldehyde-3-phosphate dehydrogenase (3PGDH) and that mediate cell-cycle control (CDK) were also up regulated along with starch synthase and fatty acid biosynthesis genes involved in starch and lipid synthesis. In addition, transmission electron micrographs show increased accumulation of starch granules in PHOT mutant compared to wild type, which is consistent with the higher expression of starch synthase genes. Collectively, the altered patterns of gene expression in the PHOT mutants were associated with a two-fold increase in growth and biomass accumulation compared to wild type when grown in environmental photobioreactors (Phenometrics) that simulate a pond environment. In conclusion, our studies suggest that phototropin may be a master gene regulator that suppresses rapid cell growth and promotes gametogenesis and sexual recombination in wild type strains.

### CROSS-REFERENCE TO RELATED APPLICATIONS

This application is a continuation-in-part of U.S. patent application Ser. No. 15/831,178, entitled “Productivity and Bioproduct Formation in Phototropin Knock/Out Mutants in Microalgae”, filed Dec. 4, 2017, which is a continuation of International Patent Application No. PCT/162016/054466, entitled “Improved Productivity and Bioproduct Formation in Phototropin Knock/Out Mutants in Microalgae”, filed on Jul. 26, 2016, which claims priority to and benefit of U.S. Provisional Patent Application No. 62/171,176 entitled “Improved Productivity and Bioproduct Formation in Phototropin Knock/out Mutants in Microalgae” filed on Jun. 4, 2015, and the specification and claims thereof are incorporated herein by reference.

### STATEMENT REGARDING FEDERALLY SPONSORED RESEARCH

This invention was made with government support under grants Nos. Prime Contract No. DE-AC52-06NA25396 and NMC subcontract No. 277529. The U.S. government has certain rights in the invention.

### SEQUENCE LISTING

The instant application contains a Sequence Listing which has been submitted electronically in ASCII format and is hereby incorporated by reference in its entirety. Said ASCII copy, created on Mar. 2, 2018, is named PHOT_US_Sequences_031620_ST25.txt and is 314 Kbytes in size.

### TECHNICAL FIELD

Disclosed embodiments of the present invention are in the field of improved performance of microalgae in the production of biological products such as but not limited to biofuels, biomass, pigments, starch, oils and the like through selection, mutagenesis or engineering to reduce expression or knockout the phototropin gene for example.

### BACKGROUND

Phototropin is a blue light receptor, which mediates a variety of blue-light elicited physiological processes in plants and algae. In higher plants these processes include phototropism, chloroplast movement and stomatal opening. In the unicellular green alga Chlamydomonas reinhardtii, phototropin (PHOT) plays a vital role in the progression of the sexual life cycle and in the control of the eye spot size and light sensitivity. Phototropin is also involved in blue-light mediated changes in the synthesis of chlorophylls, carotenoids, and chlorophyll binding proteins. The UV-A/blue light sensing phototropins mediate a variety of light responses and are responsible in higher plants for optimization of photosynthetic yields (Chen, Chory et al. 2004).

Phototropins are commonly composed of two domains, an amine terminal photosensory domain and a carboxy terminal serine/threonine protein kinase domain. The photosensory domain is a flavin mononucleotide binding domain, the LOV domain. Plants and green algae contain two of these domains in the phototropin regulatory sequence, LOV1 and LOV2 (Chen, Chory et al. 2004). LOV domain is a member of PAS domains and are about 110 amino acids. There is a conserved sequence within the LOV domain identified at amino acid position 238-245 of SEQ ID NO: 1 for example (Gly Arg Asn Cys Arg Phe Leu Gln Gly). (Salomon et al. 2000). A diagram of the phototropin protein is:

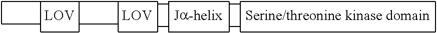

Phototropin knock-out mutants (PHOT K/O) have been made previously in plants (Suetsugu and Wada 2007, Moni, Lee et al. 2015) and algae (Zorin, Lu et al. 2009; Trippens, Greiner et al. 2012). However, all the PHOT K/O mutant prior art that has been located to date did not show improved productivity of the plant or alga.

In plants two phototropins have been reported, phot1 and phot2, these phototropins share sequence homology and have overlapping functions. These blue-light-sensitive receptors consist of two parts: a C-terminal serine-threonine kinase and two LOV domains that bind flavin mononucleotide as chromophores at the N-terminus. Recently, in the unicellular green alga, Chlamydomonas reinhardtii, a phototropin homolog was identified. It exhibits photochemical properties similar to those of higher plant phototropins and is also functional in Arabidopsis. Studies show that the basic mechanism of phototropin action is highly conserved, even though its apparent physiological functions are quite diverse.

### Phototropin in Higher Plants:

Plants utilize several families of photoreceptors to better react to their environment, allowing them to fine tune pathways controlled by the photoreceptors—phototropin, phytochrome, and cryptochrome (Chen, Chory et al. 2004).

In higher plants phototropin mediates a variety of blue-light elicited physiological processes (Sullivan, Thomson et al. 2008). Phototropins are UV-A/blue light sensing photoreceptors that are known to optimize photosynthetic yields (Chen, Chory et al. 2004). The involvement of phototropin in photomovement in higher plants is well documented (Suetsugu and Wada 2007, Kagawa, Kimura et al. 2009). Studies involving Arabidopsis mutants lacking the phot1 and phot2 genes have revealed that in addition to regulating hypocotyl curvature of seedlings towards blue light, phototropins also regulate a diverse range of responses in flowering plants. These responses include chloroplast movements, nuclear positioning, stomatal opening, leaf expansion, leaf movements and leaf photomorphogenesis.

Phototropin knock-out mutants (PHOT K/O) have been made previously in plants (Suetsugu and Wada 2007, Moni, Lee et al. 2015). For instance in Physcomitrella patens (a moss) there are three PHOT genes and they have all been knocked out in different mutants (Suetsugu and Wada 2007). The focus of the P. patens study was the effect of PHOT K/O on phototropism (movement toward light) and the phenotypes they observed allowed them to determine which of the genes were necessary for phototropism (Suetsugu and Wada 2007).

PHOT expression was higher in darkness than in light, and phot1 Arabidopsis mutants was shown to increase the number of lateral roots produced (Moni, Lee et al. 2015). phot was also demonstrated to mediate phototropism, chloroplast relocation and leaf expansion (Matsuoka, Iwata et al. 2007). Using phot deficient Arabidopsis mutants, phototropin 2 was linked to palisade parenchyma cell development of leaves (Kozuka, Kong et al. 2011).

Another study looked at the role of phototropin under low photosynthetically active radiation (Takemiya, Inoue et al. 2005). They found that the wild-type and the PHOT1 mutant both showed increased but similar growth in low radiance blue light super imposed on red light. In white light there was no increase in biomass in both phot1 and phot2 mutants as well as in the double phot mutant.

A study by Folta and colleagues investigated the relationship between phot1 and phototropism and growth inhibition in Arabidopsis (Folta, Lieg et al. 2003). They found that the onset of phototropism and the phot1-mediated growth inhibition coincided and postulated that both were due to phot1 expression.

There is a substantial amount of patent literature around phototropin in higher plants. However, the focus has been on the commercial utility of the upstream, light regulated areas rather than on the phototropin gene itself. These light control domains that regulate PHOT expression—the light-oxygen-voltage-sensing (LOV) domains—have been carefully evaluated for potential commercial application in higher plants.

Shu & Tsien application (US20130330718) focused on using the LOV domain for control of proteins that generate singlet oxygen (SOGs). These fusion protein tags could be used for imaging under blue light for research purposes.

Other patents use light switchable regulatory sequences and contemplate the use of the phototropin LOV domain such as Yang and colleagues (EP2682469).

Hahn & Karginov (WO2011133493) focused on allosteric regulation of kinases using the light activated domains for control of expression in engineered fusion proteins (such as the LOV domains).

Hahn and colleagues (U.S. Pat. No. 8,859,232) demonstrated that the LOV domain of phototropin can be used as a light activated switch for the activation or inactivation of fusion proteins of interest. They contemplated using a LOV domain that could contain substantial portions of the phototropin molecule in addition to the LOV domain. They contemplated using the LOV domain isolated from algae and gave the specific example of Vaucheria frigida, a stramenopile or heterokont alga.

Kinoshita and colleagues (WO2014142334) demonstrated that overexpression of phototropin had no impact of stomatal opening in higher plants.

Bonger and colleagues (US20140249295) used the LOV domain as a fusion with another functional protein wherein the light switching ability of the LOV domain was used to control the stability and/or function of the fusion protein.

Folta and colleagues (WO2014085626) using mutants of phototropin 1 were able to show that the function of phot1 is mediation of the pathway in which green light reverses the effects of red and/or blue light on plant growth.

Schmidt & Boyden (US20130116165) describe a new group of fusion proteins with light regulatory regions derived from Avena sativa phototropin 1. These regulatory domains are used for altering channel function in membranes.

To date there is no disclosure of the use of PHOT knockout or knockdown (suppression) technology to improve or algae plant productivity.

### Phototropin in Algae:

Phototropin has already been well studied in several different algae including Chlamydomonas reinhardtii (Briggs and Olney 2001). However, there are indications that phototropins have diverged significantly or that the genes that function as phototropin are not very homologous to plant phototropin genes. For instance it was reported that in Thalassiosira pseudonana (a diatom) and Cyanidioschyzon merolae (unicellular red alga) no genes were found encoding the phototropins (Grossman 2005). However putative genes with photosensory LOV domains, aurechromes, have been reported for these and other photosynthetic stramenopiles (Table 1). Most aureochromes contain a single LOV domain and function as transcription factors that regulate cell division, chloroplast movement, pigment production, and phototropism. (Takahashi. J Plant Res (2016) 129:189-197)

In Chlamydomonas reinhardtii, phototropin plays a vital role in progression of the sexual life cycle (Huang and Beck 2003), control of the eye spot size and light sensitivity (Trippens, Greiner et al. 2012). Phototropin is also involved in blue-light mediated changes in the synthesis of chlorophylls, carotenoids, chlorophyll binding proteins. Phototropin has been localized to the flagella of Chlamydomonas reinhardtii (Huang, Kunkel et al. 2004). Phototropin is also known to be involved in expression of genes encoding chlorophyll and carotenoid biosynthesis and LHC apoproteins in Chlamydomonas reinhardtii Eberhard et al. 2006). The Chlamydomonas reinhardtii phototropin gene has been cloned and shown to function when expressed in Arabidopsis (Onodera, Kong et al. 2005).

Phototropin has been shown to control multiple steps in the sexual life cycle of Chlamydomonas reinhardtii (Huang and Beck 2003). PHOT knockdowns using RNAi were generated (Huang and Beck 2003). The entire focus of this study was on sexual mating and no mention of improved biomass, starch accumulation or photosynthesis rate was observed. It is also involved in the chemotaxis that is the initial phase of the sexual cycle of Chlamydomonas reinhardtii (Ermilova, Zalutskaya et al. 2004). However, no cell cycle implications of phototropin knockout or knockdowns have been published.

Detailed studies have carefully analyzed the function of the LOV domain in several algal species. An example is the Chlamydomonas reinhardtii mutant LOV2-C250S where careful studies of the light activation and regulation of this domain were carried out to better understand the mechanism of action (Sethi, Prasad et al. 2009).

Phototropin knock-out mutants (PHOT K/O) have been made previously in algae (Zorin, Lu et al. 2009 Trippens, Greiner et al. 2012). PHOT minus strains had larger eyespots than the parental strain (Trippens, Greiner et al. 2012). This study focused on the impact of PHOT on eyespot structure function. These authors used a knock-out mutant of PHOT to reduce expression of phototropin (Trippens, Greiner et al. 2012).

Novel phototropins have been described in the green alga Ostreococcus tauri and with a focus on their LOV domain structure/function (Veetil, Mittal et al. 2011).

Abad and colleagues (WO2013056212) provide the sequence for phototropin from a green alga, Auxenochiorella protothecoides, and indicate that the gene would be important for photosynthetic efficiency. However, they do not discuss the impact of deletion or inhibition of this gene on the alga.

### DEFINITIONS

Unless otherwise defined, all technical and scientific terms have the same meaning as commonly understood by one of ordinary skill in the art to which this invention pertains. Although methods and materials similar or equivalent to those described herein can be used in the practice or testing of the exemplary embodiments, suitable methods and materials are described below. All publications, patent applications, patents, and other references mentioned are incorporated by reference in their entirety. In case of conflict, the present specification and definitions will control. In addition, the materials, methods, and examples are illustrative only and not intended to be limiting for the practice of this invention.

Unless specifically referred to in the specification singular forms such as “a,” “an,” and “the,” include their plural forms. As an example, “an alga” includes its plural form “algae” and “a plant” includes the plural “plants.”

The term “algae” will refer to all organisms commonly referred to as algae including the prokaryotic cyanophyta (commonly called blue-green algae and cyanobacteria), prochlorophyta, glaucophyta, rhodophyta, heterokontophyta, haptophyte, cryptophyta, dinophyta, euglenophyta, chloroaracniophyta, chlorophyta, and those organisms of indeterminate nomenclature normally referred to as algae. A full description of these is found in the book “Algae An Introduction to Phycology” by Van Den Hoek, Mann & Jahns (1995), which is included by reference.

The term “expression” as used herein refers to transcription and/or translation of a nucleotide sequence within a host cell. The level of expression of a desired product in a host cell may be determined on the basis of either the amount of corresponding mRNA that is present in the cell, or the amount of the desired polypeptide encoded by the selected sequence.

The term “overexpression” as used herein refers to excessive expression of a gene product (RNA or protein) in greater-than-normal amounts.

The term “homologous” refers to the relationship between two proteins that possess a “common evolutionary origin”, including proteins from superfamilies (e.g., the immunoglobulin superfamily) in the same species, as well as homologous proteins from different species.

As used herein, “identity” means the percentage of identical nucleotide or amino acid residues at corresponding positions in two or more sequences when the sequences are aligned to maximize sequence matching, i.e., taking into account gaps and insertions.

The term “sequence similarity” refers to the degree of identity or correspondence between nucleic acid or amino acid sequences that may or may not share a common evolutionary origin (Reeck, de Haen et al. 1987). However, in common usage and in the current invention, the term “homologous”, when modified with an adverb such as “highly”, may refer to sequence similarity and may or may not relate to a common evolutionary origin.

In specific embodiments, two nucleic acid sequences are “substantially homologous” or “substantially similar” when at least about 75%, and more preferably at least 80%, and more preferably at least 85%, and more preferably at least about 90% or at least about 95% of the nucleotides (or any integer value in between) match over a defined length of the nucleic acid sequences, as determined by a sequence comparison algorithm such as BLAST, CLUSTAL, MUSCLE, etc. An example of such a sequence is an allelic or species variant of the specific phototropin gene of the present invention. Sequences that are substantially homologous may also be identified by hybridization, e.g., in a Southern hybridization experiment under stringency conditions as defined for that particular system. The homology may be as high as about 93-95%, 98%, or 99% (or any integer value in between). For example, the sequence to which homology is matched is a wild-type parental line and the length of the sequence is the full length of the sequence from wild-type parental line.

Similarly, in particular embodiments of the invention, two amino acid sequences are “substantially homologous” or “substantially similar” when greater than 75% of the amino acid residues are identical wherein identical contemplates a conservative substitution at a nucleic acid position. In a preferred embodiment there is at least 80%, and more preferably at least 85%, and more preferably at least about 90% and more preferably at least about 90-95% of the amino acid residues are identical (or any integer value in between). Two sequences are functionally identical when greater than about 95% of the amino acid residues are similar. Preferably the similar or homologous polypeptide sequences are identified by alignment using, for example, the GCG (Genetics Computer Group, Version 7, Madison, Wis.) pileup program, or using any of the programs and algorithms described above. Conservative amino acid substitutions are among: acidic (negatively charged) amino acids such as aspartic acid and glutamic acid; basic (positively charged) amino acids such as arginine, histidine, and lysine; neutral polar amino acids such as glycine, serine, threonine, cysteine, tyrosine, asparagine, and glutamine; neutral nonpolar (hydrophobic) amino acids such as alanine, leucine, isoleucine, valine, proline, phenylalanine, tryptophan, and methionine; amino acids having aliphatic side chains such as glycine, alanine, valine, leucine, and isoleucine; amino acids having aliphatic-hydroxyl side chains such as serine and threonine; amino acids having amide-containing side chains such as asparagine and glutamine; amino acids having aromatic side chains such as phenylalanine, tyrosine, and tryptophan; amino acids having basic side chains such as lysine, arginine, and histidine; amino acids having sulfur-containing side chains such as cysteine and methionine; naturally conservative amino acids such as valine-leucine, valine-isoleucine, phenylalanine-tyrosine, lysine-arginine, alanine-valine, aspartic acid-glutamic acid, and asparagine-glutamine. A further aspect of the homologs encoded by DNA useful in the transgenic plants or algae of the invention are those proteins that differ from a disclosed protein as the result of deletion or insertion of one or more amino acids in a native sequence.

The term “knockout” or “gene knockout” refers herein to any organism and/or its corresponding genome where the gene of interest has been rendered unable to perform its function. This can be accomplished by both classical mutagenesis, natural mutation, specific or random inactivation, targeting in cis or trans, or any method wherein the normal expression of a protein is altered to reduce its effect. For example but not to limit the definition 1) one can use chemical mutagenesis to damage the gene and then select for organisms not expressing the gene, 2) one can target the gene and remove a portion or all of the gene by homologous recombination, 3) one can use RNAi methods to produce an inhibitor molecule for a particular protein and similar methods and 4) one can use genome editing tools (i.e. CRISPR-Cas) to specifically modify the gene.

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature (Sambrook, Fritsch et al. 1989, Ausubel, Brent et al. 1997, Green and Sambrook 2012).

The term “transcriptome” refers to the set of RNA molecules present in a population of cells. It often reflects how an organism responds to particular situations and is looking at what genes are regulated under a particular condition. Examples of transcriptome analyses on algae are found in the following references (Hwang, Jung et al. 2008, Rismani-Yazdi, Haznedaroglu et al. 2011, Fu, Wang et al. 2014, Koid, Liu et al. 2014).

The term “biofuel” refers to any fuel made through the application of biological processes not on a geological timescale. Examples include but are not limited to conversion of algal biomass to biocrude through hydrothermal liquefaction, anaerobic digestion of spent algal biomass for conversion to methane, extraction of lipid from algal biomass to convert to biodiesel, and conversion of water to biohydrogen through biological processes.

The term “bioproduct” is any product produced from biological processes either in whole or in part.

The term biomass productivity or production as used herein refers to the rate of generation of biomass in an ecosystem. It is usually expressed in units of mass per unit surface (or volume) per unit time, for instance grams per square metre per day (g m⁻² d⁻¹). The mass unit may relate to biologically produced dry matter generated.

The term “sink molecules”, “sink compounds”, sink materials” refers to molecules used by an organism to store captured carbon. These can be but are not limited to sugars, starch, glycogen, lipids, fats, waxes, and similar biomolecules.

The publications discussed above are provided solely for their disclosure before the filing date of the present application. Nothing herein is to be construed as an admission that the invention is not entitled to antedate such disclosures by virtue of prior invention.

### SUMMARY OF THE INVENTION

This and other unmet needs of the prior art are met by exemplary compositions and methods as described in more detail below.

One embodiment of the present invention provides for a method for increasing a biomass productivity of an algal strain wherein the expression or function of a Chlamydomonas reinhardtii phototropin gene, a gene substantially similar to the Chlamydomonas reinhardtii phototropin gene or a sequence substantially similar to SEQ ID NO 1-14, 51-66 and 69-128 is reduced or eliminated. In a preferred embodiment the gene substantially similar has greater than 75% homology, more preferably greater than 80%, or 85%, or 90% or 95% homology to the Chlamydomonas reinhardtii phototropin gene or the sequence identified in SEQ ID NO 1-14, 51-66 and 69-128.

For example, the biomass productivity of the algal strain is increased by greater than around 2-fold. The biomass production of storage product(s) in the algal strain is increased by greater than around 2-fold, for example the storage product(s) is selected from starch, lipid, pigments and other sink molecules and for example the productivity of biomass is increased by greater than around 2-fold. Further, the biomass productivity may be increased for bioproducts chosen from lipids, waxes, polysaccharides (e.g., starch, glycogen, mannans, glycans, cellulose, hemicellulose), pigments (e.g., xanthophyll). In a preferred embodiment the expression of the Chlamydomonas reinhardtii phototropin gene, the gene substantially similar to the Chlamydomonas reinhardtii phototropin gene or the sequence substantially similar to SEQ ID NO 1-14, 51-66 and 69-128 is reduced by example chemical mutagenesis and selection, genome editing, trans acting elements (e.g., RNAi), and/or an inducible basis through an inducible promoter.

Another embodiment of the present invention provides for an algal strain wherein relative to the wild-type parental line the expression of the phototropin gene or a substantially similar gene is reduced, the photosynthetic pigments making up the antenna complex are reduced, and/or the content of sink molecules is increased. In a preferred embodiment the phototropin gene or a substantially similar gene been rendered to be non-functional. In a preferred embodiment the non-functional gene has been substantially deleted or is rendered to be non-functional on an inducible basis through an inducible promoter. In a preferred embodiment the algal line having the phototropin gene deletion would generate sterile and stable diploid population of polyploid algae to avoid recombination of genetic material during sexual reproduction or in another embodiment would be used to generate stable transgene-stacking traits in polyploid algal strains. In a preferred embodiment the phototropin gene or a substantially similar gene is selected from SEQ ID NO 1-14, 51-66 and 69-128. In another preferred embodiment the gene or the gene substantially similar has greater than 75% homology, or greater than 80%, or 85%, or 90% or 95% homology to the Chlamydomonas reinhardtii phototropin gene or the sequence identified in SEQ ID NO 1-14, 51-66 and 69-128.

In another embodiment a method for increasing a biomass productivity of an algal strain wherein the expression or function of a Chlamydomonas reinhardtii NTR2 or NTRC gene, a gene substantially similar to a Arabidopsis NTR2 or NTRC gene or a sequence substantially similar to SEQ ID NO 35-50 and 67-68 is over expressed in the algal strain is provided. In a preferred embodiment the gene substantially similar has greater than 75% homology, or more than 80%, 85%, 90%, or 95% homology to the Arabidopsis NTR2 or NTRC gene or the sequence identified in SEQ ID NO 35-50 and 67-68.

For example, the biomass productivity of the algal strain is increased by greater than around 2-fold. The biomass production of storage product(s) in the algal strain is increased by greater than around 2-fold, for example the storage product(s) is selected from starch, lipid, pigments and other sink molecules and for example the productivity of biomass is increased by greater than around 2-fold. Further, the biomass productivity may be increased for bioproducts chosen from lipids, waxes, polysaccharides (e.g., starch, glycogen, mannans, glycans, cellulose, hemicellulose), pigments (e.g., xanthophyll).

In yet another embodiment a method for increasing a productivity of an algal strain wherein the expression or function of a Chlamydomonas reinhardtii KIN10 or KIN11 gene, a gene substantially similar to a Arabidopsis KIN10 or KIN11 gene or a sequence substantially similar to SEQ ID NO 15-34 is over expressed in the algal strain is provided. In a preferred embodiment the gene substantially similar has greater than 75% homology, or greater than 80%, 85%, 90%, or 95% homology to the Arabidopsis KIN10 or KIN11 gene or the sequence identified in SEQ ID NO 15-34. For example, the biomass productivity of the algal strain is increased by greater than around 2-fold. The biomass production of storage product(s) in the algal strain is increased by greater than around 2-fold, for example the storage product(s) is selected from starch, lipid, pigments and other sink molecules and for example the productivity of biomass is increased by greater than around 2-fold. Further, the biomass productivity may be increased for bioproducts chosen from lipids, waxes, polysaccharides (e.g., starch, glycogen, mannans, glycans, cellulose, hemicellulose), pigments (e.g., xanthophyll).

Exemplary embodiments of the compositions, systems, and methods disclosed herein wherein algae are treated so as to reduce or eliminate the expression of phototropin or a heterologous gene with the same function such that improved productivity is achieved.

In one aspect, embodiments of the present invention provide an organism and the method to use such organism where the phototropin gene is knocked out and the photosynthetic rate is improved and the biomass productivity improves.

In a further aspect, the mutant is produced from Chlamydomonas reinhardtii and the biomass productivity is doubled.

Another embodiment of the present invention provides an organism with reduced PHOT expression wherein the sexual cycle is arrested and the genetic stability of the algal cell culture line is improved.

In a further embodiment the organism is derived from Chlamydomonas reinhardtii and has reduced promiscuity resulting in a more stable genotype and phenotype.

In one aspect, embodiments of the present invention provide an organism with reduced phototropin gene expression and the method to use such organism which as improved non-photochemical quenching providing the ability for better response to high light levels.

In one aspect, embodiments of the present invention provide an organism with reduced phototropin expression and the method to use such organism that results in higher levels of sink molecules, such as but not limited to lipid and starch.

In a further embodiment the organism has enhanced cell division compared to wild-type.

In a further embodiment the organism is derived from Chlamydomonas reinhardtii.

In another embodiment of the method wherein the expression of the Chlamydomonas reinhardtii phototropin gene is reduced by genome editing (i.e. CRISPR/Cas).

In another embodiment of the method wherein the expression of the Chlamydomonas reinhardtii phototropin gene is reduced by trans acting elements (e.g., RNAi).

In a further embodiment the gene downstream of PHOT has substantial homology to the Arabidopsis KIN10 or KIN11 genes or a portion thereof (Snf1 related kinases, SNRK) and can be overexpressed to increase the productivity of an algal strain.

In yet a further embodiment the KIN10 and KIN11 genes or a portion thereof are chosen from genes substantially homologous to a nucleic acid sequence identified in SEQ ID NO 15 to 34 or a nucleic acid sequence encoding for an amino acid sequence identified in SEQ ID NO15 to 34.

In a further embodiment the gene downstream of phot has substantial homology to the Arabidopsis NTRC and NTR2 gene(s) or a portion thereof and can be overexpressed to increase the productivity of an algal strain.

In yet a further embodiment the NTRC and NTR2 genes or a portion thereof are chosen from genes substantially homologous to a nucleic acid sequence identified in SEQ ID NO 35 to 50 or a nucleic acid sequence encoding for an amino acid sequence selected in SEQ ID NO 35 to 50.

### BRIEF DESCRIPTION OF THE DRAWINGS

A better understanding of the exemplary embodiments of the invention will be had when reference is made to the accompanying drawings, and wherein:

FIG. 1A-D Comparison of chlorophyll a/b ratios and chlorophyll content of PHOT K/O lines (PHOT K/O line G5 and parent cw15) and (PHOT K/O line A4 and parent UVM4): (A) chlorophyll a/b ratios in low light, (B) chlorophyll a/b ratios in low light and high light, (C) chlorophyll content in low light grown cells of cw15 parent and G5 mutant, and (D) chlorophyll content in low light grown cells of UV4 parent and A4 mutant.

FIG. 2A-D—Carotenoid pigment comparison of low light (LL) and high light (HL) grown cultures of Chlamydomonas reinhardtii PHOT K/O lines compared to wild-type. LL=Low light, HL=high light, CW15=Parent for G5 PHOT K/O line, UV4=parent for A4 PHOT K/O line, Neo=neoxanthin, Lutein=lutein, Viola=violaxanthin, Anthera=antheraxanthin, and Zea=zeaxanthin.

FIG. 3A-B—Xanthophyll cycle carotenoid de-epoxidation in Chlamydomonas reinhardtii PHOT K/O (lines G5 and A4) and their corresponding parental lines (CW15 and UVM4) grown at low and high light intensities.

FIG. 4A-D—Chlorophyll fluorescence induction kinetics of low-light grown Chlamydomonas reinhardtii PHOT K/O lines and respective wild-type parental strains. Cultures were either dark adapted or pre-illuminated with 715 nm light (photosystem I (PSI) actinic light) prior to measurement. For Chl fluorescence induction measurements, Chl fluorescence was measured under continuous, non-saturating illumination every microsecond.

FIG. 5A-B—Photosynthetic rate comparison of Chlamydomonas reinhardtii PHOT K/O lines and parent lines under increasing light intensity. CW15 and UV4 are parental wild-type lines while G5 and A4 are the PHOT K/O lines.

FIG. 6—KEGG pathway graphical data on photosynthetic electron transport chain related gene expression Chlamydomonas reinhardtii PHOT K/O lines and parent lines. Star indicates fold change in transcript abundance relative to parent line.

FIG. 7A-D—Growth and biomass comparison of Chlamydomonas reinhardtii PHOT K/O lines and parent lines in environmental photobioreactors from Phenometric (ePBRs).

FIG. 8—KEGG pathway graphical data on carbon fixation related gene expression Chlamydomonas reinhardtii PHOT K/O lines and parent lines. Hatched line and/or star indicates fold change in transcript abundance relative to parent line.

FIG. 9—Cell cycle pathway diagram. N/MA (Never in mitosis), NEK2, NEK6 (N/MA related kinases), Cyclin and CDK (Cyclin-dependent kinases), RB (retinoblastoma)/mat3 (mating type-linked) genes are up-regulated in cell cycle pathway.

FIG. 10—Starch synthesis pathway.

FIG. 11A-B—Thylakoid membrane structure and starch accumulation comparison of PHOT K/O line with parent line. Inserts are a magnification of the thylakoid grana stacks.

FIG. 12—KEGG pathway graphical data on terpenoid synthesis related gene expression Chlamydomonas reinhardtii PHOT K/O lines and parent lines. Star indicates up-regulated genes relative to parent line.

### DETAILED DESCRIPTION

While there have been numerous studies on algal phototropin (Huang and Beck 2003, Ermilova, Zalutskaya et al. 2004, Huang, Kunkel et al. 2004, Im, Eberhard et al. 2006, Sethi, Prasad et al. 2009, Veetil, Mittal et al. 2011, Trippens, Greiner et al. 2012) to date there has been no correlation of the reduction or knock-out of phototropin to higher levels of biomass production and increased production of sink molecules/products such as starch and lipid.

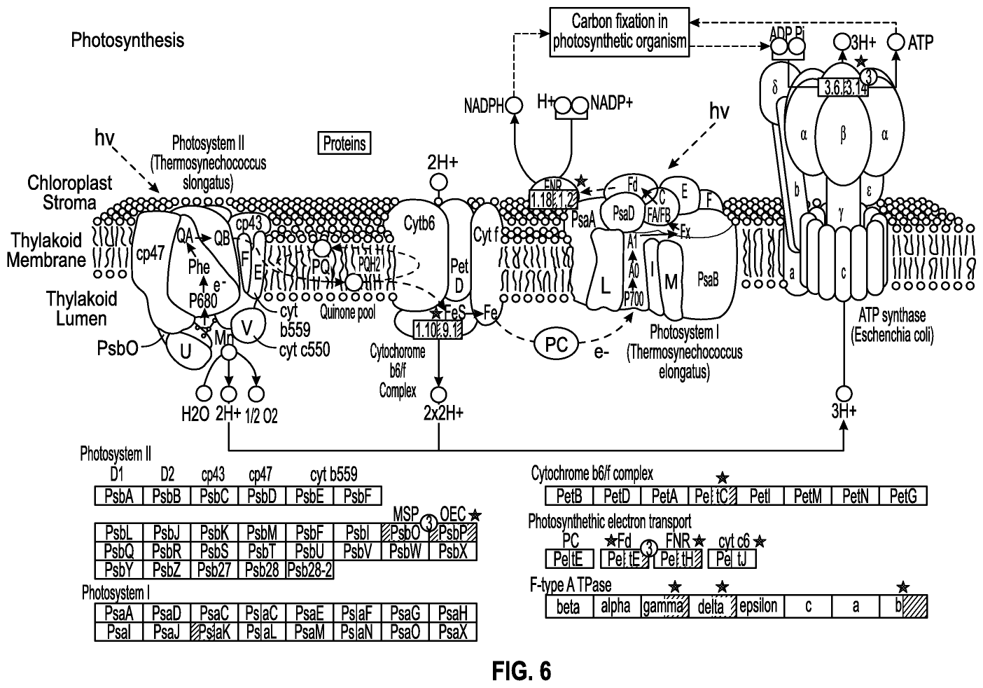

The transcriptome of a Chlamydomonas reinhardtii phototropin knock out (PHOT K/O) mutant and the wild-type parent were compared to analyze differences in gene expression in high light grown cultures (500 μmol photons m⁻² s⁻¹). An up-regulation of genes involved in photosynthetic electron transport chain, carbon fixation pathway, starch, lipid, and cell cycle control genes was observed in the PHOT K/O mutants. Referring now to FIG. 6, with respect to photosynthetic electron transport genes, genes encoding proteins of the cytochrome b6f and ATP synthase complex were up regulated potentially facilitating rate limitations in proton-coupled electron transfer. In addition genes involved in the rate limiting steps in the Calvin cycle, including Ribulose-1,5-bisphosphate carboxylase/oxygenase (RuBisCO), sidoheptulose 1,7 bisphosphatase (SBPase), glyceraldehyde-3-phosphate dehydrogenase (3PGDH) and that mediate cell-cycle control (CDK), were also up regulated in the PHOT K/O mutants as well as the starch synthase and fatty acid biosynthesis genes involved in starch and lipid synthesis. In addition, transmission electron micrographs show increased accumulation of starch granules in PHOT K/O mutant compared to wild-type, which is consistent with the higher expression of starch synthase genes. Collectively, the altered patterns of gene expression in the PHOT K/O mutants were associated with a two-fold increase in growth and biomass accumulation compared to wild-type when grown in environmental photobioreactors (PBR101 from Phenometrics, Inc., Lansing, Mich.) that simulate a pond environment as evidence of increase productivity of algae. These surprising results suggest that phototropin may be a master gene regulator that suppresses rapid cell growth and promotes gametogenesis and sexual recombination in wild-type strains. Therefore, down regulating expression or eliminating the phototropin genes (e.g., PHOTO K/O mutants) provides a valuable means to increase productivity of algae that has commercial applications.

Using a variety of methods exemplary embodiments of the invention are directed at improving the productivity of algal systems based on control of the phototropin gene and genes similar to phototropin in algal systems. This is particularly applicable to improving biomass productivity in algal mass culturing either for production of algal biofuels or bioproducts.

Productivity is a central issue in algae production and a doubling of the productivity could be very attractive to groups who hope to cross the threshold of commercial viability. However, one should note that widespread adoption of transgenic algae as a production system is not yet embraced. Several companies (for example Algenol, Ft. Meyers, Fla.) are using transgenic algae (cyanobacteria) in closed tube reactors outdoors and, presumably, have a track to (national) regulatory approval. Use of transgenic algae has been approved in Florida and approvals have recently been granted by the US EPA for GMO field trials for Sapphire Energy Company.

Production of bioproducts using this invention, owing to the observed doubling of productivity in biomass and sink molecules/compounds, could be pivotal in reaching commercial viability. The observed increase in starch production by this invention is especially important as it shows sink molecules/compounds are enhanced by the methods of this invention.

Alternative genome editing technologies such as CRISPR/Cas 9, Talen and Zinc finger nuclease approaches could also be used to inhibit expression of phototropin (Gaj, Gersbach et al. 2013, Sizova, Greiner et al. 2013).

It is possible to make PHOT knockouts using non-GMO approaches such as classical mutagenesis using chemical mutagens such as methylnitronitroso guanidine and ethyl methane sulfonate (Yan, Aruga et al. 2000).

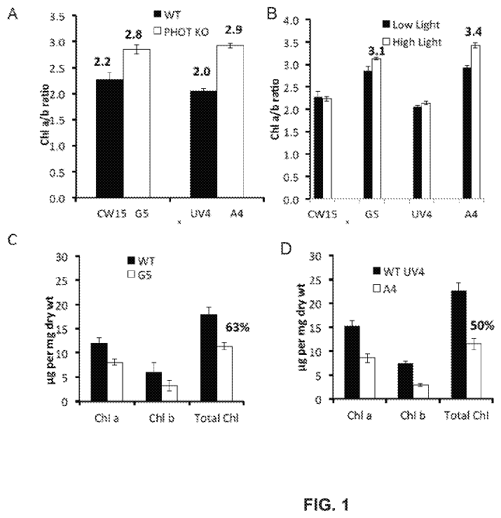

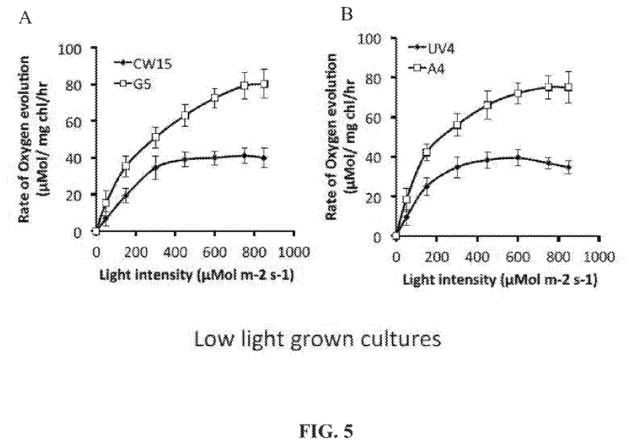

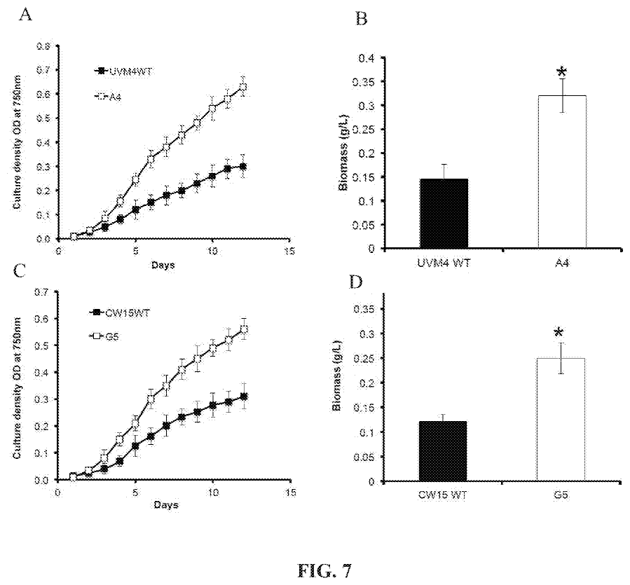

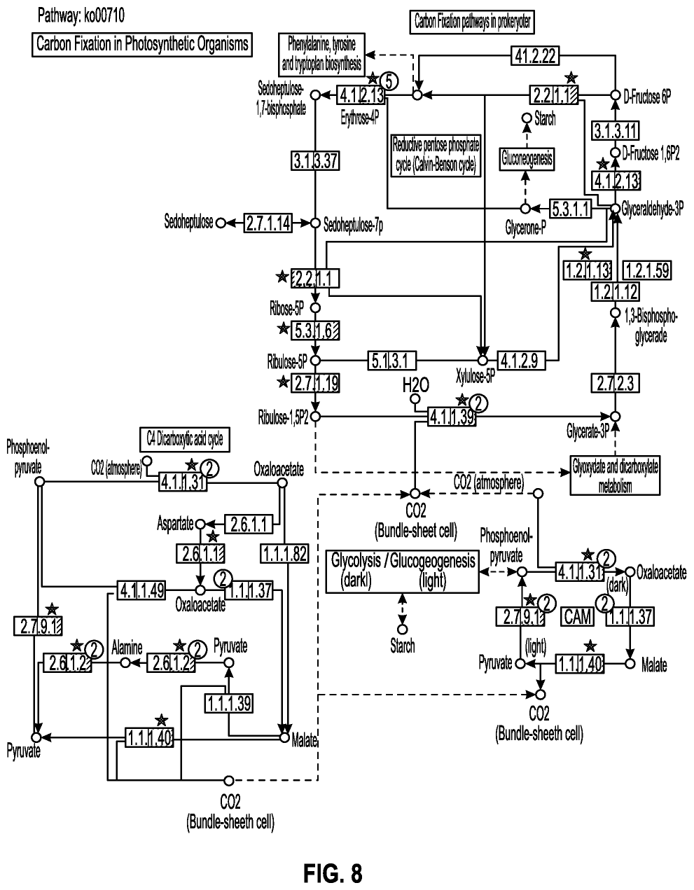

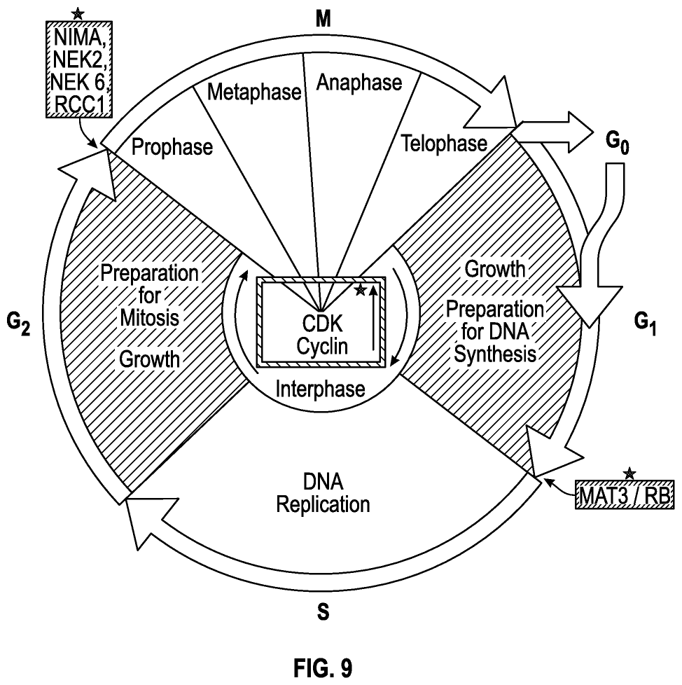

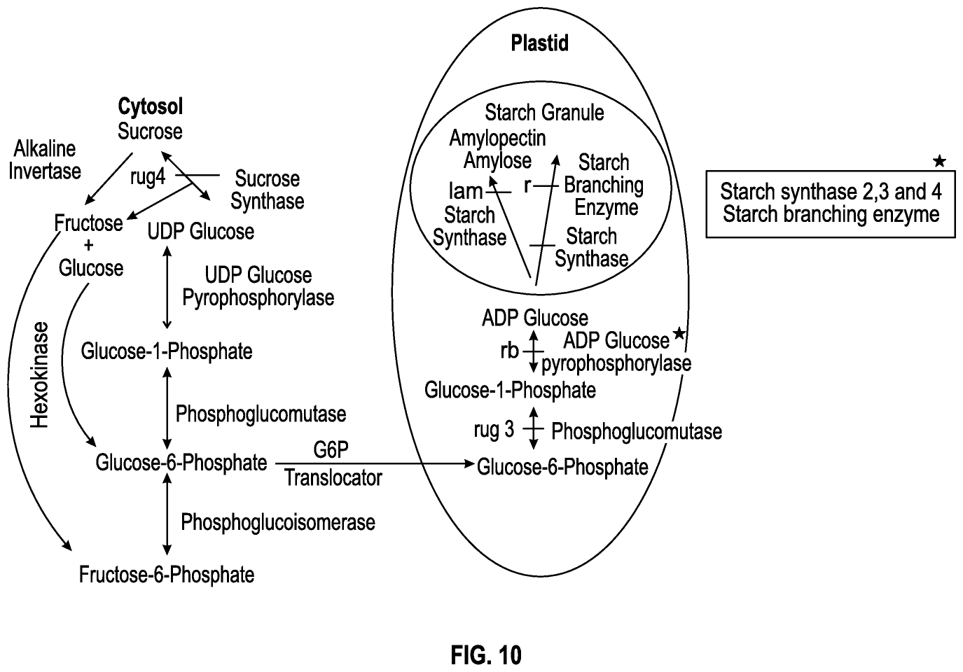

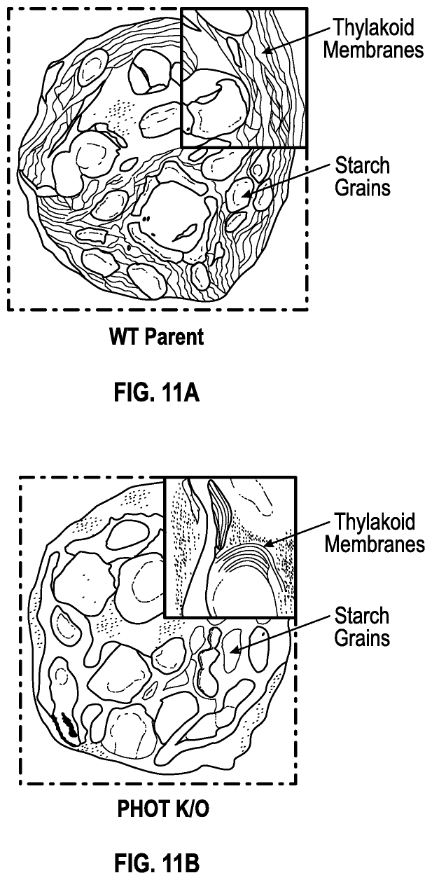

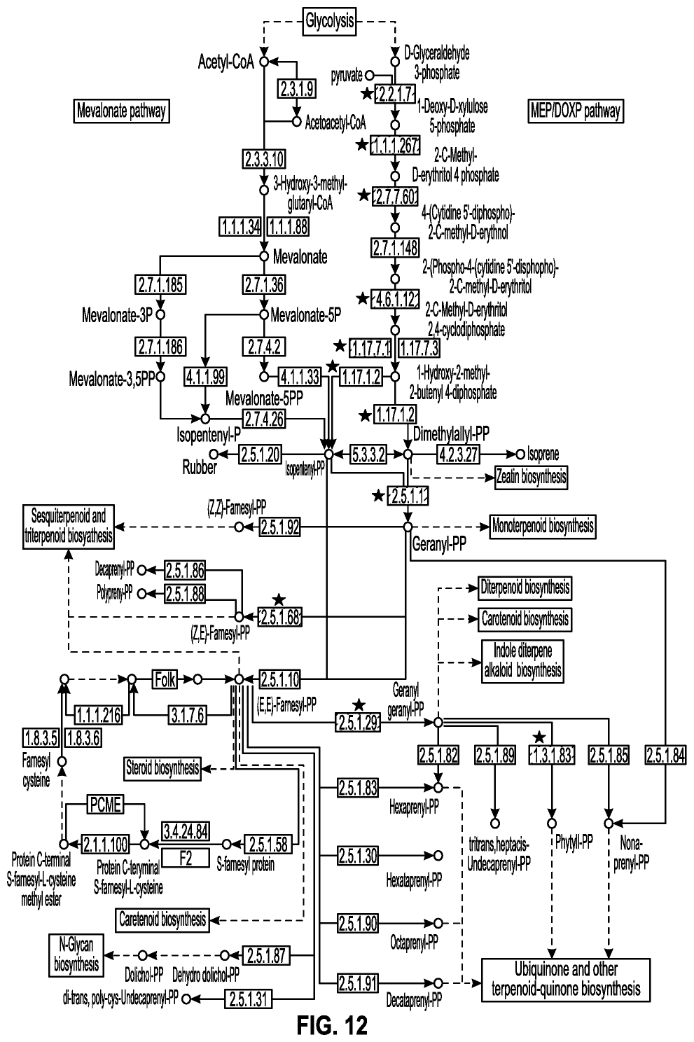

To date, supporting data for this invention have been limited to the green alga, Chlamydomonas reinhardtii. Compared to wild-type C. reinhardtii, PHOT K/O mutants of the invention show:
1. Reduction in chlorophyll and carotenoid pigments (see FIG. 1).
2. Reduced light harvesting antenna size (see FIG. 1).
3. 2-fold increase in photosynthesis rate (see in FIG. 5).
4. Increased expression of genes that control rate limiting steps in photosynthetic electron transfer and Calvin Cycle activity (see FIG. 6 and FIG. 8).
5. 2-fold increase in growth and biomass (see in FIG. 7.)
6. Increased expression of starch synthesis genes (see in FIG. 10.)
7. Increased accumulation of xanthophyll cycle pigments (see in FIG. 12).
8. Higher accumulation of starch grains (see in FIG. 11B).
9. Increased expression of the chloroplast localized MEP terpenoid synthesis pathway but not the cytoplasmic MVA terpenoid synthesis pathway (see in FIG. 12)
10. Increased expression of cell cycle control genes potentially accelerating rates of cell division (see in FIG. 9).
11. Increased expression of glycolysis pathway genes.
12. Increased expression of Kin10/Kin11 (SNRK) genes.
13. Increased expression of NTR2 and NTRC genes.

Additionally, PHOT K/O mutants were unable to undergo sexual mating, which was attributed to an impact of the PHOT K/O on the cell cycle—effectively blocking meiosis while accelerating photosynthetic and cell division rates.

### PHOT Knockout (K/O) Mutants of Chlamydomonas Reinhardtii

Chlamydomonas reinhardtii PHOT knockout lines were generated in different parental backgrounds. PHOT K/O line G5 was made in cw15 parental background and A4 mutant line was made in UV4 background (Zorin, Lu et al. 2009).

### Pigment Analysis of Phototropin Knock Out Lines

Chlorophyll (Chl) and carotenoids are the central pigments of the photosynthetic apparatus. These pigments are associated with light-harvesting complexes and reaction-center complexes in photosynthetic organisms. The light environment plays a major role in governing the pigment composition of pigment-protein complexes of the photosynthetic apparatus. Blue light is especially important in modulating the synthesis of Chl and carotenoids, as well as the biogenesis of the photosynthetic apparatus in microalgae and vascular plants. Consistent with phototropin regulation of pigment biosynthetic pathways C. reinhardtii PHOT K/O lines showed:
Chlorophyll content: Higher chlorophyll a/b (Chl a/b) ratios compared to their respective wild-types when grown under low light intensities. As shown in FIGS. 1A and 1B, the G5 mutant line has Chl a/b ratios of 2.8 and 3.1 in low and high light, respectively while its parent CW15 has a Chl a/b ratio of 2.2 in low light with no significant increase in high light. Similarly, the mutant A4 line has Chl a/b ratios of 2.9 and 3.4 in low light and high light respectively, and its parent has a Chl a/b ratio of 2 in low light with no significant change in high light. Chl a/b ratios are also higher in PHOT K/O lines under high light grown cultures, which is consistent with a reduction in chlorophyll antenna size at high light. FIGS. 1C and 1D shows a 50-60% reduced chlorophyll content per gram dry weight in the PHOT mutants compared to parent wild-type.

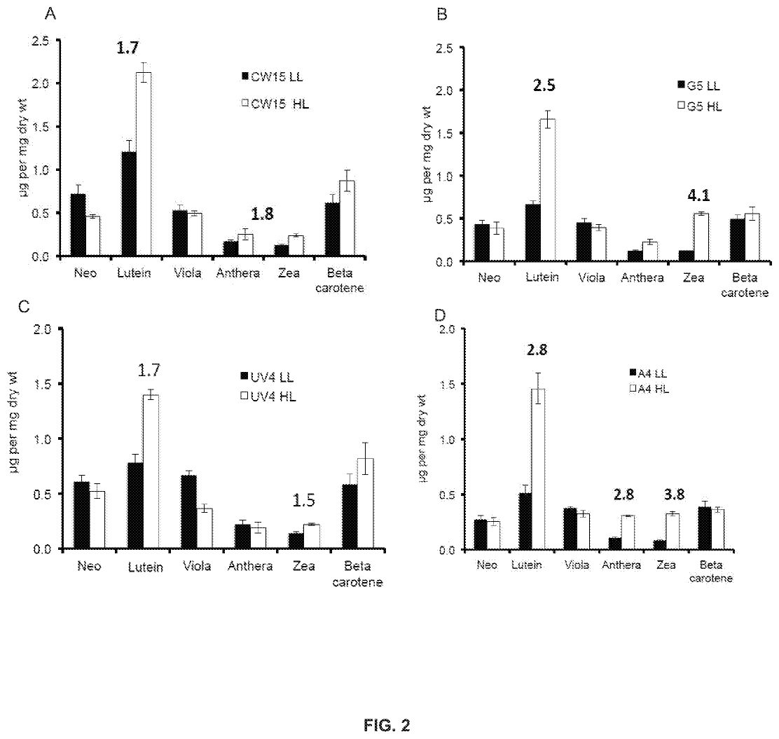

Carotenoid content: When grown under low light intensities PHOT K/O lines showed a 30-40% reduction in carotenoid content compared to parent wild. The changes in xanthophyll cycle pigments were analyzed since the xanthophyll cycle pigments play an important role as antioxidants and for non-photochemical quenching of excess energy captured by the light harvesting complex. Both PHOT K/O lines show higher accumulation of photoprotective pigments in high light compared to their respective WT parents. Referring now to FIG. 2B, G5 PHOT accumulates 2.5 fold more lutein and 4.1 fold more zeaxanthin compared to the parental line as shown in FIG. 2A. Referring now to FIG. 2D, A4 PHOT K/O accumulates 2.8 lutein and 3.8 fold zeaxanthin as well as 2.8 fold antheraxanthin compared to its respective parent as shown in FIG. 2C. These results are consistent with the better photosynthetic performance of these lines when grown in high light intensities.

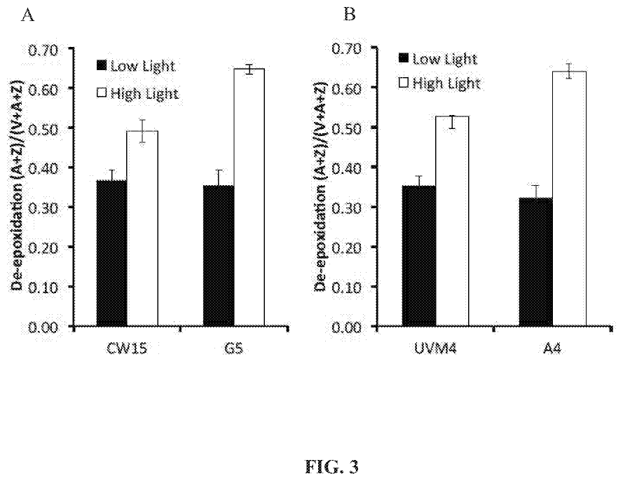

De-epoxidation rates: Consistent with the xanthophyll cycle pigment accumulation PHOT K/O lines show higher De-epoxidation in high light conditions as compared to their respective wild-type under high light (FIG. 3A-B). These data are consistent with the better performance of PHOT K/O lines in high light intensities as they have more robust photoprotection mechanisms.

### Photosynthetic State Transition Analysis in Parent and PHOT K/O Lines:

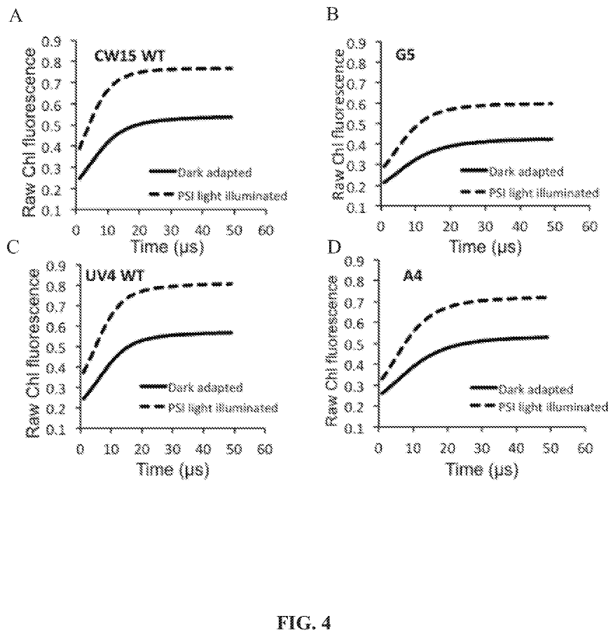

In C. reinhardtii, the peripheral PSII antenna is able to migrate laterally between PSII and PSI, in a process known as state transitions, to balance the excitation energy distribution between the two photosystems and to regulate the ratio of linear and cyclic electron flows. Linear electron transfer produces ATP and NADPH, while cyclic electron transfer driven by PSI produces only ATP. Increasing the antenna size of the PSI complex facilitates cyclic electron transfer and has been shown to enhance ATP production and support the optimal growth of Chlamydomonas. To assess the impact of reduced pigment content on the ability to carry out state transitions, chlorophyll (Chl) fluorescence induction kinetics were measured in low-light grown parent wild-type (FIGS. 4A and C) and PHOT K/O cells (FIG B and D), that were either dark adapted (sold line) or pre-illuminated with PSI (715 nm) actinic light (broken line). PSI actinic light pre-illumination promotes light harvesting complex II (LHCII) migration from PSI to PSII. An increase in the PSII antenna size would accelerate Chl fluorescence rise kinetics and increase the maximal Chl fluorescence level at sub-saturating light intensities. Wild-type strains (FIGS. 4A and C) and PHOT K/O lines (FIGS. 4B and D) all had faster Chl fluorescence rise kinetics and achieved greater maximum Chl fluorescence levels following pre-illumination with PSI light as compared to dark adapted cells consistent with robust state transitions.

### Photosynthetic Rates in WILD-TYPE and PHOT K/O Lines:

Referring now to FIG. 5A and FIG. 5B, the photosynthetic rates of the PHOT lines were determined under increasing light conditions and PHOT K/O lines (open boxes) show 2 fold higher photosynthetic rates compared to their respective parent strains (filled circles). Rate limiting genes in photosynthetic electron transport genes were up-regulated in high light grown cultures (FIG. 6). Up-regulation of these genes may play a role in higher photosynthetic efficiency of PHOT K/O mutants.

### Photosynthetic Electron Transport Pathway Genes:

The transcriptomic analysis of the PHOT K/O mutants compared to wild-type parental strains provided information on the different genes impacted by the elimination of phototropin expression (FIG. 6). These data are reported in the KEGG (Kyoto Encyclopedia of Genes and Genome) pathway format (Kanehisa and Goto 2000, Kanehisa, Goto et al. 2014) found on the world wide web at genome.jp/kegg/mapper.html last visited May 25, 2016. Rate limiting genes in photosynthetic electron transport pathway were up-regulated in high light grown cultures. Up-regulation of these genes may play a role in higher photosynthetic efficiency of PHOT K/O mutants.
1. PetC: Is a nuclear gene encoding the Rieske protein of the cytochrome b₆/f (cyt b₆/f complex. The cytochrome b₆f complex catalyzes the rate-limiting step in photosynthetic electron transport. Increases in its expression levels or stoichiometry relative to the PSI and PSII reaction centers would be predicted to increase rates of electron and proton transfer. A 2-fold increase on petC expression was observed for the PHOT K/O mutants (see FIG. 6).
AtpD: Encodes the delta subunit for ATPase. A 3-fold increase on AtpD expression was observed for the PHOT K/O mutants (see FIG. 6).
F type ATPase genes: The delta and gamma subunits of the F type ATPase gene were evaluated. Increases in expression of the ATPase complex would facilitate proton flux, increase ATP synthesis and reduce feedback inhibition on proton coupled electron transfer by accelerating dissipation of the delta pH gradient across the thylakoid membrane. A 3-fold increase was observed for the PHOT K/O mutants (see FIG. 6).
PGRL1: Is an important gene for efficient cyclic electron flow. A 2.2 fold increase was observed for PHOT K/O mutants
PGR7: Is a gene necessary for efficient photosynthetic electron transport. A 6.4 fold increase was observed for PHOT K/O mutants.

### Growth and Biomass Analysis in Parent and PHOT K/O Lines:

Most importantly, phototropin knock out lines (open boxes), had twice the cell density (FIGS. 7A and 7C) and accumulated twice the biomass (FIGS. 7B and 7D) of their respective parental wild-type strain (solid boxes) when approaching the stationary phase of growth (after 12 days) (FIG. 7). These results are consistent with higher photosynthetic rates in phototropin knock out lines also impact biomass yield of cells grown under conditions mimicking the pond simulating conditions (ePBRs). These results are in concert with up-regulation of the genes involved in carbon fixation and cell cycle as determined by transcriptomic analysis.

### Carbon Fixation Pathway Genes Upregulated:

Carbon fixation is the main pathway for storing energy and accumulating biomass in algae and plants. Many rate limiting genes were up-regulated in PHOT K/O lines (FIG. 8). SBPase and RuBisCO are limiting enzymes in the Calvin Cycle and their overexpression would increase carbon flux through the carbon reduction pathways. Carbonic anhydrase (CA), an enzyme active in the interconversion of bicarbonate and CO2 facilitating CO2 fixation.
1. Ribulose-1,5-bisphosphate carboxylase/oxygenase (RuBisCO) A 3-fold increase was observed for the PHOT K/O mutants (see FIG. 8).
2. Sidoheptulose 1,7 bisphosphatase (SBPase): A 3-fold increase was observed for the PHOT K/O mutants (see FIG. 8).
3. Glyceraldehyde-3-phosphate dehydrogenase (3PGDH): A 2-fold increase was observed for the PHOT K/O mutants (see FIG. 8).
4. α carbonic anhydrases: A 2.6 to 5 fold increase was observed for the PHOT K/O mutants.
5. β carbonic anhydrases: A 8 fold to 6 fold increase was observed for the PHOT K/O mutants.

### Thioredoxin Reductase Genes are Up-Regulated in PHOT K/O Lines:

Thioredoxins are small ubiquitous redox proteins, which are crucial components of the regulatory redox networks in all living cells. Thioredoxins are reduced by different reductases, depending on their subcellular localization. Among these reductases, NADPH-dependent thioredoxin reductases (NTR) genes are known to regulate multiple gene targets involved in photosynthesis, non-photochemical quenching (NPQ), Calvin-Benson cycle, starch biosynthesis, cold stress tolerance and thermotolerance.
1. NADPH-dependent thioredoxin reductase C (NTRC): A 2.4 fold increase was observed for the PHOT K/O mutants
2. NADPH-dependent thioredoxin reductase 2 (NTR2): A 4 fold increase was observed for the PHOT K/O mutants

### Key Growth Regulatory Genes are Up-Regulated in PHOT K/O Lines:

KIN10 or KIN11 ((Snf1 related kinases, SNRK) are one of the very well-studied central regulators of energy and stress metabolism in plants. SNRK1 proteins play central roles in coordinating energy balance and nutrient metabolism in plants. A 10-fold increase was observed for the PHOT K/O mutants.

### Cell Cycle Pathway Genes Up Regulated:

Cell cycle genes are up regulated in Chlamydomonas reinhardtii PHOT K/O mutants may enhance cell division in these lines contributing to the higher biomass in these lines (FIG. 9).
1. NIMA (Never in mitosis), NEK2, NEK6 (NIMA related kinases): Cell cycle progression (G2/M progression) 15, 5 and 5 fold increase, respectively, was observed for the PHOT K/O mutants.
2. RCC1 (Regulator of chromosome condensation): A16 fold increase was observed for the PHOT K/O mutants. Cyclin and cyclin-dependent kinases (CDK): Cyclin-dependent kinases are involved in overall regulation of cell cycle progression and demonstrated a 2-fold increase for the PHOT K/O mutants.
3. A 3-fold increase in MAT3 a homolog of retinoblastoma protein (MAT3/RB) was observed for the PHOT K/O mutants: These genes regulate the cell cycle at two key points: 1.) early/mid G1 control point, and 2) the size checkpoint for the dividing cell.

### Glycolysis Pathway Genes are Up-Regulated in PHOT K/O Lines:

Glycolysis is the first step in the breakdown of glucose to extract energy for cellular metabolism, which converts glucose to pyruvate and generates ATP (energy) and NADH (reducing power). Many important genes of this pathway show higher expression in PHOT K/O mutants.
1. Hexokinase: A 3.4 fold increase was observed for the PHOT K/O mutants.
2. Glyceraldehyde phosphate dehydrogenase: A 6 fold increase was observed for the PHOT K/O mutants
3. Fructose—bisphosphate Aldolase: A 4 fold increase was observed for the PHOT K/O mutants.
4. Pyruvate Kinase: A 16 fold increase was observed for the PHOT K/O mutants.

### Thylakoid Membrane Structure and Starch Accumulation in Parent and PHOT K/O Lines:

We compared the chloroplast ultrastructure of the parental and PHOT K/O cells to determine whether there were changes in thylakoid membrane structure and starch accumulation. Starch represents the most widespread storage polysaccharide found in the plastids of both photosynthetic and non-photosynthetic cells of plants and algae. PHOT K/O lines exhibited higher accumulation of starch grains compared to their respective parent strains as well as up-regulation of starch synthesis genes (FIGS. 10 and 11B) (discussed below).

### Starch Biosynthesis Pathway Genes Upregulated in PHOT K/O Lines:

Chlamydomonas reinhardtii PHOT K/O mutants have higher starch accumulation due to up-regulation of the following genes involved in starch biosynthesis is FIG. 10. These results were consistent with the observed increase in starch content in PHOT K/O chloroplasts by EM.
1. AGPase: ADP glucose pyrophosphorylase catalyzes the rate-limiting step and first-dedicated step for starch biosynthesis. A 2-fold increase was observed for the PHOT K/O mutants.
2. Starch synthase 2, 3 and 4: A 5-fold increase was observed for the PHOT K/O mutants.
3. Starch branching enzyme: A 3-fold increase was observed for the PHOT K/O mutants.

A structural hallmark of thylakoid membranes in plants and microalgae is the stacking of the membranes associated with the localization of the PSII complex. The stromal membranes extending from the stacks are enriched in PSI and ATPase complexes. This arrangement of LHCII complexes provides functional flexibility, enabling their primary light harvesting function as well as ability to participate in multilevel regulatory mechanisms involving highly efficient energy dissipation through pigment interactions such as chlorophyll-xanthophyll interactions. These regulatory processes require a significant reorganization in the membrane, and a substantial degree of structural flexibility in thylakoid membranes to carry out short-term adaptations and long-term acclimations in response to change in light and environmental stimuli.

An electron micrograph illustration showing the thylakoid membrane structure in both parent strain and PHOT K/O line is drastically altered in PHOT K/O lines. These results are in concert with the phototropin involvement in regulation of LHC protein biosynthesis and pigment biosynthesis. When thylakoid membranes are tightly stacked, they are densely packed with proteins and inhibit efficient protein diffusion including diffusions of the electron transport carrier protein plastocyanin. This protein mobility is required for efficient photosynthetic electron transfer, as well as regulation and repair of photodamaged photosynthetic apparatus. In parent cells thylakoid membranes are very tightly stacked giving very little space for the movement of the molecules). In contrast, PHOT K/O lines have parallel grana stacks and wide luminal spacing

### Other Important Genes Upregulated in Transcriptomic Analysis:

### Lipid Biosynthesis Pathway Genes:

The following genes involved in lipid metabolism are up regulated in PHOT K/O mutants:
1. Acyl carrier protein (ACP) is an important component in both FA and polyketide biosynthesis with the growing chain bound during synthesis as a thiol ester. A 3-fold increase was observed for the PHOT K/O mutants.
2. ω-3 fatty acid desaturase (FAD) A 4-fold increase was observed for the PHOT K/O mutants.
3. Fatty acid biosynthesis (FAB). A 3-fold increase was observed for the PHOT K/O mutants.

### Terpenoid Biosynthesis Pathway Genes:

The methyl erythritol 4-phosphate (MEP) pathway is the source of isoprenoid precursors for the chloroplast. The precursors lead to the formation of various isoprenoids having diverse roles in different biological processes. Some isoprenoids have important commercial uses. Isoprene, which is made in surprising abundance by some trees, plays a significant role in atmospheric chemistry. Multiple genes involved in MEP/DOXP pathway were up regulated in PHOT K/O mutants (FIG. 12). In contrast, the mevalonate terpenoid pathway (cytoplasmic) genes were not up regulated in PHOT K/O mutants.

Note that all data so far were generated in cell wall free mutants of Chlamydomonas reinhardtii. Metabolomic analyses in C. reinhardtii clarified the pathways and gene up-regulation in high light in C. reinhardtii PHOT K/O mutants of this invention:

### Heterologous Algal Phototropin Genes

The Chlamydomonas reinhardtii phototropin gene has already been sequenced and a provisional version is available publically (GenBank 5718965). Additional algal genes are available that have either been shown to be a phototropin, contain blue light receptors, have some homology to phototropin or are putative blue light receptors similar to phototropin (Table 1). Additional phototropin genes in two other production strains of microalgae are known.

Chlorella sp. Strain1412. Is a strain developed by the National Alliance of Biofuels and Bio-products (NAABB) consortium and is housed at UTEX Culture Collection Of Algae at the University of Texas at Austin (UTEX). The amino acid sequence is provided as SEQ ID NO. 1 and the nucleotide sequence as SEQ ID NO. 2. The phototropin B gene of Chlorella sorokiniana. Strain 1412 is provided as SEQ ID NO. 3 and nucleotide as SEQ ID NO. 4.

Chlorella sp. sorokiniana strain 1230. Is a UTEX strain. The amino acid sequence of phototropin A is provided as SEQ ID NO. 5 and the nucleotide sequence as SEQ ID NO. 6. The amino acid sequence of phototropin B is provided as SEQ ID NO. 7 and the nucleotide sequence as SEQ ID NO. 8.

Chlorella sp. sorokiniana strain 1228. The amino acid sequence of phototropin A is provided as SEQ ID NO. 9 and the nucleotide sequence as SEQ ID NO. 10. The amino acid sequence of phototropin B is provided as SEQ ID NO. 11 and the nucleotide sequence as SEQ ID NO. 12.

Picochlorum soloecismus (DOE101). The amino acid sequence is provided as SEQ. ID NO. 13 and the nucleotide sequence as SEQ. ID NO. 14.

TABLE 1
List of publically available sequences that may be phototropins or
heterologous to phototropin genes based upon homology or function.

| GenBank # | Alga | Description | Aliases |
|---|---|---|---|
| 9688782 | Micromonas pusila CCMP1545 | Phototropin, blue | MICPUCDRAFT_49739 |
| | | light receptor | |
| 9617508 | Volvox carteri f. nagariensis | Phototropin | VOLCADRAFT_127319 |
| 23616146 | Auxenochlorella protothecoides | Phototropin 2 | F751_4755 |
| 23614975 | Auxenochlorella protothecoides | Phototropin-1B | F751_3584 |
| 19011210 | Bathycoccus prasinos | Phototropin | Bathy16g02310 |
| 9831018 | Ostrecoccus tauri | Putative blue light | Ot16g02900 |
| | | receptor | |
| 8249220 | Micromonas sp, RCC299 | Blue light receptor | MICPUN_105003 |
| 16998047 | Cyanidioschyzon merolae 10D | Serine/threonine | MICPUT_105003 |
| | | kinase | |
| 17089759 | Galdieria sulphuraria | Serine/threonine | Gasu_15820 |
| | | kinase | |
| 17087623 | Galdieria sulphuraria | Serine/threonine | Gasu_38210 |
| | | kinase | |
| 17041755 | Coccomyxa subellipsoidea C-169 | Putative blue light | COCSUDRAFT_63287 |
| | | receptor | |
| 17350696 | Chlorella variabilis | Hypothetical protein | CHLNCDRAFT_141214 |
| 5005771 | Ostreococcus lucimarinus | Hypothetical protein | OSTLU_40751 |
| | CCE9901 | | |
| 17304390 | Guillarida theta CCMP2712 | Hypothetical protein | GUITHDRAFT_162563 |
| 7452793 | Thalassiosira pseudonana | Hypothetical protein | THAPSDRAFT_33193 |
| | CCMP1355 | | |
| 7442442 | Thalassiosira pseudonana | Hypothetical protein, | THAPSDRAFT_261631 |
| | CCMP1355 | PAS domain | |
| 7200921 | Phaeodactylum tricornutum CCAP | Hypothetical protein; | PHATRDRAFT_51933 |
| | 1055/1 | one PAS domain | |
| CBJ25875 | Ectocarpus siliculosus | aureochrome 1 | AUR1; Esi_0017_0027 |
| | CCAP: 1310/4 | | |
| XP_005854445 | Nannochloropsis gaditana | PAS and BZIP | GA_0015702 |
| | CCMP526 | domain containing | |
| | | protein, putative | |
| | | aureochrome | |
| BAF91488 | Vaucheria frigida | aureochrome1 | AUREO1 |

### Alternative Targets

Additional PHOT downstream signal transduction targets can be use as alternatives to the knockout or reduction in phot expression to generate the desirable phenotypes of this invention, including but not limited to improved photosynthetic efficiency, higher biomass productivity, increase yield of sink molecules/compounds, and improved genetic stability. An example of this could be the algal gene homologous to the Arabidopsis KIN10 and KIN11 kinases (Baena-Gonzalez, Rolland et al. 2007). Genes substantially homologous to the Chlorella genes in SEQ ID 15 to 27 and the Chlamydomonas genes in SEQ ID 28-34 would be applicable to this current invention.

Additional gene targets can be used as alternatives to the knockout or reduction in phot expression to generate the desirable phenotypes of this invention with desirable phenotypes having but not limited to improved photosynthetic efficiency, higher biomass productivity, increase yield of sink molecules. These genes could include the algal genes homologous to the Arabidopsis NADPH thioredoxin reductase C (NTRC) and NADPH thioredoxin reductase 2 genes (Toivola et al. 2013) Genes substantially homologous to the Chlorella genes in SEQ ID NO 35-40, 43-44 and 47 to 50 and the Chlamydomonas genes in SEQ ID 67-68 would be applicable to this current invention

TABLE 2
Sequence ID and Type

| Sequence No. ( ) | protein/dna(<212>); Organism/Strain(<213>)/protein |
|---|---|
| 1 | <212> PRT |
| | <213> Chlorella sorokiniana, strain 1412; phototropin A |
| 2 | <212> DNA |
| | <213> Chlorella sorokiniana, strain 1412; phototropin A |
| 3 | <212> PRT |
| | <213> Chlorella sorokiniana, strain 1412; phototropin B |
| 4 | <212> DNA |
| | <213> Chlorella sorokiniana, strain 1412; phototropin B |
| 5 | <212> PRT |
| | <213> Chlorella sorokiniana, strain 1230; Phototropin A |
| 6 | <212> DNA |
| | <213> Chlorella sorokiniana, strain 1230; Phototropin A |
| 7 | <212> PRT |
| | <213> Chlorella sorokiniana, strain 1230; phototropin B |
| 8 | <212> DNA |
| | <213> Chlorella sorokiniana, strain 1230; phototropin B |
| 9 | <212> PRT |
| | <213> Chlorella sorokiniana, strain 1228; Phototropin A |
| 10 | <212> DNA <213> Chlorella sorokiniana, strain 1228; phototropin A |
| 11 | <212> PRT |
| | <213> Chlorella sorokiniana, strain 1228; phototropin B |
| 12 | <212> DNA |
| | <213> Chlorella sorokiniana, strain 1228; phototropin B |
| 13 | <212> PRT |
| | <213> Picochlorum soloecismus, strain DOE101, phototropin |
| 14 | <212> DNA |
| | <213> Picochlorum soloecismus, strain DOE101; phototropin |
| 15 | <212> PRT |
| | <213> Chlorella sorokiniana, strain 1228; KIN11 SNF1-related |
| 16 | <212> DNA |
| | <213> Chlorella sorokiniana, strain 1228; KIN11 SNF1-related |
| 17 | <212> PRT |
| | <213> Chlorella sorokiniana, strain 1228; KIN11 SNF1-related protein kinase |
| | catalytic subunit alpha |
| 18 | <212> DNA <213> Chlorella sorokiniana, strain 1228; KIN11 SNF1-related protein kinase |
| | catalytic subunit alpha |
| 19 | <212> PRT |
| | <213> Chlorella sorokiniana, strain UTEX 1230; KIN11 SNF1-related protein kinase |
| | catalytic subunit alpha |
| 20 | <212> DNA |
| | <213> Chlorella sorokiniana, strain UTEX 1230; KIN11 SNF1-related protein kinase |
| | catalytic subunit alpha |
| 21 | <212> PRT |
| | <213> Chlorella sorokiniana, strain UTEX1230; KIN11 SNF1-related protein kinase |
| | catalytic subunit |
| 22 | <212> DNA |
| | <213> Chlorella sorokiniana, strain UTEX 1230; KIN11 SNF1-related protein kinase |
| | atalytic subunit |
| 23 | <212> PRT |
| | <213> Chlorella sorokiniana, strain 1412; KIN11 SNF1-related protein kinase |
| | catalytic subunit |
| 24 | <212> DNA |
| | <213> Chlorella sorokiniana, strain 1412; KIN11 SNF1-related protein kinase |
| | catalytic subunit |
| 25 | <212> PRT |
| | <213> Chlorella sorokiniana, strain 1412; KIN11 SNF1-related protein kinase |
| | catalytic subunit homolog |
| 26 | <212> DNA |
| | <213> Chlorella sorokiniana, strain 1412; KIN11 SNF1-related protein kinase |
| | catalytic subunit homolog |
| 27 | <212> PRT |
| | <213> Chlamydomonas reinhardtii; SNF-1 KIN10/11 homolog |
| 28 | <212> DNA |
| | <213> Chlamydomonas reinhardtii; SNF-1 KIN10/11 homolog |
| 29 | <212> PRT |
| | <213> Chlamydomonas reinhardtii; SNF-1 KIN10/11 homolog |
| 30 | <212> DNA |
| | <213> Chlamydomonas reinhardtii; SNF-1 KIN10/11 homolog |
| 31 | <212> PRT |
| | <213> Chlamydomonas reinhardtii; SNF-1 KIN10/11 homolog |
| 32 | <212> DNA |
| | <213> Chlamydomonas reinhardtii; SNF-1 KIN10/11 homolog |
| 33 | <212> PRT |
| | <213> Chlamydomonas reinhardtii; SNF-1 KIN10/11 homolog |
| 34 | <212> DNA |
| | <213> Chlamydomonas reinhardtii; SNF-1 KIN10/11 homolog |
| 35 | <212> DNA |
| | <213> Chlorella sorokiniana, strain UTEX 1230; NTR2 |
| 36 | <212> PRT |
| | <213> Chlorella sorokiniana, strain UTEX 1230; NTR2 |
| 37 | <212> DNA |
| | <213> Chlorella sorokiniana, strain 1412; NTR2 |
| 38 | <212> PRT |
| | <213> Chlorella sorokiniana, strain 1412; NTR2 |
| 39 | <212> DNA |
| | <213> Chlorella sorokiniana, strain 1228; NTR2 |
| 40 | <212> PRT |
| | <213> Chlorella sorokiniana, strain 1228; NTR2 |
| 41 | <212> DNA |
| | <213> Picochlorum soloecismus, strain DOE101; NTR2 |
| 42 | <212> PRT |
| | <213> Picochlorum soloecismus, strain DOE101; NTR2 |
| 43 | <212> DNA |
| | <213> Chlorella sorokiniana, strain 1228; NTRC |
| 44 | 212> PRT <213> Chlorella sorokiniana, strain 1228; NTRC |
| 45 | <212> DNA |
| | <213> Picochlorum soloecismus, strain DOE101; NTRC |
| 46 | <212> PRT |
| | <213> Picochlorum soloecismus, strain DOE101; NTRC |
| 47 | <212> DNA |
| | <213> Chlorella sorokiniana, strain UTEX 1230; NTRC |
| 48 | <212> PRT |
| | <213> Chlorella sorokiniana, strain UTEX 1230; NTRC |
| 49 | <212> DNA |
| | <213> Chlorella sorokiniana, strain 1412; NTRC |
| 50 | <212> PRT |
| | <213> Chlorella sorokiniana, strain 1412; NTRC |
| 51 | <212> PRT |
| | <213> Chlorella variabilis; phototropin A |
| 52< | <212> PRT |
| | <213> Chlamydomonas reinhardtii, strain CC-503; phototropin |
| 53 | <212> PRT |
| | <213> Botryococcus terribilis; phototropin A homolog |
| 54 | <212> PRT |
| | <213> Tetraselmis striata; phototropin A |
| 55 | <212> PRT |
| | <213> Micromonas pusilla, strain CCMP 1545; phototropin A |
| 56 | <212> PRT |
| | <213> Dunaliella salina; phototropin A |
| 57 | <212> PRT |
| | <213> Chlorella variabilis; phototropin B homolog |
| 58 | <212> PRT |
| | <213> Haematococcus lacustris; phototropin B homolog |
| 59 | <212> PRT |
| | <213> Tetraselmis striata; phototropin B homolog |
| 60 | <212> PRT |
| | <213> Coccomyxa subellipsoidea, strain C-169; phototropin B homolog |
| 61 | <212> PRT |
| | <213> Micromonas pusilla, strain CCMP1545; phototropin B homolog |
| 62 | <212> PRT |
| | <213> Vaucheria frigida; aureochrome1 |
| 63 | <212> PRT |
| | <213> Fucus distichus; AUREOChrome-like protein |
| 64 | <212> PRT |
| | <213> Nannochloropsis gaditana; aureochrome1-like protein |
| 65 | <212> PRT |
| | <213> Nannochloropsis gaditana; aureohrome1-like protein |
| 66 | <212> PRT |
| | <213> Sargassum fusiforme; putative aurochrome, LOV domain-containing protein |
| 67 | <212> PRT |
| | <213> Chlamydomonas reinhardtii; NTR2 |
| 68 | <212> PRT |
| | <213> Chlamydomonas reinhardtii; NTRC |

Below are SEQ ID NO 69-128
SEQ ID NO: 128
>KT321711.1 Mesotaenium endlicherianum phototropin (PHOT) mRNA
GACCTCAAGGACGTTCTCACAGCTTTCCAACAGACATTTGTGCTGTCTGATGCCGCCAAACCGGATAGTC
CGATTATGTTTGCCAGCGAGGGGTTCTACAACATGACGGGTTACACTCCCAAGGAAGTCATTGGCTACAA
TTGCCGCTTTCTTCAAGGGCCAGACACAGACCGCAACGAGGTGGCGCGGCTGAAGCAGGCCCTGGCTGCA
GGAGAGAGCTACTGCGGCCGCCTGCTCAACTACAAGAAGGACGGCACCCCCTTCTGGAACCTGCTCACAG
TGTCGCCTGTCAAGGACGACAATGGCCGTGTCGTTAAGTTTGTTGGCATGCAAGTGGAGGTGTCCAAGTA
CACGGAGGGCACCAAGGACCAGGACGTGCGCCCCAACAACATGCCCGTCTCCCTCATCAAATACGACGCT
CGGCAGCGCGAGGTGGCGTCCAGCATGGTGGGCGAGCTCGTGGAGACGGTCAAGAAGCCCGGCGCTGAGG
AGAGCGGCGGCGGCCTCGCGCCGCTCTATGCGCTGCCCGTGGCCGAGGGCGGCGCCGGTCAGAGCGGTGC
CGGCGCCGGCTCCTCCTCCATGCCGGCCGCGCTCACGCCCAAGAACGCGCGCCGCACCTCCGGCTTCCGC
TCCCTTCTTGGCATGAAGGGCGGCAAGCCCGACGAGGGCGGCGAGCCTGACCGCGTCGCCGCCGTTCCCG
AGGTGGTGGAGGAGGTGGAGGTGGGCGACGTGGAGCGCAAGGCGCGGCGCGGGATCGACCTGGCCACCAC
GCTGGAGCGTATCCAGAAGAACTTTGTCATCACCGACCCCCGCCTCCCCGAGAATCCCATCATCTTTGCC
TCCGACGACTTCCTGGAGCTCACGGAGTACTCGCGCGAGGACATCCTGGGGAAGAATTGCCGGTTCTTGC
AAGGGCCGGAGACGAACCGCGACACAGTGAAGAAGATCCGCGACGCCATCGACGCGGGCCAGGACATCAC
AGCGCAGCTGCTCAACTACACCAAGAGCGGCAAGAAGTTCTGGAACCTGTTCCATCTGCAGGCCGTGCGC
GACAACAAGGGCGAGCTGCAGTACTTCATCGGAGTGCAGCTGGATGCCAGCCAGTACGTGGACCCCGACG
CGCGCCGCCTGCCCGACGCCAACGTGAACGAGGGCACCAACATGATCGTGGATGCGTCCAACAAGATCGA
CGGCGCCCTCAAGGAGCTGCCTGATGCTGGCGCTACAAAGGAGGACCTGTGGGCCATCCACAGCCTGCCA
GCTGTGCCCAAGCCTCACAAGGTGCAGGACCCCCTGTGGACCGCCATCAACCAGGTGAAGCAGCGGGAGG
GCAAGCTGGGGCTGAAGCACTTCCGGCCCATCAAGCCGCTGGGCTGCGGCGACACGGGCAGAGTGCACCT
GGTGGAGCTGCGCGACACCGGCAAGCTGTTTGCCATGAAGGCCATGGACAAGGAGGTCATGATCAACCGC
AACAAGGTGCACCGCGCGTGTACTGAGCGCGAGATTCTGGGCCGCATCGACCACCCCTTCCTCCCCACCC
TCTACGCCTCCTTCCAGACGGCCACGCACGTGTGCCTCATCACGGACTTCTGCGACGGGGGCGAGCTCTA
CATGCTGCTGGAGCGTCAGAAGGGCAAGCGCTTCGCCGAAGAGGCTGTCCGCTTCTTTGGGTCCGAGATC
CTGCTGGCGCTGGAGTACCTGCACTGCCAGGGCGTAATCTACCGCGACCTCAAGCCCGAGAACATCCTGC
TGACAGCTGGCGGCCACGCGCTGCTCACCGACTTCGACCTCTCGTTCCTCACCACCGCGGAGCCGCGCGT
CATCCGGCCGGAGCCCGCACCCGGCGTGAAGAAGGGCAAGAAGAAGAAGAAGGGCGAGCCCGAGCCGCGC
CCGCAGTTTGTGGCGGAGCCCGTGGCACAGTCCAACTCGTTTGTCGGCACGGAGGAGTACATTGCGCCCG
AGATCATCAGCGGCGCCGGCCACAGCAGCGCCGTCGACTGGTGGGCCTTTGGCATCTTCCTGTACGAGAT
GACGTACGGGCGCACGCCCTTCCGCGGCAAGAACAGGCAGCGCACGTTCACCAACATCCTCATGAAGGAG
CTCGCCTTCCCCACAAACCCACCCGTGAGTGCAAATGCCAAGGCGCTGATGAAGGCTCTGCTGGAGCGCG
ACCCCGCGGTGAGGCTGGGAGGGACACGTGGCGCGTCGGAGATCAAGGAGCACCCCTTCTTCGAGTCCAT
CGACTGGGCCCTCGTCCGCCACAAGGGAGGGCCGAGCCTGGACGTGCCCATCAAGAAGATCGGCACAGAC
CCCGACACGAGCCGCGCTTCCATCAGCAGCGAGGCCACGGAGGACCTCGACTGGGACGACCAGGAGGCGC
TCACGCCCTCCACCAACCGCTCCATGGAGTACGGCTACCAGTAG
SEQ ID NO: 69
>ANC96836.1 phototropin, partial [Mesotaenium endlicherianum]
DLKDVLTAFQQTFVLSDAAKPDSPIMFASEGFYNMTGYTPKEVIGYNCRFLQGPDTDRNEVARLKQALAA
GESYCGRLLNYKKDGTPFWNLLTVSPVKDDNGRVVKFVGMQVEVSKYTEGTKDQDVRPNNMPVSLIKYDA
RQREVASSMVGELVETVKKPGAEESGGGLAPLYALPVAEGGAGQSGAGAGSSSMPAALTPKNARRTSGFR
SLLGMKGGKPDEGGEPDRVAAVPEVVEEVEVGDVERKARRGIDLATTLERIQKNFVITDPRLPENPIIFA
SDDFLELTEYSREDILGKNCRFLQGPETNRDTVKKIRDAIDAGQDITAQLLNYTKSGKKFWNLFHLQAVR
DNKGELQYFIGVQLDASQYVDPDARRLPDANVNEGTNMIVDASNKIDGALKELPDAGATKEDLWAIHSLP
AVPKPHKVQDPLWTAINQVKQREGKLGLKHFRPIKPLGCGDTGRVHLVELRDTGKLFAMKAMDKEVMINR
NKVHRACTEREILGRIDHPFLPTLYASFQTATHVCLITDFCDGGELYMLLERQKGKRFAEEAVRFFGSEI
LLALEYLHCQGVIYRDLKPENILLTAGGHALLTDFDLSFLTTAEPRVIRPEPAPGVKKGKKKKKGEPEPR
PQFVAEPVAQSNSFVGTEEYIAPEIISGAGHSSAVDWWAFGIFLYEMTYGRTPFRGKNRQRTFTNILMKE
LAFPTNPPVSANAKALMKALLERDPAVRLGGTRGASEIKEHPFFESIDWALVRHKGGPSLDVPIKKIGTD
PDTSRASISSEATEDLDWDDQEALTPSTNRSMEYGYQ
SEQ ID NO: 70
>AB206963.1 Mougeotia scalaris PHOTA mRNA for phototropin
TTTGACATCTAAACGGGCAGTTACGCTTCACGGTTAAAGAGTTTTCGATACTACGGAGGTAACTTTTCCA
CGACCCAGTTTTCACCTGCTTCACCCGCCTGTATTAAAGAAACGTTGTCTTCTCTTTCGTTCAGAGCATG
GCGGCATTAGTCAACCTTCCTATTTCGAGGTATCCTCAGCCCTTACTTGGAGAAGGGGTTGATGTCATTC
ATAAATCCGAAAAAGTCCTGGGTGAAGCTTCCCAGGGCCTGAAAGATGCCCTCACGGCTTTCCAACAGAC
ATTTGTAATGTGTGATGCCACAAAGCCAAACACTCCCGTCATGTTTGCCAGTGAGGGTTTCTACAGGATG
ACTGGCTACAGTGCTAAAGAGGTTATTGGCAAAAACTGTCGCTTCCTCCAAGGTCCCGAGACTGACCGCA
GTGAGGTGGAGAAGTTGAAGCAAGCACTTTTGGATGGTCAGTCATGGTGTGGCCGACTTCTGAACTACAG
GAAAGATGGTAGCAGTTTCTGGAACCTTCTTACAGTCTCTCCCGTAAAGGATGACAGTGGGAGAGTTGTG
AAATTTATCGGGATGCAGGTGGAGGTGTCTAAGTTTACAGAAGGAAAGAATGATGACATCAAGCGGCCCA
ATCAGCTCCCTGTCTCCCTGATTCGTTATGATGATAGGCAGAAGGATGAAGCAGAAGTCAGAGTGGAGGA
ACTACTGCAGGACATGAAGGAATCAGAATCACCAGCAGAGGTAGAAGCCAAGGTGCAAACAGTTCAGGTT
AGCGTGCCAGCTCAGCCCAGCAAGCTGTCAAAGGAGGCACCTGCAGAGACAAAGAAGACTCGCAGATCTT
CTTACTTTGGGAAGAATGCGGCTCCAAAGGCTGAAGAAGTACCCCCAGTCTTCGAGCCAGGAGTGGAAGT
CAGCCTGCTGATGGAAGACGAGCTGGATACCATGGCGGTAGAAAAGAAGCACAGACATGGTATCGATCTG
GCCACTACTTTGGAACGAATCCAGAAGAACTTTGTCATTACAGATCCGAGGCTTCCTGACAACCCAATCA
TTTTTGCGTCTGACGATTTCTTGGAGCTAACTGAGTACACTCGCGAGGAGATCATTGGTCGGAATTGTCG
ATTTCTGCAAGGAAAGGACACAGACAAAGAGACAGTAGCCAAAATCAGACATGCCATCGATAACCATCAA
GATATCACCGTGCAGCTACTCAATTACACCAAGAGTGGAAAGCCGTTCTGGAACTTATTCCATCTCCAGG
CTGTCAGGGACACCAAGGGTCGGTTGCAATACTTCATTGGAGTGCAGCTGGATGCCAGCACATATGTGGA
GCAGGCTTCAAAGAACATTCCAGATAATCTGAAGAAGATGGGGACAGAGGAGATCCACAACACTGCAAAT
AACGTCGACTTTGGACTGAAAGAGCTCCCGGATACAAACACAGGAAATAAGGACGATATCTGGACTCTAC
ACTCAAAGCAAGTCACTGCACTGCCCCACAAAAGCAACACTGAGAACTGGGATGCCATTCGCAAGGTAAT
TGCTTCAGAGGGGCAGATATCCCTGAAGAACTTCCGGCCGATAAAGCCCCTCGGGTACGGAGACACGGGG
AGTGTCCACCTGGTGGAGCTCCGTGATTCCGGAGTGTTCTTTGCCATGAAGGCCATGGACAAGGAGGTGA
TGGTCAACAGAAATAAGGTCCATCGAGCGTGCACAGAGCGGGAGATTCTGGAGCTTCTGGACCATCCGTT
CCTGCCGACGCTCTACGGATCCTTCCAGACACCCACCCATGTCTGCCTGATCACCGACTTCTGTCCCGGG
GGGGAGCTGTTTGCCCACCTGGAGAATCAGAAACAGAAACGGCTCAAGGAGAATGTGGCCAAGGTGTACG
CTGCGCAGATCCTGATGGCACTCGAGTACCTGCACCTGAAGGGAGTCATCTATCGAGATCTGAAGCCGGA
GAACATCCTCATCTGTGAAGGGGGGCATCTGCTGCTGACCGACTTCGACCTGTCATTCAGGACAGAGACA
GAAGTGAAGGTGGCCATGGTGCCCATTCCTGAGGAGGAGGGGGCACCTGTCGTCGAGAAGAAGAAGAAGA
AGAAAGGGAAGGCCCCTGCAGCTGCTGCCATGGCTCCCAGGTTCATCCCCCAGCTGGTTGCCGAACCGTC
AGGCACCAGCAACTCCTTTGTGGGCACAGAGGAGTACATCGCACCGGAGATTATCAGCGGAGTCGGCCAT
GGCAGCCAGGTGGATTGGTGGGCGTTTGGCATTTTTATCTATGAAATGTTGTACGGGAAGACGCCGTTCC
GAGGGAAGAATCGGAAGCGGACTTTCACAAATGTGCTGACCAAGGAGCTGGCGTATCCCACCGTCCCTGA
AGTGAGCCTGGATGTGAAGCTTCTCATCAAGGATCTTCTGAATCGCGATCCGTCTCAGCGACTGGGTGCC
ACTCGGGGGGCGTCTGAGATCAAGGAGCATCCATGGTTCAATGCCATTCAATGGCCTCTTATTTGCAAGG
ATGTGCCAGAATCAGACGTTCCTGTCAAGTTTATGCAGGTGGAGAATGAGCGCAGGGACTCCACTGCGGA
TGATGATGCTGACTGGGAGTCTAATGATGGTCGCAATTCTCTGTCGCTTGATCTGGGCAGGCAGTAGTTG
GTGGGTAGAGGGTTCGTTTGTTGGAGTTTCGTAGGTTGGTGTATGGACTTGTAGTTGGTTAGAGTCAGGA
ACAAACAAAGTTAGACCTATTGGTTTGAATAGTAACTTTATATGGAATTTTGTATTGTCCGGTTTTGAAT
ATTAGAACCTTTTTAATGGTATTCCAACATTCTGGTTTCAAAAAAAAAAAAAAAAAAA
SEQ ID NO: 71
>BAE20160.1 phototropin [Mougeotia scalaris]
MAALVNLPISRYPQPLLGEGVDVIHKSEKVLGEASQGLKDALTAFQQTFVMCDATKPNTPVMFASEGFYR
MTGYSAKEVIGKNCRFLQGPETDRSEVEKLKQALLDGQSWCGRLLNYRKDGSSFWNLLTVSPVKDDSGRV
VKFIGMQVEVSKFTEGKNDDIKRPNQLPVSLIRYDDRQKDEAEVRVEELLQDMKESESPAEVEAKVQTVQ
VSVPAQPSKLSKEAPAETKKTRRSSYFGKNAAPKAEEVPPVFEPGVEVSLLMEDELDTMAVEKKHRHGID
LATTLERIQKNFVITDPRLPDNPIIFASDDFLELTEYTREEIIGRNCRFLQGKDTDKETVAKIRHAIDNH
QDITVQLLNYTKSGKPFWNLFHLQAVRDTKGRLQYFIGVQLDASTYVEQASKNIPDNLKKMGTEEIHNTA
NNVDFGLKELPDTNTGNKDDIWTLHSKQVTALPHKSNTENWDAIRKVIASEGQISLKNFRPIKPLGYGDT
GSVHLVELRDSGVFFAMKAMDKEVMVNRNKVHRACTEREILELLDHPFLPTLYGSFQTPTHVCLITDFCP
GGELFAHLENQKQKRLKENVAKVYAAQILMALEYLHLKGVIYRDLKPENILICEGGHLLLTDFDLSFRTE
TEVKVAMVPIPEEEGAPVVEKKKKKKGKAPAAAAMAPRFIPQLVAEPSGTSNSFVGTEEYIAPEIISGVG
HGSQVDWWAFGIFIYEMLYGKTPFRGKNRKRTFTNVLTKELAYPTVPEVSLDVKLLIKDLLNRDPSQRLG
ATRGASEIKEHPWFNAIQWPLICKDVPESDVPVKFMQVENERRDSTADDDADWESNDGRNSLSLDLGRQ
SEQ ID NO: 127
>KJ195120.1 Cylindrocystis cushleckae phototropin (PHOTA) mRNA
ATGGCGAGAATACCCCAGTCAAATTATCCTGCGAGGCTGAGTGATGTATCATCCACTCCAGGCGCTGGCA
AGGTGCTTGGTCAGGCCTCTGAAGGACTGAAGGATGTGCTCACTACGTTCCAGCAGACATTTGTTATGTG
TGATGCTACCAAACCTGACATTCCTGTCATGTTTGCCAGTGAGGGATTTTACGAAATGACTGGCTACAAT
GCCAAGGAAGTGATTGGCAAGAATTGCCGTTTCCTCCAAGGTACAGAAACAGACCGTGCTGAGGTGGCAA
AAATGAAGCAGGCCCTCATGGCCGGCGAGGGTTGGTGTGGCCGCCTTCTCAACTACCGAAAAGATGGAAC
TCCCTTCTGGAATCTTCTTACCGTATCGCCCGTGAAGGACGACAATGGGAGGGTGGTCAAGTTCATTGGA
ATGCAGGTGGAGGTTACCAAGTTCACGGAAGGCAAACAGGACGAGAATAAGCGCCCAAACCAGCTTCCGG
TCTCTCTCATTCGCTATGATGCTCGGCAGAAGGAGGAGGCTGAGCTTGGCGTCCAGGAGCTGGTGCACGC
AGTGCAGCGCCCCAAGCAGGGGGGTGGGATGGACAGCCTCATGGCCCTTCCCAAGGCGGGCGAGATGCCA
GCCTCAGAGCTGGAGGCAGAAACCCCCGGAAAGAAGAAGGGCAGGCGTGCATCGGGCATGAAAATGTTTG
GGGGAAAAGACAAGGCCCAGGAGGCAGAGCCGGAGGTGGAAACAGTAGACAGCGACGACGAGATCTCAGA
GAAGAAGCAACGTCACGGAATCGACCTGGCCACTACCCTGGAGCGTATTCAGAAAAATTTCGTCATCACG
GATCCTCGCCTGCCCGACAACCCCATTATCTTTGCATCCGACGACTTTCTGGAGCTTACGGAATACTCTC
GCGAGGAGGTGCTGGGCCGGAATTGTCGGTTCCTGCAAGGCAAGGACACAGACCGTGCCACTGTGGCCCG
CATCAGGGACGCCATCGATAACGCGCAGGACATCACTGTGCAGCTCCTCAATTACACCAAAAGCGGCAAA
CCTTTCTGGAACCTGTTCCACTTGCAAGCTGTGCGGGATAGCAAGGGTCAACTGCAGTACTTCATCGGAG
TTCAGCTGGACGCAAGCACATACGTTGAGCCCGTCACTCACGAGCTTCCCCAGAAGACCAAAACAGAGGG
CACTGAGGAGATCGTGAACACGGCCAACAATATCGATGTGGGGCTCAAGGAACTTCCCGACCCAAACAAT
AAAAAAGATGACATGTGGAACGGCCACTCCCAGGAGGTCTCCCCCCTTCCCCACCGCGTTGGCGACCCCA
GCTGGGAGGCTGTCCAGAAGGTCAAGGCCAGCGATGGTCGCCTGGCTCTGAAACATTTCCGGCCAATCAA
ACCCCTCGGTTGTGGAGACACAGGTAGCGTCCACCTTGTCGAGCTTCGCGATACGGGAAAACTTTTCGCC
ATGAAGGCTATGGACAAGGACGTGATGATCAATCGCAACAAGGTCCACAGAGCGTGCACCGAGCGCCAAA
TCTTGGGCGATCTCGACCATCCGTTCCTCCCCACACTCTACGGATCCTTCCAGACGGCCACCCACGTCTG
CCTCATCACCGACTTCTGTCCGGGCGGCGAACTCTACACCCACCTGGAGCACCAGAAGGGGAAAAGGTTT
CCTGAAGCTGCGGCAAAATTTTACGCTGCCGAGATTCTTCTGAGTTTGGAATACCTCCACTGCAAGGGCG
TGATTTACCGCGATCTCAAGCCAGAGAACATTCTCATCACCTCCTCGGGACACCTGGTGTTGACCGACTT
TGACCTGTCCTTCCTCAGCTCCACTATCCCCCAGCTCCTGAGGCCCAACCCCACAGAGGTGAGCGGCAAG
AAGAAGAAGAAGGGCAAGGGGGCGGCGCAGCCCTTGCCGCAGTTTGTGGCGGAGCCCACAGGGAGCAGCA
ACTCCTTCGTGGGCACAGAGGAGTACATCGCGCCGGAGATTATCAGCGGCACGGGCCACAGCAGCCAGGT
GGACTGGTGGGCTTTTGGCATCTTCGTGTATGAGATGCTGTACGGCAAGACCCCCTTCCGCGGGCGCAAC
CGCCAAAAGACCTTCACCAATGTGCTGATGAAAGAGCTGGCCTTCCCCAACAGCCCCCCCGTAAGTCTGG
AGGCCAAGCTCCTGATCAAGGCGCTGCTCACCCGGGATCCCCAGCAGCGCCTGGGCTCCGCGCGCGGCGC
CAGCGAGATCAAGGACCACCCCTGGTTTGCTGGGGTCAACTGGGCCCTCACCCGCTCCCAGCCCCCCCCC
GAGCTGGAGGTCCCGGTCACCTTCACCAGCGGCGAGCCCGACACGCACCGCCCGTCAACCACAGACGAAG
ACCTGGAGTGGGATAGCAACGAAGCACGGGACTCCAGCTCATCACTCTCATTTGACCAGAGCTAA
SEQ ID NO: 72
>AHZ63921.1 phototropin [Cylindrocystis cushleckae]
MARIPQSNYPARLSDVSSTPGAGKVLGQASEGLKDVLTTFQQTFVMCDATKPDIPVMFASEGFYEMTGYN
AKEVIGKNCRFLQGTETDRAEVAKMKQALMAGEGWCGRLLNYRKDGTPFWNLLTVSPVKDDNGRVVKFIG
MQVEVTKFTEGKQDENKRPNQLPVSLIRYDARQKEEAELGVQELVHAVQRPKQGGGMDSLMALPKAGEMP
ASELEAETPGKKKGRRASGMKMFGGKDKAQEAEPEVETVDSDDEISEKKQRHGIDLATTLERIQKNFVIT
DPRLPDNPIIFASDDFLELTEYSREEVLGRNCRFLQGKDTDRATVARIRDAIDNAQDITVQLLNYTKSGK
PFWNLFHLQAVRDSKGQLQYFIGVQLDASTYVEPVTHELPQKTKTEGTEEIVNTANNIDVGLKELPDPNN
KKDDMWNGHSQEVSPLPHRVGDPSWEAVQKVKASDGRLALKHFRPIKPLGCGDTGSVHLVELRDTGKLFA
MKAMDKDVMINRNKVHRACTERQILGDLDHPFLPTLYGSFQTATHVCLITDFCPGGELYTHLEHQKGKRF
PEAAAKFYAAEILLSLEYLHCKGVIYRDLKPENILITSSGHLVLTDFDLSFLSSTIPQLLRPNPTEVSGK
KKKKGKGAAQPLPQFVAEPTGSSNSFVGTEEYIAPEIISGTGHSSQVDWWAFGIFVYEMLYGKTPFRGRN
RQKTFTNVLMKELAFPNSPPVSLEAKLLIKALLTRDPQQRLGSARGASEIKDHPWFAGVNWALTRSQPPP
ELEVPVTFTSGEPDTHRPSTTDEDLEWDSNEARDSSSSLSFDQS
SEQ ID NO: 73
>KJ195119.1 Zygnemopsis sp. MFZO phototropin (PHOTA) mRNA
ATGGCTAGTCTTCCCCCTTCTCGCTATCCTGCCCGGTTAAACAATGAGGCTCCATTGCCGACAGCAAGCA
AAGTGCTGGGACAGGCCTCCGAAGGGCTCAAGGATGTGCTGACCACCTTCCAGCAGACCTTTGTGATGTG
TGATGCGACAAAGCCCGACATACCTGTAATGTTTGCCAGCGAAGGTTTTTACGAGATGACCGGATACACC
GCCAAAGAGGTCATCGGCAAGAACTGTCGGTTTCTGCAGGGGCCGGAAACGGACAAGGCTGAGTTGGGCA
AACTGAAGCAGGCCCTGATGGCCGGCGAGGGGTGGTGCGGCCGGCTGCTCAACTACCGCAAGGACGGCAC
TCCCTTCTGGAACCTGCTCACCATCTCCCCCGTCAAGGACGACAATGGCAGGGTGGTGAAATTCATCGGA
ATGCAAGTGGAGGTGACCAAGTTCACAGAAGGCAAGCAGGATGAGAACAAGCGGCCCAACCAGTTGCCCG
TGTCGCTCATTCGCTATGATGCTCGCCAGAAGGAGGAGGCCGAGCTGGGCGTGCAGGAGCTGGTGGACGC
GGTGCAGAAGCCGGCGATCAAGCAGGGTGGGGGCATGGAGAGCCTGATGGCGCTGCCCAAGGTGGAGGAG
ACCCCCGCGTCTCCCGACACTCCGGGGAGGAAGAAGGGCAAGCGCTCGTCCCTGCTGCTCTCACGCCTCA
GTGTGTCGTCCAGGCAGGCGCCCAAGCCCGAAGACTTGATCACGACTGAGGAGGACAAGCGGGACAGCTT
TGACGACATGTCGGAGAAGAAGCAGCGCCACGGCATCGACCTGGCCACCACTCTGGAGCGCATCCAGAAG
AACTTTGTCATCACAGATCCCAGACTGCCGGATAACCCCATTATTTTCGCCTCCGATGATTTCCTGGAGC
TCACCGAGTACAGCCGAGAGGAGGTCTTGGGCCGCAACTGTCGGTTTCTGCAGGGCAAGGACACCGACCG
CAACACGGTGGCCAAGATCCGGGCAGCCATTGACAGCCAGCAGGATATCACGGTCCAGCTGCTCAACTAC
ACCAAGAGCGGCAAGCCTTTCTGGAATCTCTTTCATCTGCAAGCCGTCCGTGATAGCAAGGGTCAGCTCC
AGTACTTCATTGGAGTGCAGCTGGACGCCAGCACGTACATCGAGCCCAGCTCGAAGCAGCTGCCTGAGCA
AACAGCCCTGCAGGGAACTGAGGAGATTGTGAACACTGCCCACAACGTCGATGTGGGATTGAAGGAGCTG
CCAGATGCGAATGCGCCCAAGGAGGACCTGTGGGCCGCACACTCCAAGCCCGTGTCAGCGCGGCCGCACC
ACCTGCTGGACCCCAACTGGGCGGCCATTGAACAGATCAAGGCCAAGGATGGCCGCCTGGGCCTGAAGCA
TTTCCGACCCATCAAGCCCCTCGGATGCGGAGACACCGGCAGCGTCCATCTTGTGGAGCTGCGCGATTCC
GGCAAGCTGTTTGCCATGAAGGCCATGGACAAAGAAGTGATGATTAACCGCAACAAGGTGCATCGCGCCT
GCACCGAGCGTCAGATCTTGGAAGATCTGGACCATCCGTTCTTGCCCACTCTGTACGGGTCGTTCCAGAC
GGCCACTCACGTCTGCTTGATCACTGATTTCTGCCCTGGGGGGGAGCTCTACGCCCACCTCGAGAACCAG
AAGGGCAAGAGGTTCCCCGAAGAGGTGGCCAAGTTCTACGCCGCAGAGATCCTCCTGAGTCTGGAGTACT
TGCATTGCCGCGGCGTCATCTACCGCGACCTCAAGCCCGAGAACATCCTCATCACAGAGACCGGCCACCT
GCTGTTGACCGATTTCGACCTTTCCTTCCTGAGCACCACCACTCCCAAGCTTCTGAGGCCCAGCCCCGTG
GAAAGCCCCGTGGGGAAGAAGAAGTCGAGGAAGAGCAGCAAGAATAGCGAGCCCCCGCCCCTGCCCCAGT
TTGTGGCTGAACCCTCCGGCAGCAGCAACTCGTTCGTGGGAACGGAGGAGTACATTGCGCCCGAGATCAT
CAGTGGAACCGGCCACAGCAGCCAGGTGGACTGGTGGGCCCTGGGCATCTTCATGTACGAGATGCTCTAT
GGCAAGACCCCCTTCCGAGGCCGCAACCGGCAACGCACCTTCACCAACGTGCTGATGAAGGAGCTGGCCT
TCCCCAACAGCCCCCCCGTGAGCCTGGAGGCCAAGCTGCTGATCAAGGCCCTGCTGGTGCGGGACCCGCA
GCAGCGCCTGGGAGCTGCCCGGGGGGCCAGCGAGATCAAGGACCACCCGTGGTTCGCGGGGCTGCAGTGG
CCCCTCATTCGCTGCAAGAGCCCACCAGGCTGCGAGGTCCCTGTGACCTTCATCAATGCGGAGGCTGAAA
ACCACCGCACATCTGCAACAGACGAGGAGTTGGATTGGGACACCAGCGAATCGCGAGACACCAACTCCAT
GTCGTTATCCTTTGACATGGCCTAG
SEQ ID NO: 74
>AHZ63920.1 phototropin [Zygnemopsis sp. MFZO]
MASLPPSRYPARLNNEAPLPTASKVLGQASEGLKDVLTTFQQTFVMCDATKPDIPVMFASEGFYEMTGYT
AKEVIGKNCRFLQGPETDKAELGKLKQALMAGEGWCGRLLNYRKDGTPFWNLLTISPVKDDNGRVVKFIG
MQVEVTKFTEGKQDENKRPNQLPVSLIRYDARQKEEAELGVQELVDAVQKPAIKQGGGMESLMALPKVEE
TPASPDTPGRKKGKRSSLLLSRLSVSSRQAPKPEDLITTEEDKRDSFDDMSEKKQRHGIDLATTLERIQK
NFVITDPRLPDNPIIFASDDFLELTEYSREEVLGRNCRFLQGKDTDRNTVAKIRAAIDSQQDITVQLLNY
TKSGKPFWNLFHLQAVRDSKGQLQYFIGVQLDASTYIEPSSKQLPEQTALQGTEEIVNTAHNVDVGLKEL
PDANAPKEDLWAAHSKPVSARPHHLLDPNWAAIEQIKAKDGRLGLKHFRPIKPLGCGDTGSVHLVELRDS
GKLFAMKAMDKEVMINRNKVHRACTERQILEDLDHPFLPTLYGSFQTATHVCLITDFCPGGELYAHLENQ
KGKRFPEEVAKFYAAEILLSLEYLHCRGVIYRDLKPENILITETGHLLLTDFDLSFLSTTTPKLLRPSPV
ESPVGKKKSRKSSKNSEPPPLPQFVAEPSGSSNSFVGTEEYIAPEIISGTGHSSQVDWWALGIFMYEMLY
GKTPFRGRNRQRTFTNVLMKELAFPNSPPVSLEAKLLIKALLVRDPQQRLGAARGASEIKDHPWFAGLQW
PLIRCKSPPGCEVPVTFINAEAENHRTSATDEELDWDTSESRDTNSMSLSFDMA
SEQ ID NO: 75
>AB206964.1 Mougeotia scalaris PHOTB mRNA for phototropin, complete cds
CTATTGCCTACACGACACTGTGCGCCATGAATTCGCCGCTATCGCCCTCTCGCGCGATTCAAACATCGGA
AGGAAAGATCTTGGAGCAGAAATCGGAGCTCAAGGATGTTCTCACTTCGTTCCACCAGACATTTGTTATA
TCAGATGCCACTAAGCCAGACATTCCTATAGTCTTTGCTAGTGAGGGTTTTTACGAGATGACCGGATATG
GTCCAGAGGAAGTTATTGGATACAACTGCCGATTCTTACAAGGCGAGGGTACAAGTCGTGACGAGGTCAC
CCGATTGAAGCAATGCCTTGTCGAGGGACAGCCATTTTGTGGTCGATTACTGAATTATCGTAAAGATGGG
ACCCCATTCTGGAATCTCCTCACTGTGTCTCCTGTAAGGAGTGCCACTGGTAAAGTTGTTAAATTTATTG
GTATGCAAACAGAGGTTTCTAAGTTCACAGAAGGAGCCGCGGATGGTATAAAGCGCCCCAATGACCTTCC
TGTTTCCCTCATCCGATATGATGCCCGACAGAAGGACGAGGCCGAAGTCTCAGTGACAGAAATCGTGCAT
GCAGTGGCTCACCCGGAGAAGGCCATAGCCAGACTGAGCACGGCTGTCACAGAGAGCAGTAAGAAGCACC
AACAGCAGTCTGTCAGCCCTGAATTTGGCGCTGAGGGTCTGAAGACGCCATTGATCACCATCAACGAAAA
GGAGGCAGTTGACGAAGTGGAAGTTGAGGAAGAAGGAAGGGACAGTTTTGAAATTACAGGAGAGAAAAAG
ATTCGCAGGGGTCTGGACCTGGCCACTACCCTTGAACGCATTCAGAAGAACTTTGTGATTACTGACCCCA
GACTCCCAGAGAACCCAATTATTTTCGCCTCTGACGACTTCCTAGAGCTGACAGAGTATTCACGAGAGGA
AGTCATTGGTCGTAACTGCAGATTCCTTCAGGGTCCAGATACAGACCAGGACACAGTGCAGAAGATCCGT
GATGCCATCAGAGACTGCAGAGACGTGACTGTTCAGCTCCTTAACTATACAAAGAGTGGGAAGCCATTCT
GGAACATGTTCCACCTACAGGCTGTCAAGAACAGCAAGGGAGAGCTGCAGTACTTTATTGGTGTCCAGCT
GGATGCCAGCACATACATTGAACCTAAACTGCAGCCGCTTTCAGAGAGTGCAGAGAAGGAAGGCACCAAA
CAAGTGAAGACAACGGCTGACAATGTTGACTCCAGCCTGAGGGAGCTGCCAGATCCCAATGTGTCCAAAG
AAGACATCTGGGGCATCCATTCCTCCGTTGCAGAGCCAAAGCCCCATCAGAAGAGAGGATACTCGTCAAA
GTGGGATGCAGTGCTGAAGATCAAAGCCAGAGATGGAAAAATAGGACTGAAGCACTTCCGACCAGTGAAA
CCCTTGGGCTGCGGAGACACTGGAAGCGTCCATTTGGTGGAGTTGAAAGACACGGGCAAGTTCTTTGCCA
TGAAGGCCATGGACAAGGAAGTTATGATCAACAGAAATAAGGTGCACAGGACTTGCACAGAGCGGCAAGT
TTTAGGGCTGGTGGACCATCCCTTCCTGCCTACGCTGTATGCCTCATTTCAGACTACAACACACATCTGT
CTCATCACTGATTTCTGCCCTGGAGGTGAGCTGTACATGCTACTGGACAGACAGCCATCTAAGAGGTTCC
CTGAATATGCAGCCAGGTTCTATGCTGCTGAGATTCTGCTGGCACTTGAGTACCTGCACCTGCAGGGTGT
TGTGTACCGAGACCTGAAGCCAGAGAACATTCTGATTGGCTATGACGGTCACCTGATGCTCACTGACTTT
GACCTCTCCTTTGTGTCAGAAACTGTTCCTGAGTTGGTGTTCCCCCCCAATTACAATAAGGATAAGCCCA
AGAGTAAGAATAAGAAGGACAGGGAAGGAAATCTGCCTGTTCTGGTGGCGCGTCCCTCTGGGACAAGCAA
TTCTTTTGTGGGTACTGAGGAGTACATCTGCCCAGAAATAATAAGTGGAATTGGTCACAACAGCCAAGTG
GATTGGTGGTCGTTTGGTATTTTCCTTTATGAGATGCTGTATGGAAAGACACCTTTTAGAGGTCGCAATC
GGCAGCGAACATTCTCCAACGCCCTCACAAAGCAGCTGGAGTTCCCACCAACACCACATATCAGTCAAGA
GGCCAAGGATCTGATCACTCTCCTCTTAGTGAAGGACCCAAGCAAGCGACTGGGAGCCATTTTTGGTGCC
AATGAAGTCAAGCAACATCCATTTTTCCGTGACTTTGACTGGACCCTCATTCGATGCAGACAACCTCCAT
CCTTAGATGTTCCTGTCAAGTTCAACAACCATTCGCCACAACGGACTTCAGGAGATGAGGAAGAAATGGA
GTGGGATGAAGATGAGAACATAAGTACATCCACAACTGTGTCTTTGGACTTTGACTAGTCGCACATATTT
TTAGCTTATAGCACACACGTATATATAAATAATAGATACATACTTATTACATAGTAGTGTTGTATAGTAA
GCATAATATTTTTGGTAATAATGTTTTGGTTTTGGTTTTGTTTTC
SEQ ID NO: 76
>BAE20161.1 phototropin [Mougeotia scalaris]
MNSPLSPSRAIQTSEGKILEQKSELKDVLTSFHQTFVISDATKPDIPIVFASEGFYEMTGYGPEEVIGYN
CRFLQGEGTSRDEVTRLKQCLVEGQPFCGRLLNYRKDGTPFWNLLTVSPVRSATGKVVKFIGMQTEVSKF
TEGAADGIKRPNDLPVSLIRYDARQKDEAEVSVTEIVHAVAHPEKAIARLSTAVTESSKKHQQQSVSPEF
GAEGLKTPLITINEKEAVDEVEVEEEGRDSFEITGEKKIRRGLDLATTLERIQKNFVITDPRLPENPIIF
ASDDFLELTEYSREEVIGRNCRFLQGPDTDQDTVQKIRDAIRDCRDVTVQLLNYTKSGKPFWNMFHLQAV
KNSKGELQYFIGVQLDASTYIEPKLQPLSESAEKEGTKQVKTTADNVDSSLRELPDPNVSKEDIWGIHSS
VAEPKPHQKRGYSSKWDAVLKIKARDGKIGLKHFRPVKPLGCGDTGSVHLVELKDTGKFFAMKAMDKEVM
INRNKVHRTCTERQVLGLVDHPFLPTLYASFQTTTHICLITDFCPGGELYMLLDRQPSKRFPEYAARFYA
AEILLALEYLHLQGVVYRDLKPENILIGYDGHLMLTDFDLSFVSETVPELVFPPNYNKDKPKSKNKKDRE
GNLPVLVARPSGTSNSFVGTEEYICPEIISGIGHNSQVDWWSFGIFLYEMLYGKTPFRGRNRQRTFSNAL
TKQLEFPPTPHISQEAKDLITLLLVKDPSKRLGAIFGANEVKQHPFFRDFDWTLIRCRQPPSLDVPVKFN
NHSPQRTSGDEEEMEWDEDENISTSTTVSLDFD
SEQ ID NO: 77
>KJ195118.1 Cylindrocystis cushleckae phototropin (PHOTB) mRNA
ATGGGACGAGATCCGGACGTGGATCAGCTTGGTCAGAATGTGTCTGGGCTATCAGTAGAGACGAATGGAA
ATAATAGTCAGGTTGCGCGTGGTACAGGCTTGGCCACACCCGACAAAGACAAAATCTTAACACAAACCGA
AGGGCTGACAGATGTGCTCACGACATTTCAACAAACGTTTGTCATGTCTGACGCTACCAAGCCCGATATC
CCAATCACATTCGCTAGTGAGGGATTCTACAAGATGACAGGCTACAGCCCTAAGGAGGTCATCGGGCGAA
ATTGCCGTTTTCTTCAAGGTGAAGGCACCGACCGTGCAGAAGTTGCCCGCCTGAAGCAATGTCTGGTCTC
CGGGGAAAGCTTCTGCGGCCGTCTGCTGAACTACAGAAAAGATGGAACACCTTTTTGGAATCTTCTCACG
GTATCTGCTGTCAAAAATGACGATGGCAAGATCGTGAAGTTTGTCGGAATGCAAGTGGAGGTGACTAAGT
ACACAGAGGGCAAAGCGGACGAGCAGAGGCGTCCCAATGACATGCCTGTTTCTCTCATCCGCTACGACGC
TCGGCAAAAGGAGGAGGCGGAGACTTCAGTGGCAGAAATTCTTCATGCTGTCAAGTTGCCAGAGCAAGCT
AAGGCGCGTCTCAGTATGACACCTGTCCTGGACGAATCTATATCCCAGAGGGAACAGGAGGTGAGCCAAG
AAGATGCGGCCGCAAAACGGAAACGGGAACGGAGGACGTCAGGATTCATGACTCTATTAGGGAACGGGGC
CACAAAGGAGGAGCTGACACCTGTCATTTCGGAGCCTTCCACGCCCCAACCCGTAGAGAAGGAGGAGGTT
CGAGACAGTTTCGAGCTAACCGGAGAGAAAAATGGGCGGCGAGGGCTGGATCTAGCAACGACCCTTGAAC
GTATCCAGAAAAATTTTGTCATCACTGACCCTCGACTTCCCGAAAACCCAATTATTTTCGCGTCAGACGA
CTTTTTGGAGTTGACCGAGTACTCAAGAGAGGAGGTCCTGGGCAGAAACTGCAGATTCCTACAGGGCAAG
GATACTGACCAGAAAACAGTTCAGGAGATCCGGGACGCTATCCGAGAGCAGAGAGACGTCACAGTGCAGC
TGCTCAACTACACCAAGGGCGGTCGTCCCTTCTGGAACCTGTTCCATCTGCAGGCTGTCAAGGACAGCAA
GGGGGACCTGCAGTACTTCATCGGGGTCCAGCTGGACGCCAGCACGTACGTGGAACCAGCCGCCAAACGC
CTCTCCGAAAAAACGGCAGCAGAAGGCAAGCAGCAGGTGGAGAATACTGCGGCCAATGTGGGGTTTGGAC
TCAAGGAGCTCCCAGATCCCAATGCTGCCAAAGAAGATTTGTGGGCTGCCCATTCAGTCCTGGTGGATCC
AAAGCCACATCGGAGGCAGGATTCAAACTGGGAAGCTATCTTAAAGATCCGCAAGCGGGATGGACGCCTG
GGTCTGAAGCACTTTCGGCCCATCAAGCCCCTCGGGTGCGGGGATACGGGCAGCGTGCACCTGGTGGAGC
TCCGGGACAGCGGAAAGCTCTTTGCCATGAAGGCCATGGACAAGGATGTCATGATCAACCGCAACAAGGT
CCATCGTGCGAGCACAGAGAGAGAAATCTTGGGTCTCATAGACCATCCCTTCCTTCCCACCCTGTACGCC
TCTTTCCAGACTGGCACTCACGTGTGCCTCATCACGGACTTTTGTCCGGGCGGTGAGCTCTACCTCCTGC
TGGAGCGGCAGCCACAAAAACGTTTCCCAGAACATGCTGCCAGATTTTTTGGGGCCGAAATTCTTCTTGC
TCTAGAATATCTCCACTGCCAGGGCGTCATCTACCGCGATCTGAAGCCCGAAAACATTTTGATCTCGCGA
AGCGGCCACCTCCTATTGACCGACTTTGACCTCTCTTTCCTCTCCGAAACGACACCCAAGCTTATCTTCC
CCCCCTCGGACAAAAAGAGGAGGCGGAAGAGGGAGGAGGAGGGCGACCATCAGAGGCCTACTTTTGTTGC
GGAGCCCATGGGCAGCAGCAATTCTTTTGTGGGGACCGAGGAGTACATTGCTCCAGAAATTATCAGCGGG
ATGGGGCACACCAGCCAGGTGGACTGGTGGGCCTTCGGTATTTTTCTGTACGAGATGATGTACTCCAAGA
CCCCCTTCCGCGGCCGCAATCGGCAACGCACCTTCACCAACATCCTCATGAAGGACCTCGCCTTCCCATC
CTCTCCCCCGGTGAGCGCGGCCGCCAAGCATCTGATTCGCGGCCTCCTGGAGCGCGACCCCCAGCGGCGG
CTGGGCGCCCAGCGCGGCGTGTCAGAAATTAAGGAGCACGCCTTCTTCCATGGCCTCCAGTGGTCCCTCA
TTCGCTGCCGGCAACCTCCCGAGCTGGAGACCCCGGTGAAGTTTACGAACACGGAGCCGGAACGAGAGGC
CGCAGAACAAGACGAAGAGGATCTTGAATGGGACGACACAGAGGCGAGGAGCGCTTCCACTTCCTTGGAT
TACTGA
SEQ ID NO: 78
>AHZ63919.1 phototropin [Cylindrocystis cushleckae]
MGRDPDVDQLGQNVSGLSVETNGNNSQVARGTGLATPDKDKILTQTEGLTDVLTTFQQTFVMSDATKPDI
PITFASEGFYKMTGYSPKEVIGRNCRFLQGEGTDRAEVARLKQCLVSGESFCGRLLNYRKDGTPFWNLLT
VSAVKNDDGKIVKFVGMQVEVTKYTEGKADEQRRPNDMPVSLIRYDARQKEEAETSVAEILHAVKLPEQA
KARLSMTPVLDESISQREQEVSQEDAAAKRKRERRTSGFMTLLGNGATKEELTPVISEPSTPQPVEKEEV
RDSFELTGEKNGRRGLDLATTLERIQKNFVITDPRLPENPIIFASDDFLELTEYSREEVLGRNCRFLQGK
DTDQKTVQEIRDAIREQRDVTVQLLNYTKGGRPFWNLFHLQAVKDSKGDLQYFIGVQLDASTYVEPAAKR
LSEKTAAEGKQQVENTAANVGFGLKELPDPNAAKEDLWAAHSVLVDPKPHRRQDSNWEAILKIRKRDGRL
GLKHFRPIKPLGCGDTGSVHLVELRDSGKLFAMKAMDKDVMINRNKVHRASTEREILGLIDHPFLPTLYA
SFQTGTHVCLITDFCPGGELYLLLERQPQKRFPEHAARFFGAEILLALEYLHCQGVIYRDLKPENILISR
SGHLLLTDFDLSFLSETTPKLIFPPSDKKRRRKREEEGDHQRPTFVAEPMGSSNSFVGTEEYIAPEIISG
MGHTSQVDWWAFGIFLYEMMYSKTPFRGRNRQRTFTNILMKDLAFPSSPPVSAAAKHLIRGLLERDPQRR
LGAQRGVSEIKEHAFFHGLQWSLIRCRQPPELETPVKFTNTEPEREAAEQDEEDLEWDDTEARSASTSLD
Y
SEQ ID NO: 79
>KJ195114.1 Cylindrocystis brebissonii phototropin (PHOT1) mRNA
ATGGATCCGCCTCAAGGAATCAGGAAAATGCCGTTTCAGTCCGACAGCTCTGATGTCTCCCAAGGCGCCA
AGAAGCGCCACAATGGGAGTGGGCGCCCTTCAAGTGCGGACAGCGGAGCGGCCAAGGTGTTGGTGGCGGC
CGGTGGGCTGCGCGACATTCTCTCCACCTTCACACAGACGTTCGTCATGTCCGATGCCACCAAGCCGGAC
GTGCCCATCATGTTTGCAAGCGAAGGCTTCTACAAAATGACCGGCTACGGAGTGGACGAAGTGATTGGAC
GGAACTGCCGCTTCCTCCAGGGGCCGGAGACCGACCGTGCTGAAGTCGCGCGCTTGAGGGAGTGCGTTGC
GCGCGGGGCTCCCTTCTGCGGACGCCTCCTCAACTACCGGAAGGACGGGGCTCCCTTCTGGAACTTGCTC
ACGGTGTCGCCTATCAAGGATGACGACGGGAGAGTGGTGCGCTTTGTGGGCATGCAGGTGGAGGTGACCA
AATCAACTGAGGGCCGTGCAGAGCTGATGAAACGTGCCGATAACGAGGCGTCTGTTTCTCTCATCAATTA
CGAGTCCCGACAGCAGGAGGAGGCCAGTCGGCGTGCGCAGGAGCTGGTGGAGGCCGTTGCCCAGAGCGAG
CAGCCGCAGGCGCAGGCAAGCGGCAGCCCGCGCCCGTCAGGGGATGAGGGCGGAGGCAGCCTGCGCAGCG
CCAGCAGTGCCAGCAGCGGCTTCTTCACCCCGCCGGAAACGGCCACGGCCCGGAACACAACGTCAACTCA
ACGGAGATCGTTTCGCCAAAGCGCGTCCAGCTTGGGGGCCCCAGAGGCGGAGGCGGAGGCGATGGCGGCG
GATGACGAAGGGAAGAAGCGCCTGGGGCGGCGCGGGCTGGACCTGGCCACCACGCTGGAGCGGATCCAGA
AGAACTTTGTGATCACCGACCCTCGCCTGCCGGATAACCCAATTATCTTTGCCTCGGATGACTTTCTCCA
GCTGACGGAGTACTCTCGAGAAGAGGTGCTGGGCCGCAACTGCAGGTTCCTTCAGGGGAAGGACACGGAC
CGAGGGACTGTAAAGCAAATTCACACAGCGATCGAGACGCGAGGCGACATAACGGTTCAACTCCTCAACT
ACACCAAGAGCGGAAAGCCATTCTGGAATCTTTTTCATCTTCAGGCAGTCAAAGATGGCCAGGGTGCGCT
GCAGTACTTCATTGGGGTGCAGCTGGATGCCAGCGAGTACGTAGAGCCCAGGCCCAGCGCAGACGAAAGA
AAGTTGCCAGAAAGCGTGGAGGCCCAGGGCAGCAAAGAGGTTGAGCAAACAGCAAGCAACGTGGGCGCAG
GCTTGAAGGAGCTGCCCGATGCACACCAGCCAAAGGAGGACCTTTGGAAGTTCCACTCCGAACCCGTGGC
ACCCCTGCCGCACGGGCGAATGACAACAAATTGGGGGCCAATTTTGAAGATTCTGGAACGAGATGGGCGG
ATAGGGCTGAAGGATTTTCGCCCAGTGCGACCGCTGGGCTGTGGAGACACGGGCAGCGTGCACCTGGTGG
AGCTCAAGGCGGAAGATGTGCCGGACGATTCTGCCGCTTCTGCTGAGGGGATGGAGGACGGACAGCAACG
ACCTTCTCAGAAGTTCCTGTACGCCATGAAGGCCATGGACAAGGTGGTTATGATCAAGCGCAACAAGGTC
CATCGCGCGTGCATGGAACGCTGCATTCTGGGGCTGACCGACCACCCACTCTTGCCTACTCTCTACGCAT
CCTTTCAGACCAGCACTCACGTGTGCCTCATCACCGACTATGCTCCGGGGGGGGAGCTCTTCCAGCTTCT
CGATGAACAACCCCACAAGCAGTTCCCAGAAGATGTTGCACGGTTTTTTGCGTCCGAAGTTCTCGTGGCA
CTCGAATATCTGCACTTTAAGGGGGTGGTGTATCGGGACTTGAAGCCCGAGAACATCCTGATCAGAGAAT
CCGGGCATCTCATGCTCACCGATTTTGACCTTTCCTTCATGGGAACCACAGTTCCGCAGAGGAGGAAAGG
CAGCGCAGCGCACTTCACCTCATTGCCAGAGTCACTGAAAGAAGGCGAGGAAGAGCTACTGCACGTGTTT
TTTGCTGAGCCGGAGGGCACCAGCAACTCCTTTGTCGGCACGGAAGAGTACATCGCACCGGAGATCATCA
AGGGTGTAGGCCACGGCTTTCAAGTCGACTGGTGGGCATTTGGGATTCTTCTGTATGAGCTCCTCTACGG
GCGCACGCCCTTCCGGGGCAGCTGCCGCACCAAGACCTTTTCCAGCATCCTCAACAAAGAGCTGGTCTTC
CCCAAGCTACCCGAGACGAGCGCTGCCGCCAAGGACCTGATGACGCGCCTCCTCGAACGCGACCCGGATC
TGCGCTTGGGGGGCTCCGGGGGCGTCCACGAGATCAAAGCGCACCCCTTCTTCAGCACCACCCACTGGCC
GCTGGTCCTGTGCCAACCTGTTCCGGATCTTGTCCTCTTGAAGACTTCGCCAAGCGCCGAAGCTGGTCCA
GGCGAAGGAGAGGGGGAAGGCCAAGAAGGGGACGATGCGGAGGATTGGGAGGAAGGTGACGGGAAAAAAA
CTCTCTCGCTGTCCCTGGAAGGCTGA
SEQ ID NO: 80
>AHZ63915.1 phototropin [Cylindrocystis brebissonii]
MDPPQGIRKMPFQSDSSDVSQGAKKRHNGSGRPSSADSGAAKVLVAAGGLRDILSTFTQTFVMSDATKPD
VPIMFASEGFYKMTGYGVDEVIGRNCRFLQGPETDRAEVARLRECVARGAPFCGRLLNYRKDGAPFWNLL
TVSPIKDDDGRVVRFVGMQVEVTKSTEGRAELMKRADNEASVSLINYESRQQEEASRRAQELVEAVAQSE
QPQAQASGSPRPSGDEGGGSLRSASSASSGFFTPPETATARNTTSTQRRSFRQSASSLGAPEAEAEAMAA
DDEGKKRLGRRGLDLATTLERIQKNFVITDPRLPDNPIIFASDDFLQLTEYSREEVLGRNCRFLQGKDTD
RGTVKQIHTAIETRGDITVQLLNYTKSGKPFWNLFHLQAVKDGQGALQYFIGVQLDASEYVEPRPSADER
KLPESVEAQGSKEVEQTASNVGAGLKELPDAHQPKEDLWKFHSEPVAPLPHGRMTTNWGPILKILERDGR
IGLKDFRPVRPLGCGDTGSVHLVELKAEDVPDDSAASAEGMEDGQQRPSQKFLYAMKAMDKVVMIKRNKV
HRACMERCILGLTDHPLLPTLYASFQTSTHVCLITDYAPGGELFQLLDEQPHKQFPEDVARFFASEVLVA
LEYLHFKGVVYRDLKPENILIRESGHLMLTDFDLSFMGTTVPQRRKGSAAHFTSLPESLKEGEEELLHVF
FAEPEGTSNSFVGTEEYIAPEIIKGVGHGFQVDWWAFGILLYELLYGRTPFRGSCRTKTFSSILNKELVF
PKLPETSAAAKDLMTRLLERDPDLRLGGSGGVHEIKAHPFFSTTHWPLVLCQPVPDLVLLKTSPSAEAGP
GEGEGEGQEGDDAEDWEEGDGKKTLSLSLEG
SEQ ID NO: 81
>KJ195118.1 Cylindrocystis cushleckae phototropin (PHOTB) mRNA
ATGGGACGAGATCCGGACGTGGATCAGCTTGGTCAGAATGTGTCTGGGCTATCAGTAGAGACGAATGGAA
ATAATAGTCAGGTTGCGCGTGGTACAGGCTTGGCCACACCCGACAAAGACAAAATCTTAACACAAACCGA
AGGGCTGACAGATGTGCTCACGACATTTCAACAAACGTTTGTCATGTCTGACGCTACCAAGCCCGATATC
CCAATCACATTCGCTAGTGAGGGATTCTACAAGATGACAGGCTACAGCCCTAAGGAGGTCATCGGGCGAA
ATTGCCGTTTTCTTCAAGGTGAAGGCACCGACCGTGCAGAAGTTGCCCGCCTGAAGCAATGTCTGGTCTC
CGGGGAAAGCTTCTGCGGCCGTCTGCTGAACTACAGAAAAGATGGAACACCTTTTTGGAATCTTCTCACG
GTATCTGCTGTCAAAAATGACGATGGCAAGATCGTGAAGTTTGTCGGAATGCAAGTGGAGGTGACTAAGT
ACACAGAGGGCAAAGCGGACGAGCAGAGGCGTCCCAATGACATGCCTGTTTCTCTCATCCGCTACGACGC
TCGGCAAAAGGAGGAGGCGGAGACTTCAGTGGCAGAAATTCTTCATGCTGTCAAGTTGCCAGAGCAAGCT
AAGGCGCGTCTCAGTATGACACCTGTCCTGGACGAATCTATATCCCAGAGGGAACAGGAGGTGAGCCAAG
AAGATGCGGCCGCAAAACGGAAACGGGAACGGAGGACGTCAGGATTCATGACTCTATTAGGGAACGGGGC
CACAAAGGAGGAGCTGACACCTGTCATTTCGGAGCCTTCCACGCCCCAACCCGTAGAGAAGGAGGAGGTT
CGAGACAGTTTCGAGCTAACCGGAGAGAAAAATGGGCGGCGAGGGCTGGATCTAGCAACGACCCTTGAAC
GTATCCAGAAAAATTTTGTCATCACTGACCCTCGACTTCCCGAAAACCCAATTATTTTCGCGTCAGACGA
CTTTTTGGAGTTGACCGAGTACTCAAGAGAGGAGGTCCTGGGCAGAAACTGCAGATTCCTACAGGGCAAG
GATACTGACCAGAAAACAGTTCAGGAGATCCGGGACGCTATCCGAGAGCAGAGAGACGTCACAGTGCAGC
TGCTCAACTACACCAAGGGCGGTCGTCCCTTCTGGAACCTGTTCCATCTGCAGGCTGTCAAGGACAGCAA
GGGGGACCTGCAGTACTTCATCGGGGTCCAGCTGGACGCCAGCACGTACGTGGAACCAGCCGCCAAACGC
CTCTCCGAAAAAACGGCAGCAGAAGGCAAGCAGCAGGTGGAGAATACTGCGGCCAATGTGGGGTTTGGAC
TCAAGGAGCTCCCAGATCCCAATGCTGCCAAAGAAGATTTGTGGGCTGCCCATTCAGTCCTGGTGGATCC
AAAGCCACATCGGAGGCAGGATTCAAACTGGGAAGCTATCTTAAAGATCCGCAAGCGGGATGGACGCCTG
GGTCTGAAGCACTTTCGGCCCATCAAGCCCCTCGGGTGCGGGGATACGGGCAGCGTGCACCTGGTGGAGC
TCCGGGACAGCGGAAAGCTCTTTGCCATGAAGGCCATGGACAAGGATGTCATGATCAACCGCAACAAGGT
CCATCGTGCGAGCACAGAGAGAGAAATCTTGGGTCTCATAGACCATCCCTTCCTTCCCACCCTGTACGCC
TCTTTCCAGACTGGCACTCACGTGTGCCTCATCACGGACTTTTGTCCGGGCGGTGAGCTCTACCTCCTGC
TGGAGCGGCAGCCACAAAAACGTTTCCCAGAACATGCTGCCAGATTTTTTGGGGCCGAAATTCTTCTTGC
TCTAGAATATCTCCACTGCCAGGGCGTCATCTACCGCGATCTGAAGCCCGAAAACATTTTGATCTCGCGA
AGCGGCCACCTCCTATTGACCGACTTTGACCTCTCTTTCCTCTCCGAAACGACACCCAAGCTTATCTTCC
CCCCCTCGGACAAAAAGAGGAGGCGGAAGAGGGAGGAGGAGGGCGACCATCAGAGGCCTACTTTTGTTGC
GGAGCCCATGGGCAGCAGCAATTCTTTTGTGGGGACCGAGGAGTACATTGCTCCAGAAATTATCAGCGGG
ATGGGGCACACCAGCCAGGTGGACTGGTGGGCCTTCGGTATTTTTCTGTACGAGATGATGTACTCCAAGA
CCCCCTTCCGCGGCCGCAATCGGCAACGCACCTTCACCAACATCCTCATGAAGGACCTCGCCTTCCCATC
CTCTCCCCCGGTGAGCGCGGCCGCCAAGCATCTGATTCGCGGCCTCCTGGAGCGCGACCCCCAGCGGCGG
CTGGGCGCCCAGCGCGGCGTGTCAGAAATTAAGGAGCACGCCTTCTTCCATGGCCTCCAGTGGTCCCTCA
TTCGCTGCCGGCAACCTCCCGAGCTGGAGACCCCGGTGAAGTTTACGAACACGGAGCCGGAACGAGAGGC
CGCAGAACAAGACGAAGAGGATCTTGAATGGGACGACACAGAGGCGAGGAGCGCTTCCACTTCCTTGGAT
TACTGA
SEQ ID NO: 82
>AHZ63919.1 phototropin [Cylindrocystis cushleckae]
MGRDPDVDQLGQNVSGLSVETNGNNSQVARGTGLATPDKDKILTQTEGLTDVLTTFQQTFVMSDATKPDI
PITFASEGFYKMTGYSPKEVIGRNCRFLQGEGTDRAEVARLKQCLVSGESFCGRLLNYRKDGTPFWNLLT
VSAVKNDDGKIVKFVGMQVEVTKYTEGKADEQRRPNDMPVSLIRYDARQKEEAETSVAEILHAVKLPEQA
KARLSMTPVLDESISQREQEVSQEDAAAKRKRERRTSGFMTLLGNGATKEELTPVISEPSTPQPVEKEEV
RDSFELTGEKNGRRGLDLATTLERIQKNFVITDPRLPENPIIFASDDFLELTEYSREEVLGRNCRFLQGK
DTDQKTVQEIRDAIREQRDVTVQLLNYTKGGRPFWNLFHLQAVKDSKGDLQYFIGVQLDASTYVEPAAKR
LSEKTAAEGKQQVENTAANVGFGLKELPDPNAAKEDLWAAHSVLVDPKPHRRQDSNWEAILKIRKRDGRL
GLKHFRPIKPLGCGDTGSVHLVELRDSGKLFAMKAMDKDVMINRNKVHRASTEREILGLIDHPFLPTLYA
SFQTGTHVCLITDFCPGGELYLLLERQPQKRFPEHAARFFGAEILLALEYLHCQGVIYRDLKPENILISR
SGHLLLTDFDLSFLSETTPKLIFPPSDKKRRRKREEEGDHQRPTFVAEPMGSSNSFVGTEEYIAPEIISG
MGHTSQVDWWAFGIFLYEMMYSKTPFRGRNRQRTFTNILMKDLAFPSSPPVSAAAKHLIRGLLERDPQRR
LGAQRGVSEIKEHAFFHGLQWSLIRCRQPPELETPVKFTNTEPEREAAEQDEEDLEWDDTEARSASTSLD
Y
SEQ ID NO: 83
>KJ195111.1 Planotaenium ohtanii phototropin (PHOT) mRNA
ATGAGTACCTTGAAGGACGCCCTCTCATCGGGCACCACCCATGCAGACGTCAGAGGAGGAGGTAGCGTCC
CAACGGCGCGGCGCTACTCGCTCAAGATTGAGCAGACTCCTGCCGGCGGGTCTGGCGCTTCGAAAGTCCT
CAGCTCGAAATCAGAACTCAAAGATGCTCTCAGCGCGTTTCAGCAGACTTTCGTTATGGCCGACGGGACC
AAGCCTGATTTCCCCATCATGTTCGCGAGCGAGGGGTTTTACCAGATGACCGGATATACGCCATTAGAAA
CCATTGGAAAGAACTGTCGCTTCCTCCAGGGCCCTGAAACAGACCGTGCCGAGGTGAAGAAGCTTAAGGA
GGCGCTCGACCAGGGCCGCAGCTTTTGCGGTCGCATTCTGAATTACAAGAAAGATGGCACAAAGTTCTGG
AACCTTCTCACCATCTCTCCCGTCAAGGACGACAACGGAAAGGTCGTCAAGTTCATCGGGATGCTGACGG
AGGTGACCAAGTACACCGAGGGGGCGCACTCCGCCGACGTGCGGTCGAACCAACTCCCCATCTCGCTCAT
CAAATATGACGCGCGTCAGAAGGAGGAGGCCGAGAGCAGCGTCACTGAGCTCCTCGAAGCCGCCAAGGGC
CCGCACCCGCTCCTCGCGCCGCTCGGCCCGGGCAGCGTGTCGGCCGGTGGCGGCGGCATGGAGAAGTTGA
TGCAGCTCCCCAAGGTCGACGAAGGGGGCGCGGAGGACGACGTGGCCGCGAAGCCGAGTCGCAAGTCGGG
GCTCTTCAACATGCTCAGCAAGAAGGAGAGGCAGAGCATGAGCGCCGCGCCCGCAAAGAAGAAAGAGGAG
GATGACGACGACATGATCGACGATGAGTCGAAGAAGAAGGCACGACGGGGGCTCGATCTGGCGACCACTT
TGGAGCGTATCCAAAAGAATTTCGTCATCACGGACCCAAGGCTGCCAGAGAACCCAATTATTTTTGCTTC
TGACGATTTCTTGGAGCTCACCGAATACTCGAGAGAGGAAATCATTGGGAGGAACTGCAGGTTCCTTCAG
GGCAAAGACACCGACGAGAAGACCGTTCAGAAAATCAGGGACGCGATCAAAAACGAAGAAGATATCACTG
TGCAATTGTTGAACTACACCAAGAGCGGGAAGCCATTTTGGAACCTTTTTCATCTTCAGGCCGTGCGCGA
CAACAAGGGTGTGCTTCAATACTTCATCGGGGTCCAATTGGACGCGTCACAATACGTTGACCCTTCCATT
CATGGGCTTGACGCCACAGTCGCCAAGGAGGGCGAGCAGCTGATCATTGAGGCCGCCAATAGCGTAGAAG
GGGCCGTCAAGGAGTTGGCTGATCCAGGAAATTCCTCTCAAGACTTATGGGAGATCCATTCGCGCCCTGC
TGTCGCCAAGCCTCACAAAATGCAAGACGAGTCCTGGAAGTTCATCAAACAGGTCATTGAGAGAGAGGGT
AAGTTGGGGCTAAAGCATTTCAAGCCGATCAAACCTTTGGGGTGCGGTGACACCGGCAGCGTTCACCTGG
TCGAGCTTCGCGACACGGGCAAAATGTTCGCGATGAAGGCCATGGACAAGGAGGTCATGATCAACAGGAA
CAAGGTCCACCGTGCATGTACGGAAAGAGAGATCCTCGGAATGATCGACTTCCCGTTCCTGCCTACGCTG
TATGCTTCCTTTCAGACTGCCACTCACGTGTGTCTCATCACTGAGTTTTGCTCTGGAGGCGAACTATACG
GAGTGCTGGAGAAGCAAAAGGGAAAAAGATTCACGGAGGAAGTGGCCAAGTTCTTCACGGCTGAAGTGCT
CCTCGCTTTGCAGTACCTGCACTGTCACGGAATTGTGTACAGAGACCTGAAACCAGAAAACATCCTTCTC
ACGGGAGACGGGCACGCGATTCTGACGGACTTCGACCTTTCCTTTCTCACGCAATCAGCCACGCCGCAGG
TTCTCATGCCTCCCCCCGAAGCTTCCTCTGGCAAGAAGAAGAAGAAGAAGAAGGGCTCTGCGGACTCCGA
GCCGCGACCCAAATTCGTCTCCGAACCGAACGCGACGTCGAACTCCTTCGTCGGTACGGAAGAGTACATC
GCACCTGAAATCATCAGCGGCGCGGGGCACAGCGCGCCCGTCGACTGGTGGGCTCTTGGTATATTCATTT
ACGAAGTTTTGTACGGAAAGACCCCTTTCCGCGGTAGAAACCGACAGCGCACGTTCACGAACGTGCTGAT
GAAGGAATTGAACTTTGCTGAAAACCCTCCTGTTTCTGCCAACGCTAAGAGCATCATTCGAGCGTTGCTC
GAGAGGGACCCTGCGAAGCGGCTCGGCTCTGCGAGAGGCGCCACGGAGATCATGGACCATCCGTGGTTCT
CGGACATCAATTTCCCCCTCATCAAGAACAGGAAATTGCCGCCCCTGAGTGTAGCCGTGAAGAGCATCAG
TTCCGAACCTGACTCCGCTCGTCAGTCAGTGGCGGATGAAGAGTTGGAGTGGGACGAAAATGATGGAAGA
CCGTCCATTTCCTCTGATTACGGCTACTAG
SEQ ID NO: 84
>AHZ63912.1 phototropin [Planotaenium ohtanii]
MSTLKDALSSGTTHADVRGGGSVPTARRYSLKIEQTPAGGSGASKVLSSKSELKDALSAFQQTFVMADGT
KPDFPIMFASEGFYQMTGYTPLETIGKNCRFLQGPETDRAEVKKLKEALDQGRSFCGRILNYKKDGTKFW
NLLTISPVKDDNGKVVKFIGMLTEVTKYTEGAHSADVRSNQLPISLIKYDARQKEEAESSVTELLEAAKG
PHPLLAPLGPGSVSAGGGGMEKLMQLPKVDEGGAEDDVAAKPSRKSGLFNMLSKKERQSMSAAPAKKKEE
DDDDMIDDESKKKARRGLDLATTLERIQKNFVITDPRLPENPIIFASDDFLELTEYSREEIIGRNCRFLQ
GKDTDEKTVQKIRDAIKNEEDITVQLLNYTKSGKPFWNLFHLQAVRDNKGVLQYFIGVQLDASQYVDPSI
HGLDATVAKEGEQLIIEAANSVEGAVKELADPGNSSQDLWEIHSRPAVAKPHKMQDESWKFIKQVIEREG
KLGLKHFKPIKPLGCGDTGSVHLVELRDTGKMFAMKAMDKEVMINRNKVHRACTEREILGMIDFPFLPTL
YASFQTATHVCLITEFCSGGELYGVLEKQKGKRFTEEVAKFFTAEVLLALQYLHCHGIVYRDLKPENILL
TGDGHAILTDFDLSFLTQSATPQVLMPPPEASSGKKKKKKKGSADSEPRPKFVSEPNATSNSFVGTEEYI
APEIISGAGHSAPVDWWALGIFIYEVLYGKTPFRGRNRQRTFTNVLMKELNFAENPPVSANAKSIIRALL
ERDPAKRLGSARGATEIMDHPWFSDINFPLIKNRKLPPLSVAVKSISSEPDSARQSVADEELEWDENDGR
PSISSDYGY
SEQ ID NO: 85
>KT321719.1 Phymatodocis nordstedtiana phototropin (PHOT) mRNA
ATGGGTCCGCCAGGAAGTTCTAGCGTTCCGTCAATGGTCCCGGGCACGACTCACACGCACGTGACGGGCG
GGGGCAGCGTGCCTACAGCCCGGCGCTACTCGCTGGGGCTCACTCCGGAACCTGCGGCCCCGCAGAAGGT
GTTGGGCTCCAAGGCGGAGCTCCGCGACGCCCTCACCGCTTTTCAGCAGACCTTCGTGATGGTGGACGCT
ACGAAGCCCGACTACCCTGTTATGTTCGCCAGCGAGGGATTCTATCAAATGACAGGATACTCGGCCCTGG
AGACCATTGGGAAGAACTGCCGTTTTCTGCAGGGACCCGAAACTGACCGTGCTGAGGTGGCGAAGCTGAA
GCAGGCGATCCTGGCCGGGGAAAGCTGGTGCGGGCGGCTCCTGAACTACAAAAAGGACGGCACGGCCTTC
TGGAACCTCCTCACCGTCTCCCCAGTCAAGGACGATGATGGCACTGTCGTGCGATTCATCGGGATGCAAG
TGGAGGTGACCAAGTACACGGAGGGGTCCAAGGACAAGGAGACGCGTCCCAACGCCCTGCCCGTGTCCCT
CATCAAGTACGACGCACGGCAGAAGGAAGAGGCGGAGAGCACGGTGAGCGAGCTGGTGGTTGAGGCGACA
AAGCATCCGCTGCTGGAGTCTATGGGGGGCGGGGGCACTTTGGGGGGAGGAGGGATGGAGAAGCTGATGC
AGCTGCCCAAGGTTGAGGAAGGCGGGGAGGACGCCGTGGACGACCGCAGGTCTAAGTCGGACCGCCGCAA
GTCCGGCCTGATGACGCTCCTCTCGAAAAAGGAGAAGGCGGCGCCGTCGGAGGGGAAGCTAGCGGAGGCG
CCGAAGGCGGCAGAGACCGCAGAGGAGGACGTCGGGGACGACCGCAAGGCGAGGAAGGGAATGGACCTGG
CCACGACGCTGGAACGTATACAGAAGAATTTTGTCATCACGGATCCCCGCCTCCCCGACAACCCCATTAT
TTTTGCATCGGACGACTTCCTGGAACTCACGGAATACTCTCGAGAAGAAATTATCGGGAGGAATTGCAGG
TTCCTGCAGGGCCCGGACACCAACCCAAAGACGGTGCAGAAAATCCGTGAGGCGATCAACAACCAGGAGG
ATATCACCGTGCAGCTCCTCAACTACACAAAGAGCGGGAAGCCGTTTTGGAACCTCTTCCATCTGCAGGC
CGTGAAGGACAACAAGGGTTTGCTGCAGTACTTTATCGGCGTGCAGCTGGACGCCAGCCAGTATGTGGAC
CCGAACATCCAGGGCCTGGAGGACCGGTTCGCACAGGAGGGGGAGAAGATTGTGCTGGAGACGGCCGCCA
ACATCGATGGTGCTGTGCGCGAGTTGGCCGATCCGGGGGCGGCCCCGCAGGACCTCTGGGCCATCCACTC
CATGCAAGCTGTCCGCAAGCCACATAAGGCCACGGATCCTGCCTGGAAGGCCATCCTTGAGGTGATGGAG
AAGGACGGCAAGCTGGGGCTGAAGCACTTCCGCCCCATCAAGCCCCTGGGCGCGGGGGACACAGGCAGTG
TGCACCTGGTGGAGCTGCGGGACACGGGCCGCCTGTTTGCCATGAAAGCCATGGACAAGGAGGTCATGAT
CACGCGCAACAAGGTCCACCGTGCGTGCACGGAGCGCGACATCCTCGGGCGCCTGGACCACCCCTTCCTG
CCCACCCTCTACGCCTCCTTCCAGACGGCCACGCACGTGTGCCTGATCACGGAGTTCTGCGCGGGCGGGG
AGCTGTACGGGGTGCTGGAGAAGCAGAAGGGGAAGCGCTTCCCCGAGAGTGTGGCCAAGTTCTTCGGGGC
GGAGGTGCTCCTCTCCCTCGAGTACTTGCATTGCCAGGGCGTTGTATACCGCGACCTGAAGCCGGAGAAC
GTGCTGATCACCGAAAAGGGCCACGCGATGCTCAGCGACTTCGACCTCTCCTTCCTCACCCAGTCCACCG
TGCCCCGGGTTGAGATGCCCCCTCCGGAGGCGCTGGAGATGCTGAAGAAGAAGAAGGGGGGAGGAGGGAA
CAAGAAGAAGAAGGGCAGCAAGGGAGGGGGCGGCGACGTCGAGGCCAAGCTGGCGGCCCTGCGGGCCATC
ACTCCCACGCTGGTCGTGGAGCCGGTCAGCTCGTCCAACTCCTTTGTGGGGACGGAGGAGTACATTGCCC
CCGAGATCATCAACGGCACGGGGCACAGCAGCCCCGTCGATTGGTGGGCCTTCGGAATCTTTCTGCACGA
AATGCTGTACGGAAAGACGCCATTCCGGGGCCGCAACCGGCAGCGCACCTTCACAAACGTCCTCATGAAG
CCCCTCACCTTTCCGGACACTCCTCAGGTGAGTAGCGAGGCCAAGGCGCTGATGATGGCTCTGCTGGAGA
AGGATCCGGAGAAGCGGCTGGGGAGCAAGAAGGGGGCTGCGGAGATCAGAGGGCACCCCTTCTTCAGAGA
CCTCAACTGGGCGCTGCTGCGCCACCGGGCCCCTCCCCCTCTCAGCGTGCCAGTGAAGCCCATCACCACG
GAGTCCGACTCGGCGCGCCAGTCGATCTCTGAGGAGGAGTTGGACTGGGATGAAAACGAGGCCCGGCCTT
CCACGTCCATATCCAC
SEQ ID NO: 86
>ANC96844.1 phototropin, partial [Phymatodocis nordstedtiana]
MGPPGSSSVPSMVPGTTHTHVTGGGSVPTARRYSLGLTPEPAAPQKVLGSKAELRDALTAFQQTFVMVDA
TKPDYPVMFASEGFYQMTGYSALETIGKNCRFLQGPETDRAEVAKLKQAILAGESWCGRLLNYKKDGTAF
WNLLTVSPVKDDDGTVVRFIGMQVEVTKYTEGSKDKETRPNALPVSLIKYDARQKEEAESTVSELVVEAT
KHPLLESMGGGGTLGGGGMEKLMQLPKVEEGGEDAVDDRRSKSDRRKSGLMTLLSKKEKAAPSEGKLAEA
PKAAETAEEDVGDDRKARKGMDLATTLERIQKNFVITDPRLPDNPIIFASDDFLELTEYSREEIIGRNCR
FLQGPDTNPKTVQKIREAINNQEDITVQLLNYTKSGKPFWNLFHLQAVKDNKGLLQYFIGVQLDASQYVD
PNIQGLEDRFAQEGEKIVLETAANIDGAVRELADPGAAPQDLWAIHSMQAVRKPHKATDPAWKAILEVME
KDGKLGLKHFRPIKPLGAGDTGSVHLVELRDTGRLFAMKAMDKEVMITRNKVHRACTERDILGRLDHPFL
PTLYASFQTATHVCLITEFCAGGELYGVLEKQKGKRFPESVAKFFGAEVLLSLEYLHCQGVVYRDLKPEN
VLITEKGHAMLSDFDLSFLTQSTVPRVEMPPPEALEMLKKKKGGGGNKKKKGSKGGGGDVEAKLAALRAI
TPTLVVEPVSSSNSFVGTEEYIAPEIINGTGHSSPVDWWAFGIFLHEMLYGKTPFRGRNRQRTFTNVLMK
PLTFPDTPQVSSEAKALMMALLEKDPEKRLGSKKGAAEIRGHPFFRDLNWALLRHRAPPPLSVPVKPITT
ESDSARQSISEEELDWDENEARPSTSIST
SEQ ID NO: 87
>KT321720.1 Penium exiguum phototropin (PHOT) mRNA
ATGGCTCCGCCCCCGAATGCGGAAATAGCGGCGTTCGCCAAGGGGGCCACGCACGAGCGAGTCACGGGCG
GAGGCAGTGTGCCCACTGCGCGGCGGTACTCGCTGGGGCTGGGGCAGGAGGATGCTGCCCCGCGCACGAG
CGGCGGCGGGCAGAAGGTGCTTGGCGCCAAGGCGGAGCTGAGGGATGCTCTGACCGCGTTCCAGCAGACC
TTCGTTATGGTTGACGCCACCAAGCCCGACTACCCGGTCATGTTCGCCAGCGAAGGTTTCTACCAGATGA
CTGGATACTCCGCCCTCGAAACCATCGGCAAGAACTGCCGCTTCCTGCAGGGCCCGGACACGGACAGGGA
GGAGGTGGGGAAGCTGAAGCAGGCCATTATGGGCGGGGAGAGCTGGTGTGGCAGACTGCTCAACTACAAA
AAAGACGGCACGCCCTTCTGGAATCTGCTGACGGTGTCGCCCGTGAAGGACGACAACGGCAAAGTGGTCA
AGTTCATTGGAATGCAAGTGGAAGTCACAAAATATACTGAAGGGTCCAAAGACAAAGAGACCCGCCCCAA
CGCCCTTCCAGTATCTCTCATTAAATATGATGCCCGGCAGAGGGAGGAGGCAGAGAGCTCAGTGAGTGAG
CTGCTGGCAGAGGCGTCCAAGCATCCCCTGCTGGACGAGGCAGGGGCAGGGGCCGCAGGGGGGGGCATGG
AGAAGCTCATGCAGCTGCCCAAAGTGGACGAGTCTGCTTCCGCTGCAGCTGAGGCCAAAGGAGATCGCCG
CAAGTCCGGCCTCATGTCCATGCTCTCGAAGAAGGAGCAGAAGGGACAGGGCAAGGGGGCGCAGGAGAAG
GTGGAGGAGGAGGATGATGGTGGGGATGTGGAGCACAAGACGAGAAAGGGGCTTGATCTCGCGACAACCC
TGGAACGTATTCAAAAGAACTTTGTCATCACGGATCCGCGCCTGCCCGACAACCCCATCATTTTTGCGTC
AGATGACTTTTTGGAGCTGACAGAGTACACCCGCGAAGAAATCATAGGCCGCAACTGCAGGTTCCTGCAG
GGGCCAGACACGAACCCGAAGACGGTGCAGAAGATCCGAGATGCCATCAACAGTCAGGAGGACATCACAG
TGCAGCTGCTGAACTACACTAAGAGCGGCAAGCCCTTTTGGAATCTGTTTCATCTTCAGGCTGTGAAGGA
CAACAAGGGTACTCTGCAGTACTTTATCGGAGTCCAGCTGGATGCCAGCCAATACCTCGACCCCAACATC
CAGGGCCTTGAGGATCGCTTTGCAACAGAGGGAGAGAAGATTATTGTGGAGGCTGCAAGCAACATTGACT
CGGCCGTGAAAGAGCTGGCAGACACTGGAGCTGCTCCTCAGGATCTGTGGGCTATTCACTCAGTCCCGGC
AGCTGTAAAGCCCCACAAAAGACAAGACCCAGCCTGGCAGGCCGTGCAGGAGGCCATCTCCAAGGACGGG
AAGCTGGGGCTGAAACACTTTCGACCCATCAAGCCATTGGGAGCCGGGGACACTGGAAGCGTGCACTTGG
TTGAGCTTCGTGACAGTGGGTGCCTGTTTGCAATGAAGGCCATGGACAAAGAAGTCATGATCAACCGCAA
CAAGGTGCACCGTGCTGTGACTGAAAGGGAGATTCTGGGGCGCATAGACCACCCCTTCCTGCCCACGCTG
TTCGCCTCCTTCCAAACGGCGACGCATGTGTGCCTAATCACCGAGTTCTGTGAGGGCGGAGAGCTGTACG
GCGTTCTGGAAAAGCAGAAGGGCAAACGCTTTCCGGAGCCCGTCGCAAAGTTCTTCGCAGCGGAAGTGCT
GTTGGCTTTGGAGTACCTGCACTGCCAAGGCGTGGTGTACCGAGATCTGAAGCCGGAGAATGTGCTCATT
GCCAAGTCAGGCCATGCTGTACTCAGTGACTTCGACCTTTCCTTCCTCACCCAGGCCACGCCCAAGCTGG
AGATGCCCCCTCCTTCGGCAGCGGAGGGGAAGAAGAAGAAGAAGGGGGCTGGCAAGAAGAAGAAGAAGGG
GGGCACAGGGGACAAGGCTGGGGACAGGGACCCCGGGGAGCCCCTGCCAATGCTCATTGCAGAGCCTGAC
TCGTCCTCCAACTCCTTCGTTGGCACAGAAGAGTACATTGCGCCTGAAATCATCAATGGTACCGGGCACA
GCAGCCCCGTCGACTGGTGGGCCTTTGGCATCTTCCTGCACGAAATGCTGTACGGCAAAACTCCGTTCCG
GGGCCGCAACAGACAGCGCACGTTCACAAATGTGCTCATGAAGGAACTTACCTTCTCTGACTCAGTACCA
GTGTCCAACGAGGCAAAGAACTTGATGAAGAAGCTTCTTGAGAAGGAACCAGAGAAGAGGCTGGGGGGCA
AAAAAGGAGCAGCAGAAATTCGAGCCCACCCTTTCTTCAGAGACATTGATTGGGCACTCGTCCGCCACCA
TAAACCCCCTGGTCTGGCGGTGCCGGTGAAGCCCATCACAACGGAGCCAGATTCAGTGCGCCAGTCGTCC
GAAATGGAGGAACTCGATTGGGACGAGAACGAGGCCCGGCCATCCACATCGTTGTCGATGGATTATGGGT
ATTAA
SEQ ID NO: 88
>ANC96845.1 phototropin, partial [Penium exiguum]
MAPPPNAEIAAFAKGATHERVTGGGSVPTARRYSLGLGQEDAAPRTSGGGQKVLGAKAELRDALTAFQQT
FVMVDATKPDYPVMFASEGFYQMTGYSALETIGKNCRFLQGPDTDREEVGKLKQAIMGGESWCGRLLNYK
KDGTPFWNLLTVSPVKDDNGKVVKFIGMQVEVTKYTEGSKDKETRPNALPVSLIKYDARQREEAESSVSE
LLAEASKHPLLDEAGAGAAGGGMEKLMQLPKVDESASAAAEAKGDRRKSGLMSMLSKKEQKGQGKGAQEK
VEEEDDGGDVEHKTRKGLDLATTLERIQKNFVITDPRLPDNPIIFASDDFLELTEYTREEIIGRNCRFLQ
GPDTNPKTVQKIRDAINSQEDITVQLLNYTKSGKPFWNLFHLQAVKDNKGTLQYFIGVQLDASQYLDPNI
QGLEDRFATEGEKIIVEAASNIDSAVKELADTGAAPQDLWAIHSVPAAVKPHKRQDPAWQAVQEAISKDG
KLGLKHFRPIKPLGAGDTGSVHLVELRDSGCLFAMKAMDKEVMINRNKVHRAVTEREILGRIDHPFLPTL
FASFQTATHVCLITEFCEGGELYGVLEKQKGKRFPEPVAKFFAAEVLLALEYLHCQGVVYRDLKPENVLI
AKSGHAVLSDFDLSFLTQATPKLEMPPPSAAEGKKKKKGAGKKKKKGGTGDKAGDRDPGEPLPMLIAEPD
SSSNSFVGTEEYIAPEIINGTGHSSPVDWWAFGIFLHEMLYGKTPFRGRNRQRTFTNVLMKELTFSDSVP
VSNEAKNLMKKLLEKEPEKRLGGKKGAAEIRAHPFFRDIDWALVRHHKPPGLAVPVKPITTEPDSVRQSS
EMEELDWDENEARPSTSLSMDYGY
SEQ ID NO: 89
>KJ195103.1 Coleochaete scutata phototropin (PHOT) mRNA
ATGGAAGGGGCATCCCAACGTGAGCAAATGCAAAAGCAACTTGACGAGAACTTTGGACCTCATTTGAAGG
CTTCCCGGGGTCCATCATTGTCCGCTGAGATAGAGAAGGCTGGCCAACAGGAGACATCTTTGCCTGCAAC
ACAGCTCGCAGTTGGGAGTGTTAGGCTATTAAATTCAGCCTCCAGGTCAGAAATTACCACCCTTTCTTCC
CCACATTCAGTTCTCTGGCAGGGTGGAGCCGGAGGCAAATCGAGCCTGACTGACGCAAAGGCAACAGCTC
GTTCATCGACATCGGCGGAGTATTCCAGTGATACGCATACGTACTTTGGAGGCCGCACATCGTCATCTTC
TTTCTCTAACACACCAGAACTTCTTTCGCCGTACGGAGTAGCTCCTACAGTGAGACGGAGCATGGATGCC
CCTCAAGTTTCGAAGGGAGGGACGGATGCACAAGGAAAAAATGCTGTCTCTTCGTCCGAAGGGATTGTGG
GAGACAGTGGTCGGAAGCAGCTGCCGCAGCTGTCTATCCAGATTCAGTCTGGAACCAGGAACTCAGGTGA
ACGGCCAGGGTCTGCTACATCTGCTGGATCCTATTCCGAAGGCCCAGGGGGAGTGTCATCCTACTTTGAT
GAGGGTTGGGCTCGGTACAGTATGAAGGTGAATGATACCATTGGTGCTTTCCAGGGCGGTGGTCCAGTAA
AATCAAACTCAAGTGGTGCATCAAAGTCAAACTCGGAAGCAAGTGTAGGAGGCAGCAGCCGGAGTGTGCC
TCCGATGGCAGACGAGCTCAAGGACATTTTGTCAACCTTCAGACAGACCTTCGTTGTGTCAGATGCCACA
AAGTCTGAATGTCCCATCATGTATGCGAGCGAGGGCTTCTACCACTTAACAGGCTACACTCCGGACGAAG
TAATCGGCCATAATTGTCGGTTTCTGCAAGGTCCTGGGACGGATGTAAAAGAAGTGGCAAAGATTCGAGC
CGCAATTCGGGATGGGAAAAGCTACTGCGGACGGCTGATGAATTACCGGAAGGACGGAACACACTTCTGG
AACCTTCTCACCGTCGCACCCGTCAAGAATGAGCGAGGGAATGTGATTAAGTTCATCGGAATGCAAGTGG
AAGTGTCAAAGTTCACCGAGGGGCACCACGGAGACACAACCCGGCCAAATGGACTTCCCTCCGGACTCAT
CGCCTATGACGCAAGAGCGAAGGACAGGGTGGCTCCTGCGGTCTCTGAACTCGTTGACGTAGTGTCAAAG
CCGCACCCTCTGCTGGAGCTCCCTCCCGCTCAGCCACAGGAGGGGAGTGGCCTTGCCAAGCTCTTCTCCT
CCCTCCCCCCTCCACAGCAAAACGTACCCCCAGCGAGTGAGCTTCTCATGAACCAGATGCCCGAGACTTT
CCCCGGCCGCCCCTCAGCGACTGTCGCGGAAAGAAAGGATTGGGGCATGGAGCTGGACACTCCGAGAACA
GTGGAAGAAAAGAAGAAGGGACGGACAGCCGCCTTTTTAACCCTCTTGGGATTCTCTGGAAAAGACGCAA
GTGCAACTTCGACCTCCGTTGGGGTCCCCACGTTGGATCTGCCTGTGGTGGAAGCTACCCCTGCCCAAGA
ATCTCGAGAGAGAGACAGTGTGGAGACGGACGGCGGGGACTACATTCCGGAGGCGCGCCGGGGCATGGAT
CTCGCAACCACGCTGGAGCGCATACCGAAAAACTTTGTCATCACCGATCCCCGCCTGGATGAGAATCCTA
TCATTTTTGCTTCCGACAGCTTCTTAGAGCTTACGGAGTACTCACGAGAGGAGGTGCTTGGCCGCAATTG
CAGATTTTTGCAGGGGCCGGACACGGACCCAGAAACAGTGAAGAAAATCCGAGAGGCAATCCGGGACTGC
CGGGATGTCACGGTCCAGCTCTTGAACTACACCAAGTCGGGAAAACCATTCTGGAATCTTTTTCACTTGC
AAGCTGTGAGGGACAGATCGGGTGAGCTGCAATACTTCATAGGGGTACAGCTGGATGCGAGCCTTCCAGC
TGACCGTGAGGGCCTCAAAGTTCAGATCCCCGGCTCACGACTCTCCGACAACACAGCGAGCAAAGGCACC
AAGATTGTACAAGAGACAGCAAGAAACATTGACGGAGCAGTGCGCGAACTTCCAGACGCTAACTTGCATC
CCGAGGACTTGTGGGCGGGCCATAGTGTGACGGTGTTGGCGAAGCCGCATAAGAATAACGACGCATCGTG
GCAGGCTATCCGTGGGATCAAAACTAGCAGTGGACGACTGGGCTTGAGACACTTTAAACCTATTCGACCA
CTTGGAGCCGGCGACACAGGCAATGTGCACTTGGTGGAGCTCAAGGGCAGCAACTGTTTGTTTGCGATGA
AGGCGATGGACAAGGAGTCCATGATCAGCAGAAACAAGGTCCACCGTGCATGCACAGAGAGACAGATCAT
CTCAGTCCTCGACCATCCTTTCCTCCCAACGCTCTACGCTTCCTTCCAGACTGCGACACATGTTTGCCTT
ATCACTGACTTCTGCCCTGGAGGGGAGCTGTATAGCTTGCTTGAGAAGCAACCCGGCAAGATCTTTAGTG
AAGAGAGTGCCAGATTTTACGCTGCCGAGGTTCTCCTTGCACTGGAGTACTTGCACTACAAAGGTGTGAT
ATACCGAGACTTAAAACCAGAGAACGTCCTCTTGCAAGAGAACGGCCACATCTTGCTGACGGACTTCGAT
CTCTCCTTCCTCACATCCACCAGTCCTACTGTCGTCAAGAGGACACAACCAGGCTCGAGGCAGTCAAAGC
GCAAGGACAGAGAGGTCAACGAGATGATTGCGCAGCCCATCTCCTCCTCCAACTCCTTTGTCGGCACTGA
GGAGTACATCGCACCTGAGATCATTAACGGCGTAGGCCACGGCAGTGCCGTCGACTGGTGGGCGTTCGGT
GTCTTCCTCTACGAGATGCTCTTTGGCAGGACACCCTTTCGCGCCAAGCATCGCCAGCGCACCTTCCAAA
ACATTCTCGAAAAGGATCTCCACTTTCCTGACAGGCCTCAGGTGAGCCTGGCGGCCAAGCAGCTCCTCCG
TGGCCTGCTCACCCGAGAGCCGGAGAAACGACTGGGTTCTAAACGCGGGTCAAACGAGCTCAAGGAGCAT
GCTTTCTTCAAAGACATCAGCTGGGCGCTCATACGATCCCGAAGTGTGCCGGAGCTGGTGGTCCCCTTGA
AAATCTCCACACCACCACCCATCCAAGAAGCAGAACTCGACTGGGATGAAAAAGAAGCCAGAACACCACC
GGCTGGGGAATGA
SEQ ID NO: 90
>AHZ63904.1 phototropin [Coleochaete scutata]
MEGASQREQMQKQLDENFGPHLKASRGPSLSAEIEKAGQQETSLPATQLAVGSVRLLNSASRSEITTLSS
PHSVLWQGGAGGKSSLTDAKATARSSTSAEYSSDTHTYFGGRTSSSSFSNTPELLSPYGVAPTVRRSMDA
PQVSKGGTDAQGKNAVSSSEGIVGDSGRKQLPQLSIQIQSGTRNSGERPGSATSAGSYSEGPGGVSSYFD
EGWARYSMKVNDTIGAFQGGGPVKSNSSGASKSNSEASVGGSSRSVPPMADELKDILSTFRQTFVVSDAT
KSECPIMYASEGFYHLTGYTPDEVIGHNCRFLQGPGTDVKEVAKIRAAIRDGKSYCGRLMNYRKDGTHFW
NLLTVAPVKNERGNVIKFIGMQVEVSKFTEGHHGDTTRPNGLPSGLIAYDARAKDRVAPAVSELVDVVSK
PHPLLELPPAQPQEGSGLAKLFSSLPPPQQNVPPASELLMNQMPETFPGRPSATVAERKDWGMELDTPRT
VEEKKKGRTAAFLTLLGFSGKDASATSTSVGVPTLDLPVVEATPAQESRERDSVETDGGDYIPEARRGMD
LATTLERIPKNFVITDPRLDENPIIFASDSFLELTEYSREEVLGRNCRFLQGPDTDPETVKKIREAIRDC
RDVTVQLLNYTKSGKPFWNLFHLQAVRDRSGELQYFIGVQLDASLPADREGLKVQIPGSRLSDNTASKGT
KIVQETARNIDGAVRELPDANLHPEDLWAGHSVTVLAKPHKNNDASWQAIRGIKTSSGRLGLRHFKPIRP
LGAGDTGNVHLVELKGSNCLFAMKAMDKESMISRNKVHRACTERQIISVLDHPFLPTLYASFQTATHVCL
ITDFCPGGELYSLLEKQPGKIFSEESARFYAAEVLLALEYLHYKGVIYRDLKPENVLLQENGHILLTDFD
LSFLTSTSPTVVKRTQPGSRQSKRKDREVNEMIAQPISSSNSFVGTEEYIAPEIINGVGHGSAVDWWAFG
VFLYEMLFGRTPFRAKHRQRTFQNILEKDLHFPDRPQVSLAAKQLLRGLLTREPEKRLGSKRGSNELKEH
AFFKDISWALIRSRSVPELVVPLKISTPPPIQEAELDWDEKEARTPPAGE
SEQ ID NO: 91
>KT321723.1 Chaetosphaeridium globosum phototropin (PHOT) mRNA
TCGGGGTCCTCAAGTGGGGAGCCCCGAGAGCCGCTCCCCCAAGTGGCTGCAGAGGTTCGGGACGTCCTCT
CGTCCTTCCGGCAGGCATTTGTCATCTCCGACGCAACTCTGAAGGATACTCCAATCATGTTTGCAAGCGA
GGAGTTCTATCGAATGACTGGGTATGGGCCATCCGAGGTCATCGGGAAGAACTGCCGCTTCCTCCAAGGC
AAGGATACAAAGAAGGAGGATGTCGACAAGATCCGGCAGTGTGTCAAGAAGGGCGAGCACTTCTGCGGGC
GCATCCTAAACTACCGCAAGAACGGAGAGCCCTTCTGGAACCTCCTCACAGTGGCGCCAGTCAAGAACTC
CCGGGGGGAGTGCGTCAAGTTCATTGGCATGCAAGTGGAAGTGAGCAAGTACACAGAGGGTTCGGCAGCA
GAGCAGACACGGCCTGGAGGGCNNNNNNNNNNNNNNNNNNNNNNNNNNNNNNNNNNNNNNNNNNNNNNNN
NNCCTCGTGCCAGCCGTGGAAGACATCATGAGTGCTGTCACTGCTCCCCCCGCCAAGAACCCCNNNNNNN
NNNNNNNCCCCCCAGCAGGGGCGCTGGCGCCGGCGGCCTCTCCTCTCTCCTCAACCTCCCCACCGGCACA
AGTGGGGGTCCGGGTACCGGGAAGCACGGCTTTGTGAGCTCGCTGCCGCTTGTGAATGACCTCCTGAGTC
CCAATCTGGGGATTGGCAACCACAAGGCGACGCCCCTCTTCCTCGGGCCTGTCCCCCCAAGGGGCACACC
CTCGCCGGTGAATGGGGGGGGGAAGGCTGGGGAATCGAGGGGGNNNNNNNNNNNNNNNNNNNNNNNNNNN
NNNNNNNNNNNNNNCAAGTTCGGCTTGCGGCGCTCCAAGGACATGGGCAGCCCCAGCGGAAGCGGCAGAA
ACTTGGCCGGTCAGGGGCCTGCGGCGCACATCCCCGAGGATGGGGAGGTGGAGCGGCAGCCGGCCCCCGA
CGCCAAGACCCCAGACCTGAGGGACTCCACCGACTCCTCGGGCATGGAACTCGGGGAGTGCCGCATCAAG
GAGATGCGGCGGGGCATTGACATTGCAACCACGCTCGAGCGCATTCAGAAGAACTTTGTCATCACTGACC
CCCGCTTGCCCGACAATCCCATTATTTTTGCATCGGACAGCTTCCTGGAGCTGACTGAGTACACCAGGGA
GGAGATCATCGGTCGGAACTGCCGGTTCCTGCAAGGGGAGGGCACTGATCGGGCCACGGTTCAGCGCATC
CGGGACGCCATCCGTACAGAGAAGGACGTGACGGTGCAGCTGCTGAACTACACAAAGTCAGGGAAGCCCT
TCTGGAATCTCTTCCACTTGCAGGCCGTCAAGGACCAACAGGGTTTGTTGCAGTATTTCATTGGGGTCCA
GCTCGACGGGAGTCTGTACTTGGATAAGAACAAGAAGCTGTCAGAAGACACGGCCAGCAAGGGCACTGTC
CTGATCAGAGAAACAGCGTCCAAGGTGGACACTGCGGTCAAGGAGCTGCCAGACGCAGCGCTGAAAAAAG
AAGACCTGTGGGCGGGCCACCAGGTATTGGTGCTTCCAAAGCCACACAAGTGCAACAGCAGCAGCTGGGA
GGCAGTGCGCAGGGTTGCGGGCGTTGACACACGGCTCCGGCTGAAGCACTTTCGGCCTGTCAAGCCCCTG
GGGGCTGGTGACACTGGCAACGTTCACCTGGTGGAGCTCCGGGATACGGGCAAGCTCTTTGCAATGAAGG
CCATGGACAAGAACTCGATGATTGCGCGCAACAAGGTCCACAGAACAAACATGGAGCGCGAGATCCTGGG
CTCTCTCGACCACCCCTTCTTGCCCACACTGTACTCGAGCTTCACCACCAAGACACATGTGTGCCTCATC
ACTGACTACTGCTCGGGGGGGGAGCTGTTCACGCTCATGGACCGGCAGCCGGAGAAGCGCTTCTCGGAGG
CCAGCGCAAGGTTCTACTGTGCCGAGGTCCTGCTCGCCCTCGAGTACCTGCATCTCAAAGGCGTGATCTA
CCGCGACCTGAAGCCTGAGAACGTGCTTCTGATGGATACAGGCCACATCCAACTGACGGATTTTGACCTG
TCTTTCCTCACACGATCCAGCTCTACGGTCTTCAAGAAGACCGTGCCCGCGCCCCGATCGTCGCCTGTGG
TGATGAGTAGAAAGGCGCGGATGCGGCGGAAGAGGAGCCTCCGCAAGAGCAAGGCGCGGGGAGAAGAGGG
TGAGCTGTCCTCTTCAATGAGCGTGATGGTGAGCGAGCTGGTGGTGGAGCCGGCGGGGACGTCCAACTCG
TTCGTGGGGACCGAGGAGTACATTGCGCCGGAGGTGATCACGGGCAGCGGCCACACGGGCACGATCGACT
GGTGGGCGTTTGGCGTGCTCCTGTACGAGCTGCTGTGTGGGAAGACGCCCTTCCGGGGCCGGAACCGGCA
GAGGACGTTCCGGAACATCCTGGAGAAACCTGTCATTATGCCGCCCAACATTGAGATCTCGAGCGAGGGG
CAGGACCTCATCCAGAAGCTCTTGATCCGGGACCCCCTGCGTCGGCTGGGCAGCCAGCGTGGGGCCAATG
AGATCAAGGAGCACCCCTTCTTCAGAGCCATCAACTTCCCACTCATCCGCACTATGGTCCCCCCCCCGCT
CAAGGTCCCGGCCAAGTTTGTGTACCCTGACGTCAGCTCCCTCTCCCCGGACGTGGACTGGGACGACTTG
GAGGCGCGCACGCCGTCGCCTGTCGCCACTGACTACTTCTAG
SEQ ID NO: 92
>ANC96848.1 phototropin, partial [Chaetosphaeridium globosum]
SGSSSGEPREPLPQVAAEVRDVLSSFRQAFVISDATLKDTPIMFASEEFYRMTGYGPSEVIGKNCRFLQG
KDTKKEDVDKIRQCVKKGEHFCGRILNYRKNGEPFWNLLTVAPVKNSRGECVKFIGMQVEVSKYTEGSAA
EQTRPGGXXXXXXXXXXXXXXXXXPRASRGRHHECCHCSPRQEPXXXXXPPSRGAGAGGLSSLLNLPTGT
SGGPGTGKHGFVSSLPLVNDLLSPNLGIGNHKATPLFLGPVPPRGTPSPVNGGGKAGESRGXXXXXXXXX
XXXXXKFGLRRSKDMGSPSGSGRNLAGQGPAAHIPEDGEVERQPAPDAKTPDLRDSTDSSGMELGECRIK
EMRRGIDIATTLERIQKNFVITDPRLPDNPIIFASDSFLELTEYTREEIIGRNCRFLQGEGTDRATVQRI
RDAIRTEKDVTVQLLNYTKSGKPFWNLFHLQAVKDQQGLLQYFIGVQLDGSLYLDKNKKLSEDTASKGTV
LIRETASKVDTAVKELPDAALKKEDLWAGHQVLVLPKPHKCNSSSWEAVRRVAGVDTRLRLKHFRPVKPL
GAGDTGNVHLVELRDTGKLFAMKAMDKNSMIARNKVHRTNMEREILGSLDHPFLPTLYSSFTTKTHVCLI
TDYCSGGELFTLMDRQPEKRFSEASARFYCAEVLLALEYLHLKGVIYRDLKPENVLLMDTGHIQLTDFDL
SFLTRSSSTVFKKTVPAPRSSPVVMSRKARMRRKRSLRKSKARGEEGELSSSMSVMVSELVVEPAGTSNS
FVGTEEYIAPEVITGSGHTGTIDWWAFGVLLYELLCGKTPFRGRNRQRTFRNILEKPVIMPPNIEISSEG
QDLIQKLLIRDPLRRLGSQRGANEIKEHPFFRAINFPLIRTMVPPPLKVPAKFVYPDVSSLSPDVDWDDL
EARTPSPVATDYF
SEQ ID NO: 93
>KJ195105.1 Interfilum paradoxum phototropin (PHOT) mRNA, complete cds
ATGGCTGGTCAGTATATAGTTGACCCTGCACTGAATGGGGCAAACAGGGGCCCTAGTGCAGACTACAGTG
AGGACGGGGGCAGCAAACGCAGCTCAGGGTCGACCTCTACATTGCCACGCATCTCACATGACTTGAAAGA
TGCTCTGTCCACGTTCAAGCACACATTTGTGGTTGCGGATGCAACCAAGGACATGGCTATCATGTATGCA
AGCGCAGGCTTCTATGACATGACGCAGTATGGGCCAGAGGACGTCATTGGGAAGAACTGCCGCTTCTTGC
AAGGGCCTGGCACGGACCAGGAGGAAGTTGCTCGGATAAGAAGAGCGATCAAGAATGGGGAGAGCCACTG
CGGTCGCCTCCTCAACTTCAAGAAGGACGGGACGCCCTTCTGGAATTTGTTGACCCTTGCACCAATCAAG
AATGAGCAGGGACAAGTTGTCAAGTTCATCGGGATGCAAGTGGAGGTCACACAGTTTACAGAGGGCGAAC
TTGAGAAGGCAATGCGGCCCAATGGGATGTCAACATCCCTCATCAAATATGATTCTCGTCAAAAGCAGGG
TGCAACAGAGTCGGTCCTCGACATCGTGGATGCTGTCAAGAACCCAAGCCAGAAGGGCCAAGGGCCAGCG
CCTAGCCCCTTCCAGCCAGGAGCGGGTTTGGCTAGTCTTCTTGCTGCTGTGCCGAAGAGCACGCCCTCAG
CAGACCCCAGCAAAGATGAGCTAGCTACGCTCTATGAAAGTGAAGGGGGCTTGGCGGACAGGAAGGAGGG
CGCTGGGAAGAGGCGCACGTCAGGATTCATGAACCTGCTGAAGAGTGGAGGAAAGCCGCTGCAGGCAGAC
TCACCGATTGCTACGTTGACCCGGCCGCAAAGTCTGAACCTCAGCGCAGAGCTGGTGCCAACCCAGGGGA
CCACTCCTGATGCACAAGGCGCTCTGAACTTTGGGGATGACAGGGCAGCAGAGGAGAGGAAGGGGCTGGA
CCTTGCCACCACCCTGGAGCGTATCCAGAAGAACTTTGTCATCACAGACCCCAGGCTGCCAGACAACCCC
ATCATTTTTGCGTCCGATGACTTCCTGACCCTGACAGAGTACTCGCGAGAGGAGATCCTGGGGCGCAATT
GCCGCTTCCTGCAAGGGCCTGAGACAGACCAGAAGACTGTGGAGGAGATTCGCGTTGCGATCAGGGAGGA
GAAGGATATCACAGTGCAGCTGCTCAACTACAAGAAGAGCGGCGTTCCGTTCTGGAACATGTTCCACTTG
CAGCCTGTCAGGGACAAGCGGGGCGAGCTGCAGTACTTCATTGGGGTGCAGCTGGATGCTAGTGCCTGGG
ACTCCATGGGCGACCAAGCCCCGCAAGCGCCTCCTCAGACCAAGGCAGCACAGAAGAGCATTGTCAAAGA
CTCTGCATTGGAAGCCGCTGCCGCTGTACAAGAATTACCAGATCCAGGCCAGCGGCCAGAGGATGTGTGG
GCTGGTCACAGCAAGCCTGTGCTCACTAAACCCCACAAGCGGGACGCAGAGGCGTGGAAGGCCATCAAGC
TGATTAAGCAGAGGGATGGCCGTCTGGGGCTTCGACACTTCCGGCCAATCAGGCCTTTGGGTTCAGGCGA
CACTGGCAGTGTGCACCTAGTGGAGCTAAAGGGAACGAAGCACCTCTTTGCAATGAAGGCCATGGACAAG
CAAGTCATGGTCAACAGAAACAAGGTGCACCGTGCCATCACAGAGAGGGACATTCTGGCTGCCCTGGACC
ACCCATTCCTCCCAACCCTCTACGCTTCCTTCCAGACTGCCACCCACGTCTGTCTCGTAACAGACTACTG
TCCGGGAGGCGAGCTCTACTACCTCTTGGAGCAGCAGCCACAGAAGAGGTTCTCAGAAGAAGTCGTCAGG
TTCTTTGCGGCTGAGGTGCTCCTGGCGCTCGAGTACCTCCATCTCCAGGGCGTTGTGTACCGCGACCTGA
AGCCCGAGAACGTTCTGCTGCAAGAGACCGGGCACATCCTGCTGACCGACTTCGACCTCTCCTTCCTAAC
CTCCTCCAGCCCTACGATGGTGCGGCCTCCACAGACTGCGGGCAAGAAGAAGCGGAAGCAGCAGAACGGC
TTTGTGCGGCCCGAGCTGGTGGCAGAGCCGACCACCAACTCCAACTCATTTGTGGGCACCGAAGAGTACA
TTGCTCCTGAGATCATCAGTGGCTCGGGGCACAGTGGGTCGGTGGACTGGTGGGCGTTTGGCATCTTCAT
TTACGAGATGCTGTATGGCAAGACGCCCTTCCGGGGGCGCAACAGGCAGCGCACGTTCACCAACATTCTT
CTCAAGGACCTTACCTTCCCACCGCAGCCCCAGGTCAGCCTAGCTGCGCGGCGGTTTATCCGCGGGCTGT
TGGAAAGGGACCCCAACAAGCGGCTGGGGGCAGGCAAGGGCGCCACCGAATTGAAAGCGCACCCATTCTT
CGAGGGCCTCAACTGGCCCCTGATCCGCTTTGATCACCCTCCCAACCCGGAGAAGCCCGTCCAAGTGTCC
AAGGTGGAGGTCCGAGAGTCTCTGGACGAGAAGGAGGAACTAGACTGGGAGGAAGTTGACGAGCAGGGCC
ATCTGATGCAGGAGCAAATTGTGCCCACTTCAATGTAG
SEQ ID NO: 94
>AHZ63906.1 phototropin [Interfilum paradoxum]
MAGQYIVDPALNGANRGPSADYSEDGGSKRSSGSTSTLPRISHDLKDALSTFKHTFVVADATKDMAIMYA
SAGFYDMTQYGPEDVIGKNCRFLQGPGTDQEEVARIRRAIKNGESHCGRLLNFKKDGTPFWNLLTLAPIK
NEQGQVVKFIGMQVEVTQFTEGELEKAMRPNGMSTSLIKYDSRQKQGATESVLDIVDAVKNPSQKGQGPA
PSPFQPGAGLASLLAAVPKSTPSADPSKDELATLYESEGGLADRKEGAGKRRTSGFMNLLKSGGKPLQAD
SPIATLTRPQSLNLSAELVPTQGTTPDAQGALNFGDDRAAEERKGLDLATTLERIQKNFVITDPRLPDNP
IIFASDDFLTLTEYSREEILGRNCRFLQGPETDQKTVEEIRVAIREEKDITVQLLNYKKSGVPFWNMFHL
QPVRDKRGELQYFIGVQLDASAWDSMGDQAPQAPPQTKAAQKSIVKDSALEAAAAVQELPDPGQRPEDVW
AGHSKPVLTKPHKRDAEAWKAIKLIKQRDGRLGLRHFRPIRPLGSGDTGSVHLVELKGTKHLFAMKAMDK
QVMVNRNKVHRAITERDILAALDHPFLPTLYASFQTATHVCLVTDYCPGGELYYLLEQQPQKRFSEEVVR
FFAAEVLLALEYLHLQGVVYRDLKPENVLLQETGHILLTDFDLSFLTSSSPTMVRPPQTAGKKKRKQQNG
FVRPELVAEPTTNSNSFVGTEEYIAPEIISGSGHSGSVDWWAFGIFIYEMLYGKTPFRGRNRQRTFTNIL
LKDLTFPPQPQVSLAARRFIRGLLERDPNKRLGAGKGATELKAHPFFEGLNWPLIRFDHPPNPEKPVQVS
KVEVRESLDEKEELDWEEVDEQGHLMQEQIVPTSM
SEQ ID NO: 95
>KJ195106.1 Entransia fimbriata phototropin (PHOT) mRNA, complete cds
ATGGGGATTGTAGTTCAAGCACCTGGGAAGGGGGCACTGAAAGGGGCGAAAATGCAGGATCAGGCCACGG
CCACTGGCAGGGGGTCAGCTGTGGGTCAGCCCTCATCTCGAAACACCTCCTTGGACAGCGAGGGGGGCAG
CAGAGGGACCTCTGGAGTGTCCCTGCCACGGGTGTCGAGTGAGGTGAAGCTTGCCCTTTCCAGCTTCCGC
CACACGTTTGTGGTCACGGACGCGCTATCCGAAGACATGCCAATCTTGTATGCCAGCGACGGTTTTTACA
AGATGACGGGGTACGCTCCTGCGGAGACGGTTGGGATGAATTGTCGCTTCCTCCAGGGCAAGCACACCGA
CCCATCCACCAAGGCCAAGATCAAGGCGGCGGTGGCGGCAGGCCACGGCTTCTGCGGCCGCATCCTCAAC
TACCGCAAGGACGGGTCCTCTTTCTGGAACCTGCTCACCATCTCCCCCATCAAGGACAATAATGGCAATG
TCGTGCGGTTCATCGGTATGCAAGTGGAGGTTACCAAGACGACCGAAGGGGACAAGCACGATGACCTCAG
GCCCTCTGGGATGCCCACGTCAATGGTCAACTATGATGCCCGGCTGCAGGCAGGGGCCCGGACATCGGTT
GTGGAGCTGTTGCAAGCCCTCCAGGACCCCTCGCCCTTTGCTATGCATGCTGAGGAGCCGCTGCCACCGC
CGCAGGCCTTGGGGGGCCTGGCCTCCCTGCTGGCACTTCCCAGGGTTGATGACACCGCAGCTATGTTTAC
AGCTGGGGATGCGTCAGTGCAGGAGTACGACGGGATTGATCCATCCGGCAAGCCCACGGCCGGGTTCATG
TCCCTGTTGAAATTCGGAGGCCTCCCGGTTCCGCGCAAGTCAGAGCGCTTGTTTCGCCGCGCGGTGGCGG
AGCAGGCTCCCACTGAGGAGGAGCGGGAGCCGGTGGTGGACCGCAAGGCAATGGATCTTGCCACCACGTT
GGAACGGATTGAGAAGAACTTTGTCATCACTGATCCCCGCCTGCCGGACAACCCAATTATCTTCGCATCC
GACGCCTTCCTTCAACTCACCGAGTACGGCCGTGAGGAGATCCTAGGACGTAACTGCAGGTTCTTACAGG
GCCCCGACACGGACCCCCATGTGGTGTTGGAGATCCGCGCTGCGATCAAGGAAGGCCGCGAGTGCACAGT
GCAGCTTCTCAACTACAAGAGGAGCGGCACTCCGTTCTGGAACATGTTCCACTTGCAGCCGGTGCGGACA
AGACAGGGCGAGATCCAGTTCTTCATCGGTGTCCAGTTGGATGCGTCCAACTGGGGCCCCCCGGAGGAGC
ACCATCGGGAGAAGGCAGCGATTGTTCAGGCCACCGCTGGCGATGTGGGCGAGGCAGTGAAGGACTTCCC
AGACCCAGAGAAGAAACCGGAGGATCTGTGGGAGCCTCACACCCGGCCAGTGCGGATGAAGCCACACCAG
CAGCGAAAGGGGTCGTGGGCAGCCATTTTGAAGGTCCAAGAGGATGCAGGAGAGCTGAACCTGCAGCACT
TCACACCCATTCGGCCGCTGGGCTGTGGTGACACGGGCAGTGTACACCTCGTAGAGCTCAAGGGGACTGG
AGCGCTTTTCGCTCTCAAGGCAATGGACAAGGCGGCCATGATCGCCCGCAATAAGGTCCATCGCGTCCTC
ACCGAGAGGGAAGTGCTGGCCGCTGTCGACCACCCTTTCCTTCCAACTCTCTACACATCCTTTCAGACCA
AGACCCACGTCTGTCTCATCACTGATTTCTGTCCCGGGGGTGAACTCTACTATGTTCTGGACCGTCAGCC
ACACAAGCGCGTGTCAGAAGATGCCGCAAGGTTCTACATTGCTGAGGTGATCCTTGCCGTTGAGTACCTG
CACCTCATGGGTGTCACCTACCGTGACCTTAAGCCTGAGAACATCCTCATCCGCCAGGACGGCCACATCC
TCCTCACCGACTTCGACCTCTCGTTCCTCTCCTCCTCAGCCCCCCAGATCAAGGCCGGTCCGCCAGTTGC
CCGTTTCCTCTGCGCTCCTTCCCCGCCGTCTTTGCCTCAGCTCCTCGCTGAGCCGACGGCTAAGTCCAAC
TCCTTTGTCGGCACCGAGGAGTACATTGCTCCGGAGATCATTAGTGGCAAGGGGCACAGCAGCATGGTGG
ACTGGTGGGCACTAGGTATCTTCTTGTACGAGATGTTATATGGGCGCACCCCCTTCCGCGGCCGGAACCG
GCAGCGGACATTTGCTAACATCCTCGTGAAGGAGCTCGCCTTCCCGTTACAGCCACCGGTGAGTGCGGCG
GCCCGACGTCTCATCCACCAACTGCTTAGAAGAGACCCCCTGGAGCGTCTTGGGGCCCGCCATGGTGCTC
CAGAGATAAAGGAGCACCTATTCTTTGAGGACATTGACTGGCCCCTCATCCGCAGCATGCCCGCCCCCAA
ACTTGATGTGCCAATCACGCTCATTCCTTGTGTGCCCCGCTCCGCCCAACAAGGTGCCCAGGGTGACCTG
GAATGGGATGACGGGGAGGGGTCGGTCCATTTGCATGATGTGTTCTAA
SEQ ID NO: 96
>AHZ63907.1 phototropin [Entransia fimbriata]
MGIVVQAPGKGALKGAKMQDQATATGRGSAVGQPSSRNTSLDSEGGSRGTSGVSLPRVSSEVKLALSSFR
HTFVVTDALSEDMPILYASDGFYKMTGYAPAETVGMNCRFLQGKHTDPSTKAKIKAAVAAGHGFCGRILN
YRKDGSSFWNLLTISPIKDNNGNVVRFIGMQVEVTKTTEGDKHDDLRPSGMPTSMVNYDARLQAGARTSV
VELLQALQDPSPFAMHAEEPLPPPQALGGLASLLALPRVDDTAAMFTAGDASVQEYDGIDPSGKPTAGFM
SLLKFGGLPVPRKSERLFRRAVAEQAPTEEEREPVVDRKAMDLATTLERIEKNFVITDPRLPDNPIIFAS
DAFLQLTEYGREEILGRNCRFLQGPDTDPHVVLEIRAAIKEGRECTVQLLNYKRSGTPFWNMFHLQPVRT
RQGEIQFFIGVQLDASNWGPPEEHHREKAAIVQATAGDVGEAVKDFPDPEKKPEDLWEPHTRPVRMKPHQ
QRKGSWAAILKVQEDAGELNLQHFTPIRPLGCGDTGSVHLVELKGTGALFALKAMDKAAMIARNKVHRVL
TEREVLAAVDHPFLPTLYTSFQTKTHVCLITDFCPGGELYYVLDRQPHKRVSEDAARFYIAEVILAVEYL
HLMGVTYRDLKPENILIRQDGHILLTDFDLSFLSSSAPQIKAGPPVARFLCAPSPPSLPQLLAEPTAKSN
SFVGTEEYIAPEIISGKGHSSMVDWWALGIFLYEMLYGRTPFRGRNRQRTFANILVKELAFPLQPPVSAA
ARRLIHQLLRRDPLERLGARHGAPEIKEHLFFEDIDWPLIRSMPAPKLDVPITLIPCVPRSAQQGAQGDL
EWDDGEGSVHLHDVF
SEQ ID NO: 97
>KT321724.1 Spirotaenia minuta phototropin (PHOT) mRNA, partial cds
ATGGGGTCCGACGGGGCGTACGATGCGTATGGCTTTCCAACGGAGAAGTCTAGGACGCGTGGGGATTCCG
TCTCATTGGCGACTGGCCTTCCGGCTTTCTCGTCGGAGACGACGGGCCTGTTGGGCTCCTTCCGCCATTC
CTTTATCCTAACTGATCCCTCAAAGCCCGATTTCCCGATTGAATATGCAAGCGATGGGTTTTACGAACTT
ACCGGCTACACTCCCTCCGAGACTATGGGACGAAATTGTCGTTTTCTACAAGGGCCAGGCACAGACCGGC
TAGAGGTTGAGAAGCTGAAGGAAGCAATCATGGAAGGCAGGCCTATCTCCCTGCGGTTGCTAAACTACAA
GAAGAGCGGCGAGGCATTCTGGAATCTGCTGACGGTCTCTCCCTTTGACGTGGGGGGCAAGAGGAAGTTT
CTTGGAGTGCAGCTGGACGTGACCAAGCACACGGAGGGCGAGAAGGTGCCCTTGGTTTCCGCCGGGGAGG
TGCCTCTCCTAGTGCGCTATGAGACGCGCCTCATGGCAAAGACGCAAGCTACCGCTGATGATCTCATGTC
CGTGATCAAGCATGTGGATAGGAAACAGTCCATCAACGAGGACGAGGACCCAGAAGGAGACGACGAGTTT
GGTTACCCAACCATGTCCTTCGATGCCTATGGAAATCCCCGCATGTCCGATGTGGATGCTTTGCTCAGCC
GGTCACTGGAGAAGCCAAAGTTCCGTCACAGGCGTGTTGCCTTCGATTTGGCCACCTCGCTCGAGCGAGT
GCAGAGGAATTTCTGTATCACAAATCCCTACTTGCCAGACCATCCCATTGTCTTCTGCTCGGACGATTTC
TTGGACCTAACCGGGTATACCAGAGAGGAGGTCATCGGCAGGAACTGCCGCTTCTTGCAAGGCCCTTTGA
CTGACAGAGCCCAGGTCGCCAAGATCCGCGAGGCCATTGACAACGAATCAGAGTGTACTGTACAGCTGCT
CAACTACCGCAAGGATGGCTCCTGCTTCTGGAATATGTTCCACTTGGCTCCCATCTTCGACAACAGTGGG
AAGGTGCAGTTCTTTGTCGGAGTGCAGACCGACGTGTCGGACCACGAGGTGCTTCCCAGTGAGGACGACC
GGGATGCGCCACGGCCGAGCCTGGCGCCTGAGCTAGCAGCTAGGGATAGCAGCGTCTCCATTGCTGGTGC
CCAAATAGTTGCGGGGGCGGTAAATAATATGAAGGTAGCATGGACGGGAGCAACCGATCAAGTCAAGTCG
TCTTATCGAGCATGGCTGCCTCACACTCGCAGGTTGGAGAAGATCCACGCTCACAACAGCACTGCGGTGC
CATGGGATGCAATCCGCATGATAACTGGAGGCACTTACCGCTTGAGCATGCTGAATATCGTCCCCATCAA
GCTACTAGGACGAGGCGATACGGGCAGCGTCCTGCTGATTAGGCTAGCGGGGACACCGCTGTACCTTGCG
ATGAAAGTCCTGGAGAAGAGGAACCTTCTTGAGAGGAACAAGGTGCAACGTGCTTTTACGGAGAGGGAGA
TCTTGGCGTCATTGGATCATCCTTTCCTGCCCACTCTATTTGACTGCTTTCAAACAGAGAGCCATTTGTG
CTTCTTGACGGAATTCTGCTCCGGCGGCGAGCTGTATTCTATGCTCAGCGGGCTGCCTGGCAATTGCGTG
CCGGAGCCGGTGGGAAAGCTGTACATTGCAGAGGTGTTGCTGTCATTGGAATACCTGCACTTAAAGGGTG
TAGTCTACCGTGATTTGAAGCCAGAGAACATCATGATTCAGGATGATGGCCATCTCCTGCTCACTGATTT
CGACTTGTCATTCCGCGCCGGCTGCACACCTGACGTGTTCTTCATCGAGAGGAGAGTGGGCAAGCACGTG
TTCAAATTCCCATGTGTTGTGGCTGAGCCTCGTGGCAAGACCAACTCCTTCGTGGGTACTGCGGAATACT
TGGCCCCAGAGGTGATCAACAACACCGGCCACTCTGCCGCTGTCGATTGGTGGGCTCTCGGCATTCTGCT
GTACGAGTTGTTGTATGGCTTCTCGCCCTTCTTCTCCGACACTCGCGCCGTGACTTTCGACAACATCCTC
CACTGCGACGTGGAATTCCCCAGCCATCCCGTCGTCTCTGCCGAGGGCAAGTCTCTGATTTGCGAGCTGC
TTGTCAAGGATACTGCGCGTCGTCTGGGCAGCAGATACGGCGCGGACGAGATCAAGAAACATCCTTTCTT
CTATGGCGTCAAGTGGGCTTTGATTCGGTCCCAGCGGGCTCCGTATGTGCCAGGCGAGGATGTTCCATCC
ATTTTCGGCCCAGAGGATGAGCGAGGAACCACCTTCGCCGGTTTTTAG
SEQ ID NO: 98
>ANC96849.1 phototropin, partial [Spirotaenia minuta]
MGSDGAYDAYGFPTEKSRTRGDSVSLATGLPAFSSETTGLLGSFRHSFILTDPSKPDFPIEYASDGFYEL
TGYTPSETMGRNCRFLQGPGTDRLEVEKLKEAIMEGRPISLRLLNYKKSGEAFWNLLTVSPFDVGGKRKF
LGVQLDVTKHTEGEKVPLVSAGEVPLLVRYETRLMAKTQATADDLMSVIKHVDRKQSINEDEDPEGDDEF
GYPTMSFDAYGNPRMSDVDALLSRSLEKPKFRHRRVAFDLATSLERVQRNFCITNPYLPDHPIVFCSDDF
LDLTGYTREEVIGRNCRFLQGPLTDRAQVAKIREAIDNESECTVQLLNYRKDGSCFWNMFHLAPIFDNSG
KVQFFVGVQTDVSDHEVLPSEDDRDAPRPSLAPELAARDSSVSIAGAQIVAGAVNNMKVAWTGATDQVKS
SYRAWLPHTRRLEKIHAHNSTAVPWDAIRMITGGTYRLSMLNIVPIKLLGRGDTGSVLLIRLAGTPLYLA
MKVLEKRNLLERNKVQRAFTEREILASLDHPFLPTLFDCFQTESHLCFLTEFCSGGELYSMLSGLPGNCV
PEPVGKLYIAEVLLSLEYLHLKGVVYRDLKPENIMIQDDGHLLLTDFDLSFRAGCTPDVFFIERRVGKHV
FKFPCVVAEPRGKTNSFVGTAEYLAPEVINNTGHSAAVDWWALGILLYELLYGFSPFFSDTRAVTFDNIL
HCDVEFPSHPVVSAEGKSLICELLVKDTARRLGSRYGADEIKKHPFFYGVKWALIRSQRAPYVPGEDVPS
IFGPEDERGTTFAGF
SEQ ID NO: 99
>XM_003063488.1 Micromonas pusilla CCMP1545 phototropin, blue light receptor
(PHOT), mRNA
CGCACCCGCGTCGCGCACGGACGACGAGCGCCGAGCGCCGGTCCTCGATCACGCGCGCGCGCGTCGAATC
TCGCGTCGAGCGCCGGAGCGTCGCGTCGGGGACGACGCGCGTCGAACGCGTCGCGCGCGCGAACGTTATC
CGGAGCTTTCCGTCCGATCCGCCCGGCGCGGCGCCAGCTGGATCGATCGATCTCCGCGTCGTCAGTCGAT
CGATCTCTCCCCGGCGTCGTCGCGTTCGAATCTAGGGCCGATCGCGGCGGCGCGGCGCGGCGCGTCATGG
CGGCGATGTCCGGTCAGGTCCCGCCGGATAAGATGCCGCAGGGTGTGTCATACACCGTCGACGAGAGCGG
CGGGATCGCCGCGCCCGAGGCGTCGAAAGGGTTGACGATGGCGCTGGCGTCGGTCCGGCACACGTTCACG
GTCAGCGACCCGACGCTGCCGGATTGTCCGATCGTGTACGCGTCCGACGGGTTCTTGAAGATGACCGGGT
ACTCCGCGGAGGAGGTGATCAACCGCAACTGCAGGTTCCTGCAGGGCGAAGACACCGATCGCGACGACGT
GCAAAAGATTCGCGACGCCGTGCAAAAAGGCGAGCGTTTGACCATCAGACTCCAAAACTACAAGAAGGAC
GGGACGCCGTTCTGGAACCTTCTCACGATCGCGCCGGTGAAGATGGAGGACGGCACGGTCGCGAAGTTCA
TCGGCGTGCAGGTGGACGTAACGGACCGGACGGAGGGCGAGGTGGGACGAACCGTCGGCGACGGCGGCGT
CGTCGGCGCCAAAGACGAGAAAGGCTTGCCGCTGCTCGTTCGGTACGACCAGAGACTCAAGGACCAGAAC
TACCCGGGCGTGGAGGACGTGGAGAAGGCGGTCATGAAGGGCGAGGGGATCGACGCGGACGCGACGAGGA
ACTCGCGCGCGAGAGAGGGGCTGGACATGGCGACGACGATGGAACGCATTCAGCAGTCGTTTCTCATCAG
CGACCCGTCGCTGCCGGATTGCCCGATCGTGTTCGCGTCCGACGGGTTCTTGGATTTCACCGGGTACGGC
CGCGAGGAGATCTTGGGGCGGAACTGCCGGTTCTTGCAGGGCGCGGGGACGGACCGCGACGCGGTGAAGG
AGATTCGGAACGCGATCAAAGACAACCGAGAGTGCACGGTTCGCCTGCTCAACTACACGAAGCAAGGGAA
ACCGTTCTGGAACATGTTCACGCTCGCGCCCGTCAGGGACCACGCGGGCGAGGTCAGGTTCTTCGCGGGG
GTGCAGGTGGACGTGACCGTGTACACGGACGCGGACGGCCGCCGCCTTGACAGCGTCGAGCTTCTGAGGC
AGACGAAGGCGCCGACGCCGCGGCACTCGGGCGACGACGAGGGCAAGTCAAAGTCGAAAGCCGCGACGAA
AAAAGTCTTGGAAGCGATCGGCGGGCTCACTGCAGCGGACGGCGAGCTGCCGTGGGCGAGGATGGTCGGC
CGCCTCGGCGCGCCGAAGCCGCACCAGGCCGGAGACGCGAACTGGGCGGCGCTGCGGAAGATCGTGGCCG
CGCACAAGGCGGCGGGGAGACCAGAGCGTTTGGCGCCGGAGGATTTCACGCCGTTGACGCGGCTCGGGCA
CGGCGACGTCGGCGCGGTGCACCTCGTGAGCCTGCGCGACGCGCCGAGCGCGAAGTTCGCGATGAAAGTT
CTCGTGAAGCAGGAGATGGTGGATCGAAACAAGCTTCATCGCGTGCGGACGGAGGGTCGAATTCTCGAGG
CGGTCGATCACCCGTTCGTCGCGACGCTGTACTCGGCGTTTCAGACGGACACGCACCTGTACTTTTTGAT
GGAGTACTGCGAGGGCGGCGAGCTGTACGAGACGCTGCAAAAGCAGCCCGGGAAGCGCTTCACCGAGGCG
ACGACCAAGTTTTACGCCGCGGAGGTTCTGTGCGCGCTGCAGTACCTCCACCTGATGGGCTTCATCTATC
GCGACTTGAAGCCGGAGAACATTTTGTTGCGTCGGAACGGACACGTCATCGTGACGGACTTTGACCTCTC
CTACTGCGCGTCGAGCCGCGCGCACGTCGTCATGATCGACGGCAAGGGCGAGGACGTCGTGGCCGGCGGC
GGGAGCGCGACGACGAGCGGGAGCGGGAGAGGGAGCGGCGGCGGGGGGGGAAGCGGCGGCGGCGGGAAGA
AGGAGCGTCGGCCGTCGGACGCCGGCTCGGAGAGTTCGAGTTCAAGAGGTGGGGGGGGCTTCTGCGGCAA
GGGCGGCGGCGGCGGCTCGAACCCCGCGACCCGCCGCGACACCCCGCGCCTCGTCGCGGAGCCGTTCGCG
TTCACCAACTCCTTCGTCGGCACGGAAGAGTACCTCGCCCCGGAGGTGTTGAACAGCACGGGGCACACGA
GCTCGATCGACTGGTGGGAGCTCGGCATCTTCATCCACGAGTGCGTGTTCGGGCTGACGCCGTTTCGCGC
GTCGAAACGCGAGCAGACGTTTCAGAACATCATCTCTCAGCCGCTCAGCTTCCCGTCGAACCCGCCGACG
AGCCCGGAGCTGAAGGATTTGCTCTCGCAGCTGCTGCGACGCGATCCGAGCGAGCGGTTGGGGACGAGAG
GGGGCGCGGAGGAGGTCAAGGCGCACCCGTTTTTCAAAGGGGTGGACTGGGCGTTGCTGCGTTGGAAAGA
CGCGCCGCTCGCGAAGAAGCCCGATCCGCCGAGGGCGGACGGCGGCGGCGACGAGGTGTTCGAGATCGAA
GTCTGAGAGAAGTCTGAGAGGTCTGTTTGGGGAGAAGAGAAGAGAAGTCTCAGTCTCTGGATGGAGACGT
CTGAGGCGGGCGGGCGGGCGGCGGGACGTCCCCTCGACGACGCGAGGGAGGAGCGTTTGCATAGCATACA
ATAGTAGATTCGCATCATTCACGAGCGCGTCGTTC
SEQ ID NO: 100
>XP_003063534.1 phototropin. blue light receptor [Micromonas pusilla
CCMP1545]
MAAMSGQVPPDKMPQGVSYTVDESGGIAAPEASKGLTMALASVRHTFTVSDPTLPDCPIVYASDGFLKMT
GYSAEEVINRNCRFLQGEDTDRDDVQKIRDAVQKGERLTIRLQNYKKDGTPFWNLLTIAPVKMEDGTVAK
FIGVQVDVTDRTEGEVGRTVGDGGVVGAKDEKGLPLLVRYDQRLKDQNYPGVEDVEKAVMKGEGIDADAT
RNSRAREGLDMATTMERIQQSFLISDPSLPDCPIVFASDGFLDFTGYGREEILGRNCRFLQGAGTDRDAV
KEIRNAIKDNRECTVRLLNYTKQGKPFWNMFTLAPVRDHAGEVRFFAGVQVDVTVYTDADGRRLDSVELL
RQTKAPTPRHSGDDEGKSKSKAATKKVLEAIGGLTAADGELPWARMVGRLGAPKPHQAGDANWAALRKIV
AAHKAAGRPERLAPEDFTPLTRLGHGDVGAVHLVSLRDAPSAKFAMKVLVKQEMVDRNKLHRVRTEGRIL
EAVDHPFVATLYSAFQTDTHLYFLMEYCEGGELYETLQKQPGKRFTEATTKFYAAEVLCALQYLHLMGFI
YRDLKPENILLRRNGHVIVTDFDLSYCASSRAHVVMIDGKGEDVVAGGGSATTSGSGRGSGGGGGSGGGG
KKERRPSDAGSESSSSRGGGGFCGKGGGGGSNPATRRDTPRLVAEPFAFTNSFVGTEEYLAPEVLNSTGH
TSSIDWWELGIFIHECVFGLTPFRASKREQTFQNIISQPLSFPSNPPTSPELKDLLSQLLRRDPSERLGT
RGGAEEVKAHPFFKGVDWALLRWKDAPLAKKPDPPRADGGGDEVFEIEV
SEQ ID NO: 101
>KU698737.1: 704-2884 Tetraselmis cordiformis
ATGTCTGCAATGATCCCCGAGACCTCCACGGAGCTTACTTCCGTGCTTTCAAACCTAAAGCATACTTTCG
TCGTTGCGGATGCAACTCTTCCGGACTGTCCACTGGTGTTTGCTAGCGAGTCTTTCTATGAGATGACGGG
ATACAGTAAGGACGAGGTTCTCGGGCATAACTGCAGGTTCTTGCAAGGGGAGGGAACCAGTCCAAAGGAG
ATTCAGAAATGTCGCGAGGCGGTGAAGAATGGGACTGTCGTTTCTGTCCGTCTCCTCAATTACCGCAAGG
ACGGCACGCCTTTCTGGAATTTGCTGACCTTGACACCGGTCAAAACATCGACTGGTCAGGTCACAAAGTT
CGTTGGCGTCCAGGTTGACGTGACGGGCCGCACAGAAGGCAAGAACTTCGTTGATGGGGAGGGGGTTCCC
CTCCTAGTCCATTATGATAATCGCCTGAAGGAAAACGTTGCAAAGAACATAGTCAGCGAGGTCGTGGACA
CCGTGGACAGAGTGGAGAACAAGGGTGCTGGCCGTGCAACGAAGCCCAAAGCCTTCCCTCGCGTGGCACT
TGATCTCGCCACCACCGTTGAGCGCATTCAGCAGAACTTCTGCATCTCGGATCCCACCCTGCCCGACTGT
CCCATCGTCTTCACCTCGGACGCCTTCCTGGAACTCACAGGCTACACGCGAGAGGAGGTTCTGGGTAGAA
ACTGCCGCTTTCTCCAGGGCCCCAGCACAGACCAAAGGACGGTGGACCAGATCCGCGAGGCCGTGACCAA
CAGGGAGGAGCTTACCGTCCGTATTCTGAACTATACAAAGCAGGGCATCCCATTCTGGAACATGTTAACC
CTCGCGCCGATCCGAGACGTGGACGGAACTTGCCGATTCATGGTCGGTGTACAGGTAGACGTCACCGCAG
CGGATGCCACCTCTGCGCCTGGCGAGATCCCAGCGCAGAAAGATCTGGGGCCTGTCTCGTCGGCTGCGTC
TGCTAGCAACGTTATTGGGAGCGCCCTCAAGAACCTTGGCATGGGAAATGCTGTCATGAAGAACCCTTGG
ACGCAGCTCACCATCGGCAAGGTTTACAGGAAGCCACATATGTCAGAAAACAAATCACTTCTGGCTCTCC
GTGCTACCGAGGCAGAGCATGGAACTCTGAAGGTCGTGCACTTCAAGCGTCTAAAGCAGGTTGGCAGCGG
AGATGTTGGTCTGGTGGACCTCGTGAGCCTGATCGGCACCAACCACGAGTTTGCCATGAAGTCTCTGGAC
AAGCAAGAAATGATCGAGCGCAACAAGGTAGCCCGTGTACTCACAGAGGAGTCGATTCTCTCACGGATCG
ATCACCCCTTCCTCGCTAACCTCTACTGCACACTCGAGACGCCTAGCCACCTGCACTTCCTGATGCAAAT
CTGCTCCGGTGGGGAGCTCTACGGGCTTCTTAACGCCCAGCCAAAGAAACGCTTGAAGGAGGCCCACGTC
CGCTTCTATGTTGCGGAGGTCCTCCTTGCGTTGCAGTACCTCCACCTTATCGGCGTCATATACCGCGACC
TGAAACCAGAGAATATTCTGCTCCACGGCAGCGGACATGCCATGCTCACAGACTTCGATCTCTCCTTCTC
GAAGGGTGAAACAGTCCCTCGGATAGAGAAACAGTCGGCCTCTGCTTGGAGCTCTCCAAAGGAGACCGCT
GGCTGCACCAAGTCGAGCTCGAATCTACCGGTAAAGCCCCACGACAAATACCTGCTGATCGCCGACCCGG
TCGCAAGGTCAAACTCGTTTGTGGGAACGGAGGAATACCTGGCGCCGGAGGTAATTAACGGCACAGGCCA
CGGCTCTGAGGTCGACTGGTGGGCGCTCGGCATCCTGACGTACGAGCTCATTTTTGGCACCACGCCATTT
CGGGGCATGCGTCGAGACGAGACCTTCGAGAACGTACTGCGTCTTCCTCTCACTGTCCCGCAGAAGCCCA
TTATCAGCGCCGAATGCAAGGACTTTATCCAGCAGCTCCTGATTAAGAACCCCGAGAAGCGTCTAGGTGC
CAAGAGGGGGGCTGAGGACATCAAGGCTCACCCCTGGTTTGCAAGTATCGAGTGGTCCCTGATTCGGAAT
GAGCAGCCGCCATTTGTGCCCAACAATGTAGCTACGCCAAGCAACACGGCCGGAGCCNCGACAACTACTG
ATGGCTCGTAG
SEQ ID NO: 102
>AML76833.1 putative LOV domain-containing protein [Tetraselmis cordiformis]
MSAMIPETSTELTSVLSNLKHTFVVADATLPDCPLVFASESFYEMTGYSKDEVLGHNCRFLQGEGTSPKE
IQKCREAVKNGTVVSVRLLNYRKDGTPFWNLLTLTPVKTSTGQVTKFVGVQVDVTGRTEGKNFVDGEGVP
LLVHYDNRLKENVAKNIVSEVVDTVDRVENKGAGRATKPKAFPRVALDLATTVERIQQNFCISDPTLPDC
PIVFTSDAFLELTGYTREEVLGRNCRFLQGPSTDQRTVDQIREAVTNREELTVRILNYTKQGIPFWNMLT
LAPIRDVDGTCRFMVGVQVDVTAADATSAPGEIPAQKDLGPVSSAASASNVIGSALKNLGMGNAVMKNPW
TQLTIGKVYRKPHMSENKSLLALRATEAEHGTLKVVHFKRLKQVGSGDVGLVDLVSLIGTNHEFAMKSLD
KQEMIERNKVARVLTEESILSRIDHPFLANLYCTLETPSHLHFLMQICSGGELYGLLNAQPKKRLKEAHV
RFYVAEVLLALQYLHLIGVIYRDLKPENILLHGSGHAMLTDFDLSFSKGETVPRIEKQSASAWSSPKETA
GCTKSSSNLPVKPHDKYLLIADPVARSNSFVGTEEYLAPEVINGTGHGSEVDWWALGILTYELIFGTTPF
RGMRRDETFENVLRLPLTVPQKPIISAECKDFIQQLLIKNPEKRLGAKRGAEDIKAHPWFASIEWSLIRN
EQPPFVPNNVATPSNTAGAXTTTDGS
SEQ ID NO: 103
>KJ195127.1 Bolbocoleon piliferum phototropin (PHOT) mRNA, complete cds
ATGGCACAATTGCCTCCTCCGGCAGCGCAGTTAACGCAGGTGTTGTCGAGTCTTCGCCACACATTTGCCG
TTGCCGATGCAACACTTCCAGATTGTCCCCTGGTGTACGCCAGCGAAGGGTTCTACCAGATGACTGGGTA
CACGAAGGACGAGGTTCTGGGTCACAACTGCCGTTTCCTCCAAGGTGAAGCCACAGATCCGGTAGAGGTT
GAGAAAATTCGTGATGCTGTTAAGAACGGCCGGAGTACCGCTGTTCGCCTTCTCAACTATCGCAAGGATG
GAACACCATTCTGGAATCTCCTTACTGTCACGCCCGTCTATGCAGCGGACGGGACGCTGTCCAAGTACAT
TGGAGTCCAAGTGGATGTCACCTCCAAAACCGAAGGATCAGCTTACACAGACCGCAGTGGTGTACCTCTT
CTAGTCAAGTACAATGACCGCCTGAAGCAGAACGTTGCTCATGACATTGTCGCGGATGTCAAAGATGCGG
TTGAAAGTGCTGAGCCGTCCCTGCAAAACAAGGCTGTTGGGACAGCGCCCAAGGCATTTCCACGTGTTGC
CATTGATCTTGCTTCGACAGTCGAGCGTATTCAACAAGCCTTTGTTGTGTCGGATCCAAACCTGCCAGAC
TGCCCAATCGTCTTCGCCTCGGACGCCTTTCTTGAGATGACGGGTTTTTCCCGGTTTGAAGTGCTCGGTC
GCAACTGTAGATTTCTCCAGGGAAAGCACACGGATGCGCATGCCATTGATGAGATCCGAGCAGCCGTGAA
AGAGGGCTCGGAGTGCACAGTGCGTCTGCTCAACTACAAAAAGGATGGCACCCCCTTCTGGAACATGCTT
TCTGTGGCGCCCATGATGGACGTCGACGGCACAGTGTGCTTCTTCATTGGCGTGCAAGTGAATGTCACCG
CTGAGACACCTGCACAAGACGGCCTGCCTGCAGTTGACCAGGGAGCTGTGAAGAAAGCATTGGACACTGC
ACAGATCCAGTCTGCAGTGTCACACCTGCACACAAAGCCGTCGTCGCCCGGGCGTGACCCGTTTTCTGCA
ATTCCGCATGCTAAGCTGCGTATCAAGCCGCACCGCAGCATGGACCGCGCCTGGCACGCGCTGCACAAGC
TCCAGCAGGCAGAGGGCACGATCGAGCTGCGGCATTTCAAGCGCGTGCAGCAGCTTGGTTCGGGTGACGT
GGGGCTGGTTGACCTTGTCCGCATCCAGGGGTCAGATGTCACTGTTGCCATGAAGACGATCGACAAAGTA
GAGATTTTGGAGCGCAACAAGCTGCACAGACTTCTCACCGAAGAGAATATTCTGCAGCAGTGTGACCATC
CGTTCCTCGCTGCATTATACTGTACCATCCAGAGTGAGCACTATCTGCATTTTGTCATGGAGTACTGCCC
CGGCGGAGAGCTGTACAAGCTGCTGTATGCACAGCACAACAACCGGTTTGAGGAGCGAGATGTGCAGTTC
TATGCTGCGGAAGTGCTCATGTCCCTGCAATATCTGCACATTCTCGGGTGTGTCTACCGAGATCTCAAGC
CTGAGAACATATTGATCATGGCGGATGGCCATGTGCGAGTGACAGACTTCGATCTTTGCATTCTTACGTC
AGATTTCAAGCCACAGTTGGTCAAGGGGCCACGCGAGCTTGCTGCAAATGCCAATGTAGCCCGGCACTCC
AAGACGGGGAAGGTTGGCGGAAAAGGATGCTATGGTGGCAGCGGAGTGCAGTTAGGTGAAGGGCTTGTGT
TGTCAGGGGAGCCGCAGATGCGAACCAACAGTTTTGTTGGGACTGAAGAGTATCTGTCGCCCGAGGTGAT
TCAGGGGAACTCGCACGGTGCTGCAGTTGACTGGTGGTCGTTGGGTATCCTGATATATGAGCTTTCCTTC
GGAACGACTCCATTCAAAGGGCAACGCCGGTCTGAGACCTTCTCCAGTATTGTCAAGAAGGACGTCAAGT
TTCCGGACGAACCTGTGGTCAGCTCGCAATGCAAGGACATCATTTTGCAGCTGCTTGTCAAGGATGAGAC
CAAGCGGCTGGGGAACAAGTATGGAGCGGAGGAGATCAAGCGGCACCCTTTCTTCAAAGACGTAGATTGG
CAGTTCTTGCGATCCCGAACACCACCGTGGGTGCCCCGAGGAACTGTGGCGTCAGGCAATATTGCTGGGT
TCTGA
SEQ ID NO: 104
>AHZ63928.1 phototropin [Bolbocoleon piliferum]
MAQLPPPAAQLTQVLSSLRHTFAVADATLPDCPLVYASEGFYQMTGYTKDEVLGHNCRFLQGEATDPVEV
EKIRDAVKNGRSTAVRLLNYRKDGTPFWNLLTVTPVYAADGTLSKYIGVQVDVTSKTEGSAYTDRSGVPL
LVKYNDRLKQNVAHDIVADVKDAVESAEPSLQNKAVGTAPKAFPRVAIDLASTVERIQQAFVVSDPNLPD
CPIVFASDAFLEMTGFSRFEVLGRNCRFLQGKHTDAHAIDEIRAAVKEGSECTVRLLNYKKDGTPFWNML
SVAPMMDVDGTVCFFIGVQVNVTAETPAQDGLPAVDQGAVKKALDTAQIQSAVSHLHTKPSSPGRDPFSA
IPHAKLRIKPHRSMDRAWHALHKLQQAEGTIELRHFKRVQQLGSGDVGLVDLVRIQGSDVTVAMKTIDKV
EILERNKLHRLLTEENILQQCDHPFLAALYCTIQSEHYLHFVMEYCPGGELYKLLYAQHNNRFEERDVQF
YAAEVLMSLQYLHILGCVYRDLKPENILIMADGHVRVTDFDLCILTSDFKPQLVKGPRELAANANVARHS
KTGKVGGKGCYGGSGVQLGEGLVLSGEPQMRTNSFVGTEEYLSPEVIQGNSHGAAVDWWSLGILIYELSF
GTTPFKGQRRSETFSSIVKKDVKFPDEPVVSSQCKDIILQLLVKDETKRLGNKYGAEEIKRHPFFKDVDW
QFLRSRTPPWVPRGTVASGNIAGF
SEQ ID NO: 105
>KT321732.1 Ulvella endozoica phototropin (PHOT) mRNA, partial cds
GCAGCGGCACCTCAGTTGACGCATGTGCTGTCCTCGCTGAGGCACACATTCGCTGTGGCGGACGCCACCC
TCCCGGACATGCCCCTGGTTTACGCCAGCGAAGGCTTTTATCAGATGACAGGATACACCAGGGAGGAGGT
GCTCGGGCACAACTGCCGGTTCCTGCAAGGGCAAGCGACAGACCTGAACGAGGTTGCAAAGATCAGAACA
GCCATAGAACAAGGCAAGGGTGCTGCTGTGCGTTTGCTCAACTATAGAAAGGACGGAACCCCTTTCTGGA
ACCTGCTGACAGTGATGCCCGTGTATGCTGCCGATGGCTCCTTGTCCAAGTTCATCGGTGTTCAAGTTGA
CGTGACATCTCGAACAGAAGGCTACGCGTACGTGGACAACTCGGGCGTTCCTCTTTTGGTCAAGTACAAT
GACCGCCTGAAACAGAATGTCGCACATGACATCGTCGAGGATGTTGTGAGTGCTGTGCAAGATGCCGAGA
CTGCCAAGGAACCTCAACCTAGCCAACCTAAAATTGGTGCAGCCCCAAAGGCCTTCCCCCGTGTGGCTAT
TGATCTGGCTACGACAGTTGAACGTATTCAGCAAGCATTCGTCATTTCCGATCCCAATCTGCCAGACTGC
CCCATCGTCTTTGCTTCAGATGCCTTTCTGCAGATGACCGGATTCTCCCGATATGAGGTCCTGGGACGTA
ATTGTCGGTTTCTCCAGGGCACACAAACGGACCCGCGCGCGGTCGATGAGATCCGTTCAGCGATCAGGGA
TGGCACAGAGTGCACAGTCCGCATCCTGAACTACAGAAAGGATGGCTCGCCCTTCTGGAACATGTTCTCG
CTTGCGCCCATGTCAGATATCGATGGCACGATCTGCTTCTTCATCGGTGTACAAGTTGATGTGACTGCAT
ACAACAACAGGGCTGCGTCAGGGGCAGACATAGTGCCCAATGTTGATGACAATGCAGCGAAGCTGGCATC
GGATACAGCCACCATCAAGCATGCCGTGAGCCATCTGGGAACTAGCCACGGTCCTCAAGTGGGTGACCCC
TTCGCTGTGATTCCCACCTCTGAGCTGAGTATCAAGCCCCACAGCAGCATGGACCGTGCCTGGCAAGCTC
TGCACAAGCTGCAGCAAACGCATGGCACCATCTCGTTAAAGCACTTCAAGCGTGTGCAACAGTTGGGTTC
GGGAGATGTGGGGCTTGTTGATCTGGTGCGCATTCAGGGATCGGAGGAGCTCGTTGCGATGAAGACAGTC
GACAAAGCTGAGATCCTTGAGCGCAACAAGCTCCACCGTCTGATCACCGAGGAGAGCATCCTGCGGCGCT
GTGACCACCCTTTCCTTGCGATGCTGTACTGTACGGTGCAGAGTGAGCACTATCTGCACTTCGTTATGGA
GTACTGCCCAGGTGGTGAGCTGTACAAGCTCTTATACGCTCAGAAGGGGAACCAGTTTGCAGAGCCTGAC
GTGGCGTTCTTCTCGTCAGAGGTTCTCCTGGCGCTGCAGTACCTACATGTCATCGGTTGTGTATATCGCG
ATCTGAAGCCGGAGAACATTCTGATAATGGGTGATGGCCACGTGCGCCTGACCGACTTTGACTTGTGCAT
ACTGAACCCGGACTTCCAGCCTGAAATGGTGCCACTCACTGGTGATACCAGTCCTACAGCTAGGGCGCGC
CAGATGAAGGGGAGGAGGCCCGGGGCTCCATGTGTGGGGGGGCGGAGCGGGAGCCCAAGGCAGCCACTGG
TGCTATCAGGAGAACCACAGCTTCGTACCAACAGCTTCGTTGGTACGGAGGAGTACCTGTCACCTGAGGT
CATCCAAGGCAACTCGCACGGTGCAGCTGTTGACTGGTGGTCGCTCGGCATTCTCATCTATGAACTCATA
TACGGAACTACACCTTTCAAGGGACAGCGGCGCTCTGAGACTTTCTCCAACATTGTGAAGAATCCTGTCA
AGTTCCCAGAGGAACCAGCCGTCACACCAGCATGCAAGGACATCATCACGCAGCTGCTTGTGAAAGATGA
GACGAAACGCCTCGGTACCAGGCTGGGTGCGGAAGAGATTAAGCAGCATCCTTTCTTCGCAAGCGTCCAC
TGGCAACTGCTGCGCTCCCGAAGCAACCCACCTTACATCCCTCGCGCAAAGGCGCTGACGGGTGATCACG
TGCCATCGTTCTGA
SEQ ID NO: 106
>ANC96857.1 phototropin, partial [Ulvella endozoica]
AAAPQLTHVLSSLRHTFAVADATLPDMPLVYASEGFYQMTGYTREEVLGHNCRFLQGQATDLNEVAKIRT
AIEQGKGAAVRLLNYRKDGTPFWNLLTVMPVYAADGSLSKFIGVQVDVTSRTEGYAYVDNSGVPLLVKYN
DRLKQNVAHDIVEDVVSAVQDAETAKEPQPSQPKIGAAPKAFPRVAIDLATTVERIQQAFVISDPNLPDC
PIVFASDAFLQMTGFSRYEVLGRNCRFLQGTQTDPRAVDEIRSAIRDGTECTVRILNYRKDGSPFWNMFS
LAPMSDIDGTICFFIGVQVDVTAYNNRAASGADIVPNVDDNAAKLASDTATIKHAVSHLGTSHGPQVGDP
FAVIPTSELSIKPHSSMDRAWQALHKLQQTHGTISLKHFKRVQQLGSGDVGLVDLVRIQGSEELVAMKTV
DKAEILERNKLHRLITEESILRRCDHPFLAMLYCTVQSEHYLHFVMEYCPGGELYKLLYAQKGNQFAEPD
VAFFSSEVLLALQYLHVIGCVYRDLKPENILIMGDGHVRLTDFDLCILNPDFQPEMVPLTGDTSPTARAR
QMKGRRPGAPCVGGRSGSPRQPLVLSGEPQLRTNSFVGTEEYLSPEVIQGNSHGAAVDWWSLGILIYELI
YGTTPFKGQRRSETFSNIVKNPVKFPEEPAVTPACKDIITQLLVKDETKRLGTRLGAEEIKQHPFFASVH
WQLLRSRSNPPYIPRAKALTGDHVPSF
SEQ ID NO: 107
>KJ195129.1 Coccomyxa pringsheimii phototropin (PHOT) mRNA, complete cds
ATGCCCGCTCAGACCGGGCAGGCTGAAAAGCAGCAGAAGGATGCGCAGCTGCATCCTGAGCTGCAGCGGC
CTGGGCAAAAGGTGCCAGGCCCTGCACCACAGCTCACAAAGGTTCTGGCGGGATTGCGGCATACTTTCGT
GGTAGCGGATGCCACGCTACCGGATTGCCCTTTGGTGTTCGCCAGCGAAGGATTCCTCTCGATGACAGGA
TACTCGGCTGAGGAGGTGCTGGGACACAACTGCCGCTTCCTGCAAGGGGAGGGTACAGACCCCAAGGAGG
TGGCAATCATCAGGGATGCAGTGAAGAAGGGGGAGGGCTGCTCTGTGCGCCTGCTCAACTACAGGAGGGA
TGGCACTCCCTTCTGGAACTTGCTCACCATGACGCCCATCAAGACAGAGGACGGCAAGGTGTCAAAGTTT
GTGGGAGTGCAGGTCGATGTGACCTCAAAGACAGAAGGGAAGGCCTTCTCAGATGCCACTGGTGTGCCAC
TGCTGGTGAAGTATGACACACGGCTGAGGGAAAATGTAGCAAAGAACATCGTCCAGGATGTCACGTCGCA
AGTGCAGGAAGCGGAGGAGGAAGACTCGGAGGCTACCAGGGTTGCCGGCCTGAAAGGCTTCAACAAGCTG
TGGCACAAGATGGGCAACAAGTCATCAGCCAACGACCCACAGCTGCAGAAGCAGGGAGAGCGGCTAGGCA
AGAAAATGACAGCCCCCAAAACGTTTCCCAGGGTGGCCATGGATCTGGCAACAACAGTGGAGCGCATCCA
GCAGAATTTCTGCATCTGCGATCCCAACCTGCCGGACAACCCGATAGTCTTCGCGTCAGACGGCTTCCTG
GAGATGTCCCAGAACGACCGCTTTGAGGTCCTGGGTCGCAACTGCCGCTTCCTGCAGGGGCCGGACACTG
ACCCCAAGGCGATCACTATCATCCGGGACGCGATCAAGAGCCAGAGCGAGGCGACCGTGCGCATTCTCAA
CTACCGCAAGAACGGGCAGCCCTTCTGGAACATGCTCACCATTGCACCCATGGCTGACGTTGGCGGCACC
TCCCGTTTCTTCATCGGAGTCCAGGTGGATGTGACGGCAGAGGATGTGCCGATGACGGGCGGCATTCCGG
CGGTTGACCAGAAGGCCGTCAAGGCGGCGGACCCGATGGGGAGCGTGCTGGGCATGGCACAGCGGCAGAT
GGGCGCTGGCTGGGCCGTGCACGACCCTTGGCAGGCCATCCATGCAGGCGTCTCTAGCCGCAAGCCACAC
AAGGCCCAGGAGAAGCCGTGGGCGGCGCTGCAGGCGACGAATGAGAAGACTGGTCGGCTGGGGCTGTCGC
AGTTCCGCCGCCTGAAGCAGCTGGGCACCGGCGACGTCGGCCTTGTGGACATGGTGGAGCTGCAGGACGG
CTCTGGCAGGTATGCGATGAAGACACTGGAGAAGGCGGAGATGCTGGAGCGCAACAAGGTGATGCGTGTG
CTGACGGAGGCCAAGATCCTGTCGGTGGTGGACCACCCCTTCCTGGCCAGCCTCTACGGCACCATCGTGA
CCGACACCCACCTCCATTTCCTCATGCAGATCTGCGAGGGCGGCGAGCTCTACGCGCTGCTCACCTCGCA
GCCCTCCAAGCGCTTCAAGGAGAGCCACGTCCGCTTCTACACTGCAGAGGTGCTGATTGCGCTGCAGTAC
CTGCACCTGATGGGCTTTGTGTACCGGGACCTGAAGCCCGAGAACATTCTGCTGCACAGCAGCGGCCACA
TCCTGCTTACCGACTTTGATCTCTCCTACTGCCAGGGCTCCACCGAAGTTAAGTTTGAGAAGAAGAAGAA
CGGCCACGCCAAGCCGCAGCTCGGGGCTGGGCAGGTGAGACCCTCAGAGGAGATCACGCTGATCGCTGTG
CCGGACGCGCGCGCCAAATCCTTTGTGGGCACTGAGGAGGACCTTGCGCCAGAGGTCATAAACGGTGTCG
GCCACGGGCCAGGAGTGGACTGGTGGAGTTTTGGGATCCTGATCTATGAGCTGTTGTACGGATTCACCCC
TTTCCGGGGCAAGAAGCGTGACGAGACATTCAACAACATCCTCAAGCGACCGCTCAACTTCCCTGAATTG
CCGGAGGTCTCCGACGAGTGCAAGGACCTGATTTCGCAGCTGCTGGAGCGCGACCCGGCCAAGCGGCTGG
GCGCGCACGCGGGCGCAGAGGAGATCAAGGCGCACCCCTTCTATGAGTCCATCAATTGGGCCCTCCTGCG
CAACACGCGGCCGCCCTACATCCCCCGCCGCAATGTGCGCAAGGCCACCCCCTCCCCCGCCGCGGAGGCC
AATTTCGGCGACTTCTGA
SEQ ID NO: 108
>AHZ63930.1 phototropin [Coccomyxa subellipsoidea]
MPAQTGQAEKQQKDAQLHPELQRPGQKVPGPAPQLTKVLAGLRHTFVVADATLPDCPLVFASEGFLSMTG
YSAEEVLGHNCRFLQGEGTDPKEVAIIRDAVKKGEGCSVRLLNYRRDGTPFWNLLTMTPIKTEDGKVSKF
VGVQVDVTSKTEGKAFSDATGVPLLVKYDTRLRENVAKNIVQDVTSQVQEAEEEDSEATRVAGLKGFNKL
WHKMGNKSSANDPQLQKQGERLGKKMTAPKTFPRVAMDLATTVERIQQNFCICDPNLPDNPIVFASDGFL
EMSQNDRFEVLGRNCRFLQGPDTDPKAITIIRDAIKSQSEATVRILNYRKNGQPFWNMLTIAPMADVGGT
SRFFIGVQVDVTAEDVPMTGGIPAVDQKAVKAADPMGSVLGMAQRQMGAGWAVHDPWQAIHAGVSSRKPH
KAQEKPWAALQATNEKTGRLGLSQFRRLKQLGTGDVGLVDMVELQDGSGRYAMKTLEKAEMLERNKVMRV
LTEAKILSVVDHPFLASLYGTIVTDTHLHFLMQICEGGELYALLTSQPSKRFKESHVRFYTAEVLIALQY
LHLMGFVYRDLKPENILLHSSGHILLTDFDLSYCQGSTEVKFEKKKNGHAKPQLGAGQVRPSEEITLIAV
PDARAKSFVGTEEDLAPEVINGVGHGPGVDWWSFGILIYELLYGFTPFRGKKRDETFNNILKRPLNFPEL
PEVSDECKDLISQLLERDPAKRLGAHAGAEEIKAHPFYESINWALLRNTRPPYIPRRNVRKATPSPAAEA
NFGDF
SEQ ID NO: 109
>KJ195128.1 Prasiola crispa phototropin (PHOT) mRNA, complete cds
ATGGCGTCTCAAAGAAAGGTGCCGGCCCCCGCAGCTCAGCTCACAAAGGTGCTTGCGGGTTTACGGCATA
CGTTTGTGGTAGCTGACGCAACTCTACCGGATTGTCCACTGGTCTACGCGAGCGAAGGGTTCCTGCAGAT
GTCTGGCTACACTGCTGACGAGGTGTTGGGGCACAACTGTCGGTTTCTGCAAGGAGAGGGCACCGACCCA
AAGGAGGTCGCGGTCATTCGAGATGCTGTAAAACACGGTACCAGCTGCTCTGTGAGGCTGCTGAATTATC
GCAAGAATGGCAGCCCCTTTTGGAATCTGCTGACTATGACGCCTATCAAAACGGACGATGGCAAAGTGAC
CAAGTATGTTGGCGTCCAAGTGGATGTAACGAGTAAAACCGAGGGGCTTTCAACTGGCGATCAATCAGGC
GTGCCTTTACTGGTGAAGTATGATACCAGGCTCAAGGAAAGTGGGAAGAATGCAGTCAACGAAATCAACG
CGACAGTCCAGGAGGCAGAGCCGAGCAAGCTGCCCAAGAAGTCTAAAGCACCCAAGGCTTTCCCTCGTGT
CGCCATGGACTTGGCGACGACTGTCGAACGCATCCAGCAGAACTTTGTGATCTCTGACCCCCACTTGCCC
GACTGCCCCATCGTGTTCGCATCCGACGGGTTCTTGGACCTCACAGAGTATAGCCGCGAGGAGATTCTCG
GCCGCAACTGCCGCTTCTTGCAGGGCCAAGACACAGATCCTGCAGCGGTGTCTGAGATTCGGGATGCTGT
GCGGAACGGCAGCGAGGCGAGTGTCAGGCTGCTGAACTACAAGAAGTCCGGGACACCCTTCTGGAACATG
TTCACTTTGGCGCCCATGGCAGACGTGGATGGCAATCTGCGCTTCATCATCGGAGTCCAGGTCGATGTGA
CGGCAGCGGATACAACGGCTCCTGGGAAGCTGCCAGCTGTCGATCCGCAGGCAGCTGTCAGTGCTCAGAC
GACTGGGATGATTAACACCGCGCTCCAACATATGGGGCTGGGTCCTGACCCCTGGAAAGCTATTAGGGTC
GGGGTGGCATCGACTAAGCCACATTCTTCAGCAGCTCCGGAATGGAAGAAGTTGCGCAGACTACAGGACA
GCGATGTTGCCCTCAAGCTGTCCCACTTTCGAAGAGTGAAACAGCTCGGCTCGGGTGATGTCGGCCTGGT
TGATCTCGTCCAAATTCAGGGCGACTCCGAATCAAGGTATGCTATGAAGACACTAGAGAAGCGAGAGATG
GTAGAACGCAACAAGGTGATGCGCGTCCTCACTGAGGAGCGAATCCTGGCTGCCGTGGACCACCCCTTCG
TTGCACATCTATACGCCACCATTCAAACCGAGACACACCTCCACTTCCTCATGCAGTACTGTGGGGGAGG
TGAGCTATACGGCCTCCTGATGAGTCAGACTCACAAGCGGCTATCAGAGAGTCACATGCAGTTTTATGTC
GCTGAAGTGCTGCTGGCTCTCCAATATCTTCACCTTCTCGGTTTTGTATACCGGGATCTGAAGCCGGAGA
ATATTCTGATCAGTGCCTCCGGACATGCGCTGCTGACGGATTTCGATCTGTCTTTCTGCTCAAATGGCAC
CAAGCCTCGCATTGAGCGGTCAGCGCCATCGCATCTGAGGGAGCAGAGCAGTCGCAACAGCAGCAAGGTG
CAGAAGAACGGACAGAACAAGTCGGAGAGGTGGAACGCAATGGAGGCAGCTTCTCTGACTCTGGTAGCTG
AGCCCGAGGGTCGTGCCAATTCCTTTGTGGGCACAGAGGAGTATTTGGCCCCTGAAATCATCAACGGCAC
TGGCCACGGTCCCGGAGTTGATTGGTGGTCTTTTGGTATCCTAATGTATGAGCTGGTGTACGGGTTCACA
CCCTTCCGTGGGGCCAAACGAGACCAGACTTTCGAGAACATCCTCAAGTCCCCTCTCATTTTCCCACCCA
AGCCAGAGATCAGCAAGTCCTGTCAGGATTTGATATGTGCACTTCTGGTGCGACAACCAGAGTCGCGGCT
AGGCGCCTACGCCGGAGCTGAGGAAATCAAGCTGCATCCTTTCTTCAGCAACATCAACTGGCCGCTGATC
CACAACAGCAAGCCTCCCTATGCGCCCTCATCCTCTGGTGGCGGCCTCCGACAGAACCCAGCGTTTGACA
ACTTCTGA
SEQ ID NO: 110
>AHZ63929.1 phototropin [Prasiola crispa]
MASQRKVPAPAAQLTKVLAGLRHTFVVADATLPDCPLVYASEGFLQMSGYTADEVLGHNCRFLQGEGTDP
KEVAVIRDAVKHGTSCSVRLLNYRKNGSPFWNLLTMTPIKTDDGKVTKYVGVQVDVTSKTEGLSTGDQSG
VPLLVKYDTRLKESGKNAVNEINATVQEAEPSKLPKKSKAPKAFPRVAMDLATTVERIQQNFVISDPHLP
DCPIVFASDGFLDLTEYSREEILGRNCRFLQGQDTDPAAVSEIRDAVRNGSEASVRLLNYKKSGTPFWNM
FTLAPMADVDGNLRFIIGVQVDVTAADTTAPGKLPAVDPQAAVSAQTTGMINTALQHMGLGPDPWKAIRV
GVASTKPHSSAAPEWKKLRRLQDSDVALKLSHFRRVKQLGSGDVGLVDLVQIQGDSESRYAMKTLEKREM
VERNKVMRVLTEERILAAVDHPFVAHLYATIQTETHLHFLMQYCGGGELYGLLMSQTHKRLSESHMQFYV
AEVLLALQYLHLLGFVYRDLKPENILISASGHALLTDFDLSFCSNGTKPRIERSAPSHLREQSSRNSSKV
QKNGQNKSERWNAMEAASLTLVAEPEGRANSFVGTEEYLAPEIINGTGHGPGVDWWSFGILMYELVYGFT
PFRGAKRDQTFENILKSPLIFPPKPEISKSCQDLICALLVRQPESRLGAYAGAEEIKLHPFFSNINWPLI
HNSKPPYAPSSSGGGLRQNPAFDNF
SEQ ID NO: 111
>KT321727.1 Scourfieldia sp. STK 1728 phototropin (PHOT)
ATGAATCCGGAGTATGACGACCCGCCGCCGGCGGGCGCGGAGCGCGTCACCAAGGACGCCACCCACAATG
CGCTGATCGTGAAGAAGGTCCGCACCAAAGAGGAGCACGAGGCGCTGTCGCCCGTGACGGGCGTCGTGGC
GCCGTCCAAGCCCCTCACGATGGCGATGGCTGGCATGTGGCAGACTTTTGTCATCACAGACATGACCATC
AAGGACGGGCCCATCGTGTTCGCGTCGGAGGGCTTTTACCACATGACGGGCTACCCCGCGGATGAGGTGC
TCGGCCGCAACTGCCGCTTCCTGCAGGGGCCGGACACGAACCGCGATGACGTGACCAAGCTGCGCAATGC
CGTGATGGGCGGATTCTCCGTCAGCGTGCGGCTGCTCAACTACCGCAAGGATGGCAACCCGTTCTGGAAC
TACCTCACCATGACGCCCATCAAGAACGAGGACGGTATCGTGACCAAGTTCGTGGGCGTTCAGGTGGACG
TGTCGAGCAAGACCGAGGGCCGCGTCACGTCGGCGTTTGCGGACCGGCAGGGCGTGCCGCTGCTGATCAA
GTACGACACGCGCATCCGCGATAACGCGATGCGCGAGAACGTGGCGCCCGTCATCCAGGCCGTGGCCACC
GCTGAGGGCGGCACCGCCGCCTCGTTCCCGACGGCCGCCTCGGACGCGGTCGGCGGCGTGGCCGACTCGC
GCGCGTCGATGGGCGCGACCTCGATCGATCAGGCCGCGCAGCCGGGCTCGATGGAGGTCCGGCGCTCGGT
GGTGCCGGCCTGGGAGGCCAAGACCCGCCACGGTCTGGACCTGGCCACCACCCTGGAGCGCCTGCAGGCG
TCCTTCTGCGTGTGCGACCCGTCAGTCAAGGGCGCGCCGATCGTGTTTGCGTCCGACACGTTCTTGACGT
TGACCGAGTACCCGCGCGAGGAGGTGCTGGGCCGCGACTTTCTGTTCCTGCAGGGCCCCAAGACCGACAA
GCGGGCGCTCAAAGAGATCAGCACGGCCATCGCGGAGAACTCCGAGGCGACGGTTCGCGTGCTCAACCAG
ACCAAGTCCGGCCGCCAGTTTTGGGACATGTTCCACGTGGCGCCGATCAAGGACCTGGCGGGTAACGTGA
TGTATCTGATCGGTGTGCACATGGATGTATCCCAGATGGTGGACGACCGGTCGGCCTCCAAGGACGCCAA
CCTGGTGGGCCAGCTCGCGCCGCACCTGAAGCAGGCCATGGGCGGCATCTCCACGGCCGTCGGCGCGGTG
GCCGACAAGGCCAAGATTGCGGACCCGTTCGCGCGCATCGACGGCCGGCGCGTGCGCGCCACCAAGCCGC
ACCAGTGCAACGACCAGGGCTGGAAGGCCATCCAGGCGCTGGTGACCCGCGACGGCTACGTGGGGCCGAT
GCACTTCGAGAAGGTCCGGCGGCTCGGCTCCGGCGACGCGGGCCAGGTGTACCTGGTTCAGATCAAGGGC
GGCGGGCACCGCTACGCCATGAAGGTGCTGAGCAAGCAGGACATGCTCGAGCGCAACAAGGTGCACCGTG
TCAACACCGAGGAGTCGATCCTGTCCTCTCTGGACCACCCCTTCCTGGCCACGCTGTACGCGGCCTTCCA
GACCGAGTCGAATCTGCACTTCATCATGCAGTACTGCGGCGGCGGGCAGCTGTACGACCTGCTGCGCAAG
CAGGAGCCCAAGGGCCGGCTGCCGGAGGAGTCGACGCGCTTTTACACGGCCGAGGTGCTGCTGGCGCTGC
AGTATCTGCACCTGCAGGGCTTCATCTACCGCGACCTCAAGCCCGAGAACGTGCTGCTGCGCGAGGACGG
CCACATCATCTTGACGGATTTCGATCTGTCCTACACGGGCGTGACCAAGCCTGTGATGCTGCCGGCCGCG
GCGGGGCCCGCCGGCGCGCGCGGGCCGGCGCTGATGGCCGAGCCCGAGGCGATGGCCAACTCCTTCGTGG
GGACGGAGGAGTACCTGTCGCCCGAGGTGGTGGCGGGCGCCGGGCACTCGGCGGGGGTGGACTGGTGGTG
CCTGGGCATCTTCATGTTTGAGCTGTTTTATGGCATGACCCCGTTCAAGGGCGCCTCGCTGGACCGCACC
ATGGACAACGTGCTCAAAAAGGACGTGGTGTTCCCCGAGGTGCCCAGCGCGGGCTTCCCCGGTGTGCAGA
TGTCGCCCGAGGGCCAGGACTTTATCCGTCAGCTGCTGCAGCGCGACCCGGCCAAGCGCCTGGGCGGCAA
GGGCGGCGCCGAGGAGATCAAGGCGCACCCCTTCTTTGAGGGCGTCGACTGGGCGCTGCTGCGCAACACG
ACGCCGCCCTATGTGCCGCCGGTGGGCCGCGGGCCGGCCAAGGTGCCGGGCGCGTCGTCG
SEQ ID NO: 112
>ANC96852.1 phototropin. partial [Scourfieldia sp. STK 1728]
MNPEYDDPPPAGAERVTKDATHNALIVKKVRTKEEHEALSPVTGVVAPSKPLTMAMAGMWQTFVITDMTI
KDGPIVFASEGFYHMTGYPADEVLGRNCRFLQGPDTNRDDVTKLRNAVMGGFSVSVRLLNYRKDGNPFWN
YLTMTPIKNEDGIVTKFVGVQVDVSSKTEGRVTSAFADRQGVPLLIKYDTRIRDNAMRENVAPVIQAVAT
AEGGTAASFPTAASDAVGGVADSRASMGATSIDQAAQPGSMEVRRSVVPAWEAKTRHGLDLATTLERLQA
SFCVCDPSVKGAPIVFASDTFLTLTEYPREEVLGRDFLFLQGPKTDKRALKEISTAIAENSEATVRVLNQ
TKSGRQFWDMFHVAPIKDLAGNVMYLIGVHMDVSQMVDDRSASKDANLVGQLAPHLKQAMGGISTAVGAV
ADKAKIADPFARIDGRRVRATKPHQCNDQGWKAIQALVTRDGYVGPMHFEKVRRLGSGDAGQVYLVQIKG
GGHRYAMKVLSKQDMLERNKVHRVNTEESILSSLDHPFLATLYAAFQTESNLHFIMQYCGGGQLYDLLRK
QEPKGRLPEESTRFYTAEVLLALQYLHLQGFIYRDLKPENVLLREDGHIILTDFDLSYTGVTKPVMLPAA
AGPAGARGPALMAEPEAMANSFVGTEEYLSPEVVAGAGHSAGVDWWCLGIFMFELFYGMTPFKGASLDRT
MDNVLKKDVVFPEVPSAGFPGVQMSPEGQDFIRQLLQRDPAKRLGGKGGAEEIKAHPFFEGVDWALLRNT
TPPYVPPVGRGPAKVPGASS
SEQ ID NO: 113
>KT321734.1 Oedogonium foveolatum phototropin (PHOT) mRNA
ATGTCGGCTCCTTCCGGTGCTCCAAATGTGCCTGCACCAGCGGCTCAGTTAACTAAAGTCCTTGCTGGAT
TGCGGCACACATTCGTGGTGTCAGATGCAACACTACCTGATTTTCCGCTGGTTTTTGCTAGCGAGGGATT
TCTTCAAATGACGGGCTACACTGCGGATGAAGTCTTGGGTCATAACTGTCGCTTCCTTCAAGGAGAAGGT
ACAGATCCCAAGGAAGTGGCCAAGATTCGCGAAGCTTTAAAAAAAGGTGAACCCATCAGCGTCAGGTTGT
TAAACTATCGTAAAGATGGCACTCCGTTTTGGAACCTGCTTACGATGACGCCCATCCACACCCCTGATGG
CAAGGTGTCCAAGTTCATTGGGGTGCAGGTCGATGTGACCAGCAAGACCGAGGGCAAAGCTTACGAAGAA
AACAAGGGCATGCCGTTAATCGTCAAGTATGACGCACGTTTGCGTGAGAATGTTGCCAAGAACATCGTCG
AAGACGTCCAAACCACGGTCGAGAAGGTGGAGCTCGGCGAGCGTCCGAAAGTTCATGGTCCGAAGGCCTT
CCCCCGTGTTGCGCTAGATTTAGCCACAACAGTCGAGCGTATCCAGCAAAACTTCGTCATCTGCGATCCC
ACCCTCCCTGATTGCCCGATTGTGTTTGCATCTGATGCGTTCCTGGAGCTCACAGAGTATTCCCGCGAGG
AGGTGTTAGGTCGAAACTGCCGGTTTTTGCAAGGCAAACACACTGATGCTGCAGCAGTCGCTGAGATCAG
AGAGGCAGTCCACAATGGCCAGGAACTGACTGTGCGTCTTCTGAATTACACCAAGTCCGGCCGGCCGTTT
TGGAACATGTTCACCATGGCTCCCATGATGGATCAGGACGGTACGATCCGCTTCTTCATTGGAGTGCAAG
TCGATGTCACTGCTCAGTCTAAGGCTCAAGGCGAAGCTGCAGCATGGAAGAAGACTCCTGAGGTGCAGGC
TCAAGCGCAGCTGGGGCATCAGGCAGCTTCTGCTATTGGTGCAGCCCTTAAAATGAATGCCACTTGGGTT
GCAGATCCATGGTCTGCTATTGCTGGAAACGTTGTGAGATGCAAACCCCACAAGTCAGCTGACAGTGCGT
ACAAAGCTTTGGCGGACATATCTAAGAAGGAGGGCAAAGTAAAATTGATGCACTTTCGTCGCGTAAAGCA
ACTAGGATCTGGTGATGTTGGTTTGGTGGACTTGGTGCAGCTGCAGGGTCAGGAGCACCAGTTTGCCATG
AAAACTCTGGATAAATGGGAAATGCAAGAACGCAACAAAATTCAGCGCGTTTTGACGGAAGTGCAAATAC
TGAATCAAGTTGATCACCCATTCCTTGCAACTTTGTACTGCACCATCCAAACTGAAACCCACTTGCATTT
CATCATGGAATATTGTGAAGGTGGTGAGCTGTATGGCTTATTGCATTCACAACCCAGGAAGCGGCTCAAA
GAATCTCAAGTCAAGTTCTATGCAGCAGAGGTGCTGGTTGCTCTGCAGTACCTACACCTGCTGGGCTATG
TGTATCGGGACTTGAAGCCTGAGAATATTCTGCTGCATAGTTCAGGCCACGTGCTTCTAACTGATTTTGA
TCTGTCCTATGCTAAGGGCACCACGACTCCAGTCCTGGAAGAGCGTTCGGTTCCGAAAATGCAGGCGAAA
ACCAAGAATGGGAAGAAGGTTGTGGTGACTCCGCCACAATATGTCCTGGTTGCAGAGCCCCAGGCGAAGG
CCAACTCCTTCGTAGGCACCGAAGAGTACCTTGCACCGGAAGTCATCACTGCTCAGGGTCATTCTGCAGG
CGTTGACTGGTGGTCCTTTGGTATCTTGATGTATGAGTTATTGTACGGTTTCACGCCTTTCAGGGGTTCA
CGGCGAGATGAAACTTTCGAGAACATCCTGAAACAGCCTCTTTCATTTCCTTCCAACCCGCCAATTAGCG
ACCAGTGCAAGAACTTGATTTCTTCGCTGCTTGTCAAGGAGCCAGCCCAGCGTCTGGGGGCCAAGGCAGG
AGCTGAGGACATCAAAGCTCATCCATTTTTCGCAGGCACTAATTGGGCTCTCTTGCGCAATGAGACACCT
CCTTACGTGCCGAAGCAGGGCAAAGATCCTGCAACCCCAGGCAGTGCTCAGTTCAACAACTTTTGA
SEQ ID NO: 114
>ANC96859.1 phototropin, partial [Oedogonium foveolatum]
MSAPSGAPNVPAPAAQLTKVLAGLRHTFVVSDATLPDFPLVFASEGFLQMTGYTADEVLGHNCRFLQGEG
TDPKEVAKIREALKKGEPISVRLLNYRKDGTPFWNLLTMTPIHTPDGKVSKFIGVQVDVTSKTEGKAYEE
NKGMPLIVKYDARLRENVAKNIVEDVQTTVEKVELGERPKVHGPKAFPRVALDLATTVERIQQNFVICDP
TLPDCPIVFASDAFLELTEYSREEVLGRNCRFLQGKHTDAAAVAEIREAVHNGQELTVRLLNYTKSGRPF
WNMFTMAPMMDQDGTIRFFIGVQVDVTAQSKAQGEAAAWKKTPEVQAQAQLGHQAASAIGAALKMNATWV
ADPWSAIAGNVVRCKPHKSADSAYKALADISKKEGKVKLMHFRRVKQLGSGDVGLVDLVQLQGQEHQFAM
KTLDKWEMQERNKIQRVLTEVQILNQVDHPFLATLYCTIQTETHLHFIMEYCEGGELYGLLHSQPRKRLK
ESQVKFYAAEVLVALQYLHLLGYVYRDLKPENILLHSSGHVLLTDFDLSYAKGTTTPVLEERSVPKMQAK
TKNGKKVVVTPPQYVLVAEPQAKANSFVGTEEYLAPEVITAQGHSAGVDWWSFGILMYELLYGFTPFRGS
RRDETFENILKQPLSFPSNPPISDQCKNLISSLLVKEPAQRLGAKAGAEDIKAHPFFAGTNWALLRNETP
PYVPKQGKDPATPGSAQFNNF
SEQ ID NO: 115
>KT321737.1 Fritschiella tuberosa phototropin (PHOT) mRNA, partial cds
ATGGCAGACCCGAACGTCCAACCGGTGCCCGCGCCGGCAACGCAGCTCACCAAGGTCCTGGTTGGCCTGC
GGCACACTTTTGTCGTCGCTGATGCCACGCTGCCAGACCTCCCGCTGGTTTACGCCAGCGACGGGTTCTA
CCAGATGACGGGCTACGGCCCGGACGAGGTGCTGGGCCACAACTGCCGCTTCCTGCAAGGAGAGGGCACG
GACCCCAAGGAGGTGGCGAAGGTGCGGGCAGCCATCAAGAATGGCGAGCCCGTGAGCGTGCGCCTGCTCA
ACTACCGCAAGGACGGCACGCCCTTCTGGAACTTGCTCACCATGACGCCCATCAAGACGCCCGACGGCCG
CGTCTCCAAGATCGTGGGCGTGCAGGTCGACGTCACCAGCAAGACCGAGGGCAAGGCCGCGGCCGAGGCC
AAGGGCGTGCCGCTGCTGGTCAAGTACGACGCACGCCTGCGCGAGAACGTCGCCAAGAAGATCGTCGAGG
ACGTCACCACCGCCGTGCAGACCGCCGAGACCGGAGAGGACAAGGTCAAGGCGCAGGCGCCCAAGGCCTT
CCCGCGTGTGGCCATGGACCTGGCCACCACGGTGGAGCGCATCCAGCAGAACTTCTGCATCTGCGACCCC
ACGCTGCCCGACTGCCCCATCGTGTTCGCGTCGGACGCCTTCCTGGAGCTGACAGAGTACACGCGCGAGG
AGGTGCTGGGGCGCAACTGCCGCTTCCTGCAGGGGCCGGCCACGGACAAGCACACCATCGACGAGATCCG
GCAGGCCATCCGCATGGGCTCCGAGTGCACCGTGCGCGTGCTCAACTACACCAAGACAGGCCGCCCCTTC
TGGAACATGTTCACGCTGGCGCCCATGTGCGACCAGGACGGCACCATCCGCTTCTTCATCGGCGTCCAGG
TGGACGTGACGGCGCAGTCGGGGCAGCCGGGCATGGACGTGCCGCAGTGGTCACGCACCAAGTCGCAGGA
GGTGCAGACCGCCAAGCAGGGCCACCAGGCGGCCACCGCCATCTCGGCGGCGCTGCAGACCATGGGCTGG
CCCGCCAACCCGTGGGCGTCCATCCAGGGCGTCGTCGCGCGCCAGAAGCCGCACAAGCGCGGCGACCGCG
CGTTCCAGGCGCTGCGGGAGCTGCAGGAGCGTGAGGGCAAGCTCAAGCTGCTGCACTTCCGGCGCATCAA
GCAGCTGGGCACGGGCGACGTGGGCAACGTGGACCTGGTGCAGCTGCAGGGCACCGAGTTCCGCTTCGCG
ATGAAGACGCTGGACAAGCTGGAGATGCAGGAGCGCAACAAGGTGCAGCGCGTGCTCACAGAGGAGGGCA
TCCTGTCGCACGTCGACCACCCCTTCCTTGCCACCCTCTACTGCACCATCCAGACGGACACGCACCTGCA
CTTCGTCATGGAGTTCTGCGACGGCGGCGAGCTGTACGGCCTGCTCAACAGCCAGCCCAAGAAGCGGCTC
AAGGAGGCGCACGTGCAGTTCTACGCGGCGGAGGTGCTGCTGGCGCTGCAGTACCTGCACCTGCTGGGCT
ACATTTACCGCGACCTGAAGCCGGAGAACATCCTGCTGCAGGCGTCCGGCCACGTGCTGCTGACCGACTT
CGACCTCTCCTACGCGCAAGGCGTCACCGACGTCTCTCTGGAGAAGGTAGTCAAGCGGTCTCGCACTGGC
AAGGTGGTGCGGCGCGGCGCCGGCATCGAGAACTACACGCTGGTGGCGGAGCCGGAGGCGCGCGCCAACT
CTTTCGTGGGCACGGAGGAGTACCTGGCGCCCGAGGTGATCAACGCCAGCGGGCACGGCAGCCAGGTGGA
CTGGTGGTCCTTCGGCATCCTCATCTACGAACTCGTCTACGGCTTCACGCCCTTCCGCGGCTCCCGCCGC
GACGAGACCTTCGAGAACATCCTCAAGCGCGAGCTCACCTTCCCCCTCAAGCCCGAGATCAGCCCGGAGT
GCAAGTCGCTCATCTCGGCGCTGCTGGTCAAGGACCCCACGATGCGGCTGGGCTACAAATACGGCGCGGA
GGAGATCAAGAAGCACCCCTTCTTCGCCGGCATCGTCTGGCCCCTGCTGCGCCACCGCGCGCCCCCCTAC
GTCGTAGAGAACCAGCTGCCTGTGGGCGTGCCGCACGCCAATCAGCACTTTGACGACTACTAA
SEQ ID NO: 116
>ANC96862.1 phototropin, partial [Fritschiella tuberosa]
MADPNVQPVPAPATQLTKVLVGLRHTFVVADATLPDLPLVYASDGFYQMTGYGPDEVLGHNCRFLQGEGT
DPKEVAKVRAAIKNGEPVSVRLLNYRKDGTPFWNLLTMTPIKTPDGRVSKIVGVQVDVTSKTEGKAAAEA
KGVPLLVKYDARLRENVAKKIVEDVTTAVQTAETGEDKVKAQAPKAFPRVAMDLATTVERIQQNFCICDP
TLPDCPIVFASDAFLELTEYTREEVLGRNCRFLQGPATDKHTIDEIRQAIRMGSECTVRVLNYTKTGRPF
WNMFTLAPMCDQDGTIRFFIGVQVDVTAQSGQPGMDVPQWSRTKSQEVQTAKQGHQAATAISAALQTMGW
PANPWASIQGVVARQKPHKRGDRAFQALRELQEREGKLKLLHFRRIKQLGTGDVGNVDLVQLQGTEFRFA
MKTLDKLEMQERNKVQRVLTEEGILSHVDHPFLATLYCTIQTDTHLHFVMEFCDGGELYGLLNSQPKKRL
KEAHVQFYAAEVLLALQYLHLLGYIYRDLKPENILLQASGHVLLTDFDLSYAQGVTDVSLEKVVKRSRTG
KVVRRGAGIENYTLVAEPEARANSFVGTEEYLAPEVINASGHGSQVDWWSFGILIYELVYGFTPFRGSRR
DETFENILKRELTFPLKPEISPECKSLISALLVKDPTMRLGYKYGAEEIKKHPFFAGIVWPLLRHRAPPY
VVENQLPVGVPHANQHFDDY
SEQ ID NO: 117
>KT321742.1 Pediastrum duplex phototropin (PHOT) mRNA
ATGTCGCAACCAAGTGCATCGATACCAGCTGCGGCTGGGCAGCTGACCCAGGTGTTAGCTGGGCTGAAGC
ATACTTTCGTTGTGGCCGATGCAACGCTGCCAGACTGTCCCCTGGTGTTCGCTAGCGAAGGATTCTACCA
GATGACTGGCTATGGCCCTGATGAGGTTCTAGGGCACAACTGCCGCTTCTTGCAAGGAGAGGGCACTGAC
AAGAAGGAAGTTACAAAGCTGCGCCAAGCGATCAAGGATGGTGAGCCCATCAGCGTCCGTCTGCTGAACT
ACCGCAAGGATGGAACACCATTCTGGAACCTGCTGACCATGACCCCAATCAAGACACCTGATGGCAAGGT
GTCGAAGTTCGTGGGGGTGCAGGTGGATGTGACCAGTAAGACAGAGGGGAAGCTGCCCCACGAGAACCTG
CTGGTCAAGTATGATGCCCGCCTGCGTGACAACGTGGCCGTCAACATTGTAACAGACGTCACCAACGCTG
TGCAGAAGACAGAGACGGGGACCAACGCCCCGCTGAGTGTGATCCCTACAGGGATTGGGAAGCACGGCCC
CAAGGCGTTCCCCCGTGTGGCTATTGATCTGGCCACCACTGTGGAGCGCATCCAGCAGAACTTCTGTATC
TGTGACCCCACGCTACCGGATTGCCCTATTGTGTTTGCGTCTGATGCGTTCCTGGAGCTGACTGAGTATG
CTCGTGAGGAGGTGCTGGGCCGCAACTGCAGGTTCTTACAGGGCCCTGGCACAGACCCCAAGACCGTGCA
GGTGATCCGTGATGCCATCAAGACACGGGATGAGATCACGGTGCGCATCCTGAACTACACCCGCAGCGGG
AAGCCCTTCTGGAACATGTTCACCCTGGCCCCCATGAAGGACAGCAATGGGGAGACACGCTTCCTGGTGG
GAGTGCAGGTGGATGTGACTGCCCAGGGTGAAAAGGGTGACACCACCCTGCCCTCCTGGAACAAGACCAC
CAGTGAGGAGGTGGTGAAGGCGCAGCAGGGCAACCAGGCAGCCAGCCTTATCAGCAACGCACTGCAGAGC
ATGGGCTGGGGGGCCAACCCCTGGGCAGGCATCACAGGCACAGTTATGAGGAGGAAGCCTCACAAGGGTG
AGGACCAGGCCTATCAGACGCTGCTGAACCTCCAGGGGCGGGAGGGGAAGCTGAAGCTGGCTCACTTCAG
GCGGGTGAAGCAGCTGGGGGCGGGAGATGTGGGGCTGGTGGACCTGGTGCAGCTGCAGGGTACTGACTTG
AAGTTCGCCATGAAGACGCTGGACAAGTGGGAGATGCAGGAGCGCAACAAGGTGGCCCGCGTGCTGACGG
AGGAGAACATCCTGACTGTGGTGGACCACCCCTTCCTTGCCACCCTCTACTGCGCCATCCAGACAGACAC
ACACCTCCATTTCGTGATGGAGTACTGTGAGGGAGGGGAGCTGTATGGCCTGCTCAATGCACAGCCCAAG
AAGCGCTTGAAAGAGGCACATGTCAAGTTCTACGCTGCTGAGGTGCTGCTGGCTCTGCAGTACCTGCACC
TGCTGGGGTACATCTACCGCGACCTGAAGCCCGAGAACATCCTCCTCCACCACACTGGCCATGTACTGCT
CACTGACTTTGACCTCAGCTATGCACGTGGCACAGCCAGCGTTAAGATCCAGGCCACACCTAGTGAGGGG
GGCAAGCGGGTCAAATCTTCCAGCTGCACCAAGCCGCCAGAGGAGGCGGGGCCGGCACCGCATACTGCCC
CCAATGGGGACGAGCTGGTGCTGCTGGCAGAGCCTGCCGCCCGGGCGAACTCCTTTGTGGGGACAGAGGA
GTACCTGGCTCCTGAGGTCATTAATGCGGCTGGGCATGCAGCACCGGTGGATTGGTGGTCCTTTGGGATC
CTCATGTACGAGCTGCTGTATGGCTTCACGCCCTTCCGTGGTGCACGGCGTGAGGAGACGTTTGAGAACA
TCTTGCGTAATCCGCTGACCTTCCCCAGCAAGCCTGTGGTGTCGGAGGCTTGTCAAGATCTGATCCGGCA
GCTGCTGGTGAAGGACCCGGCAAAGCGGTTGGGGACGCGGGCGGGTGCGGAGGAGATCAAGAAGCATGAG
TTCTTCAAGGGGGTCAACTGGGCGCTGGTGCGGAATGAGCAGCCACCGTATGTGCCAAGAAAGGTGGCAG
CAGGAGGGAAGGAGGGCAGTAGTTTGAGTATGAATGCCAGTATGGATCAGGGGAGCGCTGGGTTTGACAA
CTACTGA
SEQ ID NO: 118
>ANC96867.1 phototropin [Pediastrum duplex]
MSQPSASIPAAAGQLTQVLAGLKHTFVVADATLPDCPLVFASEGFYQMTGYGPDEVLGHNCRFLQGEGTD
KKEVTKLRQAIKDGEPISVRLLNYRKDGTPFWNLLTMTPIKTPDGKVSKFVGVQVDVTSKTEGKLPHENL
LVKYDARLRDNVAVNIVTDVTNAVQKTETGTNAPLSVIPTGIGKHGPKAFPRVAIDLATTVERIQQNFCI
CDPTLPDCPIVFASDAFLELTEYAREEVLGRNCRFLQGPGTDPKTVQVIRDAIKTRDEITVRILNYTRSG
KPFWNMFTLAPMKDSNGETRFLVGVQVDVTAQGEKGDTTLPSWNKTTSEEVVKAQQGNQAASLISNALQS
MGWGANPWAGITGTVMRRKPHKGEDQAYQTLLNLQGREGKLKLAHFRRVKQLGAGDVGLVDLVQLQGTDL
KFAMKTLDKWEMQERNKVARVLTEENILTVVDHPFLATLYCAIQTDTHLHFVMEYCEGGELYGLLNAQPK
KRLKEAHVKFYAAEVLLALQYLHLLGYIYRDLKPENILLHHTGHVLLTDFDLSYARGTASVKIQATPSEG
GKRVKSSSCTKPPEEAGPAPHTAPNGDELVLLAEPAARANSFVGTEEYLAPEVINAAGHAAPVDWWSFGI
LMYELLYGFTPFRGARREETFENILRNPLTFPSKPVVSEACQDLIRQLLVKDPAKRLGTRAGAEEIKKHE
FFKGVNWALVRNEQPPYVPRKVAAGGKEGSSLSMNASMDQGSAGFDNY
SEQ ID NO: 119
>Volvox carteri f. nagariensis phototropin
ATGGCAGGGGTACCCTCCCCCGCCAGCCAGCTCACGAAGGTGCTGGCCGGCCTGCGGCATACGTTTGTCG
TTGCGGATGCAACACTCCCGGATTGCCCCCTGGTGTACGCCAGTGAAGGGTTCTACGCAATGACAGGATA
CGGTCCTGATGAGGTTCTTGGACATAACTGCCGGTTTCTGCAGGGCGAGGGTACGGACCCCAAGGAGGTT
CAAAAGATCCGCGAGGCCATCAAGAAGGGGGAGGCGTGCTCGGTGCGCCTGCTCAACTACCGCAAAGATG
GCACGCCGTTCTGGAACCTGCTCACGGTGACGCCCATCAAGACTCCGGACGGCAAGGTGTCCAAGTTTGT
GGGTGTGCAGGTCGATGTGACCAGCAAGACGGAGGGCAAGGCGCTCGCGGACAACTCCGGCGTGCCCCTG
CTCGTCAAGTACGACCACCGTTTGCGCGAAAACGTGGCCAAGAAGATTGTGGATGATGTCACCATTGCCG
TGGAGAAGGCGGAGGGTGTGGAACCTGGGGCAGCCTCGGCCGCCGCCACGGCGGCTGGTCAGGGAAAGCC
GCAGGGCGTCCGCGGCGCGGCCCCCAAGTCCTTTCCTCGTGTGGCTTTGGATCTGGCCACCACCGTGGAG
CGCATCCAGCAGAATTTCGTCATTTCAGATCCAACATTGCCGGACTGCCCCATCGTCTTTGCTTCGGATG
CATTTTTGGAGCTGACTGGCTATTCGCGCGAGGACGTGCTGGGACGTAACTGCCGCTTTCTACAGGGCCC
CGGTACTGATTCAGCCACCGTGGATCAGATCCGTGAGGCCATCCGCACGGGTACGGAGATCACGGTCCGC
ATCCTGAACTACACCAAGCAGGGCCGACCCTTCTGGAATATGTTCACCATGGCGCCCATGAGAGATCAGG
ACGGCTCAGTGCGCTTCTTTGTGGGGGTGCAGGTAGACGTGACTGCTCAGTCCGCGACGCCGGACAAGAC
TCCCACGTGGAACAAGACTCCCTCCGCGGAGGAGGAGAAGGCCAAGCAGGGAGCCGTGGCGGCGTCCATG
ATTAGCAGCGCGGTTATGGGCATGGCCACACCCATGGCCAGCAACCCCTGGGCCGCCATCAACGGGGAGG
TCATGCGGCGTAAGCCCCACAAGAGCGATGATAAGGCCTATCAGGCGCTGTTGGCGCTGCAGCAGCGTGA
CGGCAAGTTGAAGCTGATGCACTTCCGGCGTGTGAAGCAGCTAGGGGCGGGAGATGTGGGTCTGGTGGAC
CTGGTGCAGCTGCAGGGCACGGACTTCAAGTTCGCCATGAAGACCCTGGACAAGTTCGAGATGCAGGAGC
GCAACAAGGTGCCCCGTGTGCTGACCGAGTGCTCTATTCTGGCGGCTGTGGACCACCCCTTCCTGGCCAC
CCTCTACTGCACCATTCAGACCGACACGCACCTGCACTTCGTCATGGAGTACTGCGATGGTGGCGAGCTG
TACGGCCTGCTGAACAGTCAGCCCAAGAAGAGGCTCAAGGAGGAGCATGTCCGGTTTTACGCGGCGGAGG
TCCTCCTGGCCCTGCAGTACCTGCACCTACTCGGCTACGTGTACAGGGACCTAAAGCCCGAGAACATCCT
TCTTCACCACTCGGGGCACGTGCTATTGACGGACTTTGACTTGTCGTACAGCAAGGGCGTTACGACACCG
CGGCTAGAGCGCGTGGCGGCGCCGGACGGCAGCGGTGGCGGCTCGGCGCCGGCGCCGGCGGGGTCGGCGG
GGTCAAAGTCTTCGCGCAAGTCCTTCCTGCTGCTGGCGGAACCTGTGGCCCGTGCGAACAGTTTCGTGGG
CACCGAGGAGTACTTGGCACCGGAGGTCATCAACGCGGCGGGACACGGATCGGGTGTCGACTGGTGGTCG
CTAGGCATCTTGATCTACGAGCTGCTGTACGGCACTACACCCTTTCGTGGATCAAGGCGGGACGAGACCT
TTGACAACATCATCAAGTCACAGCTGCGCTTCCCGGCCAAACCTGCTGTCAGTGAGGAGGGCCGCGACCT
CATCGAGAAGCTTCTGGTCAAGGACGTGAGCCGTCGCCTCGGCAGTCGTACAGGGGCCAATGAGATTAAG
TCGCATCCCTGGTTCAAGAGCATCAATTGGGCGCTGCTGCGCAACGAGCCGCCGCCGTACGTGCCGCGCC
GGGCATCCAAGACGCAGGGCGGTGGTGGCGGCGGCGGCGGCGGCGCGGCGTTCGACAACTACTGA
SEQ ID NO: 120
>EFJ48666.1 phototropin [Volvox carteri f. nagariensis]
MAGVPSPASQLTKVLAGLRHTFVVADATLPDCPLVYASEGFYAMTGYGPDEVLGHNCRFLQGEGTDPKEV
QKIREAIKKGEACSVRLLNYRKDGTPFWNLLTVTPIKTPDGKVSKFVGVQVDVTSKTEGKALADNSGVPL
LVKYDHRLRENVAKKIVDDVTIAVEKAEGVEPGAASAAATAAGQGKPQGVRGAAPKSFPRVALDLATTVE
RIQQNFVISDPTLPDCPIVFASDAFLELTGYSREDVLGRNCRFLQGPGTDSATVDQIREAIRTGTEITVR
ILNYTKQGRPFWNMFTMAPMRDQDGSVRFFVGVQVDVTAQSATPDKTPTWNKTPSAEEEKAKQGAVAASM
ISSAVMGMATPMASNPWAAINGEVMRRKPHKSDDKAYQALLALQQRDGKLKLMHFRRVKQLGAGDVGLVD
LVQLQGTDFKFAMKTLDKFEMQERNKVPRVLTECSILAAVDHPFLATLYCTIQTDTHLHFVMEYCDGGEL
YGLLNSQPKKRLKEEHVRFYAAEVLLALQYLHLLGYVYRDLKPENILLHHSGHVLLTDFDLSYSKGVTTP
RLERVAAPDGSGGGSAPAPAGSAGSKSSRKSFLLLAEPVARANSFVGTEEYLAPEVINAAGHGSGVDWWS
LGILIYELLYGTTPFRGSRRDETFDNIIKSQLRFPAKPAVSEEGRDLIEKLLVKDVSRRLGSRTGANEIK
SHPWFKSINWALLRNEPPPYVPRRASKTQGGGGGGGGGAAFDNY
SEQ ID NO: 121
>KT321740.1 Tetradesmus dimorphus phototropin (PHOT) mRNA
ATGGCTGGACATGTCCCCGCTGCTGCATCGCAGCTGACACAAGTGCTGGCAAAGCTCAGGCACACCTTTG
TGGTGGCAGATGCTACGCTGCCTGACTGCCCTCTGGTGTATGCCAGTGAATCGTTCTACCAGATGACTGG
CTATGGGCCTGATGAGGTCCTGGGGCACAACTGCCGCTTCCTGCAAGGCGAGGGCACAGATCCGAAGGAG
GTGGCCAAGCTGCGCAATGCTATCAGGGCTGGCGAGCCGGTCAGCTGCAGGCTGCTCAATTACCGCAAGG
ATGGCACGCCCTTCTGGAACCTGCTGACAATGACACCCATCAAGACGCCTGATGGCAAGGTCTCCAAGTT
TGTGGGCGTGCAGGTGGATGTGACCAGCAAGACGGAGGGCAAGGTGGACAACAGCCACATGCTGGTCAAG
TACGATGCACGCCTGCGCGACAATGTGGCATCTGGCGTGGTGCAGGAGGTCACAGACACAGTGCAGATGA
CTGAGACGGGCACGCACATCAACCCTGGCATGATTCCCAGCGGCATCGGCAAGGTGGGGCCCAAGGCCTT
CCCCCGCGTGGCCATGGACCTGGCCACCACTGTGGAGCGCATCCAGCAGAACTTTGTCATCTGCGACCCC
AGCCTGCCGGACTGCCCGATTGTGTTTGCCAGTGATGCCTTCCTGGACCTGACGGAGTTCCCGCGCGAGG
AGGTGCTTGGGCGCAACTGCAGGTTCCTGCAGGGCCCGGGCACGGACCCCGGCACGGTGCAGACCATCCG
CGACGCGATCAAGAGCGGCGACGAGATCACCGTGCGCATCCTCAACTACAAGCGCAGCGGCACGCCCTTC
TGGAACATGTTCACGCTGGCGCCCATGAAGGACAGCGACGACACCATCCGCTTCCTGGTCGGCGTGCAGG
TGGACGTCACAGCGCAGGGCGCCGCCGGCGACACCGCCGCGCCAGCATGGACCAAGTCGCCCAGCGACGA
GGCCGAAAAGGTGCAGCAGGGCAACCAGGCAGCCTCCCTCATCAGCTCAGCGCTGCAGAACCTCGGCTGG
GGAGCCAGCCCCTGGGCTCAAATCAGCGGCAGCATTATGCGGGCGAAGCCGCACAAGGCCAGCGATGCAG
CCTTCCAGGCGCTGCTGCGGCTGCAGCAGCGCGAGGGGCAGCTGCGGCTGAACCACTTCCGGCGCGTGAA
GCAGCTGGGGGCGGGAGATGTGGGGCTGGTAGACCTGGTGCAGCTGCAGGGCACGGACATGAAGTTTGCC
ATGAAGACGCTGGACAAGTGGGAGATGCAGGAGCGCAACAAAGTGGCGCGCGTGCTGACAGAAGAAAGCA
TCCTCACAGCCATCGACCACCCCTTCCTGGCAACCTGCTACTGCTCCATCCAGACAGACTCCCACCTGCA
CTTTGTGATGGAATTCTGCGAGGGGGGCGAGCTGTACGGGCTGCTGAACGCGCAGCCACGCAAGCGGCTC
AAGGAGTCACACGTCAAGTTTTACGCTGCTGAGGTGCTCATCGCGCTGCAGTACCTGCACCTGCTGGGCT
ACATCTACCGCGACCTCAAGCCAGAGAACATCCTGCTGCACCACACCGGCCACGTGCTGCTGACAGACTT
TGACCTGAGCTACGCGCGCGGCACCACCACGCCGCGCATGCAGGCCACTAACGCGGAGTGCACGCCGCGC
CACAGCAGCAGCTGCACCAAGGTGGAGGAGCCGCTGCAGCCGGGCCAGGCGCCCAATGGCGACGAGCTGC
TGCTGCTGGCTGAGCCTGTGGCTCGCGCCAACAGCTTCGTGGGCACTGAGGAGTACCTGGCGCCCGAGGT
CATCAACGCAGCTGGCCACGCTGCGCCTGTTGACTGGTGGAGCTTTGGCATCCTCATCTACGAGCTCATG
TTTGGCACCACGCCCTTCAGGGGTGCGCGGCGCGAGGAGACGTTTGAAAACGTGCTGCGCAACCCGCTCA
CATTCCCTTCCAAGCCAGCCATCAGCCCAGAAGCGCAAGACCTCATGAGCCAGCTGCTCGCAAAGGACCC
GGCGCAGCGCTTGGGCACACGCGCAGGCGCAGAGGAGATCAAAAAGCACCCCTGGTTTGAGGGCATCAAT
TGGGTGCTTCTGCGGCACCAGCAGCCGCCGTATGTGCCGCGTATGTGCCGCGCCGCGCTGTTGCTGCTGC
TGCAAGTGGTGCTGCTGGCAGCGGCAACGCGAGCGCGGACGGCGTGCCGGGCGCGGCGGGCGGCGCCCGC
GGCG
SEQ ID NO: 122
>ANC96865.1[Tetradesmus dimorphus]
MAGHVPAAASQLTQVLAKLRHTFVVADATLPDCPLVYASESFYQMTGYGPDEVLGHNCRFLQGEGTDPKE
VAKLRNAIRAGEPVSCRLLNYRKDGTPFWNLLTMTPIKTPDGKVSKFVGVQVDVTSKTEGKVDNSHMLVK
YDARLRDNVASGVVQEVTDTVQMTETGTHINPGMIPSGIGKVGPKAFPRVAMDLATTVERIQQNFVICDP
SLPDCPIVFASDAFLDLTEFPREEVLGRNCRFLQGPGTDPGTVQTIRDAIKSGDEITVRILNYKRSGTPF
WNMFTLAPMKDSDDTIRFLVGVQVDVTAQGAAGDTAAPAWTKSPSDEAEKVQQGNQAASLISSALQNLGW
GASPWAQISGSIMRAKPHKASDAAFQALLRLQQREGQLRLNHFRRVKQLGAGDVGLVDLVQLQGTDMKFA
MKTLDKWEMQERNKVARVLTEESILTAIDHPFLATCYCSIQTDSHLHFVMEFCEGGELYGLLNAQPRKRL
KESHVKFYAAEVLIALQYLHLLGYIYRDLKPENILLHHTGHVLLTDFDLSYARGTTTPRMQATNAECTPR
HSSSCTKVEEPLQPGQAPNGDELLLLAEPVARANSFVGTEEYLAPEVINAAGHAAPVDWWSFGILIYELM
FGTTPFRGARREETFENVLRNPLTFPSKPAISPEAQDLMSQLLAKDPAQRLGTRAGAEEIKKHPWFEGIN
WVLLRHQQPPYVPRMCRAALLLLLQVVLLAAATRARTACRARRAAPAA
SEQ ID NO: 123
>KT321746.1 Pedinomonas tuberculata phototropin (PHOT) mRNA, partial cds
ATGCACAAACCGAATCTGGAGGGCGTGAAGGTCCAGCTTCCTCCCCAAGCTGGACAACTATCCAAATTAT
TAGAGGGCTTGAAGCATACATTCGTAGTGTCAGATGCTACCCTGCCTGACTGCCCGCTCGTTTTCGCTTC
GGAAAGTTTCTACAAAATGACCGGATTCAACGCTGATGAAATTCTCGGCAAAAATTGTCGTTTCCTACAA
GGAGAGCAAACAGATCGTGAAACAGTAGCAAAGATTCGAGCAGCAATTAACAAGGGGGATGGAATATCCT
GCCGCCTCCTGAACTACCGAAGGGACGGCACTCCCTTCTGGAACCTGCTCACCATCACCCCTATCAAGAA
CGCGCAGGGCAAGGTCACCAAATTCGTCGGAGTACAAGTAGACGTGACCTCGAAGACCGAGGGCAAAGTA
GAGACGGAGAGGTCGCTGGTGCACTACGATGACCGACTCCGTCAGACTGTGGCACATAAAGTAGTAACGG
ACGTCACTATGGCCGTAGAGGACGCTGAGATGTCTATGGAGGGAGGCAAGAAGGCCGCCCCTAAAGCGTT
CCCCCGTGTCGCTATTGATCTGGCCACCACTGTGGAACGTGCGCAGCAGAATTTCGTAATCGCGGACCCT
AAATTGCCCGATTGCCCTATCGTGTTCGCCTCCGATCAGTTCTTAGATTTGACTGGGTATGCACGAGAGG
AGGTGCTAGGGAGAAACTGCAGATTCCTACAGGGTCCTGATACTGACCCTAAGACCGTGGCTGAGATCAG
AGATGCCCTAGCTAACAATAAAGAGGTGACGGTGCGTATCCTCAACTACACAAAATCCGGCAAGCCCTTC
TGGAACTTGTTCACCTTAGCACCTATTCAAGATATCGATGGCACCGTAAGGTTCTTCGTGGGAGTCCAGG
TGGACGTGACTGATAAGGAGGCGCAGAAGGCGATGGAGGCTCAGGCTGAGGTGATGGCCCTGCAGTCCGC
AGTGAAGGACCTGCAGTCAGGCTGGAAGGACGATCCATGGAAGGGCCTCAGCACCGGGCTGTGTAAGAAC
AAGCCACATACCGGCGTTACAGAGCCCTACAAGGCCCTGGAGGCTATCCAGAAGCGTGACGGCGCTCTGG
GTCTGCAGCACTTCAAGCGTATTAAGCAGCTAGGCAATGGTGATGTGGGTATGGTGGACCTGGTCCAGCT
GGACGGTACCACCTTCAAATTCGCCATGAAAACTCTCGACAAAAGGGAGATGCTGGAGCGCAATAAGGTT
CACCGTGTGATGACTGAGATCAAGTGTCTAGGTATGGTCGACCACCCTTTTGTGGCCTGCATGTACGCCG
TGCTGCAGACCAAGACCCACCTGCACTTCATCCTCGAATACTGCGAGGGGGGCGAGGTATACTCCTTATT
GAACGCGCAGCCTAACAAGAGGCTCAAGGAGCAGCACGTCCAGTTCTATGCGGCCGAGGTACTTATCGCC
CTGCAGTACCTGCATCTGATGGGAATTATCTACAGAGATCTCAAGCCCGAGAACTTGCTTATCCGCGATG
ACGGCCACGTGATCATGACGGACTTCGATCTGTCTTATGTGAAGGGTACTCTGGAGTGCCGCGTGGATCA
GGTACAGACCTTCGTCCCAGCCAAGAACAACTCGAACCGAAAGATCAAGATCAACATACCCACACTGGTG
GCAGAGCCCAAGGCGCGGGCTAACTCGTTCGTTGGCACAGAGGAATACCTAGCCCCTGAGGTGATCAACG
CCGGGGGGCACTCCTCCGGGGTGGACTGGTGGTCGTTTGGTATCCTGATGTACGAGCTGCTGTATGGCAC
CACCCCTTTCCGCGGCCCCCGTCGAGACGACACGTTTGAGAACATCTTGTCAGCCCCCCTTAACTTCCCC
AGCAAGCCTCAGGTGTCGCCTCAGTGCATCGACCTGATCCAGCAGCTGCTACATAAGAACCCGGCTAAGA
GACTAGGAGCACAAAGAGGAGCAGAAGAAATCAAGGCTCATCCCTTCTGGAAGGGCATTAACTGGGCGCT
ATTGCGGAGAGAGAGGCCTCCCTTCGTGCCTAAGAAGGGAGGAGTGGGAGCGCCGGCAACCGGCGGCAGC
TCATCCTCGGGGGGAGTCCCCGGCCCGG
SEQ ID NO: 124
>ANC96871.1 phototropin, partial [Pedinomonas tuberculata]
MHKPNLEGVKVQLPPQAGQLSKLLEGLKHTFVVSDATLPDCPLVFASESFYKMTGFNADEILGKNCRFLQ
GEQTDRETVAKIRAAINKGDGISCRLLNYRRDGTPFWNLLTITPIKNAQGKVTKFVGVQVDVTSKTEGKV
ETERSLVHYDDRLRQTVAHKVVTDVTMAVEDAEMSMEGGKKAAPKAFPRVAIDLATTVERAQQNFVIADP
KLPDCPIVFASDQFLDLTGYAREEVLGRNCRFLQGPDTDPKTVAEIRDALANNKEVTVRILNYTKSGKPF
WNLFTLAPIQDIDGTVRFFVGVQVDVTDKEAQKAMEAQAEVMALQSAVKDLQSGWKDDPWKGLSTGLCKN
KPHTGVTEPYKALEAIQKRDGALGLQHFKRIKQLGNGDVGMVDLVQLDGTTFKFAMKTLDKREMLERNKV
HRVMTEIKCLGMVDHPFVACMYAVLQTKTHLHFILEYCEGGEVYSLLNAQPNKRLKEQHVQFYAAEVLIA
LQYLHLMGIIYRDLKPENLLIRDDGHVIMTDFDLSYVKGTLECRVDQVQTFVPAKNNSNRKIKINIPTLV
AEPKARANSFVGTEEYLAPEVINAGGHSSGVDWWSFGILMYELLYGTTPFRGPRRDDTFENILSAPLNFP
SKPQVSPQCIDLIQQLLHKNPAKRLGAQRGAEEIKAHPFWKGINWALLRRERPPFVPKKGGVGAPATGGS
SSSGGVPGP
SEQ ID NO: 125
>XM_002506242.1 Micromonas commoda blue light receptor mRNA
ATGAGCGAGCCGGCTCCCGCCGTCGAGCCCTCGGCGGCTGCGCCTTCGGACGAGGTGCCAAAATTCGACG
AGACCAAGACGCACGAGAGCATCGACATCGGCTTCACGGTGGACGCCGGCGGCGGCATCAGCGCGCCGCA
GGCGAGCAAGGACCTGACCAACGCGCTGGCGTCGCTCCGTCACACCTTTACCGTGTGCGACCCGACGCTC
CCGGACTGCCCCATCGTCTACGCGTCGGACGGGTTCCTGAAGATGACCGGATACCCCGCCGAGGAGGTCC
TCAACCGCAACTGCAGGTTCCTCCAGGGGGAGGAGACGAACATGGACGACGTGCGCAAGATATCCGAGGC
GGTCAAGAAGGGCGAGAGGATCACCGTCCGCCTGCTCAATTACCGCAAGGATGGCCAGAAGTTCTGGAAC
CTGCTCACCGTCGCGCCGGTCAAGCTGCCGGACGGGACCGTCGCCAAGTTCATCGGCGTGCAGGTGGACG
TCAGCGACAGGACCGAGGGCAACGCGGATAACTCCGCGGCGATGAAGGACACCAAAGGTCTCCCCCTGCT
CGTCAAGTACGATCAGCGGTTGAAGGATCAGAACTTCAACAGGGTGGACGACGTGGAGAAGGCGGTGCTG
ACGGGCGAGGGCGTCGACCTCGACGCGAACCCGGTGGCGGCGAACAGAGGAGGCCTCGACATGGCCACCA
CCCTGGAGCGCATCCAGCAGTCCTTCGTCATCGCCGACCCGTCTTTGCCCGACTGCCCCATCGTGTTCGC
GTCTGACGGGTTTTTGGACTTCACCGGGTACACCCGCGAGGAGATCTTGGGGCGGAACTGCCGGTTCCTG
CAAGGTCCGCGGACCGATCGGAGCGCGGTGGCGGAGATTCGCAAGGCGATCGACGAGGGCAGCGAGTGCA
CCGTCCGGCTCTTAAACTACACCAAGCAGGGGAAGCCGTTTTGGAACATGTTCACCATGGCGCCCGTGCG
GGACGAGCAGGGAAACGTCCGTTTCTTCGCGGGGGTTCAGGTTGACGTCACGGTGTACACCCGCGAGGAG
GGCGAGAAGGACGCCACGAGCTTGGACCTCGTGAAGGAGTACGACAAGGACAGGGACGAGAGCTCGTTCG
ATCGACAGATGAAGGAGTACTCGAAGCAGACGGCGAGCGCGGTTGCGTCGGGGGTTGCCGGGCTTAAAGA
CGGGGATTTGCCCTGGAAGAACATGGTGGGCATCCTGCGGACGCCGCAGCCGCACCAGCGGCACGATCCC
AACTGGGTGGCGCTCAAGGCGCGAGTGGACAAGCACGAGGCGGAGGGCAAGGTTGGAAGGCTGTCGCCGG
ATGATTTCGTGCCGCTGAAGCGGCTAGGCAACGGCGACGTGGGCAGCGTCCACCTGGTCCAGCTCGCGGG
GACCAATCGGCTGTTCGCGATGAAGATACTGGTCAAGCAGGAGATGCACGAAAGGAACAAGCTGCACAGG
GTCCGGACGGAGGGTCAGATTTTGGAGACGGTGGATCACCCCTTCGTCGCGACGCTGTACGCCGCGTTTC
AGACTGACACGCACCTGTACTTTGTGCTCGAGTACTGCGAAGGCGGCGAGCTGTACGAGACGCTGCAGAA
GGAACCGGAGAAGCGATTTCCGGAGACGATCGCGAAGTTCTACGCCGCGGAGGTTCTCGTCGCGCTGCAG
TACCTCCACCTCATGGGATTCATCTACCGCGACCTCAAGCCGGAGAACATCCTCCTTCGCAGGGACGGGC
ACATCATCGTGACCGACTTTGACCTCAGCTATTGCGCCTCGTCCAGAGCGCACGTCATCATGAAGGAGGG
GCGAGCGCCCGGCGCGAGGGCGAGGAACCGCAGGGTTTCGCAGCGGCGGTCGTTCGCGGGAGGCGGGCGT
CCCTCCGTCGCCATCGATGTTGGAGGGAGCGGGAAGCCGCCCGGCGAAAACGCGTCAGGTCGGTCGCCCC
GACAATCGCAGATGTCCATCGACGCCACACACAACGGCGGCGTCGCCATACCCGGCGCGTCGCCAAAATC
CGCCGGCCCCGGGCTCGACATGATCGCGTGCGGCACGTTCCTGTCCCCGAACGGCGCCAACAAGTCGGGG
AAGTTTCCGCAGATCATCGCCGAGCCCTTCGCGTACACAAACTCTTTCGTCGGCACGGAGGAGTACCTGG
CGCCCGAGGTTCTCAACTCGACGGGTCACACGAGCTCGATCGACTGGTGGGAGCTCGGCATCTTCATCCA
CGAGATGGTGTTCGGGACGACGCCGTTTCGGGCGAACAAGCGCGAGCAGACCTTCCACAACATCGTCCAC
CAGCCCCTGGACTTTCCGTCGACGCCGCCGGTGAGCGGCGAGCTGAAGGATCTGCTTCGGCAGTTGCTCC
AGCGCGATCCCAGCGTCAGGTTGGGGACGCAGGGCGGCGCGGAGGAGGTCAAGGCGCACCCGTTCTTTCG
GAACGTGGACTGGGCGCTGCTGCGGTGGGCGAAGGCGCCGTTGGCGGAGAAGATCGCGAGGAGGATGGCG
AGGGCGAGCGGGGCGGAGGCGGCGAGCGCGGCGGTGGACGCAGGGGGCGGCGGCGACGACGACGAAATGT
TTCAGATGGACGTCGAGCAGTGA
SEQ ID NO: 126
>XP_002506288.1 Phototropin-Micromonas commoda
MSEPAPAVEPSAAAPSDEVPKFDETKTHESIDIGFTVDAGGGISAPQASKDLTNALASLRHTFTVCDPTL
PDCPIVYASDGFLKMTGYPAEEVLNRNCRFLQGEETNMDDVRKISEAVKKGERITVRLLNYRKDGQKFWN
LLTVAPVKLPDGTVAKFIGVQVDVSDRTEGNADNSAAMKDTKGLPLLVKYDQRLKDQNFNRVDDVEKAVL
TGEGVDLDANPVAANRGGLDMATTLERIQQSFVIADPSLPDCPIVFASDGFLDFTGYTREEILGRNCRFL
QGPRTDRSAVAEIRKAIDEGSECTVRLLNYTKQGKPFWNMFTMAPVRDEQGNVRFFAGVQVDVTVYTREE
GEKDATSLDLVKEYDKDRDESSFDRQMKEYSKQTASAVASGVAGLKDGDLPWKNMVGILRTPQPHQRHDP
NWVALKARVDKHEAEGKVGRLSPDDFVPLKRLGNGDVGSVHLVQLAGTNRLFAMKILVKQEMHERNKLHR
VRTEGQILETVDHPFVATLYAAFQTDTHLYFVLEYCEGGELYETLQKEPEKRFPETIAKFYAAEVLVALQ
YLHLMGFIYRDLKPENILLRRDGHIIVTDFDLSYCASSRAHVIMKEGRAPGARARNRRVSQRRSFAGGGR
PSVAIDVGGSGKPPGENASGRSPRQSQMSIDATHNGGVAIPGASPKSAGPGLDMIACGTFLSPNGANKSG
KFPQIIAEPFAYTNSFVGTEEYLAPEVLNSTGHTSSIDWWELGIFIHEMVFGTTPFRANKREQTFHNIVH
QPLDFPSTPPVSGELKDLLRQLLQRDPSVRLGTQGGAEEVKAHPFFRNVDWALLRWAKAPLAEKIARRMA
RASGAEAASAAVDAGGGGDDDEMFQMDVEQ

### EXAMPLES

Certain embodiments of the invention will be described in more detail through the following examples. The examples are intended solely to aid in more fully describing selected embodiments of the invention, and should not be considered to limit the scope of the invention in any way.

### Example 1

### Growth of Chlamydomonas Reinhardtii

Chlamydomonas reinhardtii parental strains (cw15 and UV4) and the phototropin knockout (PHOT K/O) mutants (CW15 and A4) were grown at 25° C. in 250 mL Erlenmeyer flasks containing 100 mL of High-Salt (HS) or Tris-Acetate-Phosphate (TAP) media and shaken at 150 rpm (world wide web at chlamy.org/media.html). Cultures were typically inoculated from a log phase culture using 1 mL of cells. Flasks were illuminated using fluorescent light at the light intensities as indicated for each experiment.

### Example 2

### Measurement of Photoautotrophic Growth and Biomass Estimation

Photoautotrophic growth of the parent strains CW15 and UV4) and the phototropin knock out mutants (G5 and A4) was measured in environmental photobioreactors (“ePBRs”) (obtained from Phenometrics, Inc.) in 500 mL of liquid HS media. All experiments were done in triplicates for each time point and each treatment. Light intensity was programmed for a 12 h sinusoidal light period with a peak mid-day intensity of 2,000 μmol photons m⁻² s⁻¹. Temperature was a constant 25° C., and the ePBRs were stirred with a magnetic stir bar at 200 rpm. Filtered air was bubbled constantly through the growing cultures. The optical density of the cultures was monitored on a daily basis at 750 nm using a Cary 300 Bio UV—Vis spectrophotometer (Agilent). After completion of growth measurements, the total contents of individual ePBRs were harvested by centrifugation at 11,000 rpm for 15 min. Cell pellets were frozen immediately in liquid N2 and later freeze-dried using a Microprocessor Controlled Lyophilizer (Flexi-Dry). After drying, pellets were weighed for total biomass.

### Example 3

### Measurement of Chlorophyll Fluorescence

For Chl fluorescence induction analysis, cell suspensions of the parental wild-type and transgenic Chlamydomonas strains were adjusted to a Chl concentration of ˜2.5 pg/mL. Quenching of Chl fluorescence was measured using the FL-3500 fluorometer (Photon System Instruments) (Kaftan, Meszaros et al. 1999). The cells were dark adapted for 10 min prior to the measurement. Chl fluorescence was induced using non-saturating continuous illumination and Chl fluorescence levels were measured every 1 μs using a weak pulse-modulated measuring flash. For the state transition experiments, low light grown cultures were dark adapted or pre-illuminated with 715 nm light for 10 min prior to the induction of Chl fluorescence. The actinic flash duration for this experiment was set to 50 μs and Chl fluorescence was measured every 1 μs.

### Example 4

### Measurement of Photosynthetic Oxygen Evolution

CO₂-supported rates of oxygen evolution were determined for low light (50 μmol photons m⁻² s⁻¹) HS grown log-phase cultures (0.4-0.6 OD₇₅₀ nm) using a Clark-type oxygen electrode (Hansatech Instruments). Cells were re-suspended in 20 mM HEPES buffer (pH 7.4) and air-saturated rates of oxygen evolution were measured as a function of light intensity (650 nm) at 50, 150, 300, 450, 600, 750 and 850 μmol photons m⁻² s⁻¹. The same experiment was repeated in the presence of 10 mM NaHCO₃. Light saturation curves were normalized on the basis of Chl as well as cell density (A_{750 nm}). Chl was determined by method described by Arnon (Arnon 1949).

### Example 5

### Measurement of Pigment Content by HPLC

Chlamydomonas cultures were grown at low (50 μmol photons m^{31 2}s⁻¹) and high (saturating) light (500 μmol photons m^{31 2}s⁻¹) intensities for 5 days in HS media in shaker flasks. Cells were centrifuged at 3,000 rpm for 3 min and immediately frozen in liquid nitrogen and lyophilized. Carotenoids and chlorophylls were extracted with 100% acetone in the dark for 20 min. After incubation samples were centrifuged at 14,000 rpm for 2 min in a microfuge and the supernatant was transferred to a glass tube and dried under vacuum. The dried samples were re-suspended in 1 mL of acetonitrile:water:triethylamine (900:99:1, v/v/v) for HPLC analysis. Pigment separation and chromatographic analysis were performed on a Beckman HPLC equipped with a UV-Vis detector, using a C18 reverse phase column at a flow rate of 1.5 ml/min. Mobile phases were (A) acetonitrile/H20/triethylamine (900:99:1, v/v/v) and (B) ethyl acetate. Pigment detection was carried out at 445 nm with reference at 550 nm (Tian and DellaPenna 2001). Individual algal pigments were identified on the basis of their retention times and optical absorbance properties and quantified on the basis of their integrated absorbance peaks relative to known carotenoid standards. Carotenoid standards were purchased from DHI, Denmark. Pigments were standardized on the basis of dry weight of three replicates.

### Example 7

### Transmission Electron Microscopy

Cells were prepared for electron microscopy by immobilizing cells in 3% sodium alginate (w/v) and the alginate beads were then solidified by incubation in cold 30mM CaCl2 for 30min. We used alginate encapsulated algal cells to keep cells intact as well as to protect from direct and harmful effect of chemicals during fixation processes. These cells were fixed using 2% glutaraldehyde for 1.5-2 hours and after fixation, these cells were post fixed in buffered 2% osmium tetroxide for 1.5 hours. After dehydration these cells were embedded in Spurr's resin. Thin sections were stained with uranyl acetate and lead citrate. LEO 912 transmission electron microscope was used to view and collect images at 120 kv and a Proscan digital camera.

### Example 8

### Transcriptome Analysis

Total RNA was extracted from 100 mg of cells/sample, flash frozen in liquid nitrogen, grown at high light (500 μmol photons m^{31 2}s⁻¹) intensities for 5 days in HS media in shaker flasks) using the Direct-zol RNA-miniprep kit (ZYMO, P/N 2051) according to the manufacturer's instructions. Each total RNA sample was enriched for mRNA by hybridizing the poly(A) tail to oligo d(T)25 probes covalently coupled to magnetic beads, followed by elution (NEB, P/N S1419S). The enriched mRNA fractions were prepared for Illumina sequencing using the ScriptSeq V.2 RNA-seq Library Preparation Kit (Epicentre, P/N SSV21106) and sequenced on a Hi-Seq 2000 (2×150 bp), multiplexed at 6 samples per lane. The resultant sequence reads were trimmed for quality and mapped to the coding sequences present in version 9 of the Chlamydomonas reinhardtii genome annotation at web address phytozome.jgi.doe.gov/pz/portal.html#!info?alias=Org_Creinhardtii using bowtie2. The relative transcript abundance of each gene (mean of 3 biological samples) was determined using RSEM and differential expression values (UV4 vs A4) were calculated using EdgeR. All genes identified as differentially expressed were mapped to KEGG biochemical maps using the v.9 annotation assignments.

### Example 9

### Identification of Chlorella spp. Phototropin Coding Sequence

Phototropin genes were identified in three Chlorella species (herein designated as strain 1412, strain 1228 and Chlorella sorokiniana UTEX1230) and a Picochlorum soloecismus (DOE101) by conducting homologous BLASTp searches against the annotations of Chlorella species using Chlamydomonas reinhardtii phototropin genes/proteins (NP_851210) and Arabidopsis thaliana protein sequences (Accession #AED97002.1 and AEE78073) as query proteins. The Chlorella spp. and Picochlorum phototropin homologs were aligned to other phototropin amino acid sequences using CLUSTALW, then truncated based on conserved sequence alignments and phylogenetically analyzed using a Maximum-Likelihood algorithm. Each Chlorella strain contains two paralogous copies of photoropin and Picochlorum soloecismus. (DOE101) was found to contain 1 homolog of phototropin. These sequences are provided as SEQ ID Nos. 1-14. Additional phototropin sequences and functional homologs are provided in Table 1 and SEQ ID NO 51-66 and SEQ ID NO 69-128.

### Example 10

### Inducible Control of Phototropin Expression in Chlamydomonas Reinhardtii

One method to reduce expression of algal PHOT gene(s) is to use RNAi technology driving the expression of double stranded, fold-back RNA elements to reduce the PHOT expression. A strong gene promoter such as psaD or other strong constitutive gene promoters could be used to drive expression of the RNAi construct similar to methods used previously in Chlamydmonas for modulation of light harvesting antennae complex (Perrine, Negi et al. 2012).

### Example 11

### Production of a Chlorella Phototropin Minus Mutant

PHOT gene knockouts could be potentially generated by traditional mutagenesis approaches including chemical, UV, random insertional mutagenesis screened by TILLING (Comai, Young et al. 2004, Nieto, Piron et al. 2007), and by targeted knock outs using CRISPR/cas9 (Wang, Yang et al. 2013, Xiao, Wang et al. 2013, Dubrow 2014). Pooled PHOT-based PCR screening coupled with sequencing of PHOT PCR products could be used to screen for PHOT mutants.

### Example 12

### Chemical Mutagenesis for Production of a Phototropin K/O Mutant in Chlorella Sorokiniana

Classical chemical mutagenesis is carried out using N-methyl-N′-nitro-N-nitrosoguanidine (MNNG). This mutagen makes nucleotide changes in the DNA and these changes, depending on their position, can have effects that are either positive or negative in the use of the strain being treated. By careful observation of phenotypes produced, as well as implementation of selective pressure, one selects mutants with improved traits for the desired purpose. This method has been applied to algae previously (Yan, Aruga et al. 2000).

Identifying strains of algae that grow rapidly and produce high starch is used as a selection marker for PHOT K/O mutants. Because this approach does not involve adding foreign DNA (in fact is focused only on existing genetic potential of the strain being mutagenized), strains generated by chemical mutagenesis are not considered to be “genetically modified”, allowing deployment in the field without additional government regulation.

N-methyl-N′-nitro-N-nitrosoguanidine (MNNG) was chosen based on its proven use for modifying blue-green algae, as well as its ability to eliminate toxicity by degradation in dilute acid. First, the conditions required to result in approximately 99% lethality for Chlorella protothecoides are determined; this degree of lethality generated optimal mutation frequency in blue-green algae (Chapman and Meeks 1987). Two treatments, exposure to 0.25 mg/mL MNNG for 30 minutes and 0.025 mg/mL MNNG for 60 minutes, result in approximately 99% lethality for this strain (unpublished data). Both treatments are used to generate mutagenized populations of Chlorella using enrichment strategies.

Approximately 10⁸ cells are mutagenized with four concentrations of MNNG and incubated for three different durations. After rinsing out the mutagen, approximately 10⁴ cells are spread plated on nutrient plates, and the number of colonies scored after 12 days. Treatments with approximately 100 surviving colonies, representing 99% lethality, are chosen as optimal for generating mutations.

PHOT K/O mutants are expected to be more rapidly growing and to produce excess sink molecules/material. In C. protothecoides the sink is lipid which could be used as a screen for selection of cells representing high lipid cells. Numerous methods are in the literature for such selection such as Nile red (Pick and Rachutin-Zalogin 2012) and BODIPY 493/503 (Ohsaki, Shinohara et al. 2010). High lipid cells are selected by flow cytometry and then placed in flask for cell culture. Rapid growing high lipid cells will dominate the culture and should be PHOT K/O as determined in this invention.

### Example 13

### Genome Editing Using CRISPR/cas9 to Reduce Expression of Phototropin in Chlamydomonas Reinhardtii

Recently, it has been demonstrated that CRISPR/cas9 genome editing techniques can be used to knock out genes of interest in Chlamydomonas when the Cas9 gene is expressed constitutively. By incorporating multiple guide RNA elements to specifically recognize the PHOT gene high efficiencies of gene mutagenesis can occur during miss-repair of the double stranded break in the target gene catalyzed by Cas/9 by the endogenous repair enzymes. By targeting repair of a recognized restriction endonuclease site, inhibition of the digestion of the PHOT-specific PCR product by the diagnostic restriction endonuclease can be used as an effective screen for PHOT mutants. Similarly, DNA repair mistakes that occur following double stranded DNA breaks in the PHOT gene generated by TALEN complexes can be used to generate PHOT-specific mutants.

### REFERENCES CITED

The following references and others cited herein, to the extent that they provide exemplary procedural and other details supplementary to those set forth herein, are specifically incorporated herein by reference and include US published patent applications and published patents: US 20130116165; US 20140249295; US 20130330718; U.S. Pat. No. 8,859,232 and other patent related documents EP2682469; WO 2011133493; WO 201408626; and WO 2013056212 and other publications listed:

### OTHER PUBLICATIONS

Arnon, D. I. (1949). “Copper Enzymes in Isolated Chloroplasts. Polyphenoloxidase in Beta Vulgaris.” Plant Physiol 24(1): 1-15.
Ausubel, F. M., R. Brent, R. Kingston, D. Moore, J. Seidman, J. Smith and K. Struhl (1997). Short Protocols in Molecular Biology. New York, Wiley.
Baena-Gonzalez, E., F. Rolland, J. M. Thevelein and J. Sheen (2007). “A central integrator of transcription networks in plant stress and energy signaling.” Nature 448(7156): 938-942.
Briggs, W. R. and M. A. Olney (2001). “Photoreceptors in plant photomorphogenesis to date. Five phytochromes, two cryptochromes, one phototropin, and one superchrome.” Plant Physiol 125(1): 85-88.
Chapman, J. and J. Meeks (1987). “Conditions for mutagenesis of the nitrogen-fixing cyanobacterium Anabaena variabilis.” J Gen Microbiol 131: 111-118.
Chen, M., J. Chory and C. Fankhauser (2004). “Light signal transduction in higher plants.” Annu Rev Genet 38: 87-117.
Comai, L., K. Young, B. J. Till, S. H. Reynolds, E. A. Greene, C. A. Codomo, L. C. Enns, J. E. Johnson, C. Burtner, A. R. Odden and S. Henikoff (2004). “Efficient discovery of DNA polymorphisms in natural populations by Ecotilling.” Plant J 37(5): 778-786.
Dubrow, Z. (2014). The development and application of the CRISPR/CAS system as a powerful new tool for genome editing: A case study.
Ermilova, E. V., Z. M. Zalutskaya, K. Huang and C. F. Beck (2004). “Phototropin plays a crucial role in controlling changes in chemotaxis during the initial phase of the sexual life cycle in Chlamydomonas.” Planta 219(3): 420-427.
Folta, K. M., E. J. Lieg, T. Durham and E. P. Spalding (2003). “Primary inhibition of hypocotyl growth and phototropism depend differently on phototropin-mediated increases in cytoplasmic calcium induced by blue light.” Plant Physiol 133(4): 1464-1470.
Fu, X., D. Wang, X. Yin, P. Du and B. Kan (2014). “Time course transcriptome changes in Shewanella algae in response to salt stress.” PLoS One 9(5): e96001.
Gaj, T., C. A. Gersbach and C. F. Barbas, 3rd (2013). “ZFN, TALEN, and CRISPR/Cas-based methods for genome engineering.” Trends Biotechnol 31(7): 397-405.
Green, M. and J. Sambrook (2012). Molecular cloning: A laboratory manual. Cold Spring Habor, N.Y., Cold Spring Harbor Laboratory Press.
Grossman, A. R. (2005). “Paths toward Algal Genomics.” Plant Physiology 137(2): 410-427.
Huang, K. and C. F. Beck (2003). “Phototropin is the blue-light receptor that controls multiple steps in the sexual life cycle of the green alga Chlamydomonas reinhardtii.” Proc Natl Acad Sci USA 100(10): 6269-6274.
Huang, K. and C. F. Beck (2003). “Phototropin is the blue-light receptor that controls multiple steps in the sexual life cycle of the green alga Chlamydomonas reinhardtii.” Proceedings of the National Academy of Sciences 100(10): 6269-6274.
Huang, K., T. Kunkel and C. F. Beck (2004). “Localization of the blue-light receptor phototropin to the flagella of the green alga Chlamydomonas reinhardtii.” Mol Biol Cell 15(8): 3605-3614.
Hwang, Y. S., G. Jung and E. Jin (2008). “Transcriptome analysis of acclimatory responses to thermal stress in Antarctic algae.” Biochem Biophys Res Commun 367(3): 635-641.
Im, C. S., S. Eberhard, K. Huang, C. F. Beck and A. R. Grossman (2006). “Phototropin involvement in the expression of genes encoding chlorophyll and carotenoid biosynthesis enzymes and LHC apoproteins in Chlamydomonas reinhardtii.” Plant J 48(1): 1-16.
Kaftan, D., T. Meszaros, J. Whitmarsh and L. Nedbal (1999). “Characterization of photosystem II activity and heterogeneity during the cell cycle of the green alga scenedesmus quadricauda.” Plant Physiol 120(2): 433-442.
Kagawa, T., M. Kimura and M. Wada (2009). “Blue Light-Induced Phototropism of Inflorescence Stems and Petioles is Mediated by Phototropin Family Members phot1 and phot2.” Plant and Cell Physiology 50(10): 1774-1785.
Kanehisa, M. and S. Goto (2000). “KEGG: kyoto encyclopedia of genes and genomes.” Nucleic Acids Res 28(1): 27-30.
Kanehisa, M., S. Goto, Y. Sato, M. Kawashima, M. Furumichi and M. Tanabe (2014). “Data, information, knowledge and principle: back to metabolism in KEGG.” Nucleic Acids Res 42(Database issue): D199-205.
Koid, A. E., Z. Liu, R. Terrado, A. C. Jones, D. A. Caron and K. B. Heidelberg (2014). “Comparative transcriptome analysis of four prymnesiophyte algae.” PLoS One 9(6): e97801.
Kozuka, T., S. G. Kong, M. Doi, K. Shimazaki and A. Nagatani (2011). “Tissue-autonomous promotion of palisade cell development by phototropin 2 in Arabidopsis.” Plant Cell 23(10): 3684-3695.
Matsuoka, D., T. Iwata, K. Zikihara, H. Kandori and S. Tokutomi (2007). “Primary processes during the light-signal transduction of phototropin.” Photochem Photobiol 83(1): 122-130.
Moni, A., A. Y. Lee, W. R. Briggs and I. S. Han (2015). “The blue light receptor Phototropin 1 suppresses lateral root growth by controlling cell elongation.” Plant Biol (Stuttg) 17(1): 34-40
Nieto, C., F. Piron, M. Dalmais, C. F. Marco, E. Moriones, M. L. Gomez-Guillamon, V. Truniger, P. Gomez, J. Garcia-Mas, M. A. Aranda and A. Bendahmane (2007). “EcoTILLING for the identification of allelic variants of melon elF4E, a factor that controls virus susceptibility.” BMC Plant Biol 7: 34.
Ohsaki, Y., Y. Shinohara, M. Suzuki and T. Fujimoto (2010). “A pitfall in using BODIPY dyes to label lipid droplets for fluorescence microscopy.” Histochem Cell Biol 133(4): 477-480.
Onodera, A., Kong, S-G, M. Doi, K.-I. Shimazaki, J. Christie, N. Mochizuki and A. Nagatani (2005). “Phototropin from Chlamydomonas reinhaardtii is functional in Arabidopsis thaliana.” Plant Cell Physiol 46(2): 367-374.
Perrine, Z., S. Negi and R. Sayre (2012). “Optimization of photosynthetic light energy utilization by microalgae.” Algal Res 134-142.
Pick, U. and T. Rachutin-Zalogin (2012). “Kinetic anomalies in the interactions of Nile red with microalgae.” Journal of microbiological methods 88(2): 189-196.
Reeck, G. R., C. de Haen, D. C. Teller, R. F. Doolittle, W. M. Fitch, R. E. Dickerson, P. Chambon, A. D. McLachlan, E. Margoliash, T. H. Jukes and et al. (1987). “”Homology” in proteins and nucleic acids: a terminology muddle and a way out of it.” Cell 50(5): 667.
Rismani-Yazdi, H., B. Z. Haznedaroglu, K. Bibby and J. Peccia (2011). “Transcriptome sequencing and annotation of the microalgae Dunaliella tertiolecta: pathway description and gene discovery for production of next-generation biofuels.” BMC Genomics 12: 148.
Sambrook, J., E. Fritsch and T. Maniatis (1989). Molecular cloning: a laboratory manual. Cold Spring Harbor, N.Y., Cold Spring Harbor Laboratory Press.
Sethi, P., M. Prasad and S. Roy (2009). All-optical switching in LOV2-C250S protein mutant from Chlamydomonas reinhardtii green algae. Emerging Trends in Electronic and Photonic Devices & Systems, 2009. ELECTRO '09. International Conference on.
Sizova, I., A. Greiner, M. Awasthi, S. Kateriya and P. Hegemann (2013). “Nuclear gene targeting in Chlamydomonas using engineered zinc-finger nucleases.” Plant J 73(5): 873-882.
Salomon, Michael, Briggs, Winslow, and Christie, John (2000) “Photochemical and Mutational Analysis of the FMN-Binding Domains of the Plant Blue Light Receptor, Phototropin.” Biochemistry 39: 9401-9410.
Suetsugu, N. and M. Wada (2007). “Phytochrome-dependent Photomovement Responses Mediated by Phototropin Family Proteins in Cryptogram Plants†.” Photochemistry and Photobiology 83(1): 87-93.
Sullivan, S., C. E. Thomson, D. J. Lamont, M. A. Jones and J. M. Christie (2008). “In vivo phosphorylation site mapping and functional characterization of Arabidopsis phototropin 1.” Mol Plant 1(1): 178-194.
Takemiya, A., S. Inoue, M. Doi, T. Kinoshita and K. Shimazaki (2005). “Phototropins promote plant growth in response to blue light in low light environments.” Plant Cell 17(4): 1120-1127.
Tian, L. and D. DellaPenna (2001). “Characterization of a second carotenoid beta-hydroxylase gene from Arabidopsis and its relationship to the LUT1 locus.” Plant Mol Biol 47(3): 379-388.
Trippens, J., A. Greiner, J. Schellwat, M. Neukam, T. Rottmann, Y. Lu, S. Kateriya, P. Hegemann and G. Kreimer (2012). “Phototropin Influence on Eyespot Development and Regulation of Phototactic Behavior in Chlamydomonas reinhardtii.” The Plant Cell 24(11): 4687-4702.
Veetil, S. K., C. Mittal, P. Ranjan and S. Kateriya (2011). “A conserved isoleucine in the LOV1 domain of a novel phototropin from the marine alga Ostreococcus tauri modulates the dark state recovery of the domain.” Biochim Biophys Acta 1810(7): 675-682.
Wang, H., H. Yang, C. S. Shivalila, M. M. Dawlaty, A. W. Cheng, F. Zhang and R. Jaenisch (2013). “One-step generation of mice carrying mutations in multiple genes by CRISPR/Cas-mediated genome engineering.” Cell 153(4): 910-918.
Xiao, A., Z. Wang, Y. Hu, Y. Wu, Z. Luo, Z. Yang, Y. Zu, W. Li, P. Huang, X. Tong, Z. Zhu, S. Lin and B. Zhang (2013). “Chromosomal deletions and inversions mediated by TALENs and CRISPR/Cas in zebrafish.” Nucleic Acids Res 41(14): e141.
Yan, X.-H., Y. Aruga and Y. Fujita (2000). “Induction and characterization of pigmentation mutants in Porphyra yezoensis (Bangiales, Rhodophyta).” Journal of Applied Phycology 12(1): 69-81.
Zorin, B., Y. Lu, I. Sizova and P. Hegemann (2009). “Nuclear gene targeting in Chlamydomonas as exemplified by disruption of the PHOT gene.” Gene 432(1-2): 91-96.
Toivola, J., Nikkanen, L., Dahlström, K. M., Salminen , T. A., Lepistö, A., Vignols, F., and Rintamäki, E. (2013). “Overexpression of chloroplast NADPH dependent thioredoxin reductase in Arabidopsis enhances leaf growth and elucidates in vivo function of reductase and thioredoxin domains.” Frontiers in plant sciences doi: 10.3389/fpls.2013.00389
Takahashi F (2016) Blue-light-regulated transcription factor, Aureochrome, in photosynthetic stramenopiles. J Plant Res 129(2):189-97.

What is claimed is:

1. A method for increasing a biomass productivity of an algal strain wherein the expression or function of a Chlamydomonas reinhardtii phototropin gene, a gene homologous to the Chlamydomonas reinhardtii phototropin gene or a gene sequence comprising a LOV domain and a Serine/Threonine kinase domain which gene sequence functions as a phototropin is reduced or eliminated as compared to the wild-type parental line.

2. The method of claim 1, wherein the homologous gene has greater than 75% homology to the Chlamydomonas reinhardtii phototropin gene or a sequence identified in SEQ ID NO 1-14, 51-66 and 69-128.

3. The method of claim 1, wherein the biomass productivity and photosynthetic efficiency of the algal strain is increased by greater than around 2-fold.

4. The method of claim 1, wherein the biomass productivity of storage product(s) in the algal strain is increased by greater than around 2-fold.

5. The method of claim 4, wherein the storage product(s) is selected from starch, lipid, pigments and other sink molecules.

6. The method of claim 1, wherein the biomass productivity is increased for bioproducts or storage products selected from the group consisting of lipids, waxes, polysaccharides (e.g., starch, glycogen, mannans, glycans, cellulose, hemicellulose), photoprotective pigments (e.g., xanthophyll).

7. The method of claim 1, wherein the expression of the Chlamydomonas reinhardtii phototropin gene, the gene homologous to the Chlamydomonas reinhardtii phototropin gene or the sequence homologous to SEQ ID NO 1-14, 51-66 and 69-128 is reduced by one or more of the following: chemical mutagenesis and selection, genome editing, inducible promoter and trans acting elements.

8. An algal strain wherein relative to a wild-type parental line
an expression of a phototropin gene. a homologous gene or a gene sequence comprising a LOV domain and a Serine/Threonine kinase domain is reduced,
photosynthetic pigments making up an antenna complex are reduced,
and a content of sink molecules is increased.

9. The algal line of claim 8, wherein the phototropin gene, the homologous gene or the gene sequence comprising a LOV domain and a Serine/Threonine kinase domain are rendered to be non-functional.

10. The algal line of claim 8, wherein the phototropin gene, the homologous gene or the gene sequence comprising a LOV domain and a Serine/Threonine kinase domain are substantially deleted.

11. The algal line of claim 8, wherein the phototropin gene, the homologous gene or the gene sequence comprising a LOV domain and a Serine/Threonine kinase domain can be rendered to be non-functional on an inducible basis through an inducible promoter.

12. The algal line of claim 8, wherein the phototropin gene deletion would generate sterile and stable diploid population of polyploid algae to avoid recombination of genetic material during sexual reproduction.

13. The algal line of claim 8, wherein the phototropin gene deletion would be used to generate stable transgene-stacking traits in polyploid algal strains.

14. The algal line of claim 8 wherein the phototropin gene or the homologous gene is selected from SEQ ID NO 1-14, 51-66 and 69-128.

15. The method of claim 8, wherein the homologous gene has greater than 75% homology to a Chlamydomonas reinhardtii phototropin gene or the sequence identified in SEQ ID NO 1-14, 51-66 and 69-128.

16. A method for increasing a biomass productivity of an algal strain wherein an expression or function of a Chlamydomonas reinhardtii NTR2 or NTRC gene, a gene homologous to a Arabidopsis NTR2 or NTRC gene or a sequence homologous to SEQ ID NO 35-50 and 67-68 is over expressed in the algal strain as compared to a wild-type parental line.

17. The method of claim 16, wherein the homologous gene has greater than 75% homology to the Arabidopsis NTR2 or NTRC gene or the sequence identified in SEQ ID NO 35-50 and 67-68.

18. The method of claim 16, wherein the biomass productivity of the algal strain is increased by greater than around 2-fold.

19. The method of claim 16, wherein the biomass productivity of storage product(s) in the algal strain is increased by greater than around 2-fold.

20. The method of claim 19, wherein the storage product(s) is selected from starch, lipid, pigments and other sink molecules.

21. The method of claim 16, wherein the biomass productivity is increased for bioproducts or storage products selected from the group consisting of lipids, waxes, polysaccharides (e.g., starch, glycogen, mannans, glycans, cellulose, hemicellulose), photoprotective pigments (e.g., xanthophyll).


SEQUENCE LISTING (68 sequences)

SEQ ID NO: 1 - protein, 760 aa, Chlorella sorokiniana
MAPTVQGLPAPQTQVVNALSTLRHTFVVADATLPDCPLIYASEGFVQMTGYSMEEVLGHNCRFLQGEGTDPKDVKKLRDAVKNGTPVCTRLLNYRKDGTPFWNLLTMTPIKDEIGRVIKFVGVQVDVTNRTEGRAYTDSQGVPVLVHYDDRLKETVAKPIVDDVLMAVQQDDGKTPVRLSRGSPSRALPRVALDLATTVERIQSNFVIADPTLPDCPIVFASDPFLRLTGYRREEVLGRNCRFLQGPDTDRAAVLELKAAIRAGRECTVRLLNYTKTGKAFWNMLTVAPIKDIEDRPRFLVGVQVDVTEHPTVADATPVGRQAANAVGQALQSMNWVGVDPWATFPTGLRQPKPHRRLDPAAAALAAVVARDGKLRLRHFSRVKQLGSGDVGMVDLVQLVGTGQRFALKSLEKREMLERNKVGRVRTEEAILSTVDHPFLATLYGTLQTDTHLHFLLEYCNGGELYALLNSQPNKRLKEDVVRFYAAEVLLALQYLHVQGYVYRDLKPENILLHSTGHVMLTDFDLSYCQGSTTPSLLMLPGEAAAAAAAGVPRSSSGINCAGSKGERGSEAAPALPSGQQALLVAQPDGRANSFVGTEEYLAPEVITGSGHTSMVDWWSFGILIYELLGARRDATFENVLKKPLAFPDAVSVSPACKDLITKLLNKEPGKRLGSKAGADEIKRHPWFAATNWALVRQQSPPFVTPRRSSAGAEGGRPSRPLSDGSEPRVHSADSVLPDPKPAAAAAAAAAPGQQPKAKS

SEQ ID NO: 2 - DNA, 2347 bp, Chlorella sorokiniana
cccaagggtcccggctctggggccgtggcagcagcagcgagttggagggacgcagcgggctgcaaatatggccccgaccgtgcagggcttaccggcgccccagacgcaagtcgtgaacgcactatcgacccttcggcacacctttgtggtggcggatgccacgctgccagactgcccgctcatctatgccagcgaggggtttgtgcagatgacgggctatagcatggaggaggtgctggggcacaactgccgcttcctgcaaggcgagggcaccgacccaaaggatgtgaagaagctgcgggatgctgtgaagaatggcacccccgtgtgcacgcgcctgctcaactaccggaaggacggcacgccattctggaacctgctgaccatgacacccatcaaggacgagatcgggcgggtcatcaagtttgtgggcgtgcaggtggatgtgaccaaccgcacagagggccgcgcatacaccgactcccagggcgtgccggtgctggtccactacgacgaccgcctgaaggagacggtggccaagccgattgtggacgatgtgctgatggcggtgcagcaggatgacggcaagacgccggtgcggctgtcgcgcggctcgccctcgcgcgccctgccccgtgtggcgctggatctggccacgacagtggagcgcatccagtcgaactttgtgattgccgaccccacgctgcccgactgccccattgtgtttgcctccgaccccttcttgcgcctcaccggctaccggcgcgaggaggtgctgggccgaaactgccgcttcctgcaaggccccgacaccgaccgggcggcagtgctggagctcaaggcagccatccgggcggggcgcgagtgcacggtgcgcctgctcaactacaccaagacgggcaaggccttctggaacatgctcacagtggcgcccatcaaggacattgaggaccggccgcggttcctggtgggcgtgcaagtagatgtgacagagcaccccacagtggcggacgccacccctgtgggccgccaggcagccaacgcggtcggccaggcgctgcagagcatgaactgggtgggcgtggacccatgggccacgttccccacaggcctgcggcagcccaagccgcaccgccggttggacccggcggctgcggcgctggcggcagtggtggctcgcgacggcaagctgcgcctgcgccacttctcgcgggtgaagcagctgggcagcggcgacgtgggcatggtggacctggtgcagctggtgggcaccggccagcgctttgcgctcaagtcgctggagaagcgggagatgctggagcgcaacaaggtgggccgcgtgcgcactgaggaggccatcctgtccacagtggaccaccctttcctggccacgctctacggcacgctgcagacggacacgcacctccacttcctgctggagtactgcaacggcggcgagctgtacgcgctgctcaactcgcagcccaacaagcggctgaaggaggatgtggtgcgcttctacgccgccgaggtgctgctggccctgcagtacctgcacgtccagggctacgtgtaccgcgacctgaagcctgagaacatcctgctgcactccaccggccacgtcatgctgaccgactttgacctgagctactgccagggcagcaccacgccctccctgctcatgctgccgggcgaagcagcggcagcagctgcagcgggcgtgccgcgcagcagcagcggcatcaactgtgcgggttccaaaggcgagcgcggcagcgaggcggcccctgcactgccctcgggccagcaggcgctgctggtggcgcagccggatgggcgtgccaacagctttgtgggcactgaggagtacctggcaccagaggtcatcacaggctccggccacacctccatggtggactggtggtcttttggcatcctcatttatgagctgctgggcgcgcggcgagatgccacctttgagaatgtgctgaagaagcctctggccttcccggatgcggtgtccgtctcgcccgcctgcaaggacctgatcaccaagcttctgaacaaggagcctggcaagcggctgggcagcaaggcgggggcggacgagatcaagcgccacccatggtttgcggccaccaactgggcgcttgtgcggcagcagtcgccgccatttgtcacgccgcggcgctccagtgcaggagcagagggcggccgcccgtcgcgcccgctgtctgatggctcggagccacgcgtgcactctgctgactctgttctgccagatcccaagccggcggcagcagcggcagcggcagcagccccaggccagcagccgaaagccaagtca

SEQ ID NO: 3 - protein, 775 aa, Chlorella sorokiniana
LRHTFVVADATQPDCPLVYASQGFYDMTGFSPEEVIGHNCRFLQGPNTDPEHVRKLRESVQNGTCVTVRLLNYRKDGTPFWNLLTMTPVKDDTGAVVKIVGVQLDVTDTTEGLEDAAHGVPVLVRYDYRLQDKLVTPAVDDVLLGLQEDDEAATTAGAAGAAAGGEAHRLSCSALLRQHHRGQLDLGTTMERMQHNFVVSDPTLPDCPIVFASDGFLELTGYRREEVLGHNCRFLQGPDTDRAEVERLKAAINNWEEVTVKLLNYNKSGKPFWNLLTVAPILDGKGHPRLLVGVLVDVTNISTEGVAAAEHQAATAVGQALGTMGWDGGDPWEHFETALAPAKPHQANDPAAAALRALVKEDGELRLERFRRIADLGAGDAGVVTLVELQPLDGMDAVGAGGAIGRHLFALKSMDKKAMEERNKVGRVRTEETILRSVDHPYLAKLYATIHTDTHLHFLLEYCSGGVLYDVLERSPDHCIPEAEAKSIAAEVLLALQYLHLRGFIYRDLKPENILIMPSGHCQLTDFDLSFTCGTNGASVAPELVPAAVAPVPAPGTPPGSTSGRGGSGSAMLRTSSTSLRSNGSAASPMLLAAQPSVRTNSLVGTEEYLAPEIIIGEGHDSMVDWWSFGILLYELMYGTTPFKSARRDTTFDNIVKRQPHFPHRGVSPEGRDLISKLLIKDPTQRLGAQAGADEVRQHPWFADFDWALGRHSEATLARAASRAGVPKCAPSKVPTGIGSNGISGGRSSASPPTPKRAGEGGAVMGCFPLRRRRN

SEQ ID NO: 4 - DNA, 2328 bp, Chlorella sorokiniana
ctccgccacacctttgtggtggccgatgccacccagccagactgcccgctggtctatgccagccagggcttctatgacatgacaggcttttcgcctgaggaggtgattgggcacaactgccgcttcctgcaaggccccaacacagaccctgagcacgtgcgcaagctgcgggagtcggtgcagaacggcacatgcgtcactgtgcgcctgctcaactgtcgcaaggatggcacgccgttctggaacctgctgaccatgacgcctgtcaaggatgacacgggcgctgtggtcaagattgtgggcgtgcagttggatgtgacggacaccacagaaggcttggaggacgcagcacacggcgttccggtgcttgtgcgctacgactaccgcctgcaagacaagctggtcacgccagcggtggacgatgtcctgctgggcctgcaggaggacgacgaggcggcgaccacagcgggcgcagcgggcgcagcagcgggcggcgaggcgcaccgcctgtcctgctcggcgctgctgcgccagcaccaccgcggccagctggacctgggtacaaccatggagcgcatgcagcagaactttgtggtgtcggaccccacgctgcccgactgccccatcgtgtttgcctcggacggattcctggagctcaccggctaccggcgcgaggaggtgctgggccacaactgccgcttcctgcaaggccccgacaccgacagggccgaggtggaacggctgaaggcagccatcaacaactgggaggaggtgactgtgaagctgctgaactacaacaagagcggcaagcccttttggaacctgctcactgtggcccccatcctggatggcaagggccacccccgcctgcttgtgggcgtgctggtggatgtgaccaacatcagcaccgagggtgttgcagcggcggagcaccaggcagcaaccgctgtggggcaggcgctgggcacaatgggctgggacggcggcgatccctgggagcactttgagaccgctctggcacctgccaagccccaccaagccaacgaccctgccgccgccgctctgcgcgctctggtcaaggaggatggcgagctgcggctggagcgcttccgccgcattgccgacctgggagccggcgatgcgggcgtggtgactctagtggagcttcagccgctcgatgggatggatgcggtgggggcgggcggcgccatcggccgccacctgtttgccctcaagtccatggacaagaaggcgatggaggagcgcaacaaggtgggccgcgtgcgcaccgaggagacgatcctgcggtcggtggaccacccctatctcgccaagctctacgccaccatccacacggacacacacctgcacttcctgctcgagtactgctccggcggcgtgctgtacgacgtgctggagcgctcccccgaccactgcatccccgaggcagaggcgaagagcattgctgccgaggtgctgctggccctgcagtacctccacctgcgtggcttcatctacagggacctgaagcctgagaacatcctaatcatgccctccggccactgccagctcaccgattttgatctgtccttcacctgcggcaccaacggcgccagtgtggcacctgagctggtcccagcggcggtcgcacccgtgcccgcccccggcaccccaccgggcagcaccagcgggcgcggtggcagcggcagcgccatgctgcgcaccagttccaccagcctgcggtccaatggcagtgcggccagccccatgctgctggctgcccagcccagcgtgcgaaccaactcgctggtgggcactgaggagtacctggcacctgagatcatcattggcgaggggcacgacagcatggtggactggtggtcctttggcatcctgctctacgagctgatgtacggcaccacgcccttcaagtcggcgcggcgcgacaccacctttgacaacattgtgaagcggcagccgcacttcccgcaccgcggggtgtctccagaggggcgcgacctcattagcaagctgctgatcaaggaccccacgcagcgcctgggggcgcaggcgggtgctgacgaggtgcggcagcacccctggtttgccgactttgactgggctctggggcggcactcagaagccaccctggcccgggcggccagccgggccggcgtgcccaagtgtgcccccagcaaggtgccgaccggcatcggcagcaacggcatcagcggcggccgcagcagcgcatcgccgcccacgcccaagcgggctggggaggggggcgccgtcatgggctgcttcccactgcggcgccgccgcaactga

SEQ ID NO: 5 - protein, 730 aa, Chlorella sorokiniana
NCRFLQGEGTDPKDVKKLRDAVKNGTPVCTRLLNYRKDGTPFWNLLTMTPIKDELGRVIKFVGVQVDVTNRTEGRAYTDSNGVPVLVHYDDRLKETVAKPIVDDVLMAVQQDDGKTPVRLSRGSPSRALPRVALDLATTVERIQSNFVIADPTLPDCPIVFASDPFLRLTGYRREEVLGRNCRFLQGPDTDRAAVLELKAAIRAGRECTVRLLNYTKTGKAFWNMLTVAPIKDIEDRPRFLVGVQVDVTEHPTVADATPVGRQAANAVGQALQSMNWVGVDPWATFPTGLRQPKPHRRLDPAAAALAAVVARDGKLRLRHFSRVKQLGSGDVGMVDLVQLVGTGQRFALKSLEKREMLERNKVGRVRTEEAILSAVDHPFLASLYGTLQTDTHLHFLLEYCNGGELYALLNSQPNKRLKEEVVRFYACEVLLALQYLHVQGYVYRDLKPENILLHSTGHVMLTDFDLSYCQGSTTPSLLMLPAEQAAPAAAAGRTSSGINCAGSKGERGGEAAAALPSGQQALLVAQPDGRANSFVGTEEYLAPEVITGSGHTSMVDWWSFGILVYELLGARRDATFENVLKKPLGFPDGVAVSPACKDLITKLLNKASGAGLGTPFIREPGKRLGSKAGADEIKRHPWFAGINWALVRQQAPPFVTPRRSSAGEGGRPSRPLSDGSEARVHSADSVLPEPKPAAAAAAAAAGQQPKSKSEGAAAAAAVAREGPGHIDGF

SEQ ID NO: 6 - DNA, 2420 bp, Chlorella sorokiniana
tgtattcccagctttcgctggccccagcgcagctgcaggagcccaggatgaagcgtgggggggaggtggggctccctgcggcgcaatcccaggtcgtgaacgcgctggccaccctcagacatacgtttgtggtggccgatgccacgctgccagactgcccgctcatctatgccagcgaggggtttgtgcagatgacgggctacagcatggaggaggttctggggcacaactgccgtttcctgcaaggcgagggcaccgatcccaaggatgtgaagaagctgcgggatgcggtgaagaacggcacccccgtgtgcacgcgcctgctcaactaccgcaaggacggcaccccgttctggaacctgctgaccatgacgccgatcaaggacgagctggggcgcgtgatcaagtttgtgggcgtgcaggtggatgtgaccaaccgcaccgagggccgcgcgtacacagactccaacggcgtgccggtgctggtgcactacgacgaccgcctcaaggagacggtggccaagccgattgtggacgatgtgctgatggcggtgcagcaggatgacggcaagacgccggtgcggctgtcacgcggctcgccctcacgggccctgccccgtgtggcgctggatctggccacaacggtggagcgcatccagtcgaactttgtgattgccgaccccacgctgcccgactgccccatcgtgtttgccagcgacccgttcctgcggctcaccggctaccgccgagaggaggtgctgggacgcaactgccgcttcctgcaaggccccgacacggatcgtgcggcagtgctggagctgaaggcagcgatccgggcggggcgcgagtgcacggtgcgtctgctcaactacaccaagacgggcaaggccttctggaacatgctcaccgtggcgcccatcaaggacattgaggaccggccgcgcttcctggtgggcgtgcaagtggatgtgacggagcacccgactgtggcagatgccacgcccgtgggccgccaggcagccaacgcggtcggccaggcgctgcagagcatgaactgggtgggtgtggacccctgggccacgttccccacgggcctgcggcagcccaagccgcaccgccgcttggacccagccgcggcagcgctggcggcagtggtggcgcgcgacggcaagctgcgcctgcgccacttctcgcgggtgaagcagctgggcagcggtgacgttggcatggttgacctggtgcagctggtgggcaccggccagcgctttgcgctcaagtcgcttgagaagcgggagatgctggagcgcaacaaggtgggccgcgtgcgcacagaggaggcaatcctgtcggccgtggaccaccccttcctggccagcctgtatggcacgctgcagacagacacgcacctgcacttcctgctggagtactgcaacggcggcgagctgtatgcactgctcaactcgcagcccaacaagcgactgaaggaggaggtggtgcgcttctacgcctgcgaggtgctgctggcgctgcagtacctgcacgtccagggctacgtgtaccgcgacctgaagcccgagaacatcctgctgcactccacgggccacgtcatgctgaccgactttgacctgagctactgccagggcagcaccacgccctccctgctcatgctgccggcggagcaggcggcgccggcggcagccgcagggcgcaccagcagcggcatcaactgcgctgggtccaagggcgagcggggcggcgaggcggccgctgcgctgccctcgggccagcaggcgctgctggtggcgcagccagacgggcgcgccaacagctttgtgggcactgaggaatacctggcgccagaggtcatcaccggctccggccacacatccatggtggactggtggtcctttggcatcctcgtctacgagctgctgggcgcgcggcgagacgccacgtttgagaatgtgctgaagaagccgctgggcttcccggatggggtggccgtctcgcccgcctgcaaggacctcatcaccaagctgctgaacaaggcgagtggggctgggctgggcactccatttatcagggagcctggcaagcggctgggcagcaaggctggggcagacgagatcaagcggcacccctggtttgccggcatcaactgggcgctcgtgcgccagcaggcgccgccgtttgtcacgccccggcgctccagcgcgggagagggcgggcgcccctcgcggccgctgtcggacggctccgaggcgcgtgtgcactctgccgactcagtcctgccagagcccaagcctgcagcggcggcggcggcggcggcggccggccagcagcccaagtcaaagtcggaaggtgctgctgcggcggcggcggtggcccgcgagggccctggccacatcgacggattctga

SEQ ID NO: 7 - protein, 781 aa, Chlorella sorokiniana
MVSALAKLRHTFVVADATQPDCPLVYASQGFYDMTGYSPQEVIGHNCRFLQGPDTDPEHVRKLRDSVQNGTGVTVRLLNYRKDGTPFWNLLTMTPVKDDTGTVVKIVGVQLDVTDTTEGLEDAAHGVPVLVRYDYRLQDKLVTPAVDDVLLGLQEDDEAATTAGTAGAGEVHRLSCSTLLRQHHRGQLDLGTTMERMQQNFVVSDPSLPDCPIVFASDGFLELTGYRREEVLGHNCRFLQGPETDRAEVERLKTAIANWEEVTVKLQNYTKGGKPFWNLLTVAPILDGKGHPRLLVGVLMDVTNSSVEGGAAAEHQAATAVGRALGAMGWDGSDPWEHFETALAPAKPHQASDPAAAALRAVVKEDGELRLERFRRIADLGAGDAGVVTLVELLPPKGADAAAGMGAASGRHLFALKSMDKKAMEERNKVGRVRTEETILRSVDHPYLAKLYATLQTDTHLHFLLEYCSGGVLYDVLERAPDHCLPEAEAKSIAAEVLLALQYLHLHGFIYRDLKPENILIMPCGHCQLTDFDLSFAGTGAASVAPELVPAASAAALAPGTPPAAASPTRGSSSSSMLRTSSASLRSSSSTASPMLLAAQPSVRTNSLVGTEEYLAPEVIIGEGHDSMVDWWSFGILLYELMYGTTPFKSARRDTTFDNIVKRELHFPSRGPVVSAEGRDLITRLLTKDPTQRLGAQAGADEVRQHPWFAEVDWALGRHSEATLARAASRGPKRAPSKAASRAPPGIGSNGRGSSAAPPTPKRAGEGGAVMGCFPMRRRRN

SEQ ID NO: 8 - DNA, 2346 bp, Chlorella sorokiniana
atggtgtcggcgcttgcaaagctccgccacacctttgtggtggccgatgccacccagccggactgcccgctggtgtatgccagccagggtttctatgacatgaccggctacagtccacaggaggtgatcggccataactgccggttcctgcaaggcccggacacggacccagagcacgtgcgcaagctgcgcgactcggtgcaaaacggcacaggcgtcaccgtgcgcctgctcaactaccgcaaggatggcaccccgttttggaacctgctgacaatgacacctgtcaaggatgacaccggcactgtggtcaagattgtgggcgtgcagctggatgtgaccgacaccaccgaaggcctggaggatgcggcgcacggcgtgccagtgctggtccgatacgactaccgcctgcaggacaagctggtgacacctgcagtggacgacgtgcttctgggcctgcaagaggatgacgaggcggcgaccacagcgggcacagcaggggcgggcgaggtgcaccgcctctcctgctccacgctgctgcgccagcaccaccgcggccagctggacctgggcaccaccatggagcgcatgcagcagaacttcgtggtgtccgacccttccctgcccgactgccccatcgtatttgcgtccgacgggttcctggagctcacgggctaccggcgcgaagaagtgctgggccacaactgccgcttcctgcaaggccccgagactgatcgggcggaggtggagcggctgaagacagccattgccaactgggaggaggtgactgtaaagctgcagaactacaccaagggcggcaagcctttctggaacctgcttacggtggctcccattctggatgggaagggccacccccgcctgcttgtgggcgtgctgatggatgtgaccaacagcagcgtggagggcggcgcagcggcggagcaccaggcggcgacggctgtggggcgtgcgctgggagcgatgggctgggacggcagcgacccctgggagcactttgagacagccctggcgccggccaagccgcaccaggccagcgaccccgcggccgccgccctgcgcgctgtggtcaaggaggacggcgagctgcgcctggagcgcttccgccgcatcgcggatctgggtgctggcgatgcgggcgtggtgaccctagtggagctgctgccgcccaagggagcggacgcagcggcgggcatgggggccgccagcgggcgccacctgtttgcgcttaagtccatggacaagaaggcgatggaggagcgcaacaaggtgggccgcgtgcgcaccgaggagaccatcctgcgctcggtggaccacccctacctcgccaagctgtacgccaccctccagacagacacgcacctgcacttcctgctggagtactgctcgggcggtgtgctgtacgacgtgctagagcgcgcccccgaccactgcctgcccgaagcggaggccaagagcatcgcggcagaggtgctgctggcgctgcagtacctgcacctgcatggcttcatctacagggacctgaagcccgagaacatcctgatcatgccctgcggccactgccagctcaccgacttcgacctgtcctttgccggtaccggcgccgccagcgtggcgccagagctggtgccggccgcctccgcggcggcactcgcgccgggcacgccgccggctgccgcctcccccacgcgcggcagcagcagcagcagcatgctgcggaccagctcggccagcctgcggtccagcagcagcacggccagccccatgctgctggctgcacagcccagcgtgcgcaccaactcgttggtgggcactgaggagtacttggcgccggaggtcatcattggcgagggccacgacagcatggtggactggtggtcctttggcatcctgctgtacgagctgatgtacggtaccacgcccttcaagtctgcgcggcgggacaccactttcgacaacatcgtcaagcgggagctgcatttcccgtcccgcgggccggttgtgtctgcagaggggcgcgacctcatcacccgcctgctgaccaaggaccccacgcaacggttgggtgcccaggcaggggcggacgaggtgcggcagcacccctggtttgcggaggtggactgggccctggggcggcactccgaggccaccctggctcgtgccgccagccgtggacccaagcgcgcacccagcaaggccgccagcagggcgcctcccggcatcggcagcaacggccgcggcagcagcgcggcgccgcccacgcccaagcgggccggggagggcggtgcggtgatggggtgcttccccatgcgccgccggcgcaactga

SEQ ID NO: 9 - protein, 789 aa, Chlorella sorokiniana
MAPSAAGLPAPQTQVVDALSTLRHTFVVADATLPDCPLIYASEGFVQMTGYSMEEVLGHNCRFLQGEGTDPKDVKKLRDAVKNGTPVCTRLLNYKKDGTPFWNLLTMTPIKDEAGRVIKFVGVQVDVTNRTEGRAYTDSNGVPVLVHYDDRLKETVAKPIVDDVLMAVQQDDGKTPVRLSRGSPSRALPRVALDLATTVERIQSNFVIADPTLPDCPIVFASDPFLRLSGYRREEVLGRNCRFLQGPDTDRAAVLELKAAIRAGRECTVRLLNYTKTGKAFWNMLTVAPIKDIEERPRFLVGVQVDVTEHPTVADATPVGRQAANAVGQALMSMNWVGVDPWATFPTGLRQPKPHRRMDPAAAALAAVVARDGKLRLRHFSRVKQLGSGDVGMVDLVQLVGTSQRFALKSLEKREMLERNKVGRVRTEEAILSTVDHPFLATLYGTLQTDTHLHFLLEYCSGGELYALLNSQPNKRLKEDVVRFYASEVLLALQYLHVQGYVYRDLKPENILLHSTGHVMLTDFDLSYCQGSATPSLLLLPGEAAAAPAVARSNSGITCGSAKGERGGSEAAPALPSGQQALLVAQPDGRANSFVGTEEYLAPEVITGSGHTSLVDWWSFGILIYELLGARRDATFENVLKKPLGFPEAVPVSPACKDLIAKLLCKEPGKRLGSKAGADEIKRHPWFAGINWALVRQQAPPFVTPRRSSVGGEGRPSRPLSDRSEPRVHSADSVLPDSKAASAASGKQAKAKSEGAAVAAAAPAAAVPAAAAPAAAAAAPQGPGHIDGF

SEQ ID NO: 10 - DNA, 2370 bp, Chlorella sorokiniana
atggccccgagcgctgcggggctgccggccccccagacgcaagtagtagatgcgttgtcgacgctgcgacacaccttcgtggtggctgatgccaccctgccggactgcccgctcatctacgccagcgaggggtttgtgcagatgacggggtacagcatggaggaggtgctggggcacaactgccgcttccttcaaggcgagggcaccgaccccaaggacgtgaagaagctgcgggatgcggtgaagaacggcaccccagtgtgcacgcgcctgctcaactacaagaaggacggcaccccgttttggaacctgctgaccatgacccccatcaaggacgaggccgggcgcgtcatcaagtttgtgggcgtgcaggtggatgtgaccaaccgcaccgagggccgggcctacacagacagcaacggcgtgccggtgctggtccactacgatgaccgcctgaaggaaacggtggccaagccgattgtggatgatgtgctgatggcggtgcagcaggatgatggcaagacgccggtgcggctgtcgcgcggctcgccctcacgggccctgcctcgtgtggcgctggatctggccaccacggtggagcgcattcagtcgaacttcgtgattgccgaccccacgctgcccgactgccccattgtctttgcctccgaccccttcctgcgcctgagtggataccgccgagaggaggttctgggccgcaactgccgcttcctccaagggccagacacggacagggcagcagtgctggaactgaaggcggccatccgggcagggcgcgagtgcacggtgcgcctgctcaactacaccaagacgggcaaggccttttggaacatgctcactgtggcgcccatcaaggacattgaggagcggccgcgcttcctggtgggcgtgcaagtggacgtgacggagcaccccactgtggcggacgccacgccggtgggccgccaggcagccaacgcggtgggccaggcgctgatgagcatgaactgggtgggtgtggacccttgggccaccttccccacgggcctgcggcagcccaagccgcaccgccgcatggacccggccgctgcggcgctggcggcggtggtggcgcgcgacggcaagctgcgcctgcgccacttttcgcgggtcaagcagctgggcagcggcgatgtgggcatggtggacctggtgcagctggtgggcaccagccagcgctttgcgctcaagtcgctggagaagcgggagatgctggagcgcaacaaggtgggccgcgtgcgcaccgaggaggccatcctgtcaacagtggaccaccccttcctggccaccctctatggcacgctgcagacggacacgcacctccatttcctgctggagtactgcagcggcggcgagctgtacgcgctgctcaactcgcagcctaacaagcggctgaaggaggatgtggtgcgcttctacgccagcgaggtgctgctggcgctgcagtacctgcacgtccagggctacgtgtaccgcgacctgaagccggagaacatcctgctgcactccacgggccacgtcatgctgaccgactttgacctcagctactgccagggcagcgccacgccctccctgctgctgctgcccggcgaggcggcggcggcgccggcggtggcacgcagcaacagcggcatcacgtgcgggagcgccaagggcgagcgcggcggcagcgaggcggcgccggcgctgccctcgggccagcaggcgctgctggtggcgcagccggatgggcgcgcaaacagctttgtgggcactgaggagtacttggctccagaggtcatcaccggctccggccacacctccctggtggactggtggtcctttggcatcctgatttacgagctgctgggtgcgcggcgagacgccacctttgagaacgtgctgaagaagccgctgggcttcccggaagcggtgcccgtctcacccgcctgcaaggacctcatcgccaagctgctgtgcaaagagcccggcaagcggctgggcagcaaggcgggggcggacgagatcaagcggcacccctggtttgcgggcatcaactgggcgctggtgcggcagcaggcgccgccctttgtcacgccgcggcgctcgagcgtgggaggagagggccgaccgtcccgcccgctgtccgaccgctcggagccgcgcgtgcactccgccgactcagtcctgccagacagcaaggcagcatcggctgcctctggcaagcaggccaaggccaagtcggaaggggcggctgtggcggccgccgcgccggcagccgccgtgccggcagccgccgcgccggccgctgccgcggcggccccgcagggccccggccacattgacggcttttga

SEQ ID NO: 11 - protein, 779 aa, Chlorella sorokiniana
MSALAKLRHTFVVADATLPDCPLVYASQGFYDMTGFSREEVIGHNCRFLQGPDTDPEHVKKLRDAVKNGTCVTVRLLNYRKDGTPFWNLLTMTPVKDDTGTVVKIVGVQLDVTDTTEGLEDAAHGVPLLVRYDYRLQDKLVTPATDDVLLGLQEDDELATKAGAPGSVHPVHRLSTSTLLRQHHRGQLDLGTTFERMQQNFVVSDPTLPDCPIVFASDGFLELTGYRREEVLGHNCRFLQGPDTDRAEVEKLKAAITNWEEITVRLLNYTKTGTPFWNLLTVAPILDGKGHPRLLVGVLMDATNISIEGGAAAEHQAAVSVGRALGTMGWDGSDPWEHFQTALAPAKPHQASDPAAAALRAVVKADGELRLERFRRIADLGAGDAGVVTLVELRPPEAAGAAGMTASGGRFLFALKSMDKKAMEERNKVGRVRTEETILRTVDHPYLAKMYATIHTDTHLHFLLEYCSEGVLYDVLERSPDHCIPEAEAKSIAAEVLLALQYLHLHGVIYRDLKPENILLRPSGHCQLTDFDLSFASSSASSVAPELVPAAVPAPAPASAPSTPPAGAGPARSGSSMLRTSSTSLRSSGSAASPMLLAAQPSVRTNSLVGTEEYLAPEVIIGEGHDSMVDFWSFGILLYELLYGTTPFKASRRDATFDNIVKREPSFPPRGALVSGEAKDLIRRLLVKDPTQRLGAQAGADEVRQHPWFAGVDWALGRHSEATLARAASRAATMKRVPSKSSGMSGMGSSGSGRAPPTPKRAGDSTTVLGCFPVRRRRQ

SEQ ID NO: 12 - DNA, 2340 bp, Chlorella sorokiniana
atgtcggccctggccaagctccgccacacctttgtggtggccgatgccaccctgcccgactgcccgctggtgtatgccagccagggcttttacgacatgacgggtttcagccgtgaggaggtcatcggccacaactgccgcttcctccaaggaccggacacagaccctgagcatgtgaagaagctgagggacgcggtgaagaacggcacctgcgtcacagtgcgcctgctcaactaccgcaaggatggcacccccttctggaacctgctgaccatgacgcctgtcaaggacgataccggcaccgtggttaagattgtgggcgtgcagctggatgtgaccgacaccacagaaggcctggaggacgctgcgcacggcgtgccgctgctggtccgctacgactaccgcctgcaggacaagctggtgacgcctgcaacggacgatgtgctgctggggctgcaggaggacgacgagctggcgaccaaagcaggcgcaccaggcagcgtgcaccccgtgcaccgcctgtccacctccacgctgctgcggcagcaccaccgcgggcagctggatctgggcacgacctttgagcgcatgcagcagaatttcgtggtgtcggaccccacgctgcccgactgccccatcgtgtttgcctccgacgggttcctggagctcacggggtaccggcgtgaggaggtgctgggccacaactgccgcttcctgcaaggccccgatacagaccgagccgaagtggagaagctcaaggcggccatcaccaactgggaggagatcactgtgaggctgctcaactacaccaagaccggcacgcccttttggaacctgctcacggtggcgcccattctggatggcaagggccacccccgcctgctcgtgggcgtgctgatggatgcgaccaacatcagcattgagggtggtgcggcagcggagcaccaggccgctgtgtcagtggggcgtgcgctgggcacgatgggctgggacggcagcgacccctgggagcactttcagacggccctggccccggccaagccccaccaggccagcgaccccgccgccgccgctctgcgcgctgtggtcaaggcggatggcgagctgcgactggagcgcttccgccgcattgctgacctgggtgcgggcgacgcgggggtggtgaccctggttgagctgcggcctccagaggcagcgggcgcagcgggcatgaccgccagcggcgggcgcttcctgttcgcgctgaagtccatggacaaaaaggcgatggaggagcgcaacaaggtgggccgcgtgcgcacggaggaaaccattctgcggacagtggaccacccctatttggccaagatgtacgccaccatccacacagacacgcacctccacttccttctggagtactgctccgagggcgtgctgtacgacgtgctggagcgctcccccgaccactgtattccggaggcggaggccaagagcatcgccgccgaggtgctgctggcgctgcagtacctacacctacatggcgtgatctaccgggacctgaagcccgagaacatcctgctgaggccctccggccactgccagctcaccgatttcgacctgtcctttgcgagcagcagcgcctccagcgtagcacccgagcttgtgccagctgccgtccccgccccggcccccgcatccgctcccagcacgcccccagcaggcgccggccccgcacgaagcggcagctccatgctgcggaccagctcaaccagcctgcggtctagtggcagcgcagcgagccccatgctgctggcggcgcagcccagcgttcggaccaactcgctggtggggactgaggagtacctggcccctgaagtcatcattggagagggccacgacagcatggtggacttttggtcctttggcatcctgctctatgagctgctgtacggcaccacgcccttcaaggcctcccgccgcgatgccactttcgacaacattgtgaagcgggagccgagcttcccgccgcgcggcgccctggtctcaggggaggccaaggacctgatccggcgcctgctggtcaaggaccccacgcagcgcctgggcgcgcaggcgggtgctgacgaggtgcggcagcacccctggttcgccggcgtggactgggccctggggcggcactcagaggccaccctggctcgtgccgccagccgggcggcaaccatgaagcgtgtgcccagcaagtcctctggaatgagcggcatgggcagcagcggtagcggcagggcaccaccgacgcccaagcgggctggggacagcaccacggtgctggggtgcttccccgtgcgccgccggcgccagtga

SEQ ID NO: 13 - protein, 724 aa, Picochlorum soloecismus
MSPGVREGGVAPGAATKVPEPQAKLTTALAGLRHTFVVADATLPDCPLVYASEGFLTMTGYSKEEVLGHNCRFLQGEGTDPKSVDQIRSAVKKGHSLCTRLLNYKKNGTPFWNLLTITPIRDETGRVVKFVGVQVDVTSTTEGRAIKDSEGVPVLINYDDRLKENVAKPIVDDVLHAVQRDEGKSPKRLSRTGGAPGSPRSFPRVALDLATTVERIQSNFVIADPTLPDCPIVFASDAFLRLSGYRREEVLGRNCRFLQGNETDRSTVLELKAAIKAGKEITVRLLNYKKDGTPFWNMLTVASIRDVTGRLRFYVGVQVDVTAEPTVETAAPVGMKAASIVGDAMKRFDWVGVDPWISFKSGVMPLKPHRRQDPNALILHELAKKEGKLRLKNFFRSKQLGAGDVGMVDLLTLDGHKYAMKSLEKQEMIDRNKVGRVKTEQTILENIDHPFLATCYAKIQTDTHLHFVLEYCSGGELYGLMNSMPGKRLPEDWVKFYAAEVLLALQYLHLMGYFYRDLKPENILIHSSGHIKLTDFDLSYCQGETVPEVEKLDPPVRDPSDVENSIYDKREMEDYLLKLNPRGKANSFVGTEEYLAPEIIAGTGHDAMVDWWSFGILIYELSYGSSPFRGPRRDATFDNVLKKPLKFPQREEDLSDDGKDLIQRLLHKDPTLRLGHNAGADEVKRHPWFHDINWALLRNSTPPMVPDQDKTQKTQGSNTTIEGF

SEQ ID NO: 14 - DNA, 2473 bp, Picochlorum soloecismus
gacgaaataaatcaataaatttcgaccaattagtacaagagagcacactaatgggctttatgtcctgtgttgaggagtagcgaaacgggtttagaaatatcttcgtaggccgacacttgaacttctagcctcacgaagacggttgaaacacggatatctttgtgtattcatgccattcaagtctctataatctgataattgggccgttgaaatgagtcctggcgttcgagaagggggggttgcacctggcgcagcaaccaaggtgcccgaacctcaagcaaagctgacgactgcgctggcggggcttcgccatacctttgttgtggcagatgcgacgcttcctgattgtccactggtatatgctagcgaagggtttttgacaatgactggttattccaaagaagaggtgcttgggcataattgtcgatttcttcaaggggagggcactgatcccaaatctgttgaccagattcgatctgctgtgaaaaagggacactctctgtgcaccaggttgcttaattacaagaaaaatggaactccattttggaatcttctgacaataacaccaataagggatgaaactggtagagtagtaaagtttgttggcgttcaagtggatgtcacatccacaactgaaggccgagcaattaaggactctgaaggggtgcctgttttgatcaactatgatgaccgactgaaagagaatgtggcgaaacctatagtggatgatgtcctacatgcggttcaaagagatgaaggaaaatctccgaagcgactctctcgtacaggaggagctccagggtcacccaggtcatttccccgtgttgctttagatctggcaacaaccgtggaacgtatacagtccaactttgtgattgccgatccaactttaccagattgcccaatcgtttttgcatctgatgcatttttgcgcctgtctgggtatagacgagaagaggttctgggaagaaattgtcgcttccttcagggaaacgaaaccgataggtcaactgtcctggaattgaaagctgctattaaggcagggaaagagataactgttcggcttctgaactacaaaaaggacggaacaccattttggaacatgttgactgttgcttcgatacgagatgttactgggagactgaggttttatgtgggtgtacaggtggatgttactgctgagcccacggtagagacagccgctccagttgggatgaaggctgccagtattgttggcgatgcaatgaagagatttgattgggttggagtcgacccatggatctcatttaagagcggggtgatgcctctcaagcctcaccggcgccaagatccaaatgctttaattctgcatgagttggcaaagaaggaggggaaattgagattgaagaactttttccgatcgaaacaattgggcgccggtgacgtcggcatggttgatttactcacattagatggtcataaatatgcgatgaagtctctagaaaaacaagaaatgattgatagaaataaagttggtcgagtgaagacagaacagacaattctggaaaacattgaccaccccttcctggctacttgctacgcaaagatacaaacagatacacatttacatttcgtgctcgaatattgctctggaggagagctgtatggactgatgaactccatgccaggtaaaaggcttccagaggattgggtcaagttttatgctgctgaagttctgttggcgctgcagtacctgcatctcatgggctatttctacagagatttaaagccagaaaacatcttaatccatagctctggccacataaagttgacagattttgatctttcgtactgccaaggggaaactgttcctgaagttgaaaaattagatcctccagtcagggatccatctgatgtggaaaatagtatctatgataaacgagaaatggaggactacttgttgaaattgaatccaaggggtaaggccaactcctttgttggcactgaagaatacttggcaccagaaataatagcgggaaccgggcatgatgccatggttgactggtggtcctttggtattctgatctacgaactatcatacggctcttctccctttaggggcccgagaagagatgcaacgtttgacaatgttctaaagaagcctttgaaatttcctcagcgagaagaagatttgtctgacgatggaaaagaccttatccaacgtttgttgcacaaagatcccacactcagattaggccataatgctggagcggacgaagtcaagcggcatccatggttccatgacatcaactgggcgcttctacgcaatagcactcctcccatggtaccagatcaagataagacacaaaaaactcagggatctaatactactatagaaggcttttaatgttctgttcttgaattgttgtttgcattctgcacagcagcatgtccgagttgctcatagtaacaatgtaatgttgaatcaatatta

SEQ ID NO: 15 - protein, 1213 aa, Chlorella sorokiniana
MADRGLRRRDISCRRRLRCTRLPSRPPAAHPHVAACPPRPPTDAPPTWDLGAAAQGGAATSQRPVTPGLGALGDGGSRHSRQPAARAATAAPTSQKQPGFFRAAMRTTSGSLSGLLKRSGTAAPAPAGSSPLFEVRGAAMLPPDPPTRPATAAPAGPPSRTSAHQQQQQQHKAAAPPTYNVHARAAHSRPATASCGSPPVAGHRQASPAAVASHGLDPSTSPVPQPAAPTPHYQQPLRRSAVPAVTYATPSPASAAAQQLRAQQQAAAQRQAAQEPQAQRQQQQATAQPGSFSRASSLGSSAGSSSLPSSSGDYGSSSSQGAAPAAIMHQNPLFAGGSENASFAASRQQGAQPQTRPPPPVRVAPPPAATPATPPGGVLAGTAGVSPCPTPMSNLHAASNLTMLTDSRPVSPGPFLSTLPSGSERGSRPVSPQCIDSVPPTPGSFAPPGLAAAAAAAASRGMLSPIRTASSAGVAGSQHSRSLPTSPAPPAAASLPVAGGSPSACPAGMPSGTAAFRVPIFNQDGRLVGYKQNSNLIPRAGACISSAPSSPSRSAFLADPITFQTTSFSAAAGAVEGASPQPKPPVVRPPVSDSGDFAESLADPCKGFPDADNMVPGYVLGPVLGKGGFCSVRKALHEVTGQAVACKIIEKGKLKDPKDRDRVDRECRVMRNLSNHCAVIKLYEYVETRDCVYIMMEAAKRGSLLDYVRERKRLPEAEAVLIFQQLLHALQFCHRKDVVHRDIKLENILIDGAGHMKLIDFGLCGYYVAGKRLRCHCGSPSYAAPEIVARKDYLGPPVDVWSLGIVLFAMLAGYLPFHAKEKKQLSEKILAGVYKPAAWMSADAQDLLSRMLCLDPEQRISLEAVWAHPWVAGAPRWEPPGVGADRLYRCLTDPTSGAVLPDEAVMAQLEALGADTGAIRRALRSRECNPLTATYHLQLEAHVEAQRAAAAREREAAAERAAVKRAVEQQRGASSSADWQWDFAAISAHATAADRSQAAPASSQAGAAGSGAPSSPAAGSSGLRGGGSPARLRVAAAPPAGSPTRFGMEAAAAGYVASPRRPATSGAATPTLHFGGTPFSSPPPAQAPPAAQRPDAVPLAFAAAEQQQPRPVPSEPFTIKALRAPPGSGEPSVSTPVAAAAAAAELAATPGLITASAVTTTTAVVEGEGPTSPSKSPRLAPLPAVLSPKQLAGVGAPGTTSGGDHSPPLAQAV

SEQ ID NO: 16 - DNA, 3642 bp, Chlorella sorokiniana
atggccgacagggggctccgcaggcgcgatatttcttgccgccggcgcctgcgctgtacccgcctgccgagtcgcccaccggccgctcacccgcacgttgctgcctgcccaccccgcccacccacagatgcgccgcccacctgggacttaggcgcagccgcgcaaggcggcgcagccaccagccagcggccggtgacgccggggcttggagccctcggcgacggcggcagccggcactcgcggcagccggcggcgcgggcggccactgccgcgccgacctcgcagaagcagcccggattcttcagggcagccatgcgcaccacctccggctccctgagcggcctgctcaagcggtcgggcacggcggcgcccgcgccggccggctcctcgcctctctttgaggtgcgcggcgcggcgatgctgccgcccgatcccccaacgcggccagccactgcggcacctgccggaccgccgagccgcaccagcgcacaccagcagcagcagcagcagcacaaggcggcggcgccgcccacgtacaatgtgcacgcccgggcggcgcacagccggccggccaccgccagctgcgggagcccgcctgttgcgggacaccggcaggcctccccggcagcggttgcatcgcacgggctggacccatccaccagcccagtgccgcagccggcagcacccacgccgcactaccagcagccgttgcggcgatcggctgtgcccgcagtgacgtacgccacgcccagcccggccagcgcggcagcccagcagctgcgggctcagcagcaggcggccgcgcagcggcaggcagcccaggagccgcaagctcagcggcagcagcagcaggcgacggcgcagccgggctccttcagccgagcctcttccctgggcagcagcgcaggcagcagctcgctgccctccagctcaggtgactacggctccagcagcagccagggcgccgccccagccgccatcatgcaccagaacccgctgtttgcaggtggcagcgagaacgccagctttgcagcctcgcggcagcagggggcgcagccgcagacacggccaccgccgccggtgcgtgtggcgccgccgcctgcagccacgcccgccacgccaccaggcggcgtgcttgcgggcacggcaggcgtctcgccctgccccacacccatgagcaacctgcacgctgcctccaacctgacgatgctgactgacagccggcccgtcagcccgggccccttcctgtccaccctgcccagcggcagtgaacgcggcagccgccccgtcagcccgcagtgcatcgatagcgtgccgcccacgcccggctcgtttgcgccgccaggcctggcggcagcggcagcagcggccgcctcgcggggcatgctgtcccccattcgcacagcctcgtcagcaggcgttgctggcagccagcattcgcggtcgctgcccacctcgccggcgccgcctgccgctgcctccttgccggttgcgggcggctcgccgtcggcttgccctgcgggcatgccctctggcaccgccgctttccgggtgcccattttcaaccaggacggccgcctggtgggctacaagcagaacagcaacctcatcccgcgggccggcgcctgcatcagctcggcaccctcctcgccctcgcgctccgccttcctggcggaccccatcaccttccagaccacctccttctctgctgccgctggggcggtggagggcgcgtcgccgcagccaaagccgccggtggtgcgcccgcccgtgagcgacagcggcgactttgccgagtccctggccgacccgtgcaagggcttccctgacgccgacaacatggtgccgggctacgtgctggggccggtgctgggcaagggcggcttctgcagcgtgcgcaaggcgctgcacgaggtgacgggccaggcggtggcctgcaagatcatcgagaagggcaagctcaaggaccccaaggaccgggaccgcgtggaccgcgagtgccgcgtgatgcgcaacctgtccaaccactgcgccgtgatcaagctgtacgagtacgtggagacgcgcgactgcgtgtacatcatgatggaggcagccaagcgcggctcgctgctggactatgtgcgcgagcgcaagcgcctgcccgaggccgaggcggtgctcatcttccagcagcttctgcacgccctgcagttctgccaccgcaaggacgtggtgcaccgggacatcaagctggagaacatcctgattgacggcgcgggccacatgaagctgatcgactttgggctgtgcggctactacgtggcgggcaagcggctgcgctgccactgcggctccccctcctacgccgcccccgagattgtggcccgcaaggactacctgggcccgcccgtggatgtgtggtcgctgggcatcgtgctgtttgccatgctggcgggctacctgcccttccacgccaaggagaagaagcagctgagcgagaagatcctggcgggcgtgtacaagccggcggcctggatgagcgcggacgcccaggacctgctgtcccgtatgctgtgcctggaccccgagcagcgcatcagcctggaggctgtgtgggcgcacccctgggtggcgggcgcgccgcgctgggagccgcccggcgtgggcgccgaccgcctctaccgctgcctcaccgaccccacctcaggggcggtgctgcccgacgaagcagtcatggcgcagctggaggcgctgggtgccgacacgggcgccatccgccgcgcgctgcgctcgcgcgagtgcaaccccctcacagccacgtaccacctccagctggaggcgcatgtggaggcgcagcgtgcggcggctgctcgggagcgcgaggcggccgccgagcgtgccgccgtcaagcgcgcggtggagcagcagcgcggcgcctcctcctctgccgactggcagtgggactttgccgccatcagcgcgcacgccaccgcagcagaccgctcccaggctgcgcctgccagcagccaggccggcgcagccggcagcggtgccccctcctctcctgccgccggcagcagcggcctgcggggcggcggatcgccagcgcggctgcgcgtggccgctgcaccgccagcaggcagccccacccggttcggcatggaggcagctgccgctggctacgtggcgtcgccgcggcggcccgccaccagcggcgcagccacgcccacgctgcactttggcggcacgcccttctcctcgccaccccctgcccaggcgccgccggcagcgcagcggccggacgccgtgccgctcgcctttgcagctgcagagcagcagcagccgcggccggtgcccagcgagcccttcacaatcaaggcgttgcgtgcgccacctggcagcggcgagcccagcgtcagcacgccggtggcggcggctgccgcggccgccgagctggcggccacccccggcctcatcaccgcgtcggcggtcaccacgaccacggcggtggtggagggggaggggcccacctccccctccaagtcgccgcgcctggcgcccctgccagccgtgctctcgcccaagcagctggcaggcgtgggcgcccctggcaccaccagcggcggcgaccacagcccgccgctggcgcaggcggtgtga

SEQ ID NO: 17 - protein, 555 aa, Chlorella sorokiniana
MTSVSPGDAAAAAGHAGQPGAQGFATTSAEFFLQNYRLGKTLGIGSFGKVKVAEHILTGHKVAIKILNRKKIKQMDMEEKVRREIKILRLFMHPHIIRLYEVVETSNDIYVVMEYAGELFDYIVEKGRLLEDEARHFFQQARAKTTIISGVEYCHRNMVVHRDLKPENLLLDSKMNIKIADFGLSNVMRDGHFLKTSCGSPNYAAPEVISGRLYAGPEVDVWSCGVILYALLCGSLPFDDENIPNLFKKIKGGIYTLPSHLSPGARDLIPRMLLVDPLKRITIPEIRQHPWFTVHLPRYLAVMQADPVAAGMHVDEDIVRDVVRLGFTRDFVVDSLRARQQNKASVAYYLMADNRRRMPSSAYLKEEMTEATDPGLAAFPSGVMATSRSSTSLQPAPRLVELYRTLQYCGVFWKKNGPYNLKCRAVLHLAPPAAEDEAGGAAAPNGQAGAGGELTRDHSDDSMGGSMEASPAVAVQQQQGGLAAALAAEDARMAEAAAAVTGAGGGAAAMEREVKFEAQLYKMRDGEYALDFQRLSGDLFLFMDTCSSLLSVLRL

SEQ ID NO: 18 - DNA, 1668 bp, Chlorella sorokiniana
atgacctcggtgtcaccgggggatgccgccgcggccgctggccacgccgggcagccaggggcacagggcttcgccaccaccagcgccgagttcttcctgcagaactaccggctgggcaagacgctgggcatcggatcgttcggcaaggtcaaggtggcagagcacattctgacggggcacaaggtggccatcaagatcctgaaccgcaagaagatcaagcagatggacatggaggagaaagtgcggcgcgagatcaagattctccggctcttcatgcacccgcacatcatccggctgtatgaggtggtggagacgtccaacgacatctacgtggtcatggagtatgcgggcgagctgtttgactacattgtggagaaggggcggctgctggaggacgaggcgcgccacttcttccagcaggcgcgtgctaagaccacaatcatctcgggcgtggagtactgtcaccgcaacatggtggtgcaccgagacctgaagcccgagaacctgctgctggacagcaagatgaacatcaagattgcggactttggcctctccaacgtgatgcgggacggccacttcctcaaaacctcctgcggctcccccaactatgcggctccagaggtcatctccggccggctgtatgcgggcccagaggtggatgtctggtcctgcggcgtcatcctgtacgccctgctctgcggctcgctgccctttgacgacgaaaacatccccaacctgttcaagaagattaagggcggcatctacaccctgcccagccacctgagccccggcgcgcgagacttgatcccccgcatgctgctggtggacccgctcaagcgcatcaccattccggagatcaggcagcacccctggttcacggtccacctacctaggtacctggcagtcatgcaggcggacccagtggcagcaggcatgcacgttgatgaggacattgtgagggacgtggtccgactaggcttcacccgcgattttgtggtggactcgttgcgggcgcggcagcagaacaaggcctcggtggcttactacctgatggccgacaaccggcggcgcatgccctccagcgcttacctgaaagaggagatgacggaagcaacagatccggggctggctgctttcccgtcaggtgtcatggccaccagccgcagcagcacgagcctgcagccggcgccgcgtctggtggagctgtaccgcacgctgcagtactgcggcgtgttctggaagaagaacggtccttacaacctcaagtgccgggcggtcctgcacctggccccgcccgctgcggaggacgaggccggcggcgcagcagcacccaacggccaggccggggcaggcggagagctcactagggaccactcggatgatagcatgggcgggtctatggaggcctcgccagcggtggcagtgcagcagcagcagggcggcttggcggcggcgctggcggcagaggatgcgcgcatggcggaggcggcggcggcggtcacgggcgcaggcggcggcgcggccgccatggagcgtgaggtcaagtttgaggcgcagctgtacaagatgcgggacggcgaatatgcgctcgacttccagcgcctgtcgggcgacctgttcctgttcatggacacctgcagcagcctgctgagcgtgctgcgcctctag

SEQ ID NO: 19 - protein, 1192 aa, Chlorella sorokiniana
MAEGRPTRTQDLHPHARSSADAPPLWEFGAAAHRGAASQRPVTPAVGALGDGGSRQARQPAARAATAAPPASLKQPGFFRAAMRTTSDSLSGLLKRPGTAAPAPAAAGASPLFEVRGAAMLAPEPPAQLATVAPAASHGRSGVHPQQHKPAPPAYNVHARAAHSRPATATCASPPVAPSPQRQASPAAVASHGLDPSTSPAPQPAAATPHYQQPLRRPAAAVHATTNPTAASSAAAAAQQAAAQRQAAQEQQFQRQQQQTQAQGGTFSRASSLGSSAGSSSLPSSSGDYGSSSQGPAPPAVMHQNPLYTGGSESVSFGLSRQQAAQPQARPPPVRVAPPARALPATPPASMVAGTAGVSPSPTPTSNLQAASNLTMLTDSRPISPGPFMSTLPSGSERGSRPVSPQCIDSVPPTPGSFAPPGMAAAAAAAAASRGMLSPTRTASSAAVTVGQQSRSLPTSPAPPAAASLPVAGGSPSACPAGMPSGTAAFRVPIFNQDGRLVGYKQNSNLIPRPGACISSAPSSPSRSAYLADPTTFQTASFSAAGAAEGASPQPKPPVVRPPASDSGDFADYLADPCKGFPDADNMVPGYVLGPVLGKGGFCSVRKALHELTGQAVACKIIEKGKLKDPKDRDRVDRECRVMRNLSNHCAVIKLFEYVETRDCVYIMMEAAKRGSLLDYVRERKRLPEPEAVLIFQQLLHSLQFCHRKDVVHRDIKLENILIDAAGHMKLIDFGLCGYYVAGKRLRCHCGSPSYAAPEIVARKDYLGPPVDVWSLGIVLFAMLAGYLPFHAKEKKQLSEKILAGVYKPAAWMSAEAQDLLSRMLCLDPEQRITLEAVWAHPWVAGAPRWEPPGVGAGRVYRCLTDPTTGAVLPDEAVMAQLEALGADTAAIRRALRSRECNSLTASYHLQLEAHLEAQRAAASREREAAAERAAAKRLAEQQRGASCSADWQWDFAAISAHAAAAERCQAAAAAGSQAAAGGSGAPLSPAAAAAGSGGLRGSGSPARLRLAVAPPAGSPTRFGVEAAAAGYVSSPRRPATSGAATPTLHFGGTPFSSPPLAQAPPAAQQPEAVPAMPAAFVSAEARPRPADSEPFTIKALHAPPGSGEPSVSTPVAAAAAAAELAATPALITAAAATTTTAVVEGEGPTSPSKSPRLAPLPAVLSPKQLAGMGAPGTTSAGDHSPPMAQAV

SEQ ID NO: 20 - DNA, 3582 bp, Chlorella sorokiniana
atggctgaaggccggccaaccagaacgcaggacctccacccgcacgcccgtagctccgcagatgcgccgcccttgtgggagtttggcgcagccgcacacaggggcgccgccagccagcggccggtgacgccggcagtcggcgccttgggcgatggcggcagccggcaggcgcggcagccggcggcgcgggcggccaccgctgcgccgcccgcctcgctcaagcagcccggcttcttcagggcagccatgcgcaccacctccgactcactcagcggcctgctgaagcggccaggcactgcggcgcctgcacctgcagctgccggcgcctcgccgctgtttgaggtgcgcggtgccgcaatgctggcgcctgagcctccggcacagctagccaccgtagcgccagccgcctcgcacggccgctctggcgtgcatccgcagcagcacaagccggcgccgccggcgtacaatgtgcacgcccgggcggcgcacagcaggccggccaccgccacctgcgccagcccgcccgtggcgccgtccccacaacggcaggcatccccagcagcagtggcgtcgcatgggctggacccatccaccagccctgcgccgcaaccagcagcagccacgccgcactaccagcagccgctgcgccggccagctgcagcggtgcatgccacgaccaaccccaccgcagccagctctgccgccgctgcggcccagcaggcagcggcgcagcggcaggcggcccaggagcagcagttccagcggcagcagcagcaaacgcaggcacagggaggcacgttcagccgcgcctcctcgctgggcagcagcgccggcagcagctcgctgccctccagctcgggcgactacggcagcagcagccagggcccagccccgcccgctgtgatgcaccaaaaccctctgtacacgggcggcagcgagagcgtcagctttgggctgtcgcggcagcaggcggcgcagccgcaggcgcggccgccgccagtgcgggtggcgccgcctgccagggcgctgcctgccacgccgccggccagcatggttgcgggcacggctggcgtctcgcccagccccacacccactagcaacttgcaggcggcctccaacctgacgatgctgaccgatagccggcccatcagcccgggccccttcatgtccaccctgccaagcggcagtgagcgcggcagccgccccgtcagcccgcagtgcatcgatagcgtgcctcccactcccggctcgtttgcgccaccaggcatggctgcggcagcggcagctgcggccgcctcccgtggcatgctgtcccccactcggacagcatcctcagcagcggtcacagttggccagcagtcacgctcgctgcccacgtctccggcgccgcctgcagctgcctccctgccggtggcaggcggctcgccctcagcctgcccggcgggcatgccctccggcacagcggctttccgggtgcccatcttcaatcaggatggccgcctggtgggctacaagcagaacagcaacctcatcccccggcccggagcctgcatcagctcggcgccatcctcgccctcgcgctccgcctatctggcagaccccaccaccttccagaccgcctccttctccgccgctggagcggctgagggcgcgtcgccccaacccaagccgccggtggtgcgcccgcccgccagcgacagcggcgactttgccgactaccttgccgacccgtgcaagggcttccccgatgcagacaacatggtgccgggctacgtgctgggcccggtgctgggcaagggcggcttctgcagcgtgcgcaaggcgctgcatgagctgacggggcaggcggtggcctgcaagatcatcgagaagggcaagctcaaggaccccaaggaccgcgaccgcgttgaccgcgagtgccgcgtgatgcgcaacctgtccaaccactgcgccgtgatcaagctgtttgagtacgtggagacgcgcgactgcgtgtacatcatgatggaggcggccaagcgcggctcgctgctggactatgtgcgggagcgcaagcgcctgccggagcccgaggcggtgctcatcttccagcagctgctgcactccctgcagttctgccaccgcaaggacgtggtgcaccgcgacatcaagctggagaacatcctgattgacgccgcggggcacatgaagctgatcgactttggcctgtgcggctactacgtggccggcaagcggctgcgctgccactgcggctcgccctcgtatgccgcccccgagattgtggcccgcaaggactacctgggcccgccggtggacgtgtggtcgctgggcatcgtgctgtttgccatgctggcgggctacctgcccttccacgccaaggagaagaagcagctgagcgagaagatcctggcgggcgtgtacaagcccgcggcctggatgagcgccgaagcccaggacctgctgtcccgcatgctgtgcttggaccccgagcagcgcattacactagaagccgtgtgggcacacccctgggtggcgggcgcgccccgttgggagccgcccggagtgggcgccggccgcgtctaccgctgcctcaccgaccccaccacgggggcggtgctgcccgacgaagcggtcatggcccagctggaggcgctgggcgccgacacagccgccatccggcgcgcgctgcgctcgcgggaatgcaactccctgaccgccagctaccacctgcagctggaggcgcacctggaggcgcagcgggcggctgcctcccgagagcgcgaggcggcagccgagcgggccgccgccaagcgtttggcggagcagcagcgcggcgcctcctgctccgccgactggcagtgggatttcgcggccatcagcgcgcacgccgcggcagcggagcgctgccaggcggcggcagcagctggcagccaggcggcagcaggaggcagcggagcccccttgtctcccgctgccgcggctgccggcagcggtggcctgcggggcagcggctcgccggcgcggctgcgcctggcagttgcgccacctgcgggtagccccactcgatttggcgtggaggctgcggctgctggctatgtctcgtcgccacggcggcccgcgaccagcggagcagccacgcccacgctgcactttggcggcacccccttctcctcgccgccgctggcccaggcaccgcccgcagcgcagcagccagaggccgtgcccgccatgccggcggcctttgtctctgccgaggcgcggccacggccggcagacagcgagccgttcacaatcaaggcgctgcatgcgcctcccggcagcggcgagcccagcgtgagcacgcctgtggcggcagctgcggcggccgctgagctagcggccacccctgccctcatcaccgcggctgcagctgtcaccaccacgacagcggttgtggagggagaggggcccacctctccctccaagtcgccgcgcctggcgcctctcccagccgtgctgtcgcccaagcagctggcggggatgggcgccccaggcacaaccagcgccggcgaccacagcccgccgatggcgcaggccgtctga

SEQ ID NO: 21 - protein, 580 aa, Chlorella sorokiniana
MTSVSPGDGGAAAGHAGQPGAQGFVSSSAEFFLQNYRLGKTLGIGSFGKVKVAEHILTGHKVAIKILNRKKIKQMDMEEKVRREIKILRLFMHPHIIRLYEVVETTNDIYVVMEYVKAGELFDYIVEKGRLLEDEARHFFQQIISGVEYCHRNMVVHRDLKPENLLLDSKMNVKIADFGLSNVMRDGHFLKTSCGSPNYAAPEVISGRLYAGPEVDVWSCGVILYALLCGSLPFDDENIPNLFKKIKGGIYTLPSHLSPGARDLIPRMLLVDPLKRITIPEIRQHPWFTVHLPRYLAVMQASGLVQADPVAAGTHVDEEIVRDVVRLGFTRDFVVDSLRARQQNKASVAYYLMADNRRRMPSSAYLKEEMTEATDPGLAAFPSGMMATSRSNTSLQPAPRLVVERRWRLGLCSRAHPSSIMQASELYRTLQYCGVSWKKNGPYNLKCRAVLHLTPLAGAAGEAGGDAAPNGQAGVGGGLPRDASDDSMGVAMEASPAAAAQQQQGGLAAALAAEDARMAEAAAAVVGSGGGAGTLEREVKFEAQLYKMRDGEYSLDFQRLSGDLFLFMDTCSSLLSVLRL

SEQ ID NO: 22 - DNA, 1855 bp, Chlorella sorokiniana
atagcacagtcgcgttccaacgccccgccagcccgctccgacatcgcctggcccgctcgcagctctccggtcgccgcagggtcgcttatcgccgcagctgacgggaccaacaatgacctcagtgtcgcctggagacggtggcgcggccgcaggccacgcagggcagccaggggcgcagggcttcgtctcctccagcgccgagttcttcctgcagaactaccggctgggcaagacgctgggcatcggctctttcggcaaggtcaaggtggcggagcacatcctgacggggcacaaggtggccatcaagattctgaaccgcaagaagatcaagcagatggacatggaggagaaggtgcggcgcgagatcaagattctgcggctgttcatgcacccgcacattatccggctctacgaggtggtggagacgaccaacgacatctacgtggtcatggagtacgtcaaggcgggcgagctgtttgactacatcgtggagaaggggcggctgctggaggacgaggcgcgccacttcttccagcagatcatctcgggcgtggagtactgccaccgcaacatggtggtgcaccgtgacctgaagcccgagaacctgcttctggacagcaagatgaacgtgaagattgcggacttcgggctgtccaacgtgatgcgcgatgggcacttcctcaaaacgtcctgcggctcccccaactatgcggcgccagaagtcatttctgggcggctgtacgcgggccccgaggtggatgtctggtcctgcggcgtcatcctgtacgccctgctctgcggctcgctgccctttgacgacgaaaacatccccaacctgttcaagaagatcaagggcggcatctacacgctgcccagccacctgagcccgggagcgcgcgacctcatcccccgcatgctgctggtcgacccgctcaaacgcatcaccatcccggagatcaggcagcatccctggttcacggtgcacctgcctcgctacctagcggtcatgcaggcgagtgggctggtgcaggcggacccggtggcggcgggcacgcatgtggacgaagagattgtgcgagacgtggtgcggctaggcttcacgcgcgactttgtggtggactcgctgcgtgcacggcagcagaacaaggcctcagtagcgtactacctcatggctgacaaccggcgccgcatgccttctagcgcatacttgaaggaggagatgacggaggcgacagacccggggctggctgcgttcccctcaggcatgatggccaccagccgcagcaacacgagcctgcagccggcgcctcgcctggtggtggagcgacgctggcgcctgggcctgtgctcgcgtgcgcacccctcctccatcatgcaggcgagtgagctgtaccgcacgctgcagtactgcggtgtgtcctggaagaagaatggtccttacaacctcaagtgccgggcggtcctgcacctcacgccgctcgccggcgcggcaggcgaggctggcggcgatgcagcacccaacggccaggcaggggtgggcggaggcctgcctcgtgacgcctcggatgatagcatgggtgtcgcgatggaggcatcgcccgcggcagcagcgcagcagcagcagggcggcttggcggcggcgctggcggcagaagatgcgcgcatggcggaagcggcggcggcggtagtgggcagcggcggcggcgcaggcaccctggagcgggaggtcaagtttgaggcgcagctgtacaagatgcgggacggggagtattcgctcgacttccagcgcctctctggagacctgttcttgttcatggacacctgcagcagcctgctgagcgtgctgcgcctctag

SEQ ID NO: 23 - protein, 441 aa, Chlorella sorokiniana
SAPSSPSRSAYLADPTTFQTTSFSAAGGVEGASPQPRAPVARPPVSDSGDFAEYLADPCKGFPDADNMVPGYVLGPVLGKGGFCSVRKALHEVTGQAVACKIIEKGKLKDPKDRDRVDRECRVMRNLSNHCAVIKLFEYVETRDCVYIMMEAAKRGSLLDYVRERKRLPEHEAVTIFQQLLHALQFCHRKDVVHRDIKLENILIDAAGHMKLIDFGLCGYYVAGKRLRCHCGSPSYAAPEIVARKDYLGPPVDVWSLGIVLFAMLAGYLPFHAKEKKQLSEKILAGVYKPAAWMSGDAQDLLSRMLCLDPEQRITLEGVWAHPWVAAAPRWEPPGVGAGRLYRCLTDPTTGAVLPDEAVMAQLEALGADTAAIRRALRSRECNSLTATYHLQLEAHLDAQRAATAREREAERATAAAAAKRAAEQQRGACSSADWQWDFAA

SEQ ID NO: 24 - DNA, 1323 bp, Chlorella sorokiniana
tccgcgccctcctcgccctcgcgctccgcctacctggcggaccccaccaccttccagactacctccttctccgccgccggcggagtggagggcgcgtcgccgcagcccagggcgccagtggcacgcccgcctgtcagcgacagcggcgactttgccgagtacctggccgacccgtgcaagggcttccctgatgcagacaacatggtgccgggctacgtgctgggcccggtgctgggcaagggcggcttctgcagcgtgcgcaaggcgctgcacgaggtgacggggcaggcggtggcctgcaagatcatcgagaagggcaagctcaaggaccccaaggaccgcgaccgcgtggaccgcgagtgccgcgtgatgcgcaacctgtccaaccactgcgccgtgatcaagctgttcgagtacgtggagacgcgcgactgcgtgtacatcatgatggaggccgccaagcgcggctcgctgctggactatgtgcgggagcgcaagcgcctgccggagcacgaggcggtgaccatcttccagcagctgctgcatgctctgcagttctgccaccgcaaggacgtggtgcaccgcgacatcaagctggagaatatcctgattgacgccgcggggcacatgaagctgatcgactttgggctgtgcggctactacgtggccggcaaacggctgcgctgccactgcggctccccctcctacgccgcccccgagatcgtggcgcgcaaggactacctgggcccgccggtggacgtgtggtccctgggcatcgtgctgtttgccatgctggccggctacctgcccttccacgccaaggaaaagaagcagctgagcgagaagatcctggcgggcgtgtacaagcccgcggcatggatgagcggcgatgctcaggacttgctgtcccgcatgctgtgcctggaccctgagcagcgcatcacgctggagggcgtgtgggcacacccctgggtggcggccgcgccgcgctgggagccaccgggagtgggcgccggccgcttgtaccgctgcctcaccgatcccaccaccggggcggtgctgcctgacgaagcagtcatggctcagctggaggcgctgggcgccgacaccgctgccatccgaagggcgctgcgctcgcgagagtgcaactccctgaccgccacgtaccacctgcagctggaggcgcacttagatgcccagcgggcagcgactgcccgcgagcgcgaggcggagcgcgccaccgccgcagcggcagccaagcgggcagcggagcagcagcgcggcgcctgctcctctgctgactggcagtgggactttgctgcc

SEQ ID NO: 25 - protein, 572 aa, Chlorella sorokiniana
MTSVSPGDAGAAPGHAGQPGAQGFASSSAEFFLQNYRLGKTLGIGSFGKVKVAEHILTGHKVAIKILNRKKIKQMDMEEKVRREIKILRLFMHPHIIRLYEVVETTNDIYVAGELFDYIVEKGRLLEDEARHFFQQIISGVEYCHRNMVVHRDLKPENLLLDSKMNVKIADFGLSNVMRDGHFLKTSCGSPNYAAPEVISGRLYAGPEVDVWSCGVILYALLCGSLPFDDENIPNLFKKIKGGIYTLPSHLSPGARDLIPRMLLVDPLKRITIPEIRVASATFVMLVDPLKRVTIPEIRQHPWFTVHLPRYLAADPVAAGTHIDEDIIRDVVRLGFTRDFVVDSLRARQQNKASVAYYLMADNRRRMPSSAYLKEEMTEATDPGLAAFPSGVMATSRSNTSLQPAPRLVVERRWRLGLCSRAHPSSIMQARAGCVCERACAVKPAELYRTLQYCGVFWKKNGPYNLKCRAVLHLTPPADGGSGEGGGGAAPNGQAGASGQLTRDHSDDSMGVAMEASPAAAGQQQQGALAAALAAEDARMAEAAAAVVGSGGSDSAGSLEREVKFEAQLYKM

SEQ ID NO: 26 - DNA, 1824 bp, Chlorella sorokiniana
cttgtgtatcccctaccagcataccagcgcgctcccaggcccctccgacaccgctcgcaagcgcccggtcccgcaggctcgctcatcgccccagctgcagcgttcagcatgacctcggtgtcaccgggagacgctggggcggcgccaggccacgccgggcagcccggggcgcagggcttcgcctcctccagcgcggagttcttcctgcagaactaccggctgggcaagacgctgggtatcggctctttcggcaaggtcaaggtggcggagcacatactgacagggcacaaggtggccatcaagatcctgaaccgcaagaagatcaagcagatggacatggaggaaaaggtgcggcgagagatcaagatcctgcgcctgttcatgcacccgcacatcatccggctgtatgaggtagtggagaccaccaatgatatctacgtggcgggcgagctgtttgactacattgtggagaaggggcggctgctggaggacgaggcgcgccacttcttccagcagatcatttcgggcgtggagtactgccaccgcaacatggtggtgcaccgagacctcaagcccgagaacctgctgctggacagcaagatgaacgtcaagattgcagactttgggctgtcaaacgtgatgcgggacggccacttcctcaaaacctcctgcggctcccccaactatgcggcgccagaggtcatctccggccggctgtacgcggggcctgaggtggatgtgtggtcctgcggcgtcatcttgtacgccctgctctgcggctcgctgcccttcgacgacgagaacatccccaacctgttcaagaagatcaagggcggcatttacacgctgcccagccacctgagccccggggcgcgcgacctcatcccgcgcatgctgctggtcgacccgctcaagcgcatcacgatccccgagatcagagtagccagcgctacttttgtgatgctggttgacccactcaagcgcgtcaccatccccgagatcaggcagcacccctggttcacggtgcacctgccgcggtacctggccgcggacccagtagcagccggaacgcacatagacgaggacatcattcgggacgtggtgcggctaggcttcacccgcgattttgtggtggactccctgcgggcgcggcagcagaacaaggcgtccgtggcctactacctcatggccgacaaccggcggcgcatgccctccagcgcctatttgaaagaagagatgacggaggccacggatccggggctggccgcctttccctcaggcgtcatggccacgagccgcagcaacacgagcctgcagccggcgccgcgcctggtggtggagcggcgctggcgcctggggctgtgctcccgggcgcacccctcctccatcatgcaggcaagggctggttgtgtgtgcgagcgagcgtgtgctgtcaagcctgcggagctgtaccgtacgctgcagtactgcggggtgttctggaagaaaaacggcccttacaacctcaagtgccgagcggtcctgcacctcacgccgcccgccgacggcggcagcggagagggcggcggcggcgcggcgcccaacggccaggcgggcgcgagcgggcagctgaccagggaccactcagatgatagcatgggcgtggctatggaggcttcgcctgcggcagcagggcagcagcagcagggcgccttggcagcggcgctggcagcggaggatgcacgcatggcggaagcggcggcggcggtcgtgggcagcggcggcagtgatagcgcaggctcccttgagcgtgaggtcaaatttgaggcgcagctgtacaagatg

SEQ ID NO: 27 - protein, 918 aa, Chlamydomonas reinhardtii
MRRQGQPSGMDCDLLELQQHTSRARLLKAKERHIGHSSNMQDCNNQKSSARGLLSKGGEHKAAAKPINPTGGFRAKLLAAKRRAAFGGGASTTAKAGHVPAPSPSSGGSGSDAASGSGGVSNGSSHQQAPSTKTNTNTCSNKNGGSNKTAGTSTTASVRIGFPSVRRRPSASSTSSRHALPAAATQPTSCVPGAKPAAPQQSNAAAATTVAVAPATAGSSSGARRMEGRCRPYRPPMRVQGDEGGKAATAGTGPGSSSGSGLMLRSLVGCLLPHLATAAIEKAAHVAAAVARRVMAPVAAAAAAVPYRRMEAPVAAAAAAVPCRVMVAPVATAAAVPCGMVAPVATAAAVPCGMVAPVATAAAVPCGMVAPVAGAVAPKLVAFRPPPPPITPVHQHQHQHQQAGPRYVARPAVQPMMGPPVVQATAGAAAVAAPRLLVGAPVKLLMAPQLLQRQQHNIQLANRVACVPYPHPHPAVQPQPVLVHGGMPAPRQPHQQQDQSLQMRMQQLQQAQQMWQAQKKVQEAEARNQRIQEAIQEEEDIVVANVRELMQRFTSGASKPFELIRLKRHLGQGAFGCVDCWEVTERQSATASSAASSSGLAAAASCTATATSSSHTFEAAVKTCALDLEGLAAGGETPHSVEMHAKEAAAVLAVQALDSRHLVKVLGAYVDVLTPGEDVPPRGCGIANLHVGRIVMEVARESLTDVVTGHRVLAHCAAGGAAAAFDADSEDTSGYRLPETAVRVVLASVLLGLRDLHGRARLAHRDLKLDNLLVGTDRLVKITDFGLVTPLDSQGRLVSEVGGRRGTKGYQAPETLARRTFAANERDLPSWPAAKSDIYAVGVIGAALVCGTESGPEMEAFRASGELPPHRHASPALRQLLKGMAAADPAQRLGVEEALAHPALRRALSSERARRFIW

SEQ ID NO: 28 - DNA, 4314 bp, Chlamydomonas reinhardtii
gatctggcatacgtttccttgcgacgcctgcactcggccgctgtcaagcccgcggcaaggcaccgggcgcagcgcccaagcgcgggaaaacgcatccggctcagccctagctcctcaaagcgcttgctatgcaaattttgcagaatctcttgcattagcgcatcagggctgctgggcgcatggctgggctggagcaagtccgcgctgcgggctcaaagcgcctgtgtacgctttcagctctcttccagcgctttttatgcatgcacagaattcagcttttgcctttgcacccgctttgcagcaaccgcagcgcaggcgcggccacggcactacttgcaggcgcgctactgggtgcgcttggctccaagcgctgtcacttaaggctggcttagcaaattaaggtattcatatggagcagctttccagggcattccgcgcgggtcaacccacggtcaagcgggatggggctccttttcgtttgggggccattctttacttgagccacgcgcggcgttctatagcaaaagctttcagtgagctcatacacatacatacaactagctttcctctcaagccccagagcctcgcctagctaactatttctgacttgcgtcttcttcctcgcttttcctatcaaaggccctcaacttacagcatcggctcactgtgacacttgttgcttgtggcgacacgcatcgagtgggcggcactgcaagaggcgaacacgcttccgacggacggaaacgcgctcctggctctcccgttctgtatgcggcggcaagggcagcctagcggtatggattgcgacctgctggagctgcagcagcacacttcgcgagcgaggctgctgaaggccaaggagcggcacatagggcacagcagcaacatgcaggactgcaacaaccagaagtccagcgcccgcggtctcctgtccaagggcggcgagcacaaggccgcggccaaacccatcaatccgaccggcggtttccgtgccaagcttctggcggctaagcgccgtgccgcctttggcggtggcgcctcgaccacggctaaggctggccatgtgcccgcgccctctcctagcagcggcggcagcgggtctgacgcggcctccggcagcggtggggtctccaacggctcatcgcaccagcaggcgccctcgacaaaaaccaacaccaacacctgcagcaacaagaatggtggcagcaacaagactgctggcaccagcacgaccgcctcagtgaggattggcttcccatccgttcgccgtcgcccatccgcctcttccaccagcagcaggcacgcgctgcctgctgccgccacacaacccacctcctgcgtgcccggtgccaagcctgctgcaccccagcagtccaacgccgccgccgccaccaccgtcgctgttgctcctgcgactgctggctcatcatcaggagcccgccgcatggagggccgctgccgcccctaccggccgcccatgcgggtgcagggcgacgagggcggcaaggcggccaccgctggcactggccctggcagcagcagtggctcgggcctgatgctccgcagcttggtagggtgcctgctgccccacctcgccactgccgccatcgagaaggcagcgcacgtcgcggccgccgtcgcccgccgggtgatggcacccgtcgccgccgccgccgccgccgtgccctaccgccggatggaggcacccgtcgccgccgccgccgccgccgtgccttgccgggtgatggtggcacccgtcgccactgccgccgccgtgccctgcgggatggtggcccccgtcgccactgccgccgccgtgccctgcgggatggtggcccccgtcgccactgccgccgccgtgccctgcgggatggtggcccccgtcgccggcgcggtggcgcccaagctcgtggccttccggccgccgccgccgcccatcacgccagtgcaccagcaccagcaccagcaccagcaggcggggccgcggtacgtggcgcggccggcggtgcaaccgatgatggggccgcctgtggttcaggctaccgctggcgccgcggccgtggcggcgccgcggctgctcgtgggggctccggtcaagctcctgatggcgcctcagctgcttcagcggcagcagcacaacatccagctggccaaccgcgtggcttgcgtgccgtacccgcacccgcacccggcggtgcagccgcagccggtcctggtgcacggcggcatgccggctccgcgccagccgcaccagcagcaagaccaaagcctgcagatgcggatgcagcagctgcagcaggcgcagcagatgtggcaagcgcagaagaaggtgcaggaggcggaggcccggaatcagcggatccaggaggccattcaggaggaggaggacattgttgtcgccaacgtgcgcgagctcatgcagcgcttcaccagcggcgcctccaagcccttcgagctcatccgcctcaagcggcacctgggccagggcgccttcggctgcgtcgactgctgggaggtcacagagcgccagtccgccaccgcctcctccgccgcctcctcctctggcctggcggcggcggcctcctgcaccgccactgccaccagcagcagccacacgtttgaggcggcggtgaagacctgcgctctggacctggagggcctggcggcgggcggcgaaacgccgcattctgtggagatgcatgccaaggaggcggcggcggtgctggcggttcaggcgctggactcgcggcacctggtcaaggtgctgggggcctacgtcgacgtgctgacacccggggaggacgtgccgccccgaggctgcggcatcgccaacctgcacgtcgggcgcatcgtgatggaggtggcgcgggagtcgctgactgacgtggtgacgggccaccgcgtgctcgcgcactgcgccgcgggcggcgccgccgccgcctttgacgccgactcggaggacacgtccggctaccggctgcccgagaccgcggtgcgcgtggtgctggcctcggtgctgctgggcctgcgcgacctgcacggccgcgcgcggctggctcaccgtgacctgaagctggacaaccttctggttggcacggaccggcttgttaagatcaccgacttcggcctggtgacgccactagacagccagggccgcctggtctcggaggtcggggggcggcggggcacgaagggctaccaggcccctgagacgctggcgcgccgcaccttcgcggccaacgagcgcgacctgcccagctggccagccgccaagagcgacatttacgcggtgggcgtgatcggcgcggcgctggtgtgcggcaccgagtcggggccggagatggaggccttccgggccagtggcgagctgccgccgcaccggcacgcctcgccagcgctgcggcagctgctcaagggcatggcggcggcggacccggcgcagcggctaggggtggaggaggcactggcgcaccccgcgctgcgcagggcgctgagcagcgagcgggcgcggcgcttcatctggtgatggcggcagggccagatcgctcgggcatgaggcgggctagctacaggcgggcagtgcgtcagtgatgagcggcggctgtcaaatgaataaaggagtgcttacatgtgtgtcagagccagattggcctgagggaacattgattggcctcaggatagaacccttagcaagcaagccgtggatggcagcgtgcatagtcgcggctgctaatccttgctgcttccccttgattgattgatgatagacaatagaacgccgcgttgccggcttgccgcaagaggcagatattgagacgaatggatgcttgggccgtggcccaatggctccctgacgctgtgttctgaaactgtagggtaccatatgtgctggcgcttgcgtgtgcgttagttgtcggtccttcgggcgacggatggcctggtaggactaccttgattgagccctgttttgactcctccttacgtagtacgacgtgttgatctctttccgtcctgtccgcgtgattgctcgctgtatcccggatatggaccccagcagtttgagcacaggtctacctggcgttgagcttgcttgacatgtcttgtgtgaccgttttgcaactgaatccatgtgttctgtcgggcctatacggtgcaagcttagctatgtagccatgattgtgcacgcctctgaagagctgcaagctgctggtcgttcctaggtgcgcagcggcggtgtgcactgaccgctcagagcgctgactcctggaacatatacagcgggttcgtgcacatggtacgataacccgccttgta

SEQ ID NO: 29 - protein, 913 aa, Chlamydomonas reinhardtii
MDCDLLELQQHTLRARLLKAKERRHIGHSSNMQDCNNQKSSARSLLSKGGEHKAAAKPIKPTGGFRAKLLAAKRRAAFGGGASTTAKAGHVPAPSPSSGGSGSDADCNNQKSSARSLLSKGGEHKAAAKPTKPTGGFRAKLLAAKRRAAFGGGASTTAKAGHVPAPSPSSGGSGSDAAPGSGGVSNGSSRQQAPSANTNTNTNSYSTSSRHALPAATQPTPCVPSIQPAAPRSSAAAAATTVAAATAAGSSSGARRMEGRCRPYRPPMRVQGDEGGKAATAGSGPGSSSGSGLMLRSLVGCLLPHLATAAIQKTAHVAAAVARRVMAPVAAAATAVPCRVMAPVATAAAVPCRVVAPVAAAAADAGAVAPKLVAFRPPPPPIMPVHQHQHQDQHQQAGPWYVARPAVQPMMGPPVVQATAGAAAVAAPRLLVGAPVKLLMATQLLQQQQHIQLANRVACVPYPHPHPAVQPQPVLVHGGMPAPRQPHQQQDQSLQMRMQRLQQAKQMWQAQKKKVQEAEARQQRIQEAIQEEEDIVVANVRELMQRCTSGASKPVELIRLKRHLGQGAFGCVDCWEVTERQSPTASSAASSSGLAAAAASGTATATSSSRTFEAAVKTCALDLKGLAAGGATPHSVEMHAKEAAAVLAVQALDSRHLVKVLGAYVDVLTPGEDVPPRGCGIANLRVGRIVMEVARESLTDVVTGHRVLAHCAVGGAAAAFDADSEDTSGYRLPETAVRVVLASVLLGLRDLHGRARLAHRDLKLDNLLVGTDRLVKITDFGLVTPLDSQGRLVSEVGGRRGTKGYQAPETLARRTFAANERDLPSWPAAKSDVFAVGVIGAALVCGTESGPEMEAFRGSGELPPHRHASPALRQLLKGMAAADPAQRLGVEEALAHPALRRALSSERARRFIW

SEQ ID NO: 30 - DNA, 4486 bp, Chlamydomonas reinhardtii
ctacctggcacacatttcgttgcgacgcctgcactccggccgccttcaagcccgcggtaaggcaccgggcgcagcgcccaagcgcgggaaaacgcatccggctcagccctagctcctcaaagcgcctgctatgcaaactttgcagaatctcttgcattagcgcatcagcagcagggctgctgggcgcatggctgggctggagcaagtccgcgctgcgggctcaaagcgcctgtgtacgctttcagctctcttccagcgctttttttcatgcacagaattcagcttttgcctttgcacccgctttgcagcaaccgcagcgcaggcgcggccacggcacttgcaggcgcgctactgggtgcgcctggctccaagcgctggcacttaaggctggcttagcaaattaaggtattcatatggagcagctttccagggcattccgcgcgggtcaacccacggtcaagcgggatggggcttcttttcgtttgcaggccattctttacttgggccacgcgcggcgttctatagcaaaagctttcagtgagctcatacgcgcacacacatacatacaactagctttcctctcaagccccagagcctcgcctagctaactatttccgacttgcgtcttcttcctcgcttttcctatcaaaggccctcaacttgcagcatcggctcactgtggcacttgttgcttgtggcgacaagcatcgagtgggcggcactgcaagaggcgcacacgcttccgacggacggaaacgcgctcctggctctcccgtgctgtttgcggcggcaagggcagcctagcggtatggattgcgacctgctggagctgcagcagcacactttgcgagcgaggctgctcaaggccaaggagcggcggcacatcggccacagcagcaacatgcaggactgcaacaaccagaagtccagcgcccgcagtctcctgtccaagggcggcgagcacaaggccgcggccaaacccatcaaaccgaccggcggtttccgtgccaagcttctggcggcgaagcgccgtgccgcctttggcggtggcgcctcgaccacggctaaggctggccatgtgcccgcgccctctcctagcagcggcggcagcgggtctgacgcggactgcaacaaccagaagtccagcgcccgcagtctcctgtccaagggcggcgagcacaaggccgcggccaaacccaccaaaccgaccggcggtttccgtgccaagcttctggcggcgaagcgccgtgccgcctttggcggtggcgcctcgactacggctaaggctggccatgtgcccgcgccctctcctagcagcggcggcagcgggtctgacgcggcccccggcagcggtggggtctccaacggctcatcgcgccagcaggcgccctcggccaacaccaacaccaacaccaactcgtactccaccagcagcaggcacgcgctgcccgccgccacacaacccaccccctgcgtgcccagtattcagcctgctgcaccccggtccagtgccgccgccgccgccaccaccgtcgctgctgctacggctgctggctcatcatcaggagcccgccgcatggagggccgctgccgcccctaccggccgcccatgcgggtgcagggcgacgagggcggcaaggcggccaccgctggcagtggccctggcagcagcagtggctcaggcctgatgctccgcagcttggtagggtgcctgctgccccacctcgccactgccgccatccagaagacagcgcacgtcgcggccgccgtcgcccgccgggtgatggcacccgtcgccgccgccgccaccgccgtgccctgccgggtgatggcacccgtcgccactgccgccgccgtgccctgccgggtggtggcacccgtcgccgctgccgcggccgacgccggcgcggtggcgcccaagctcgtggccttccggcctccgccgccgcccatcatgccagtgcaccagcaccagcaccaggaccagcaccagcaggcggggccgtggtacgtggcgcggccggcggtgcaaccgatgatggggccgcctgtggttcaggctaccgctggcgccgcggccgtggcggcgccgcggctgctcgtgggggctccggtcaagctcctgatggcaactcagctgcttcagcagcagcagcacatccagctggccaaccgcgtggcttgcgtgccgtacccgcacccgcacccggcggtgcagccgcagccggtcctggtgcacggcggcatgccggctccgcgccagccgcaccagcagcaagaccaaagcctgcagatgcggatgcagcggctgcagcaggcgaagcagatgtggcaagcgcagaagaagaaggtgcaggaggcggaggcccggcagcagaggatccaggaggccattcaggaggaggaggacattgttgtcgccaacgtgcgcgagctcatgcagcgctgcaccagcggcgcctccaagcccgtcgagctcatccgcctcaagcggcacctgggccagggcgccttcggctgcgtcgactgctgggaggtcacagagcgccagtcccccaccgcctcctccgcggcctcctcctctggcctggcggcggcggcggcctccggcaccgccactgccaccagcagcagccgcacctttgaggcggcggtgaagacctgcgctctggacctgaagggcctggcggcgggcggcgcaacgccgcattctgtggagatgcatgccaaggaggcggcggcggtgctggcggttcaggcgctggactcgcggcacctggtcaaggtgctgggggcctacgtcgacgtgctgacacccggggaggacgtgccgccccgcggctgcggcatcgccaacctgcgcgtcgggcgcatagtgatggaggtggcgcgggagtcgctgactgacgtggtgacgggccaccgcgtgctcgcgcactgcgccgtgggcggcgccgccgccgcctttgacgccgactcggaggacacgtccggctaccggctgcccgagaccgcggtgcgcgtggtgctggcctcggtgctgctgggcctgcgcgacctgcacggccgcgcgcggctggcgcaccgggacctgaagctggacaaccttctggttggcacggaccggcttgttaagatcaccgacttcggcctggtgacgcctctggacagccagggccgcctggtctcggaggtcggggggcggcggggcacgaagggctaccaagcccctgagacgctggcgcgccgcaccttcgcggccaacgagcgcgacctgcccagctggccggccgccaagagcgatgtgttcgcggtgggcgtgatcggcgcggcgctggtgtgcggcaccgagtcggggccggagatggaggccttccggggcagtggtgagctgccgccgcaccggcacgcctcgccggcgctgcggcagctgctcaagggcatggcggcggcggacccggcgcagcggctgggggtggaggaggcactggcgcaccccgcgctgcgcagggcgctgagcagcgagcgggcgcggcgcttcatctggtgaaggcggcaaggccagatcgctcgggcatgaggcgggctatatgcggacagtgcgtcagtgatgagcggcggctgtcaaatgaataaaggagtgcgtacgtgtgtgtgccagagcttacttgcttgagagctgggtgtttttaagtataaagacacagcatgcgcagtgttgaggaccgcacagaggttcataggacatagattggcctgagggaacgttgattggcctcagggtagaacccttagcaagcaagccgtggatggcagcatgcatagttgcggctgctgatccttgctgcttccccttgattgattgatgatacggtaggcctagacattagaatgccgcgttgattgcttgccggcttgccgcaagaggcagatagagacgaatggatgcttgggccgtggcccaatggctccatgacgctgtgttctgaaactcaagggtaccacatgtgctggcgcttgcgtgtgcgttgtcggtccttcgatcaacacgtcttactacgtacgaaaattcgggcaacggactgcggtagggctacctttactaccttgagccctattttgactcctctgcacgtagtacgacgtgttgatctatttccgtcctttccgcgtgattgctgtatcccggcggacatgaccccagcagtttgagcacaggtctacctggcgttgagcttgcttgacatgtcttgtgtgaccgttcgcaactgaatccatgtgttctgtcgggtgtatacggtgcaagcttagctatgtagccatggttgtgcacgcctgtgatgaagagccgcaagctgctggtcgtctcctaggcgcgcagcggtaggtgcgcactgaccgctcagagcgctgactcctggaacatatacagcaggttcgtgcacatggtacggtaacccgccttgtagtgcaaagggacactcca

SEQ ID NO: 31 - protein, 834 aa, Chlamydomonas reinhardtii
MDCDLLSELQQHTLRARLLKAKERHIGHSSNMQDYNNQKSSARGLLSKGGEHKAAAKPKSTGGFRAKLLAAKRRAAFGGGASTTAMAGHVPAPSPSSGGSGSDAAPGSGGVSNGSSRQQAPSANANTDTNTNTGSNKTGGTNTTASPAAPQQSNAAAATTVAVAPATAGSISGARRMEGRCRPYRPPMRVQGDEGGKAATAGTGPAGSSSSGSGLMLRSLVGCLLPHLATAAIEKAAHVAAAVAPRVMAPVAAAAAAVHYRRMEAPVAAAAAVPCRMVAPVAGAVAPKLVAFRPPPPPIMPVHQHQHQDQHQQAGPRYVARPAVQPMMGPPVVQATAGAAAVAAPRLLVGAPVKLLMAPQVLQQQQHIQQLANRVACVPYPHPHPAVQPQPVLVHGGGMPAARQPHQQQDQSLQMRLEQRLQQAKQMWQATKKRVQEAKARRKQRIQEAIQEEEDIVVANVRELMQRVTSGAPKPFELIRLKRHLGQGAFGCVDCWEVTERQSATASSAASSSGLAAAAASGTATATSSSRTFEAAVKTCALDLEGLAAGGETPHTVEMHAKEAAAVLAVQALDSRHLVKVLGAYVDVLTPGEDVPPRGCGIANLRVGRIVMEVARESLTDVVTGHRVLAHCAAGGAAAAFDADSEDTSGYRLPETAVRVVLASVLLGLRDLHGRARLAHRDLKLDNLLVGTDRLIKITDFGLVTPLDSQGRLVSEVGGRRGTKGYQAPETLARRTFAANERELPSWPAAKSDVFAVGVIGAALVCGTESGPEMEAFRASGELPPHRHASPALRQLLKGMAAADPARRLGVEEALAHPALRRALSSERARRFIW

SEQ ID NO: 32 - DNA, 3849 bp, Chlamydomonas reinhardtii
gttacacatacaactagctttcctctcaagccccagagcctcgcctagctaactatttccgacttgcgtcttcttcctcgcgctcgcttttcctatcaaaggccctcaacttgcagcatcggctcactgtggcacttgttgcttgtggcgacaagcatcgagtgggcggcactgcgagaggcgcacacgcttccgacggacggaaacgcgctcctggctctcccgtgctgtttgcggcggcaagggcagcctagcggtatggattgcgacctgctgtctgagctgcagcagcacactttgcgagcgaggctgctgaaggccaaggagcggcacataggccacagcagcaacatgcaggactacaacaaccagaagtccagcgcccgcggtctcctgtccaagggcggcgagcacaaggccgcggccaaacccaaatcgaccggcggtttccgtgccaagcttctggcggccaagcgccgtgccgcctttggcggtggcgcctcgaccacggctatggctggccatgtgcccgcgccctctcctagcagcggcggcagcgggtctgacgcggcccccggcagcggtggggtctccaacggctcatcgcgccagcaggcgccctcggccaacgccaacaccgacaccaacaccaacaccggcagcaacaagactggtggcacgaacacgaccgcctcgcctgctgcaccccagcagtccaacgccgccgctgccaccaccgtcgctgttgctcctgcgactgctggctcaatatcaggagcccgccgcatggagggccgctgccgcccctaccggccgcccatgcgggtgcagggcgacgagggcggcaaggcggccaccgctggcactggccctgctggcagcagcagcagtggctcgggcctgatgctccgcagcttggtagggtgcctgctgccccacctcgccactgccgccatcgagaaggcagcgcacgtcgcggccgccgtcgccccccgggtgatggcacccgtcgccgccgccgccgccgccgtgcactaccgccggatggaggcacccgtcgccgctgccgccgccgtgccctgccggatggtggcacccgtcgccggcgcggtggcgcccaagctcgtggccttccggcctccgccgccgcccatcatgccagtgcaccagcaccagcaccaggaccagcaccagcaggcggggccgcggtacgtggcgcggccggcggtgcaaccgatgatggggccgcctgtggttcaggctaccgctggcgccgcggccgtggcggcgccgcggctgctcgtgggggctccggtcaagctcctgatggcacctcaggtgcttcagcagcagcagcacatccagcagctggccaaccgcgtggcttgcgtgccgtacccgcacccgcacccggcggtgcagccgcagccggtcctggtgcacggcggcggcatgccggctgcgcgccagccgcaccagcagcaagaccaaagcctgcagatgcggctggagcagcggctgcagcaggcgaagcagatgtggcaagcgacgaagaagagggtgcaggaggcgaaggcccggcggaagcagcggatccaggaggctattcaggaggaggaggacattgttgtggccaacgtgcgcgagctcatgcagcgcgtcaccagcggcgcccccaagcccttcgagctcatccgcctcaagcggcacctgggccagggtgccttcggctgcgtcgactgctgggaggtcacagagcgccagtccgccaccgcctcctccgcggcctcctcctctggcctggcggcggcggcggcctccggcaccgccactgccaccagcagcagccgcacctttgaggcggcggtgaagacctgcgctctggacctggagggcctggcagcgggcggcgaaacgccgcatacggtggagatgcatgccaaggaggcggcggcggtgctggcggttcaggcgctggactcgcggcacctggtcaaggtgctgggggcctacgtcgacgtgctgacacccggggaggacgtgccgccccgcggctgcggcatcgccaacctgcgcgtcgggcgcatcgtgatggaggtggcgcgggagtcgctgactgacgtggtgacgggccaccgcgtgctcgcgcactgcgccgcgggcggcgccgccgccgcctttgacgccgactcggaggacacgtccggctaccggctgcccgagaccgcggtgcgcgtggtgctggcctcggtgctgctgggcctgcgcgacctgcacggccgcgcgcggctggcgcaccgggacctgaagctggacaaccttctggttggcacggaccgtctgattaagatcaccgacttcggcctggtcacgccgcttgacagccagggccgcctggtctcggaggtcggggggcggcggggcacgaagggctaccaggcccctgagacgttggcgcgccgcaccttcgcggccaacgagcgcgaactgcccagctggccagccgccaagagcgatgtattcgcggtgggcgtgatcggcgcggcgctggtgtgcggcaccgagtcggggccggagatggaggccttccgggccagtggcgagctgccgccgcaccggcacgcctcgccagcgctgcggcagctgctcaagggcatggcggcggcggacccggcgcggcggctgggggtggaggaggcactggcgcaccccgcgctacgcagggcgctgagcagcgagcgggcgcggcgcttcatctggtgatggcggcaaggccagatcgctcgggcatgaggcgggctataggcaggcagtgcgttagtgatgagcggcggctgtcaaatgaataaaagagtgcgtacgtgtgtgtcagagcttacttgcttgagggctgggtattcataagtataaagacacagcgtgcgcagtgttgaggaccgtatggaggttcatactataacacatagattggcggcctgagggaaaattgattggcctcaggatagaacccttagcacgcaagccgtggatggcagcgtgcatagttgcggctgctaatccttgctgcttccccttgattgattgatgataggcctagacactagaacgccgcgcgtgctgctgtcttgccgcacttggcagatacgcatggataggccgtggcctaatggctccttatgcttgttccgaaacggaagggtaccatctgcttgtgcttgcgtgtgcattgtcggtactttgggcaacggactgaggccggactaccttgagccgtattttgactcatccgtgttgatctttttccgtcctgtccgcgtgattgctgacatgcatggaccccccgcagtttgagtacaggtctcgcgtagagcttgacatgtcgtgtgtgaccgttttgcgatccacgtgttccgtcaggcctatacggtccatgcttgcgagctgtgtatagccaggattgtgcgagcctctgaagagccgtaagctgctggtcttttcctaggcgcgcagcggagtgcactgacgtgcgcaaggtaacccgccttgtagtgcaaagggacactcgacaaataaatggttgtgcacgcctgtgatgaagagccgcaagctgctggtcgtctcctaggcgcgcagcggtaggtgcgcactgaccgctcagagcgctgactcctggaacatatacagcaggttcgtgcacatggtacggtaacccgccttgtagtgcaaagggacactccacacaaccaagcgtgtgagcttgggtgctaggcattgacatgccttacagggagataagtatgacacgcgagcttgattgagccgcccttcgaattgtaataagtgtttgct

SEQ ID NO: 33 - protein, 595 aa, Chlamydomonas reinhardtii
MAGQALDPAAAGFNNAAQVQGYNQSAEFFLSNYRLGKTLGIGSFGKVKVAEHVLTGHKVAIKILNRRKIQQMEMEEKVRREIKILRLFMHPHIIRLYEVIETPSDIYVVMEYVKTGELFDYIVEKGRLGEDEARHFFQQIISGVEYCHRNMVVHRDLKPENLLLDAKMNVKIADFGLSNIMRDGHFLKTSCGSPNYAAPEVISGKLYAGPEVDVWSCGVILYALLCGSLPFDDENIPNLFKKIKGGIYNLPSHLSPGARDLIPRMLLVDPLKRITIPEIRQHPWFNMHLPRYLAVMQAEPVVGVPRIDEEILEEVVRLGFDRDGLLDSLRSRAANKATVTYYLMTDNRRKMPSSGYLSADMAEGSTGAAMAAAGMSLLPSPGATASASVAAAVGGGGGGGGTPQQRLVAERRWRLGVHARGHPSALMAELYRVLQLNGVAWKKVAPYALKCRAAVRKPPPQELRRRSSAGGSGSGAAGGPARMSDDLDDHIELDGAGSGPPGGAVTPGGGGVAGVGLITSGSRRGLGGGASGSDVAGAAGVPPGTGDAETHYVTRFECQMYKVRDDEYVIDIQRVDGELFLFMDVVGRVLTDLRM

SEQ ID NO: 34 - DNA, 4405 bp, Chlamydomonas reinhardtii
cctaagctgtgcaggcagcaatccggtttgggactggtattatgaatggtcatgtggaaggaaggaaaggcgcgtccgggtggttggacatggcacacagtcgcagtctgatttgtggcagctctagtcatgggggcttactctagtagcggtgcacatctagtaacgctacgagctctaggtagcttggagcaccgtccccatgtgcccttgctgacatcagcccgcgctggccaccaagaatttgcagtctttcaccaagttcgacttcgctatagactggagatatttgtcattttaccacaagcattcgccaaaggccccaccagcaacactgcaagcgaattccttgtcaaaacacaatacgcacatatcattttcgccccgcttaatgtgacgtcttaaagagcattgcaaataaccattaacgttaggaagccggccagggcagcggaagaggaggagcggggtcgcccaagggcgaggacggctgcgaaggccacgcactctccggtgtaaggccaggcagctgtgccatggcagaagcagctcccatggcgggccaagcgctggatccggcagcggcgggcttcaataacgcagcgcaagtgcagggatacaaccagagtgcggagttcttcctcagcaattaccggctagggaaaacactcggcatggttcctttggcaaggtcaaggtcgcggagcatgtgctcacggggcacaaggtggcgattaagatcctcaaccggcgtaaaatccagcagatggagatggaggagaaagtccgtcgtgagatcaagatcctgcggctgttcatgcacccgcacatcatccggctgtatgaggtgatcgagacgccgtctgatatctacgtggtcatggagtacgtcaagaccggcgagctgtttgactacattgtggagaaggggcggctgggggaggacgaggcacgccacttcttccagcagatcatatccggagtggagtactgccaccgcaacatggtggtccaccgcgacctcaagcccgagaacctgctgctggacgccaagatgaacgtcaagatcgcggactttggcctgtccaacatcatgcgcgacggccacttcctcaaaaccagctgcggctcgcccaactacgccgcgcccgaggtgattagcggcaagctgtatgcgggcccggaggtggacgtgtggagctgcggcgtcattctgtacgcgctgttgtgtggatcactgccctttgacgacgagaacatccccaacctgttcaagaagatcaagggcggcatctacaacctgccctcacacctcagcccgggcgcgcgcgacctgatcccgcgcatgctgctggtggacccgctcaagcgcatcaccatccccgagatccggcagcacccctggttcaacatgcacctgccgcgatacctggccgtcatgcaggccgagccggtggtgggcgtgccgcgcattgatgaggagatcctggaggaggtggtgcggctgggctttgaccgggacgggcttctggactcgctgcgcagccgcgccgccaacaaggccaccgtcacctactacctcatgactgacaacaggaggaagatgcccagcagcggctacctgtctgctgacatggcggagggcagtacgggggcggccatggcggcggcgggcatgagcctactgccctcgccaggcgcgaccgcgtcagcaagcgtggcggcggcggtgggcggcggcggcggcggcggcggcacgcctcagcagcggctggtggcggagcggcggtggcggctgggcgtgcacgcgcgaggccacccctcggctctcatggcggagctatacagggtgctgcagctcaacggcgtcgcctggaagaaggtggcgccctacgcgctcaagtgccgggccgccgtgcgcaagccgccgccgcaggagctgcggcgccgcagcagcgccggcggcagcggcagcggcgcggcgggcgggccggcgcgcatgagcgacgacctggatgaccacatagagctggatggggcgggcagtggcccgccgggcggcgcagtgacccctggtggcggcggcgtcgcaggtgttggcctgatcaccagcggcagccggagagggctgggcggcggcgccagcgggtctgacgtggcgggggcggcgggcgtgccgcccggcaccggcgacgcggagacgcactacgtgacacgtttcgagtgccagatgtacaaggtccgggacgacgagtacgtgatagacattcagcgtgtggacggcgagctgtttctgttcatggacgtggtggggcgcgtgctcactgacctgcgcatgtgagggggggtgttggcagcagtggaggcagcactggaggccgcgtgctcacagacctgcgcatgtgagggggggtgttggcagcagtggaggcagcactggaggcggcgtgctcaccgatttgcgcatgtgagggggcggcggcagcagtggaggcagcactggaggccgcgtgctcacagacctgcgcatgtgaagggagggcggcagcagtggaggctgcactggaggccgcgtgctcaccgacctgcgcatgtgagggggcggcggcagcagtggaggcagcactggaggccgcgtgctcacagacctgcgcatgtgaagggagggcggcagcagtggaggctgcactggaggccgcgtgctcaccgacctgcgcatgtgagggggcggcggcagcagtggaggcagcactggaggccgcgtgctcacagacctgcgcatgtgacaagcaggcaggaggcgatgggggcgagcctggctgcgcatacgagcagtcaaggaagcaggcagttggtgcgaggagcgaggaagcggcggtggaggctgcgacggcgtgcaattgttgttgtgaaggggcagcacgcacatgggcagtcgcagcagctgtcagccgcgccccagggctcaccacagcgctaagtgcgagacagcccgtgggagaaggagcgtgagccatcagcggaggcggcaaagcaggagcaggggggagcaggaggcagtcacttcagccactgtccattatcaggaggctgtggttggctgtggtcggcggtgtgtgtgttcggtgtggtgtgggtgtgtgtgtgtgtgtgtgtgtgtgtgtgtgtgtgtgtgtatgtgtgtgtatgtgtgcgtgcgtgtgtgtgtgtgtgtgtgtgtgtgtctttgttttgcatgcatgcacgcgcgaatagcgttgaatggttgggccatgctttcaaaggcttgccgaggaggtcgcgacacaccggggccggggcacagccgtccagacggaggtggaacaattttgtagggttgaagctggtgcgggtggggaagagggggctgctgtgatttgcgtggatgaggtaggcgggtggttggaggaaggcggggagggcagcaggcgcagcagggtggggaaggatcgaaggaggttggtgaggcgacctggtgcaccatgaacggatgagatatagactcgccggttttacggcaggcatgtgaattgtgggtgctgatagtgcgtctttgctgcaaggtgttcagttaggtgatcaaaggtgccgcgttcattattagcggctaggcaccgcgacttgcacgcggagctggacaacctgattactcgtgatgccttgccactcgccagcttggctgccagggctctaacaatcgctgttgcatgcggtgattgcggtccttgcatgcaccacgtgctgtgtgtgatgcgctcaagaaggcgacggtgtgcacgccctgctgtgcactgtgcgatgtggtgcgcagtgccgtgctttgtgatgcgaagaagtgcgcagcggatggcgtgtagggtgagtggctgacaaaagtggatgcatgccgtttgtagatgttgctcgcggaggtggttgcgagggtggtgtgggggcgcattggctgctgcgagcatcaagcctcagtcgtgcggttgagccagcagtacatgctgcatgacagcatgactgcatgatagcatgacagcatgagttgggcagacggacattcctggcaccacgctcccgtggcgcagcggacctggctagcctgctgggcgagggacggttggtgttcgttgtagcgccgctaaaccttgtgtccagtatgtttgtgtgcgggtcggctgattgcagccgttgccaaagcactttgcaggaagagacagtccgttcgggccctcatcccgagcggcccagccggagagaagagaacccctctacttggcagccggcctcccgaaacttacgcaacatgagtgacttccctcgggcgagatagattgggcacttctgggggtgcattctcgccgcgtggggttgcatacggtccccaaatctcgcc

SEQ ID NO: 35 - DNA, 927 bp, Chlorella sorokiniana
atgacggtggagcagctgcggacaaaggtgtgcatcattggctccggccccgccggccacacagccgccatctatgcggcgcgtgcggagctgcagccggtgatgctggaggggtggatggcgaacggcattgccgcaggcggccagctgaccaccacgcacgaggttgagaacttccccggcttccccgagggcatcctgggcggcgagatctgcgagcgcttccgggcgcagagcctgcggtttggcaccaagatcttttcggagacggtcaccaaggtcgacttctccaagcggcccttccacatcttcaccgacgagaaggaggtggtggctgatgccgtcatcattgccacgggtgctgtggcccgccgcctgcccttcaagggttccgatgaagacaacggtttctggaacaagggcatttcagcctgcgcggtgtgcgacggcgcggctcccatgttccgcaaccagcccattgcagtgattggcggcggcgactctgccatggaagaggcccactttctgaccaagtacggcagcaagaagcgggccatggaccaccccaagatcgaaatcctgtggaacagcgtggtggaggaggcgtacggcaatgcaaaggggctgctgggcggcgtcaaggtcaagaacgtcaagaccggcgagatcacggatctgccgctggcgggcctgttcttcgcaatcggccacgagcccgccaccgcgttccttggcggccaggtggagcttgacgaggacaagtacattgtcaccgcggccgactccaccgcgaccagcgtgccgggcgtgtttgcagcgggcgatgtgcaggacaagaagtaccggcaggccatcaccgcggcaggctcgggctgcatggctgcgcttgaggtcgagcacttccttgaggcacagggcgaggcctga

SEQ ID NO: 36 - protein, 308 aa, Chlorella sorokiniana
MTVEQLRTKVCIIGSGPAGHTAAIYAARAELQPVMLEGWMANGIAAGGQLTTTHEVENFPGFPEGILGGEICERFRAQSLRFGTKIFSETVTKVDFSKRPFHIFTDEKEVVADAVIIATGAVARRLPFKGSDEDNGFWNKGISACAVCDGAAPMFRNQPIAVIGGGDSAMEEAHFLTKYGSKKRAMDHPKIEILWNSVVEEAYGNAKGLLGGVKVKNVKTGEITDLPLAGLFFAIGHEPATAFLGGQVELDEDKYIVTAADSTATSVPGVFAAGDVQDKKYRQAITAAGSGCMAALEVEHFLEAQGEA

SEQ ID NO: 37 - DNA, 1255 bp, Chlorella sorokiniana
agcggccatgacggtggagcagctgcggacaaaggtgtgcatcatcggctccggccccgccggccacacagccgccatctatgcggcgcgtgcggagctgcagccggtgatgctggagggatggatggcaaacggcattgccgcaggcggccagctgaccaccacccacgaggttgagaacttccccggcttccccgagggcatcctgggcggcgagatctgcgagcggctccgggcgcagagcctgctgtggttgggcggcgagatttgcgagcggttccgggcgcagagcctgcggtttggcaccaagatcttctcagagacggtcgccaaggtggacctctccaagcggcccttccacatctggacggatgagaaggaggtgattgcggatgcggtgatcatcgccaccggcgccgtggcccgccgcctgcccttcaagggctccgatgaggacaacggcttctggaacaagggcatctccgcctgcgcgggcagcgggttctggaacaagggcatctccgcgcgcgcggtgtgcgacggcgcggcgcccatgttccgcaaccagcccattgcagtcattggcggcggcgactcggccatggaggaggcgcacttccttaccacgtacggcagcagtgcagaataccctctttcactgaatgaggacctgttgatctccatggaggaagctcacttcctcaccaagtacggcagcaaggtgtacatcatccaccgccgcgacgagctgcgcgcatccaagatcatgcaggcaaggctgttgtgctacccgacccttgctgttgctgttggtgtgaagctgctggtgaagcgggcgctggagcaccccaagattgagatcctgtggaacagcgtggtggaggaggcatacggcaacgcaaaggggcttctgggcggcgtcaaggtcaagaacgtcaagactggcgaggtcaacgacctgctgctggcgggctgcgaggtcaccgatgtgccgctggcgggcctgttctttgccatcggccacgagccagccaccgccttcctgggcggccaggtggagctggatgaggacaagtacatcgtgactgcggccgactccactgcaaccagcgtgccgggcgtgtttgcggcgggcgtgtttgcggcaggcgatgtgcaggacaagaagtaccggcaggccatcaccgcggcaggctcaggctgcatggctgcgctggaggttgagcggttcctggaggaccatggacaggcttga

SEQ ID NO: 38 - protein, 415 aa, Chlorella sorokiniana
MTVEQLRTKVCIIGSGPAGHTAAIYAARAELQPVMLEGWMANGIAAGGQLTTTHEVENFPGFPEGILGGEICERLRAQSLLWLGGEICERFRAQSLRFGTKIFSETVAKVDLSKRPFHIWTDEKEVIADAVIIATGAVARRLPFKGSDEDNGFWNKGISACAGSGFWNKGISARAVCDGAAPMFRNQPIAVIGGGDSAMEEAHFLTTYGSSAEYPLSLNEDLLISMEEAHFLTKYGSKVYIIHRRDELRASKIMQARLLCYPTLAVAVGVKLLVKRALEHPKIEILWNSVVEEAYGNAKGLLGGVKVKNVKTGEVNDLLLAGCEVTDVPLAGLFFAIGHEPATAFLGGQVELDEDKYIVTAADSTATSVPGVFAAGVFAAGDVQDKKYRQAITAAGSGCMAALEVERFLEDHGQA

SEQ ID NO: 39 - DNA, 1329 bp, Chlorella sorokiniana
atggcatccgtagcagtatcctcggcgagcgctgtcgtgccgggcaccgctcacctgcggcagcagcagccgcggccatgcacggggcgggcggcatgggtgccgcagcaccgcctccggctgtggcgccgggcgctgagcgtgcacgcgagcagcagcaacggcaatggcaacggcaatggggcagccgtcgacaagcgcgagcgcatgaccaccagcgtggcgattgtgggcagcggccccgccgcgcacaccgctgccatctacctggcacgtgcggagctggagcccatcctgtttgaggggtggatggccaacggcctggcccccggcggccagctcaccaccaccacgtacgtggagaacttccccggcttccccgagcccatcctgggagcagacctgtgcgaccgcttccgccagcagtcaaagaactacggcacccgcatctacaccgagactgtggacaagctggatctgctgaatggcccgcccttccggctggagaccgacagccgcgtggtggaggcggatgcagtcatcattgccacgggggcggccgctcgcaagctgcccatcaaagggctggagcagtactggaacaatggcatctccgcctgcgcagtctgcgacggctcgtcgccgctcttccgcaacaagccggtggcggtggttgggggcggtgacgtggcgtgcgaagaggcgctcttcctggcccgctacgcctcaaaggtgtacatcgtgcagcgctacgactacctggagtcgtccaaggtgatggctcggcgcgcggtgagccaccccaaggtggaagtcctgttcagccacgagtgccaggaggcgtacggcggcgaggacggcaccctgagcggcattgtgctgcgcaacaaccagacgcaggaggtcacgtacctgcccgtgggcggcctcttcttcgccattggccacgcccccgccaccgccttcctggggggacagctggagctggacagccacggctacatcgtgacgccaccaggaggcaccaccaccagcgtgcccggcgtgtttgctgcgggcgacgtgcaggactggcaatggcggcaggccatcactgccgctggctcaggctgcatggcggccaaggaggcggaggactacctgtcccagctggcgctggaggcggagaagggcggcgatggcgtggtgcagctcaacgccgcctgggtggcggaggtgcgccaggaggagcgcaagctgcgcaaggagcgcaaggcagcggaagcagcagcggcggcggcagcagccgctgccgagagcgccacgcctgagcccacgcctgctgcgtga

SEQ ID NO: 40 - protein, 442 aa, Chlorella sorokiniana
MASVAVSSASAVVPGTAHLRQQQPRPCTGRAAWVPQHRLRLWRRALSVHASSSNGNGNGNGAAVDKRERMTTSVAIVGSGPAAHTAAIYLARAELEPILFEGWMANGLAPGGQLTTTTYVENFPGFPEPILGADLCDRFRQQSKNYGTRIYTETVDKLDLLNGPPFRLETDSRVVEADAVIIATGAAARKLPIKGLEQYWNNGISACAVCDGSSPLFRNKPVAVVGGGDVACEEALFLARYASKVYIVQRYDYLESSKVMARRAVSHPKVEVLFSHECQEAYGGEDGTLSGIVLRNNQTQEVTYLPVGGLFFAIGHAPATAFLGGQLELDSHGYIVTPPGGTTTSVPGVFAAGDVQDWQWRQAITAAGSGCMAAKEAEDYLSQLALEAEKGGDGVVQLNAAWVAEVRQEERKLRKERKAAEAAAAAAAAAAESATPEPTPAA

SEQ ID NO: 41 - DNA, 1081 bp, Picochlorum soloecismus
atgactggcggaacgaaagagcaactgaagacccaagtttgcatcatagggagcgggcctgctggtcatactgctgcgatttatgctgcgagagcgggtatgtcaccttaggagtgtgcaatacgtgtggatagatttagacataagtgtggtagtataggtatgtgaacctttggatcatgttgtcgcagaactgaaaccagtcatgctggaagggtggctggcgaacggtattgcagcgggtgggcagctgacaacgacttcggatgtggaaaatttcccaggatttcccgaggggattatggggggtgaaatctgcgataagtttcgtgcccaaagtgctcgtttcgggacagatattttctctgagactgtgacgtctgtggacttttcagaacggccttttaaagtggtgacggatgagaaggaggttattgccgatactgtaattatttctacaggagcggtggctcgtaagctgtcttttccaggatccgacgaggagaatgggtattggaataagggaatcagtgcatgcgcagtatgtgatggtgctgcaccaatgttcagaaacaaaccaattgctgtgattggaggtggagattctgcttgcgaggaggcaacttttttaacaaaatacgggtccaaagtttacttgattcatcgtcgtgatgagttgcgtgcgtccaagattatgcagaagagggtgattgatcatgagaaggtggagattttgtgggatagtgttgtggattctgcatatgggaatgaaaaaggtttactcgggggattgaaagttcgcaacgtcaagactggagaaataaccgatcttcctgtggctgggctgttctttgcgattggccacgagcctgcaaccaagtttttggacggtcaaatcaatcttgatgaagagggatacatacaaacagagcctggcaccacaaagacaaatattcctggcgtgtttgcagcaggtgatgtgcaagataaaaaatataggcaagcgattactgctgccggtacaggatgcatggctgcattggaagcagaacacttccttgaggcgttgcatgattcatga

SEQ ID NO: 42 - protein, 328 aa, Picochlorum soloecismus
MTGGTKEQLKTQVCIIGSGPAGHTAAIYAARAELKPVMLEGWLANGIAAGGQLTTTSDVENFPGFPEGIMGGEICDKFRAQSARFGTDIFSETVTSVDFSERPFKVVTDEKEVIADTVIISTGAVARKLSFPGSDEENGYWNKGISACAVCDGAAPMFRNKPIAVIGGGDSACEEATFLTKYGSKVYLIHRRDELRASKIMQKRVIDHEKVEILWDSVVDSAYGNEKGLLGGLKVRNVKTGEITDLPVAGLFFAIGHEPATKFLDGQINLDEEGYIQTEPGTTKTNIPGVFAAGDVQDKKYRQAITAAGTGCMAALEAEHFLEALHDS

SEQ ID NO: 43 - DNA, 1641 bp, Chlorella sorokiniana
atggcagctgtggcggcagtctcggtggcagcggcgcccgctcgctgcgcggcgacccgccaggcgcgatcggcggcttgccccgccctgcagcatggcaatgcgcgcagctgcagccgcagccggcgctcgctggcagggaccgccgctgctgcgggcccagctcgcgtgcagcggcgcctggggctgcgcctgagggcgcaggccagcggcaacggcgcccccgaggtggagaacatggtcatcattggcagtggccctgccggctacaccgccgccatctacgcggcccgtgccaacctgcggccctttgtgtttgagggcctgtccgcaggcggcgtgcgcggcggccagctgatgaccacgaccgaagtcgagaacttcccgggcttccccgagggcatcaccgggccggacctgatggaccgcatgcgcgcgcaggccgagcgctggggcgcccgcctggagactgaggatgtggtgtcggtggacctctccacccgccccttcaccgtgcgcggcactgagaacaccgtcaaggcgcactccgtgattgtggccacgggcgccaccgccaagaagctgaacctgcccagcgagcagcgcttctggtctaacggcatctccgcctgcgccatctgcgacggcgcctcaaccatcttcaagcagcaggcgagagggggcggcttcatgcgtaccacggagctggctgtggtgggcggcggcgacaccgccaccgaggaggccgtgtacctcaccaagtacgcctcccacgtgcacctgctggtgcgtggcgacaagatgcgggccagcaaggcgatgcaggaccgcgtgctggccaaccccaagatcacggtgcacatgaacacggaggtggatgatgcctacggcgacgacagcgccatgaagggcctgcacctgcgcgacgccaagaccggcgagaagcgcgacctgccggtgcgcggcctgttctacggcatcggccacaagcccaactcggacttcctggcgggccagctggagctggaccaggaggggtacgtggtggtgaagcatggcggcaagacgagcgtggagggtgtgtttgcggcgggagacctgcacgacgtggagtggcgccaggccatcaccgcggcgggcagcggctgccaggcggccctggctgcggagcgctacctcagcgccaacgggctggcccaggagttcagccaggcggtgaccgaggagaagtacggctcgacgcctgagacgcaggcggccagcagcagcggcgccgacacggaggagacctttgaccccgcagccgacaagcacaagggccagtttgcgctgcgcaagctgtaccacgagagcagccgcccgctcattgtgctgtacacggcgcccacgtgcggcccctgccgcacgctcaagcccatcctgggcaaggtggtggacgagtttgcgggcaaggtgcactttgtggagattgacattgagcaggacgcggcgctggcggagggtgcgggcgtcaacggcacgcccaccgtgcagatcttcaaggacaaggccatggtggagacgatggtgggcgtgaagcagaagagccagtaccgggcagtggtggagaaggcgctgggcgcggccaccgtcagtgcgtga

SEQ ID NO: 44 - protein, 546 aa, Chlorella sorokiniana
MAAVAAVSVAAAPARCAATRQARSAACPALQHGNARSCSRSRRSLAGTAAAAGPARVQRRLGLRLRAQASGNGAPEVENMVIIGSGPAGYTAAIYAARANLRPFVFEGLSAGGVRGGQLMTTTEVENFPGFPEGITGPDLMDRMRAQAERWGARLETEDVVSVDLSTRPFTVRGTENTVKAHSVIVATGATAKKLNLPSEQRFWSNGISACAICDGASTIFKQQARGGGFMRTTELAVVGGGDTATEEAVYLTKYASHVHLLVRGDKMRASKAMQDRVLANPKITVHMNTEVDDAYGDDSAMKGLHLRDAKTGEKRDLPVRGLFYGIGHKPNSDFLAGQLELDQEGYVVVKHGGKTSVEGVFAAGDLHDVEWRQAITAAGSGCQAALAAERYLSANGLAQEFSQAVTEEKYGSTPETQAASSSGADTEETFDPAADKHKGQFALRKLYHESSRPLIVLYTAPTCGPCRTLKPILGKVVDEFAGKVHFVEIDIEQDAALAEGAGVNGTPTVQIFKDKAMVETMVGVKQKSQYRAVVEKALGAATVSA

SEQ ID NO: 45 - DNA, 1679 bp, Picochlorum  soloecismus
atgaatcgtgggagggcattggggtctactcggagattgtcgggcagtgtaggtcttgtccgaccacaagttatcaatctggacctttcttcaaagatttctgcaaaagggaatggagcgctgagcagatgtaagcgagatgtaaaaatgctcgttagtgcttctgcagcggaagggactgggtcgaatgagaaggttgaaaatttggtgataatcgggagtggtccagcaggatacactgcggccatctacgcctcaagagcgagtctccggcctctcgtgttcgaagggtatcaggctggtggggttcgaggtggacaattaatgacaacgacagaggtggagaactttccggggtttcctgaaggtattactggcccagatttgatggataggatgaggcaacaggtgagttacctgcagactgtgaaatctggttcgagggtcgttttgcatttacagaccagtgaaaatgaaagcatttattttctatgatctgaatatgttgttcactgcaggcggagcgatggggtagcaatttgctgttggaagatgtagaatcagttgacttgtctcagaggccattcgttatcaagggaagtgaaaccactgtcaaagccaatagtttgatcattgcgactggggcaaccgcgaaaaggcttagtattccatctgaacataccttctggagtagaggtattagtgcatgcgcaatttgtgatggagcatcgccaatctttaaggatcaggaattggctgttgttggaggaggggatactgcaacggaagaggcagtgtatttgacgaaatatgggaggcacgttcatctcttggttcgtggacctacgatgcgtgcaagcaaagctatgcaggagcgtgtcctcaagaatcccaagataacagtgcactttagtactgcaattgaagatgcatacggagacaagaagggggcaatggcaggtttgcaccttgtcaataatgacaccggtgaaaagaaagacttgcaagttcgagggctgttttatggcattggccattccccaaacagtgggtttcttgatgggcaagtagaactagactcttctggttatgtgaaggtgaaggaaggagggccctggaccaatgtagaaggcgtcttctcagcaggagacctccatgatacagaatggagacaggcgattacagctgcaggcagtggctgcatggcagcactcgcagccgagagatatctcactgcaaatgaactgattgtggaggccccagaagaagaagcacctgaaccacagaaacaagcagaggcccctagcaagaaggaagaagtgaagaaagaagtgtcgatggaagaagagttcaatctatatgccgataaacaccgcggacaatacgccctaagaaagttgtatcacgaaagcgatagattgctgactgttctgtacacttctcctacctgcggtccatgtagatcattgaaacctatattgaacaaagttcttgatgagtatgctgggaaaatacacttggtggaaatagatatcgctgaggacccagaaatcgcacaggctgcgggagtgaacggtactccaaccgtccagatgtttaagaacaaggacagggttgcaaatttgcctggtgtcaaaatgaagaatgaatacaagaaacacattgaaagccatttgtag

SEQ ID NO: 46 - protein, 522 aa, Picochlorum soloecismus
MNRGRALGSTRRLSGSVGLVRPQVINLDLSSKISAKGNGALSRCKRDVKMLVSASAAEGTGSNEKVENLVIIGSGPAGYTAAIYASRASLRPLVFEGYQAGGVRGGQLMTTTEVENFPGFPEGITGPDLMDRMRQQAERWGSNLLLEDVESVDLSQRPFVIKGSETTVKANSLIIATGATAKRLSIPSEHTFWSRGISACAICDGASPIFKDQELAVVGGGDTATEEAVYLTKYGRHVHLLVRGPTMRASKAMQERVLKNPKITVHFSTAIEDAYGDKKGAMAGLHLVNNDTGEKKDLQVRGLFYGIGHSPNSGFLDGQVELDSSGYVKVKEGGPWTNVEGVFSAGDLHDTEWRQAITAAGSGCMAALAAERYLTANELIVEAPEEEAPEPQKQAEAPSKKEEVKKEVSMEEEFNLYADKHRGQYALRKLYHESDRLLTVLYTSPTCGPCRSLKPILNKVLDEYAGKIHLVEIDIAEDPEIAQAAGVNGTPTVQMFKNKDRVANLPGVKMKNEYKKHIESHL

SEQ ID NO: 47 - DNA, 1629 bp, Chlorella sorokiniana,
atggcggccgtgacggcagtctcccttccggcggcgactgctcgctgcgcggcgacccgccaggcgcgatcggcggcatgcccgcccctgcagaccggcaatgcgcggcgcagccggcctagccgctcgctggcagggaccgcggcttctgcgagccagacccgtgtgcagcggcgcctggcgctgcgcctgagggccgaggccgccggcaacggcgcccccgaggtggagaacatggtcatcattggcagcggccccgccggctacaccgccgccatctacgccgcccgcgccaacctgcggccctttgtgttcgagggcgtgtcggcaggcggcgtgcgcggcgggcagctcatgaccacgaccgaggtggagaacttccccggcttcccggagggtatcacgggcccggacctgatggatcgcatgcgcgcgcaggctgagcgctggggtgcccgcctggagactgaggacgtggtgtccgtggacctgtccacccgccccttcaccgtgcgcggcaccgacaccaccgtcaaggcgcactcggtcattgtggccacgggcgccaccgccaagaagctcaacctgcccagcgagcagcgcttctggtccaacggcatctccgcctgcgccatctgcgacggagcctccaccatcttcaagcagcaggcgagcgggcgcttgctgtttgagctggctgtggtgggcggcggcgacaccgccaccgaggaggccgtctacctgaccaagtatgcctcgcacgtgcacctgctggtgcgcggcgacaagatgcgggccagcaaggcgatgcaggaccgcgtgctgtccaaccccaagatcacggtgcacatgaacacggagattgatgatgcatatggcgacgacagcgccatgaagggcctgcacctgcgcgacgccaagacaggcgagaagcgcgacctgcccgtgcgcggcctgttctacggcatcggccacaagcccaactcggacttcctggccggccagattgcgctggacaaggaggggtacgtggtggtgcagcacggcgggcgcaccagcgtggagggcgtgtttgcggcgggagacctgcacgacgtggagtggcgccaggccatcaccgcggcaggcagcggctgccaggcggcgctggctgctgagcggtacctcagcgccaacgggctggcgcaggagttcagccaagctgtgaccgaggagaagtacggctcgacgccggagacgcgggcggccagcagcagcggtgccgacactgaggagacattcgaccccaacgccgacaagcacaagggccagttcgcgctgcgcaagctgtaccacgagagcagccgcccactcatcgtgctgtacacagcccccacctgcgggccgtgccgcacgctgaagcccatcctgggcaaggtggtggacgagttcgctggcaaggtgcactttgtggagattgacattgagcaggacgcggcgctggccgagggcgcaggcgtgaatggcacgcccaccgtgcagatcttcaaggacaaggccatggtggagacgatggtgggcgtgaagcagaagagccagtaccgcgcggtggtggagaaggcgatgggtgcggccaccgtcagcgcgtaa

SEQ ID NO: 48 - protein, 542 aa, Chlorella sorokiniana
MAAVTAVSLPAATARCAATRQARSAACPPLQTGNARRSRPSRSLAGTAASASQTRVQRRLALRLRAEAAGNGAPEVENMVIIGSGPAGYTAAIYAARANLRPFVFEGVSAGGVRGGQLMTTTEVENFPGFPEGITGPDLMDRMRAQAERWGARLETEDVVSVDLSTRPFTVRGTDTTVKAHSVIVATGATAKKLNLPSEQRFWSNGISACAICDGASTIFKQQASGRLLFELAVVGGGDTATEEAVYLTKYASHVHLLVRGDKMRASKAMQDRVLSNPKITVHMNTEIDDAYGDDSAMKGLHLRDAKTGEKRDLPVRGLFYGIGHKPNSDFLAGQIALDKEGYVVVQHGGRTSVEGVFAAGDLHDVEWRQAITAAGSGCQAALAAERYLSANGLAQEFSQAVTEEKYGSTPETRAASSSGADTEETFDPNADKHKGQFALRKLYHESSRPLIVLYTAPTCGPCRTLKPILGKVVDEFAGKVHFVEIDIEQDAALAEGAGVNGTPTVQIFKDKAMVETMVGVKQKSQYRAVVEKAMGAATVSA

SEQ ID NO: 49 - DNA, 2437 bp, Chlorella sorokiniana, strain 1412; NTRC
atgctcaccagcgcggaagccggatcggcgctgcacagcagcgcggcggcgctgccgctgccgccaccgctgcacgtactgccgggcgcctgggcgctcctggcagacctggccacggcgttgcagccctcctgcttcgacgccgagtgccagcgggagaaagacatgctgctggaggccagcctggctatcccggtgctagtgctgttcgcctcggtggcgttcctgctgcgcccgccgccgcagggcagcatcgacgaggggcggctgtttgaggaccccaagacaggcatcctgtttgaggcgccaccgggagcggcgcctgagcgggaccgcaagggcgagcttgccttccgccccatctcctacacgccctggcctgtggaggagggggcagagggcgagcggatcctgccgcagcccagccagctggtgatggccacacttgagcggccgctggggattgaatttgaggaggccaagggcgctaagcgcaccgtggtggccagcctcacgccaggcgggcacgctgagcagctggccaagcgggggcggctcaacacctcgctgctggcctcctgcccgctggagggagatgtgctgcgcggctgcacctgcaccaacatcacctggcccggcggcgacttccccaagcgagagattgtgcgccgcctggggctgcgcctgagggcagaggccgccgggaacggcgcccccgaggtggagaacatggtcatcattggcagcggccccgccggctacaccgccgccatctacgccgcccgcgccaacctgcggccctttgtgtttgagggcgtgtccgcaggcggcgtgcgcggtgggcagctgatgaccacaaccgaggttgagaacttccctggcttcccggagggcatcacggggccagacctgatggatcgcatgcgcgcgcaggctgagcgctggggtgcccgcctggagactgaggatgtggtgtctgtggacctgtcctcccgccccttcaccgtgcgcggcactgacaccactgtcaaggcgcacaccgtcattgtggccacgggcgccaccgccaagaagctgaacctgcccagcgagcagcgcttctggtccaacggcatctccgcctgcgccatctgcgacggcgcgtcaaccatcttcaagcagcaggcgagcgggcagtgtggctgccttttggctggccaggaatgttggctggcgcaggaatggctgcagggtatccaagtgttggagctggctgtggtgggcggcggcgacaccgccaccgaggaggcggtgtacctcaccaagtatgcctcccatgtgcacctgctggtgcgtggcgacaagatgcgtgccagcaaggcgatgcaggaccgcgtgctggccaaccccaagatcacggtgcatatgaacactgagatcgatgatgcctttggcgacgacagcggcatgaagggcctgcacctgcgcgacgccaagacgggcgagaagcgggacctgcctgtgcgcggcctgttctacggcatcggccacaagcccaactcggacttcctggctggccagctggagctggacaaggaggggtacgtggtggtggcgcacggcggcaggaccagcctggagggcgtgtttgcagcgggagacctgcacgacgtggagtggcgccaggccatcaccgcagcgggcagcggctgccaggcggcgctggcagcggagcggtaccttagcgccaacggcctggcgcaggagttcagccaggcagtgactgaggagaagtacggctcgacgcctgagacgcaggccgccagcagcagcggcgcggacaccgaggagacctttgaccccaacgccgacaagcacaagggccagtttgcgctgcgcaagctgtaccacgagagcagccgcccgctcatcgtgctgtacacggcgcccacgtgcggcccctgccgcacgctgaagcccatcctgggcaaggtggtggacgagtttgcgggcaaggtgcactatgtggagattgacattgagcaggacgcggcgctggcggagggcgcgggcgtcaacggcacgcccaccgtgcagatcttcaaggacaaggccatggtggaaacgatggtgggcgtgaagcagaagagccagtaccgcgcggtggtggagaaggcgattggggccgccaccgtcaacgcttgagtgatgcagcgatgcatgcagcaccccatacgccgtgcatgccggatagcagcaattggacagcaggctggctacaccgccgctgcaggcaacccctccgtcccatgccccctgaaccgctgctggtgctgcactgcgatgcgtccctccctgcattgtgcggagcgggcgcttttccaatcaaccaaccctgcgagcgatgtgccgacccctcgatgccatgacaccctcaccgcaacacccaccctgctgt

SEQ ID NO: 50 - protein, 727 aa, Chlorella sorokiniana
MLTSAEAGSALHSSAAALPLPPPLHVLPGAWALLADLATALQPSCFDAECQREKDMLLEASLAIPVLVLFASVAFLLRPPPQGSIDEGRLFEDPKTGILFEAPPGAAPERDRKGELAFRPISYTPWPVEEGAEGERILPQPSQLVMATLERPLGIEFEEAKGAKRTVVASLTPGGHAEQLAKRGRLNTSLLASCPLEGDVLRGCTCTNITWPGGDFPKREIVRRLGLRLRAEAAGNGAPEVENMVIIGSGPAGYTAAIYAARANLRPFVFEGVSAGGVRGGQLMTTTEVENFPGFPEGITGPDLMDRMRAQAERWGARLETEDVVSVDLSSRPFTVRGTDTTVKAHTVIVATGATAKKLNLPSEQRFWSNGISACAICDGASTIFKQQASGQCGCLLAGQECWLAQEWLQGIQVLELAVVGGGDTATEEAVYLTKYASHVHLLVRGDKMRASKAMQDRVLANPKITVHMNTEIDDAFGDDSGMKGLHLRDAKTGEKRDLPVRGLFYGIGHKPNSDFLAGQLELDKEGYVVVAHGGRTSLEGVFAAGDLHDVEWRQAITAAGSGCQAALAAERYLSANGLAQEFSQAVTEEKYGSTPETQAASSSGADTEETFDPNADKHKGQFALRKLYHESSRPLIVLYTAPTCGPCRTLKPILGKVVDEFAGKVHYVEIDIEQDAALAEGAGVNGTPTVQIFKDKAMVETMVGVKQKSQYRAVVEKAIGAATVNA

SEQ ID NO: 51 - protein, 796 aa, Chlorella variabilis; phototropin A
MAGAAGLAGAMVVAAVDEEGVEVPVKAQLTSALAQLRHTFVVADATLPDCPLIYASEGFVHMTGYSMEEVLGHNCRFLQGEGTDPKDVKKLRDAVRNGTPVCTRLLNYRKDGTPFWNLLTMTPIKDEMGRVIKFVGVQVDVTNKTEGRAYTDSAGVPMLVHYDDRLKETVAKPIVDDVLTAVQEADGKVPVRLSRGSPSRALPRVALDLATTVERIQSNFVIADPTLPDCPIVFASDPFLKLTGYRREEVLGRNCRFLQGRDTDRATVNELKAAIRAGRECTVRMLNYTKAGKPFWNMLTVAPIKDIEERPRFLVGVQVDVTEHATATDAAPVGAQAANIVGQALQNMNWVGVDPWATFPSGLVEPKPHRRMDPAAAALKEAVQRDGKLRLRHFARVRQLGSGDVGMVDLVQLVGGEHRFALKSLEKREMLERNKVGRVRTEESILSKVDHPFLATLYGTLQTDTHLHFLLEFCSGGELYALLNAQPNKRLKEDAVKFYASEVLLALQYLHLQGFVYRDLKPENILLHGSGHVMLTDFDLSYCQGSSSPSLLVLPADHPSVAPAGGAAAARPEGRESRRGSKDSARVSKDGGRRPLALASGQHVLLVAQPDGRANSFVGTEEYLAPEVITGSGHTSMVDWWSFGILIYELLYGTTPFRGSRRDATFENVLKKPLAFPDSVPVSAECKDLITQLLAKEASKRVGSRAGADEIKRHAWFAGLNWALVRNQKPPFVTPRKTSTSSDVPNSPMSDNAFRGKSAESPLPAAAAAVLDAQLHHKAKSEAAAAPAGPGHIDGF

SEQ ID NO: 52 - protein, 749 aa, Chlamydomonas reinhardtii
MAGVPAPASQLTKVLAGLRHTFVVADATLPDCPLVYASEGFYAMTGYGPDEVLGHNCRFLQGEGTDPKEVQKIRDAIKKGEACSVRLLNYRKDGTPFWNLLTVTPIKTPDGRVSKFVGVQVDVTSKTEGKALADNSGVPLLVKYDHRLRDNVARTIVDDVTIAVEKAEGVEPGQASAVAAAAPLGAKGPRGTAPKSFPRVALDLATTVERIQQNFCISDPTLPDCPIVFASDAFLELTGYSREEVLGRNCRFLQGAGTDRGTVDQIRAAIKEGSELTVRILNYTKAGKAFWNMFTLAPMRDQDGHARFFVGVQVDVTAQSTSPDKAPVWNKTPEEEVAKAKMGAEAASLISSALQGMAAPTTANPWAAISGVIMRRKPHKADDKAYQALLQLQERDGKMKLMHFRRVKQLGAGDVGLVDLVQLQGSELKFAMKTLDKFEMQERNKVARVLTESAILAAVDHPFLATLYCTIQTDTHLHFVMEYCDGGELYGLLNSQPKKRLKEEHVRFYASEVLTALQYLHLLGYVYRDLKPENILLHHTGHVLLTDFDLSYSKGSTTPRIEKIGGAGAAGGSAPKSPKKSSSKSGGSSSGSALQLENYLLLAEPSARANSFVGTEEYLAPEVINAAGHGPAVDWWSLGILIFELLYGTTPFRGARRDETFENIIKSPLKFPSKPAVSEECRDLIEKLLVKDVGARLGSRTGANEIKSHPWFKGINWALLRHQQPPYVPRRASKAAGGSSTGGAAFDNY

SEQ ID NO: 53 - protein, 714 aa, Botryococcus terribilis
MAAHLHPELQRPGQSVPPPAGQLTKVLASLRHTFVVADATLPDCPLVYASEGFLQMTGYSADEVLGHNCRFLQGEGTDPKEVAVIREAVRKGEGCSVRLLNYRKDGTPFWNLLTMTPIKTEDGRVSKYVGVQVDVTSKTEGKAFSDATGVPLLVKYDTRLREGVAKGIVQEVTSNIQDAELETRLGKKMTAPKSFPRVALDLATTVERIQQNFCICDPNLPDCPIVFASDGFLEMTEFGRFEVLGRNCRFLQGPETDPKAIAEIKEAIKNRSETTVRILNYKKSGKPFWNMFTLAPMADVDGTPRFLIGVQVDVTAAEAAGLTSVEPAVDTVKSVAMQQLGAGWGRADPWQNVHAGLSVIKPHKAQEKAYVALAEVEKAQKKLALYQFRRLKQLGTGDVGLVDLVELQGTDSKFAMKTLEKNEMLERNKVMRVLTEAKILSAVDHPFLATLYATLATDTHLHFLMEYCEGGELYGLLTRQPAKRFKESHMRFYAAEGLIALQYLHLLGFVYRDLKPENILLHHTGHVLLTDFDLSYCQGKTQPTIEMKAPRNPATGTVDPADVLLVAEPEGRANSFVGTEEYLAPEVINGTGHTAAVDWWSFGILMYELVYGFTPFRGSKREATFESILKRPLAFPSKPVVSPACQDLISQLLIRDASKRLGSKAGAEEIKAHPFFKGINWALLRNTVPPYVPRVSESDRPNPPAAAQAIFDAF

SEQ ID NO: 54 - protein, 719 aa, Tetraselmis striata
MASDEVPAAANNLTSVLSGLKHTFVVADATLPDCPLVFASESFYTMTGYSKDEVLGHNCRFLQGEGTSPKEIQKIRDAVKTGEICSVRLLNYRKDGTPFWNLLTVTPVKTSTGQVTKFVGVQVDVTSRTEGKAFAETGGAPLLKYDGRLRENVAKNIVAEVVDTVESVESNGKRATAPKAFPRVALDLATTVERIQQNFCICDPTLPDVPIVFTSDAFLELTEYSREEVLGKNCRFLQGPKTDPDTVATIRKAVIDKEEITVRILNYKKSGKPFWNMFTLAPIKDVDGTCRFMVGVQVDVTAADASASPDAIPQMQNDAQLKAKGHDASAVIGSALQNLGMGGKDEDPWKSIVTGVLYQKPHMSDSPAVVALRAAVEQHGALNIDSFKRQKQLGSGDVGLVDLVTLAGTNHEFAMKSLDKKEMIERNKIGRVQTEQAILASVDHPFLATLYCTLDTPSHLHFILQICAGGELYGLLNAQPKKRLREAHVRFYIAEVLLALQYLHLLGYIYRDLKPENILLHGSGHVMLTDFDLSFGKGMTEPKMQKTVTPVEAAAGCSGNPPKAKKPNENYILLAEPSAKSNSFVGTEEYLAPEVINGTGHGAEVDWWALGILTHELLYGVTPFRGQRRDETFENVLRVPLNLPTKPTVSPECRDFISQLLVKNPEKRLGAKRGAEDIKAHPWFKDLDFNMLRHEPPPFVPQASGDSGAPPPNAAFKNF

SEQ ID NO: 55 - protein, 819 aa, Micromonas pusilla, strain CCMP 1545; phototropin A
MAAMSGQVPPDKMPQGVSYTVDESGGIAAPEASKGLTMALASVRHTFTVSDPTLPDCPIVYASDGFLKMTGYSAEEVINRNCRFLQGEDTDRDDVQKIRDAVQKGERLTIRLQNYKKDGTPFWNLLTIAPVKMEDGTVAKFIGVQVDVTDRTEGEVGRTVGDGGVVGAKDEKGLPLLVRYDQRLKDQNYPGVEDVEKAVMKGEGIDADATRNSRAREGLDMATTMERIQQSFLISDPSLPDCPIVFASDGFLDFTGYGREEILGRNCRFLQGAGTDRDAVKEIRNAIKDNRECTVRLLNYTKQGKPFWNMFTLAPVRDHAGEVRFFAGVQVDVTVYTDADGRRLDSVELLRQTKAPTPRHSGDDEGKSKSKAATKKVLEAIGGLTAADGELPWARMVGRLGAPKPHQAGDANWAALRKIVAAHKAAGRPERLAPEDFTPLTRLGHGDVGAVHLVSLRDAPSAKFAMKVLVKQEMVDRNKLHRVRTEGRILEAVDHPFVATLYSAFQTDTHLYFLMEYCEGGELYETLQKQPGKRFTEATTKFYAAEVLCALQYLHLMGFIYRDLKPENILLRRNGHVIVTDFDLSYCASSRAHVVMIDGKGEDVVAGGGSATTSGSGRGSGGGGGSGGGGKKERRPSDAGSESSSSRGGGGFCGKGGGGGSNPATRRDTPRLVAEPFAFTNSFVGTEEYLAPEVLNSTGHTSSIDWWELGIFIHECVFGLTPFRASKREQTFQNIISQPLSFPSNPPTSPELKDLLSQLLRRDPSERLGTRGGAEEVKAHPFFKGVDWALLRWKDAPLAKKPDPPRADGGGDEVFEIEV

SEQ ID NO: 56 - protein, 691 aa, Dunaliella salina; phototropin A
MFPADPKCSMFLADPVTWRISYFSESFQAQMGHTKEELAQHDFFKLIEGSPDTPSCWRDISEIRDATREERPCSVCLLLHKKDCTPFLAQFALTPLRDDQGRLVHFMGILVDVTHLIGSTDPAALQDADTEQRLEGVAEGVELDTRKLAAELKLEPHLDPEHLEAHPSVPCSLKHALSTIISAFVLSDPNLPDCPIVFVSEPFLKLTGYPREQVIGRNCRFLQGPDTDPKTVDAIREAVRNQKEITVRILNYTKSGRPFWNMFTLAPMSDSDCATRFFVGVQVDVTASGMVGGPTPAWTKTVSQENAVLKQGHLTATQINSALQGMAMQNPWMAINGTVMKLKPHKCQDQAYQELLALQQREGRLKLMHFRRVKQLGAGDVGLVDLVQLQGTDKKFAMKTLDKFEMQERNKVQRVLTEELILTAVDHPFLPTLYCTIQTDTHLHFVMEYCDGGELYGLLNAQPKKRLREEHVRFYVAEVLLALQYLHLLGYVYRDLKPENILLHHTGHVLLTDFDLSYSKGVTRPRLEKRPNGRVVKVKNGKQYAVDDYVLVAEPEARANSFVGTEEYLAPEVINASGHSAPVDWWSFGILIYELVYGVTPFRGVRRDETFDNVIKAPLRFPAKPQISPECQDLISKLLIKDPAQRLGTKYGAEEVKAHPFFHGLNFALQRNERPPYVPRRESRPAPAGSS

SEQ ID NO: 57 - protein, 730 aa, Chlorella variabilis
VPAPQAQLTSALAKLRQTFVVADATHPDCPLIYASEGFYHMTGYSQEELVGKNWYSFLQGPDTDPQAVRQLDEAVEAGRPLTLRLLCYRKSGKAFWNMLTMTPIHDDEGNVVKIVGVQVDVSRTTEGRAVQCCAQGLPLLVHYDERLKERVAWPATEEVMAAVSPRASRLSRASHHGPRSFSLSMGGAGGEEEACPHRAALDLATTIERIQTNFVISDPSLPDCPIVFASDSFLQLTGYAREDILGRNCRFLQGPGTDRATVNELRAAILAGRECTVRMLNYTKAGKPFWNLLTVAPIRDGLGVLRFIVGIQVDVTEQPQPEGAAALGGAAPRGLRDAKAVGRALQSMGYEGGGGGGEDDLWAGFGGQVAPVKPHKAADGAWAALRAAAQAEGRLTEQHFTRVRQLGAGNVGKVELVELAGSCHRFALKSLDKREMVERNKVGRVHTERRVLSALDHPFLVTLYATMMETDTAVQFLLEYCPGSDLHAVLHRAPYRRLPEAAVRRYATEVVSALQYLHLQGFAYRDLNPENIMVHEESGHCMLTDFNLSYWQAGVEPELVLPPPPPPPRQQRAAGGGAPAAAAMATASSLGGAPSGSPRAGGWLLAAAPSGGRANSFVGTEEYLAPEVVKGTGHDSGVDWWSFGILLFELLFGYTPFKGLRRDETFDNIVKMELAFPKGGAHVSPQAKDLITRLLAKDPRQRLGAHAGADEVKQHPWFDGVNWALGRADQ

SEQ ID NO: 58 - protein, 712 aa, Haematococcus lacustris
MAGVQLPSAASQLTKVLAGLRHTFVVADATLPDMPLIYASDGFYAMTGYGPDEVLGHNCRFLQGEGTDPKEVLKIRDAIKKGEGVSVRLLNYRKDGTPFWNLLTVTPIKTPDGKVSKFVGVQVDVTSKTEGKAVTDNGGVPLLVKYDTRLRENVAKRIVDEVTTTVESAEPGRAQGAKGSAPKSFPRVALDLATTVERIQQNFCISDPTLPDCPIVFASDAFLDLTEYKREEVLGRNCRFLQGPGTDQNTVQMIRDAIRTGAEITVRILNYTKSGRPFWNMFTLAPMSDSDGTTRFFVGVQVDVTAVQPGAPLASSKPTAADEANVKRGVQAANMIGTALQGMGYAEANPWASLPSAVMKRKPHKTEDKAFLALLAVQARDGKLKLMHFRRVKQLGAGDVGLVDLVQLQGTEHKFAMKTLDKYEMQDRNKVQRVLTEERILTAVDHPFLPTLYCTIQTDTHLHFVMEFCDGGELYGLLNSQPKKRLREAHVRFYVAEVLLALQYLHLLGYVYRDLKPENILLQHSGHVLLTDFDLSYGKGVTTPQVERRPDARVTKNKNGKIVLMDDFVLLAEPVARANSFVGTEEYLAPEVISAAGHSAPVDWWSYGILIYELVYGTTPFRGARRDETFDNVVKAPLRFPAKPVVSPECQDLITQLLVKDPAKRLGTRTGAEEIKAHPFFKGITWSLLRHEAPPYIPHRQSKTMTASGTDA

SEQ ID NO: 59 - protein, 719 aa, Tetraselmis striata; identical to SEQ ID NO: 54

SEQ ID NO: 60 - protein, 803 aa, Coccomyxa subellipsoidea
MPAQTGQAEKQQKDVQLHPELQRPGQKVPGPAPQLTKVLAGLRHTFVVADATLPDCPLVFASEGFLSMTGYSAEEVLGHNCRFLQGEGTDPKEVAIIRDAVKKGEGCSVRLLNYRRDGTPFWNLLTMTPIKTEDGKVSKFVGVQVDVTSKTEGRAFSDATGVPLLVKYDTRLRENVAKNIVQDVTLQVQEAEEEDSGAASEAARVSSLKGFNKLWHKMGNKVTRPQCLGGPPSAPLGDPKAQASAHDPQLQKQGERVGKKMTAPKTFPRVAMDLATTVERIQQNFCICDPNLPDNPIVFASDGFLEMSQYDRFEVLGRNCRFLQGPDTDPKAISIIRDAIKSQSEATVRILNYRKSGQPFWNMLTIAPMADVDGTSRFFIGVQVDVTAEDVPMTGGIPQVDAKAVKAADPMGSVLGMAQRQMGAGWAVHDPWAAIHAGVASLKPHKAQEKVWAALRENDRKNGRLALSQFRRLKQLGTGDVGLVDMVELQDGSGRYAMKTLEKAEMLERNKVMRVLTEAKILSVVDHPFLASLYGTIVTDTHLHFLMQICEGGELYALLTSQPSKRFKESHVRFYTAEVLIALQYLHLMGFVYRDLKPENILLHSSGHILLTDFDLSFCQGSTKVKFEKKKNGHANSSQPGATQVSPAEEIMMIAVPEARANSFVGTEEYLAPEVINGVGHGAGVDWWSFGILIYELLYGFTPFRGKKRDETFNNILKRPLSFPELPEVSDECKDLISQLLERDPAKRLGAHAGAEEIKAHPFYESINWALLRNTRPPYIPRRSALRKANKPSPAAQAQFDDF

SEQ ID NO: 61 - protein, 819 aa, Micromonas pusilla; identical to SEQ ID NO: 55

SEQ ID NO: 62 - protein, 348 aa, Vaucheria frigida
MNGLTPPLMFCSRSDDPSSTSNINLDDVFADVFFNSNGELLDIDEIDDFGDNTCPKSSMSVDDDASSQVFQGHLFGNALSSIALSDSGDLSTGIYESQGNASRGKSLRTKSSGSISSELTEAQKVERRERNREHAKRSRVRKKFLLESLQQSVNELNHENNCLKESIREHLGPRGDSLIAQCSPEADTLLTDNPSKANRILEDPDYSLVKALQMAQQNFVITDASLPDNPIVYASRGFLTLTGYSLDQILGRNCRFLQGPETDPRAVDKIRNAITKGVDTSVCLLNYRQDGTTFWNLFFVAGLRDSKGNIVNYVGVQSKVSEDYAKLLVNEQNIEYKGVRTSNMLRRK

SEQ ID NO: 63 - protein, 183 aa, Fucus distichus
GVTRRRSSSKEEQAKKRRERNRVLARRTRLRKKFFFQSLQQQVNDLQYVNERLKGIINTRCANNSAEIIRSCVSRVPSMVADCANQATALLEQSDFLLVKALQSSQPSFCVTDPQLPDNPIVYASNTFIELTGYDRSQVLGRNCRFLQGPDTDPDAVAKIRKGIEEGKDTSVFLRQYKADGTV

SEQ ID NO: 64 - protein, 360 aa, Nannochloropsis gaditana
MAAIMLQQPQCKVPTSLLPVYSRAQGGQSGDLDLNMEQLLEAYVLDEGMDLDFLDGTNISDTSKDTAPNNISDLDDLDEMADGVGIDSEGDIVKTGGKSKKRKTSGWSGSGSSIPRKKSHEQMERRRERNRILARRTRLRKKFIFESLQKQVMDLKRQNSRLKSIVKDKMADQASEVLGACTMRLPSIVTESMAEASTVLDRGDFNLIKALQTTQQSFVVTDPSLPDNPIVFASQGFLEMTGYSMSQVLGRNCRFLQGPRTDQATVAQIRKGIAEGADTSVALLNYKVDGTPFWNQFFVAPLRDLNGEVVYFVGAQSKIDRPLEEIEKDAAERARQEAEERNVAGLASGEGEEEEDEEED

SEQ ID NO: 65 - protein, 465 aa, Nannochloropsis gaditana
MAAIQQPTSWGILSRNNSSTDLAGEAGGLEAPDQIKQEAAAPLWTAPLYASANNSNPMADTVDLDEIFADDFLLPGMGMVPFGTEGDMGPEDMFPGGLSCDTSVATNDNDDSAVASGMHGKGKGAQFANAAPQGETEDKTNVRAAAVVRSAASRTRSGITTRLGLNKGQGRAVTGASSMLPSAVPSRARVPTRKGSLPVEEEEDVEEEEEEYETGSEEEGEGGDGRGARRRKKAKLTLKPLTEAQRVERRERNREHAKRSRMRKKFMLESLQAQMLALRKENLRLRQLVATKLPDKADTILRGCSSIKTQNLLSSVELGHHRALADHDGRLVSALQFAQQNFTVSDPSLPDNPIIYASQGFLDLTGYTSDQIVGRNCRFLQGPGTDPAAVDIIRRGVALGEDTSVCLLNYRADGTPFWNQFFVAALRDMEGNIVNYVGVQCKVEEAPMEEELKERVKTINFDEEE

SEQ ID NO: 66 - protein, 338 aa, Sargassum fusiforme
MRTSLSQGIGIGSGGPGGVGIRGITSVAPPSGYRGRGSYAGSLPRRQRHKVSSKDLTEEQRNERRERNREHAKRSRVRKKFLLDSLQRSVDALQAENESLKGSIIGSLGQHGRELVAKCSPEDAESTLVTANPTQATKILDDPDYSLVKALQTAQQNFVITDASPPDNPIVFASNGFLELTRYKLNEVLGRNCRFLQGPETDPRAVDKIRTAIDEGYDTSVCLLNYRADDTTFWNQFFVAALRDGEGNTVNYVGVQCKVGDDYARIVVNAQKKQLARSGSAAGSTRRGPQTQREQPVLRPSTSGTAAILTVDAFSGASAAFAAESRQGQGQGQGGADI

SEQ ID NO: 67 - protein, 346 aa, Chlamydomonas reinhardtii
MVEKMHTKVCIIGSGPAAHTAAVYTARAELQPILFEGFMANGIAAGGQLTTTTDVENFPGFPEGILGAELTTRFREQSERFGTRIYSETVDSIDTSRRPFTVRTADKEVTADSLIIATGAVARRLEFPGSGEEGGFWNRGISACAVCDGAAPIFRNKPIAVIGGGDSAMEEATFLTKYGSKVYIIHRRDSFRASKIMAKRALEHPKIEVLWNSVVEEAYGNEKGLLGGVKVKDVVTGELHDLPVSGLFFAIGHQPATAFLNGQLALDAEGYIVTAPDSTATSVPGVFAAGDVQDKKWRQAITAAGTGCMAALEAEHFISAHEAEPEADGAKEPAAAAAAPVADGNL

SEQ ID NO: 68 - protein, 536 aa, Chlamydomonas reinhardtii
MIQLPATVVQRRAPIGAPSAVGACNPVAGRHRAAPALASRTRTVATPATAAPAATSTQQVADVENVVIIGSGPAGYTAAIYAARANLKPVVFEGFRNGRGGQLMTTTEVENFPGFPEGITGPDLMDRMRKQAERWGSELYTEDVEQVDLSVRPFVIRSSDRELRAHSVIIATGATAKRLGLPSENTFWSRGISACAICDGASPLFKNAEVAVVGGGDSATEEAVYVTKYAKHVHLLVRGERMRASKAMQDRVLANPRITVHFNTGIEDAFGGEVLQGLRLFDTRTGEKRSLDVQGMFYGIGHTPNSKLVAGQVELDEAGYVKVAHGAATSVPGVFSAGDLHDTEWRQAITAAGSGCMAALSAERYLTANNLVREFKQKDEPAAHGHAAAAGGNGNGNGHAAAAANGGSEAKATSSIDTPETFDLSADKHKGQYALRKLYHESDRLICVLYTSPTCGPCRTLKPIFNGVVDEYTGKVHYVEIDIEQDPEIAEAAGVMGTPTVQMFKDKARVEQLSGVKMKKDYRAIIEKYVPAAVSA